SEQUENCE LISTING (316 sequences)

SEQ ID NO: 1 - DNA, 342 bp, synthetic construct
  misc_feature 1..342: note = OMT1 variable light chain domain
  source 1..342: mol_type = other DNA
gacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaa

SEQ ID NO: 2 - protein, 114 aa, synthetic construct
  REGION 1..114: note = OMT1 variable light chain domain
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIK

SEQ ID NO: 3 - DNA, 372 bp, synthetic construct
  misc_feature 1..372: note = OMT1 variable heavy chain domain
  source 1..372: mol_type = other DNA
gaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcttca

SEQ ID NO: 4 - protein, 124 aa, synthetic construct
  REGION 1..124: note = OMT1 variable heavy chain domain
EVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSS

SEQ ID NO: 5 - DNA, 36 bp, synthetic construct
  misc_feature 1..36: note = OMT1 CDR L1
  source 1..36: mol_type = other DNA
cacagtgttttatacagctccaacaataagaactac

SEQ ID NO: 6 - protein, 12 aa, synthetic construct
  REGION 1..12: note = OMT1 CDR L1
HSVLYSSNNKNY

SEQ ID NO: 7-8 - skipped (2 entries)

SEQ ID NO: 9 - DNA, 30 bp, synthetic construct
  misc_feature 1..30: note = OMT1 CDR L3
  source 1..30: mol_type = other DNA
cagcaatattatagtactcctccgaccact

SEQ ID NO: 10 - protein, 10 aa, synthetic construct
  REGION 1..10: note = OMT1 CDR L3
QQYYSTPPTT

SEQ ID NO: 11 - DNA, 24 bp, synthetic construct
  misc_feature 1..24: note = OMT1 CDR H1
  source 1..24: mol_type = other DNA
ggattcacctttagcagctatggc

SEQ ID NO: 12 - protein, 8 aa, synthetic construct
  REGION 1..8: note = OMT1 CDR H1
GFTFSSYG

SEQ ID NO: 13 - DNA, 24 bp, synthetic construct
  misc_feature 1..24: note = OMT1 CDR H2
  source 1..24: mol_type = other DNA
attagtggtagtggtggtagcaca

SEQ ID NO: 14 - protein, 8 aa, synthetic construct
  REGION 1..8: note = OMT1 CDR H2
ISGSGGST

SEQ ID NO: 15 - DNA, 51 bp, synthetic construct
  misc_feature 1..51: note = OMT1 CDR H3
  source 1..51: mol_type = other DNA
gcgaaagaaaagttacgatattttgactggttatccgatgcttttgatatc

SEQ ID NO: 16 - protein, 17 aa, synthetic construct
  REGION 1..17: note = OMT1 CDR H3
AKEKLRYFDWLSDAFDI

SEQ ID NO: 17 - DNA, 321 bp, synthetic construct
  misc_feature 1..321: note = DB8, DB60, DB65, DB280, DB331, DB415, DB435 variable light chain domain
  source 1..321: mol_type = other DNA
gacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaa

SEQ ID NO: 18 - protein, 107 aa, synthetic construct [sequence omitted]

SEQ ID NO: 19 - DNA, 366 bp, synthetic construct
  misc_feature 1..366: note = DB8 variable heavy chain domain
  source 1..366: mol_type = other DNA
caggtgcagctggtgcagtctggggctgaggtgaagaagcctggggcctcagtgaaggtttcctgcaaggcatctggatacatcttcaccgactactatatgcactgggtgcgtcaggcccctggacaagggcttgagtggatgggatggatgagccctaacagtggtaacacaggctatgcacagaagttccagggccgtgtcaccatgacccgcgacacgtccacgagcacagtctacatggagctgagcagcctgcgttctgaggacacggccgtgtattactgtgcgagagatgcggcggattacggtgactacgttgcttttgatatctggggccaagggacaatggtcaccgtctcttca

SEQ ID NO: 20 - protein, 122 aa, synthetic construct
  REGION 1..122: note = DB8 variable heavy chain domain
QVQLVQSGAEVKKPGASVKVSCKASGYIFTDYYMHWVRQAPGQGLEWMGWMSPNSGNTGYAQKFQGRVTMTRDTSTSTVYMELSSLRSEDTAVYYCARDAADYGDYVAFDIWGQGTMVTVSS

SEQ ID NO: 21 - DNA, 18 bp, synthetic construct
  misc_feature 1..18: note = DB8, DB60, DB65, DB280, DB331, DB415, DB435 CDR L1
  source 1..18: mol_type = other DNA
cagagcattagcagctat

SEQ ID NO: 22 - protein, 6 aa, synthetic construct
  REGION 1..6: note = DB8, DB60, DB65, DB280, DB331, DB415, DB435 CDR L1
QSISSY

SEQ ID NO: 23-24 - skipped (2 entries)

SEQ ID NO: 25 - DNA, 27 bp, synthetic construct
  misc_feature 1..27: note = DB8, DB60, DB65, DB280, DB331, DB415, DB435 CDR L3
  source 1..27: mol_type = other DNA
caacagagttacagtacccctctcact

SEQ ID NO: 26 - protein, 9 aa, synthetic construct
  REGION 1..9: note = DB8, DB60, DB65, DB280, DB331, DB415, DB435 CDR L3
QQSYSTPLT

SEQ ID NO: 27 - DNA, 24 bp, synthetic construct
  misc_feature 1..24: note = DB8 and DB60 CDR H1
  source 1..24: mol_type = other DNA
ggatacatcttcaccgactactat

SEQ ID NO: 28 - protein, 8 aa, synthetic construct
  REGION 1..8: note = DB8 CDR H1
GYIFTDYY

SEQ ID NO: 29 - DNA, 24 bp, synthetic construct
  misc_feature 1..24: note = DB8 CDR H2
  source 1..24: mol_type = other DNA
atgagccctaacagtggtaacaca

SEQ ID NO: 30 - protein, 8 aa, synthetic construct
  REGION 1..8: note = DB8 CDR H2
MSPNSGNT

SEQ ID NO: 31 - DNA, 45 bp, synthetic construct
  misc_feature 1..45: note = DB8 CDR H3
  source 1..45: mol_type = other DNA
gcgagagatgcggcggattacggtgactacgttgcttttgatatc

SEQ ID NO: 32 - protein, 15 aa, synthetic construct
  REGION 1..15: note = DB8 CDR H3
ARDAADYGDYVAFDI

SEQ ID NO: 33 - DNA, 357 bp, synthetic construct
  misc_feature 1..357: note = DB60 variable heavy chain domain
  source 1..357: mol_type = other DNA
caggtgcagctggtgcagtctggggctgaggtgaagaagcctggggcctcagtgaaggtttcctgcaaggcatctggatacaccttcaccagctactatatgcactgggtgcgtcaggcccctggacaagggcttgagtggatggggtggatcaaccctaacagtggtgacacaagctatgcacagaagttccagggccgtgtcaccatgacccgcgacacgtccacgagcacagtctacatggagctgagcagcctgcgttctgaggacacggccgtgtattactgtgcgcaggatagtagtggttccggggcttttgatatctggggccaagggacaatggtcaccgtctcttca

SEQ ID NO: 34 - protein, 119 aa, synthetic construct
  REGION 1..119: note = DB60 variable heavy chain domain
QVQLVQSGAEVKKPGASVKVSCKASGYTFTSYYMHWVRQAPGQGLEWMGWINPNSGDTSYAQKFQGRVTMTRDTSTSTVYMELSSLRSEDTAVYYCAQDSSGSGAFDIWGQGTMVTVSS

SEQ ID NO: 35 - DNA, 24 bp, synthetic construct
  misc_feature 1..24: note = DB60 CDR H1
  source 1..24: mol_type = other DNA
ggatacaccttcaccagctactat

SEQ ID NO: 36 - protein, 8 aa, synthetic construct
  REGION 1..8: note = DB60 CDR H1
GYTFTSYY

SEQ ID NO: 37 - DNA, 24 bp, synthetic construct
  misc_feature 1..24: note = DB60 CDR H2
  source 1..24: mol_type = other DNA
atcaaccctaacagtggtgacaca

SEQ ID NO: 38 - protein, 8 aa, synthetic construct
  REGION 1..8: note = DB60 CDR H2
INPNSGDT

SEQ ID NO: 39 - DNA, 36 bp, synthetic construct
  misc_feature 1..36: note = DB60 CDR H3
  source 1..36: mol_type = other DNA
gcgcaggatagtagtggttccggggcttttgatatc

SEQ ID NO: 40 - protein, 12 aa, synthetic construct
  REGION 1..12: note = DB60 CDR H3
AQDSSGSGAFDI

SEQ ID NO: 41 - DNA, 369 bp, synthetic construct
  misc_feature 1..369: note = DB65 variable heavy chain domain
  source 1..369: mol_type = other DNA
caggtgcagctggtgcagtctggggctgaggtgaagaagcctggggcctcagtgaaggtttcctgcaaggcatctggatacaccttcaccggctactatatgcactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcgaaagaggaaccgatttttggagtggttatggatgcttttgatatctggggccaagggacaatggtcaccgtctcctca

SEQ ID NO: 42 - protein, 123 aa, synthetic construct
  REGION 1..123: note = DB65 variable heavy chain domain
QVQLVQSGAEVKKPGASVKVSCKASGYTFTGYYMHWVRQAPGQGLEWMGWMNPNSGNTGYAQKFQGRVTMTRDTSTSTVYMELSSLRSEDTAVYYCAKEEPIFGVVMDAFDIWGQGTMVTVSS

SEQ ID NO: 43 - DNA, 24 bp, synthetic construct
  misc_feature 1..24: note = DB65 CDR H1
  source 1..24: mol_type = other DNA
ggatacaccttcaccggctactat

SEQ ID NO: 44 - protein, 8 aa, synthetic construct
  REGION 1..8: note = DB65 CDR H1
GYTFTGYY

SEQ ID NO: 45 - DNA, 24 bp, synthetic construct
  misc_feature 1..24: note = DB65 CDR H2
  source 1..24: mol_type = other DNA
atgaaccctaacagtggtaacaca

SEQ ID NO: 46 - protein, 8 aa, synthetic construct
  REGION 1..8: note = DB65 CDR H2
MNPNSGNT

SEQ ID NO: 47 - DNA, 48 bp, synthetic construct
  misc_feature 1..48: note = DB65 CDR H3
  source 1..48: mol_type = other DNA
gcgaaagaggaaccgatttttggagtggttatggatgcttttgatatc

SEQ ID NO: 48 - protein, 16 aa, synthetic construct
  REGION 1..16: note = DB65 CDR H3
AKEEPIFGVVMDAFDI

SEQ ID NO: 49 - DNA, 321 bp, synthetic construct
  misc_feature 1..321: note = DB82 variable light chain domain
  source 1..321: mol_type = other DNA
gacatccagatgacccagtctccatcctccctgtctgcatctgtaggagaccgcgtcaccatcacttgccgggcaagtcagaccataaacaactatttgaactggtatcagcagaaaccagggaaagcccctaagctcctgatctattctgcatctactttgcaaagtggggtcccatcacgtttcagtggcagtggatctgggacagatttcactctcaccatcagcagtctgcaacctgaagattttgcaacttactactgtcaccagagttacacttcacctctcactttcggcggaggtaccaaggtggagatcaaa

SEQ ID NO: 50 - protein, 107 aa, synthetic construct
  REGION 1..107: note = DB82 variable light chain domain
DIQMTQSPSSLSASVGDRVTITCRASQTINNYLNWYQQKPGKAPKLLIYSASTLQSGVPSRFSGSGSGTDFTLTISSLQPEDFATYYCHQSYTSPLTFGGGTKVEIK

SEQ ID NO: 51 - DNA, 363 bp, synthetic construct
  misc_feature 1..363: note = DB82 variable heavy chain domain
  source 1..363: mol_type = other DNA
gaggtgcagctggtggagtctgggggaggcttggtacagcctggggggtccctgcgcctctcctgtgcagcctctggattcacctttagcagctatgccatgagctgggtccgccaggctccagggaaggggctggagtgggtctcagttattagtgccaatagtgctggtctaggccatgcggactctgtgaagggccggttcaccatctcccgcgacaattccaagaacacgctgtatctgcaaatgaacagcctgcgcgccgaggacacggccgtatattactgtgcgagagtgggctatagcagctcggctgatgcttttgatatctggggccaagggacaatggtcaccgtctcctcg

SEQ ID NO: 52 - protein, 121 aa, synthetic construct
  REGION 1..121: note = DB82 variable heavy chain domain
EVQLVESGGGLVQPGGSLRLSCAASGFTFSSYAMSWVRQAPGKGLEWVSVISANSAGLGHADSVKGRFTISRDNSKNTLYLQMNSLRAEDTAVYYCARVGYSSSADAFDIWGQGTMVTVSS

SEQ ID NO: 53 - DNA, 18 bp, synthetic construct
  misc_feature 1..18: note = DB82 CDR L1
  source 1..18: mol_type = other DNA
cagaccataaacaactat

SEQ ID NO: 54 - protein, 6 aa, synthetic construct
  REGION 1..6: note = DB82 CDR L1
QTINNY

SEQ ID NO: 55-56 - skipped (2 entries)

SEQ ID NO: 57 - DNA, 27 bp, synthetic construct
  misc_feature 1..27: note = DB82 CDR L3
  source 1..27: mol_type = other DNA
caccagagttacacttcacctctcact

SEQ ID NO: 58 - protein, 9 aa, synthetic construct
  REGION 1..9: note = DB82 CDR L3
HQSYTSPLT

SEQ ID NO: 59 - DNA, 24 bp, synthetic construct
  misc_feature 1..24: note = DB82 CDR H1
  source 1..24: mol_type = other DNA
ggattcacctttagcagctatgcc

SEQ ID NO: 60 - protein, 8 aa, synthetic construct
  REGION 1..8: note = DB82 CDR H1
GFTFSSYA

SEQ ID NO: 61 - DNA, 24 bp, synthetic construct
  misc_feature 1..24: note = DB82 CDR H2
  source 1..24: mol_type = other DNA
attagtgccaatagtgctggtcta

SEQ ID NO: 62 - protein, 8 aa, synthetic construct
  REGION 1..8: note = DB82 CDR H2
ISANSAGL

SEQ ID NO: 63 - DNA, 42 bp, synthetic construct
  misc_feature 1..42: note = DB82 CDR H3
  source 1..42: mol_type = other DNA
gcgagagtgggctatagcagctcggctgatgcttttgatatc

SEQ ID NO: 64 - protein, 14 aa, synthetic construct
  REGION 1..14: note = DB82 CDR H3
ARVGYSSSADAFDI

SEQ ID NO: 65 - DNA, 336 bp, synthetic construct
  misc_feature 1..336: note = DB83 variable light chain domain
  source 1..336: mol_type = other DNA
gatgttgtgatgactcagtctccactctccctgcccgtcacccctggagagccggcctccatctcctgcaggtctagtcagagcctcctgcatagtaatggagacaactatttggattggtacctgcagaagccagggcagtctccacagctcctgatctatttgggttctaatcgggcctccggggtccctgaccgtttcagtggcagtggatcaggcacagattttacactgaaaatcagccgtgtggaggctgaggatgttggggtttattactgcatgcaagctacacactggccactcactttcggccctggtaccaaagtggatatcaaa

SEQ ID NO: 66 - protein, 112 aa, synthetic construct
  REGION 1..112: note = DB83 variable light chain domain
DVVMTQSPLSLPVTPGEPASISCRSSQSLLHSNGDNYLDWYLQKPGQSPQLLIYLGSNRASGVPDRFSGSGSGTDFTLKISRVEAEDVGVYYCMQATHWPLTFGPGTKVDIK

SEQ ID NO: 67 - DNA, 375 bp, synthetic construct
  misc_feature 1..375: note = DB83 variable heavy chain domain
  source 1..375: mol_type = other DNA
caggtgcagctggtgcagtctggggctgaggtgaagaagcctggggcctcagtgaaggtttcctgcaaggcatctggatacaccttcactagctatgctatgcattgggtgcgtcaggcccctggacaagggcttgagtggatgggacttgttgatcctgaagatggtgaaacaatatatgcagagaagttccagggccgtgtcaccatgacccgcgacacgtccacgagcacagtctacatggagctgagcagcctgcgttctgaggacacggccgtgtattactgtgcgagacgaacgtattactatgatagtagtggttcccgttatgcttttgatatctggggccaagggaccacggtcaccgtctcttca

SEQ ID NO: 68 - protein, 125 aa, synthetic construct
  REGION 1..125: note = DB83 variable heavy chain domain
QVQLVQSGAEVKKPGASVKVSCKASGYTFTSYAMHWVRQAPGQGLEWMGLVDPEDGETIYAEKFQGRVTMTRDTSTSTVYMELSSLRSEDTAVYYCARRTYYYDSSGSRYAFDIWGQGTTVTVSS

SEQ ID NO: 69 - DNA, 33 bp, synthetic construct
  misc_feature 1..33: note = DB83 CDR L1
  source 1..33: mol_type = other DNA
cagagcctcctgcatagtaatggagacaactat

SEQ ID NO: 70 - protein, 11 aa, synthetic construct
  REGION 1..11: note = DB83 CDR L1
QSLLHSNGDNY

SEQ ID NO: 71-72 - skipped (2 entries)

SEQ ID NO: 73 - DNA, 27 bp, synthetic construct
  misc_feature 1..27: note = DB83 CDR L3
  source 1..27: mol_type = other DNA
atgcaagctacacactggccactcact

SEQ ID NO: 74 - protein, 9 aa, synthetic construct
  REGION 1..9: note = DB83 CDR L3
MQATHWPLT

SEQ ID NO: 75 - DNA, 24 bp, synthetic construct
  misc_feature 1..24: note = DB83 CDR H1
  source 1..24: mol_type = other DNA
ggatacaccttcactagctatgct

SEQ ID NO: 76 - protein, 8 aa, synthetic construct
  REGION 1..8: note = DB83 CDR H1
GYTFTSYA

SEQ ID NO: 77 - DNA, 24 bp, synthetic construct
  misc_feature 1..24: note = DB83 CDR H2
  source 1..24: mol_type = other DNA
gttgatcctgaagatggtgaaaca

SEQ ID NO: 78 - protein, 8 aa, synthetic construct
  REGION 1..8: note = DB83 CDR H2
VDPEDGET

SEQ ID NO: 79 - DNA, 54 bp, synthetic construct
  misc_feature 1..54: note = DB83 CDR H3
  source 1..54: mol_type = other DNA
gcgagacgaacgtattactatgatagtagtggttcccgttatgcttttgatatc

SEQ ID NO: 80 - protein, 18 aa, synthetic construct
  REGION 1..18: note = DB83 CDR H3
ARRTYYYDSSGSRYAFDI

SEQ ID NO: 81 - DNA, 321 bp, synthetic construct
  misc_feature 1..321: note = DB86 variable light chain domain
  source 1..321: mol_type = other DNA
gacatccagatgacccagtctccatcctccctgtctgcatctgtaggagaccgcgtcaccatcacttgccgggcaagtcagggcatcagaaatgatttaggttggtatcagcagaaaccagggaaagcccctaagctcctgatctatgctgcatccactttgcaatcaggggtcccatcacgtttcagtggcagtggatctgggacagatttcactctcaccatcagcagtctgcaacctgaagattttgcaacttactactgtcaacagagttacggtgcccccctcactttcggcggaggtaccaaggtggagatcaaa

SEQ ID NO: 82 - protein, 107 aa, synthetic construct
  REGION 1..107: note = DB86 variable light chain domain
DIQMTQSPSSLSASVGDRVTITCRASQGIRNDLGWYQQKPGKAPKLLIYAASTLQSGVPSRFSGSGSGTDFTLTISSLQPEDFATYYCQQSYGAPLTFGGGTKVEIK

SEQ ID NO: 83 - DNA, 360 bp, synthetic construct
  misc_feature 1..360: note = DB86 variable heavy chain domain
  source 1..360: mol_type = other DNA
caggtgcagctggtgcagtctggggctgaggtgaagaagcctggggcctcagtgaaggtttcctgcaaggcatctggatatatgttcagtggccattctgcacactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcgagagatagcagtggctggtacgatgtctttgactactggggccaggggaccctggtcaccgtctcctca

SEQ ID NO: 84 - protein, 120 aa, synthetic construct
  REGION 1..120: note = DB86 variable heavy chain domain
QVQLVQSGAEVKKPGASVKVSCKASGYMFSGHSAHWVRQAPGQGLEWMGWMNPNSGNTGYAQKFQGRVTMTRDTSTSTVYMELSSLRSEDTAVYYCARDSSGWYDVFDYWGQGTLVTVSS

SEQ ID NO: 85 - DNA, 18 bp, synthetic construct
  misc_feature 1..18: note = DB86 CDR L1
  source 1..18: mol_type = other DNA
cagggcatcagaaatgat

SEQ ID NO: 86 - protein, 6 aa, synthetic construct
  REGION 1..6: note = DB86 CDR L1
QGIRND

SEQ ID NO: 87-88 - skipped (2 entries)

SEQ ID NO: 89 - DNA, 24 bp, synthetic construct
  misc_feature 1..24: note = DB86 CDR L3
  source 1..24: mol_type = other DNA
caacagagttacggtgcccccctc

SEQ ID NO: 90 - protein, 9 aa, synthetic construct
  REGION 1..9: note = DB86 CDR L3
QQSYGAPLT

SEQ ID NO: 91 - DNA, 24 bp, synthetic construct
  misc_feature 1..24: note = DB86 CDR H1
  source 1..24: mol_type = other DNA
ggatatatgttcagtggccattct

SEQ ID NO: 92 - protein, 8 aa, synthetic construct
  REGION 1..8: note = DB86 CDR H1
GYMFSGHS

SEQ ID NO: 93 - DNA, 24 bp, synthetic construct
  misc_feature 1..24: note = DB86 CDR H2
  source 1..24: mol_type = other DNA
atgaaccctaacagtggtaacaca

SEQ ID NO: 94 - protein, 8 aa, synthetic construct
  REGION 1..8: note = DB86 CDR H2
MNPNSGNT

SEQ ID NO: 95 - DNA, 39 bp, synthetic construct
  misc_feature 1..39: note = DB86 CDR H3
  source 1..39: mol_type = other DNA
gcgagagatagcagtggctggtacgatgtctttgactac

SEQ ID NO: 96 - protein, 13 aa, synthetic construct
  REGION 1..13: note = DB86 CDR H3
ARDSSGWYDVFDY

SEQ ID NO: 97 - DNA, 378 bp, synthetic construct
  misc_feature 1..378: note = DB280 variable heavy chain domain
  source 1..378: mol_type = other DNA
caggtgcagctggtgcagtctggggctgaggtgaagaagcctggggcctcagtgaaggtttcctgcaaggcatctggatacagcctcaacttatactatatgcactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcgagcctcgattgtagtggtggtagctgctactccgaatatgatgcttttgatatctggggccaagggaccacggtcaccgtctcctca

SEQ ID NO: 98 - protein, 126 aa, synthetic construct
  REGION 1..126: note = DB280 variable heavy chain domain
QVQLVQSGAEVKKPGASVKVSCKASGYSLNLYYMHWVRQAPGQGLEWMGWMNPNSGNTGYAQKFQGRVTMTRDTSTSTVYMELSSLRSEDTAVYYCASLDCSGGSCYSEYDAFDIWGQGTTVTVSS

SEQ ID NO: 99 - DNA, 24 bp, synthetic construct
  misc_feature 1..24: note = DB280 CDR H1
  source 1..24: mol_type = other DNA
ggatacagcctcaacttatactat

SEQ ID NO: 100 - protein, 8 aa, synthetic construct
  REGION 1..8: note = DB280 CDR H1
GYSLNLYY

SEQ ID NO: 101 - DNA, 24 bp, synthetic construct
  misc_feature 1..24: note = DB280 CDR H2
  source 1..24: mol_type = other DNA
atgaaccctaacagtggtaacaca

SEQ ID NO: 102 - protein, 8 aa, synthetic construct
  REGION 1..8: note = DB280 CDR H2
MNPNSGNT

SEQ ID NO: 103 - DNA, 57 bp, synthetic construct
  misc_feature 1..57: note = DB280 CDR H3
  source 1..57: mol_type = other DNA
gcgagcctcgattgtagtggtggtagctgctactccgaatatgatgcttttgatatc

SEQ ID NO: 104 - protein, 19 aa, synthetic construct
  REGION 1..19: note = DB280 CDR H3
ASLDCSGGSCYSEYDAFDI

SEQ ID NO: 105 - DNA, 357 bp, synthetic construct
  misc_feature 1..357: note = DB331 variable heavy chain domain
  source 1..357: mol_type = other DNA
caggtgcagctggtgcagtctggggctgaggtgaagaagcctggggcctcagtgaaggtttcctgcaaggcatctggatacaccttcaccagctactatatgcactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcaacagatctcgcgggggaagccttgttcgacccctggggccagggcaccctggtcaccgtctcctca

SEQ ID NO: 106 - protein, 119 aa, synthetic construct
  REGION 1..119: note = DB331 variable heavy chain domain
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCATDLAGEALFDPWGQGTLVTVSS

SEQ ID NO: 107 - DNA, 24 bp, synthetic construct
  misc_feature 1..24: note = DB331 CDR H1
  source 1..24: mol_type = other DNA
ggatacaccttcaccagctactat

SEQ ID NO: 108 - protein, 8 aa, synthetic construct
  REGION 1..8: note = DB331 CDR H1
GYTFTSYY

SEQ ID NO: 109 - DNA, 24 bp, synthetic construct
  misc_feature 1..24: note = DB331 CDR H2
  source 1..24: mol_type = other DNA
atgaaccctaacagtggtaacaca

SEQ ID NO: 110 - protein, 8 aa, synthetic construct
  REGION 1..8: note = DB331 CDR H2
MNPNSGNT

SEQ ID NO: 111 - DNA, 36 bp, synthetic construct
  misc_feature 1..36: note = DB331 CDR H3
  source 1..36: mol_type = other DNA
gcaacagatctcgcgggggaagccttgttcgacccc

SEQ ID NO: 112 - protein, 12 aa, synthetic construct
  REGION 1..12: note = DB331 CDR H3
ATDLAGEALFDP

SEQ ID NO: 113 - DNA, 351 bp, synthetic construct
  misc_feature 1..351: note = DB415 variable heavy chain domain
  source 1..351: mol_type = other DNA
gaggtgcagctggtggagtctgggggaggcttggtacagcctggggggtccctgcgcctctcctgtgcagcctctggaatcaccttcagtagttatggcatgcattgggtccgccaggctccagggaaggggctggagtgggtctcaggtattagttggaatagtggtaacagagtctatgtggactctgtgaagggccggttcaccatctcccgcgacaattccaagaacacgctgtatctgcaaatgaacagcctgcgcgccgaggacacggccgtatattactgtgcgagagatactaatgatgcttttgatatctggggccaagggaccacggtcaccgtctcctca

SEQ ID NO: 114 - protein, 117 aa, synthetic construct
  REGION 1..117: note = DB415 variable heavy chain domain
EVQLVESGGGLVQPGGSLRLSCAASGITFSSYGMHWVRQAPGKGLEWVSGISWNSGNRVYVDSVKGRFTISRDNSKNTLYLQMNSLRAEDTAVYYCARDTNDAFDIWGQGTTVTVSS

SEQ ID NO: 115 - DNA, 24 bp, synthetic construct
  misc_feature 1..24: note = DB415 CDR H1
  source 1..24: mol_type = other DNA
ggaatcaccttcagtagttatggc

SEQ ID NO: 116 - protein, 8 aa, synthetic construct
  REGION 1..8: note = DB415 CDR H1
GITFSSYG

SEQ ID NO: 117 - DNA, 24 bp, synthetic construct
  misc_feature 1..24: note = DB415 CDR H2
  source 1..24: mol_type = other DNA
attagttggaatagtggtaacaga

SEQ ID NO: 118 - protein, 8 aa, synthetic construct
  REGION 1..8: note = DB415 CDR H2
ISWNSGNR

SEQ ID NO: 119 - DNA, 30 bp, synthetic construct
  misc_feature 1..30: note = DB415 CDR H3
  source 1..30: mol_type = other DNA
gcgagagatactaatgatgcttttgatatc

SEQ ID NO: 120 - protein, 10 aa, synthetic construct
  REGION 1..10: note = DB415 CDR H3
ARDTNDAFDI

SEQ ID NO: 121 - DNA, 357 bp, synthetic construct
  misc_feature 1..357: note = DB435 variable heavy chain domain
  source 1..357: mol_type = other DNA
caggtgcagctggtgcagtctggggctgaggtgaagaagcctggggcctcagtgaaggtttcctgcaaggcatctggaggcaccttcagcagctatgctatcagctgggtgcgtcaggcccctggacaagggcttgagtggatgggctggatcacccctcacaatggtaacataaagtatgcacgggagttccagggccgtgtcaccatgacccgcgacacgtccacgagcacagtctacatggagctgagcagcctgcgttctgaggacacggccgtgtattactgtgcgaaagatctgaactggaacgcagcctttgactactggggccaggggaccctggtcaccgtctcctca

SEQ ID NO: 122 - protein, 119 aa, synthetic construct
  REGION 1..119: note = DB435 variable heavy chain domain
QVQLVQSGAEVKKPGASVKVSCKASGGTFSSYAISWVRQAPGQGLEWMGWITPHNGNIKYAREFQGRVTMTRDTSTSTVYMELSSLRSEDTAVYYCAKDLNWNAAFDYWGQGTLVTVSS

SEQ ID NO: 123 - DNA, 24 bp, synthetic construct
  misc_feature 1..24: note = DB435 CDR H1
  source 1..24: mol_type = other DNA
ggaggcaccttcagcagctatgct

SEQ ID NO: 124 - protein, 8 aa, synthetic construct
  REGION 1..8: note = DB435 CDR H1
GGTFSSYA

SEQ ID NO: 125 - DNA, 24 bp, synthetic construct
  misc_feature 1..24: note = DB435 CDR H2
  source 1..24: mol_type = other DNA
atcacccctcacaatggtaacata

SEQ ID NO: 126 - protein, 8 aa, synthetic construct
  REGION 1..8: note = DB435 CDR H2
ITPHNGNI

SEQ ID NO: 127 - DNA, 36 bp, synthetic construct
  misc_feature 1..36: note = DB435 CDR H3
  source 1..36: mol_type = other DNA
gcgaaagatctgaactggaacgcagcctttgactac

SEQ ID NO: 128 - protein, 12 aa, synthetic construct
  REGION 1..12: note = DB435 CDR H3
AKDLNWNAAFDY

SEQ ID NO: 129 - DNA, 2340 bp, synthetic construct
  misc_feature 1..2340: note = OMT1 VHVL x TSC456 scFv-Fc-scFv TRI129
  source 1..2340: mol_type = other DNA
atggaagcaccagcgcagcttctcttcctcctgctactctggctcccagataccaccggtgaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcttcaggtggaggcggttcaggcggaggtggatccggcggtggcggctccggtggcggcggatctgacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 130 - protein, 759 aa, synthetic construct
  REGION 1..759: note = OMT1 VHVL x TSC456 scFv-Fc-scFv TRI129
EVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSGGGGSGGGGSGGGGSGGGGSDIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 131 - DNA, 2334 bp, synthetic construct
  misc_feature 1..2334: note = OMT1 VLVH x TSC456 scFv-Fc-scFv TRI130
  source 1..2334: mol_type = other DNA
atggaagcaccagcgcagcttctcttcctcctgctactctggctcccagataccaccggtgacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaaggtggaggcggttcaggcggaggtggatccggcggtggcggctccggtggcggcggatctgaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 132 - protein, 757 aa, synthetic construct
  REGION 1..757: note = OMT1 VLVH x TSC456 scFv-Fc-scFv TRI130
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 133 - DNA, 2313 bp, synthetic construct
  misc_feature 1..2313: note = DB8 VHVL x TSC456 scFv-Fc-scFv TRI123
  source 1..2313: mol_type = other DNA
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacatcttcaccgactactatatgcactgggtgcgtcaggcccctggacaagggcttgagtggatgggatggatgagccctaacagtggtaacacaggctatgcacagaagttccagggccgtgtcaccatgacccgcgacacgtccacgagcacagtctacatggagctgagcagcctgcgttctgaggacacggccgtgtattactgtgcgagagatgcggcggattacggtgactacgttgcttttgatatctggggccaagggacaatggtcaccgtctcttcaggcggcggcggcagcggcggcggcggcagcggcggcggaggctccggcggcggcggcagcgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 134 - protein, 750 aa, synthetic construct
  REGION 1..750: note = DB8 VHVL x TSC456 scFv-Fc-scFv TRI123
QVQLVQSGAEVKKPGASVKVSCKASGYIFTDYYMHWVRQAPGQGLEWMGWMSPNSGNTGYAQKFQGRVTMTRDTSTSTVYMELSSLRSEDTAVYYCARDAADYGDYVAFDIWGQGTMVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 135 - DNA, 2307 bp, synthetic construct
  misc_feature 1..2307: note = DB8 VLVH x TSC456 scFv-Fc-scFv TRI124
  source 1..2307: mol_type = other DNA
atggaagcaccagcgcagcttctcttcctcctgctactctggctcccagataccaccggtgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaaggcggcggcggcagcggcggcggcggcagcggcggcggaggctccggcggcggcggcagccaggtgcagctggtgcagtctggggctgaggtgaagaagcctggggcctcagtgaaggtttcctgcaaggcatctggatacatcttcaccgactactatatgcactgggtgcgtcaggcccctggacaagggcttgagtggatgggatggatgagccctaacagtggtaacacaggctatgcacagaagttccagggccgtgtcaccatgacccgcgacacgtccacgagcacagtctacatggagctgagcagcctgcgttctgaggacacggccgtgtattactgtgcgagagatgcggcggattacggtgactacgttgcttttgatatctggggccaagggacaatggtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 136 - protein, 748 aa, synthetic construct
  REGION 1..748: note = DB8 VLVH x TSC456 scFv-Fc-scFv TRI124
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSGGGGSQVQLVQSGAEVKKPGASVKVSCKASGYIFTDYYMHWVRQAPGQGLEWMGWMSPNSGNTGYAQKFQGRVTMTRDTSTSTVYMELSSLRSEDTAVYYCARDAADYGDYVAFDIWGQGTMVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 137 - DNA, 2313 bp, synthetic construct
  misc_feature 1..2313: note = DB8 VHVL x TSC456 scFv-Fc-scFv TRI137
  source 1..2313: mol_type = other DNA
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacatcttcaccgactactatatgcactgggtgcgtcaggcccctggacaagggcttgagtggatgggatggatgagccctaacagtggtaacacaggctatgcacagaagttccagggccgtgtcaccatgacccgcgacacgtccacgagcacagtctacatggagctgagcagcctgcgttctgaggacacggccgtgtattactgtgcgagagatgcggcggattacggtgactacgttgcttttgatatctggggccaagggacaatggtcaccgtctcttcaggtggaggcggttcaggcggaggtggatccggcggtggcggctccggtggcggcggatctgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcatga

SEQ ID NO: 138 - protein, 750 aa, synthetic construct; identical to SEQ ID NO: 134
  REGION 1..750: note = DB8 VHVL x TSC456 scFv-Fc-scFv TRI137

SEQ ID NO: 139 - DNA, 2304 bp, synthetic construct
  misc_feature 1..2304: note = DB60 VHVL x TSC456 scFv-Fc-scFv TRI125
  source 1..2304: mol_type = other DNA
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacaccttcaccagctactatatgcactgggtgcgtcaggcccctggacaagggcttgagtggatggggtggatcaaccctaacagtggtgacacaagctatgcacagaagttccagggccgtgtcaccatgacccgcgacacgtccacgagcacagtctacatggagctgagcagcctgcgttctgaggacacggccgtgtattactgtgcgcaggatagtagtggttccggggcttttgatatctggggccaagggacaatggtcaccgtctcttcaggcggcggcggcagcggcggcggcggcagcggcggcggaggctccggcggcggcggcagcgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 140 - protein, 747 aa, synthetic construct
  REGION 1..747: note = DB60 VHVL x TSC456 scFv-Fc-scFv TRI125
QVQLVQSGAEVKKPGASVKVSCKASGYTFTSYYMHWVRQAPGQGLEWMGWINPNSGDTSYAQKFQGRVTMTRDTSTSTVYMELSSLRSEDTAVYYCAQDSSGSGAFDIWGQGTMVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 141 - DNA, 2316 bp, synthetic construct
  misc_feature 1..2316: note = DB65 VHVL x TSC456 scFv-Fc-scFv TRI126
  source 1..2316: mol_type = other DNA
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacaccttcaccggctactatatgcactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcgaaagaggaaccgatttttggagtggttatggatgcttttgatatctggggccaagggacaatggtcaccgtctcctcaggcggcggcggcagcggcggcggcggcagcggcggcggaggctccggcggcggcggcagcgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 142 - protein, 751 aa, synthetic construct
  REGION 1..751: note = DB65 VHVL x TSC456 scFv-Fc-scFv TRI126
QVQLVQSGAEVKKPGASVKVSCKASGYTFTGYYMHWVRQAPGQGLEWMGWMNPNSGNTGYAQKFQGRVTMTRDTSTSTVYMELSSLRSEDTAVYYCAKEEPIFGVVMDAFDIWGQGTMVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 143 - DNA, 2304 bp, synthetic construct
  misc_feature 1..2304: note = DB82 VLVH x TSC456 scFv-Fc-scFv TRI127
  source 1..2304: mol_type = other DNA
atggaagcaccagcgcagcttctcttcctcctgctactctggctcccagataccaccggtgacatccagatgacccagtctccatcctccctgtctgcatctgtaggagaccgcgtcaccatcacttgccgggcaagtcagaccataaacaactatttgaactggtatcagcagaaaccagggaaagcccctaagctcctgatctattctgcatctactttgcaaagtggggtcccatcacgtttcagtggcagtggatctgggacagatttcactctcaccatcagcagtctgcaacctgaagattttgcaacttactactgtcaccagagttacacttcacctctcactttcggcggaggtaccaaggtggagatcaaaggcggcggcggcagcggcggcggcggcagcggcggcggaggctccggcggcggcggcagcgaggtgcagctggtggagtctgggggaggcttggtacagcctggggggtccctgcgcctctcctgtgcagcctctggattcacctttagcagctatgccatgagctgggtccgccaggctccagggaaggggctggagtgggtctcagttattagtgccaatagtgctggtctaggccatgcggactctgtgaagggccggttcaccatctcccgcgacaattccaagaacacgctgtatctgcaaatgaacagcctgcgcgccgaggacacggccgtatattactgtgcgagagtgggctatagcagctcggctgatgcttttgatatctggggccaagggacaatggtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 144 - protein, 747 aa, synthetic construct
  REGION 1..747: note = DB82 VLVH x TSC456 scFv-Fc-scFv TRI127
DIQMTQSPSSLSASVGDRVTITCRASQTINNYLNWYQQKPGKAPKLLIYSASTLQSGVPSRFSGSGSGTDFTLTISSLQPEDFATYYCHQSYTSPLTFGGGTKVEIKGGGGSGGGGSGGGGSGGGGSEVQLVESGGGLVQPGGSLRLSCAASGFTFSSYAMSWVRQAPGKGLEWVSVISANSAGLGHADSVKGRFTISRDNSKNTLYLQMNSLRAEDTAVYYCARVGYSSSADAFDIWGQGTMVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 145 - DNA, 2337 bp, synthetic construct
  misc_feature 1..2337: note = DB83 VHVL x TSC456 scFv-Fc-scFv TRI134
  source 1..2337: mol_type = other DNA
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacaccttcactagctatgctatgcattgggtgcgtcaggcccctggacaagggcttgagtggatgggacttgttgatcctgaagatggtgaaacaatatatgcagagaagttccagggccgtgtcaccatgacccgcgacacgtccacgagcacagtctacatggagctgagcagcctgcgttctgaggacacggccgtgtattactgtgcgagacgaacgtattactatgatagtagtggttcccgttatgcttttgatatctggggccaagggaccacggtcaccgtctcttcaggcggcggcggcagcggcggcggcggcagcggcggcggaggctccggcggcggcggcagcgatgttgtgatgactcagtctccactctccctgcccgtcacccctggagagccggcctccatctcctgcaggtctagtcagagcctcctgcatagtaatggagacaactatttggattggtacctgcagaagccagggcagtctccacagctcctgatctatttgggttctaatcgggcctccggggtccctgaccgtttcagtggcagtggatcaggcacagattttacactgaaaatcagccgtgtggaggctgaggatgttggggtttattactgcatgcaagctacacactggccactcactttcggccctggtaccaaagtggatatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 146 - protein, 758 aa, synthetic construct
  REGION 1..758: note = DB83 VHVL x TSC456 scFv-Fc-scFv TRI134
QVQLVQSGAEVKKPGASVKVSCKASGYTFTSYAMHWVRQAPGQGLEWMGLVDPEDGETIYAEKFQGRVTMTRDTSTSTVYMELSSLRSEDTAVYYCARRTYYYDSSGSRYAFDIWGQGTTVTVSSGGGGSGGGGSGGGGSGGGGSDVVMTQSPLSLPVTPGEPASISCRSSQSLLHSNGDNYLDWYLQKPGQSPQLLIYLGSNRASGVPDRFSGSGSGTDFTLKISRVEAEDVGVYYCMQATHWPLTFGPGTKVDIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 147 - DNA, 2307 bp, synthetic construct
  misc_feature 1..2307: note = DB86 VHVL x TSC456 scFv-Fc-scFv TRI128
  source 1..2307: mol_type = other DNA
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatatatgttcagtggccattctgcacactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcgagagatagcagtggctggtacgatgtctttgactactggggccaggggaccctggtcaccgtctcctcaggtggaggcggttcaggcggaggtggatccggcggtggcggctccggtggcggcggatctgacatccagatgacccagtctccatcctccctgtctgcatctgtaggagaccgcgtcaccatcacttgccgggcaagtcagggcatcagaaatgatttaggttggtatcagcagaaaccagggaaagcccctaagctcctgatctatgctgcatccactttgcaatcaggggtcccatcacgtttcagtggcagtggatctgggacagatttcactctcaccatcagcagtctgcaacctgaagattttgcaacttactactgtcaacagagttacggtgcccccctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 148 - protein, 748 aa, synthetic construct
  REGION 1..748: note = DB86 VHVL x TSC456 scFv-Fc-scFv TRI128
QVQLVQSGAEVKKPGASVKVSCKASGYMFSGHSAHWVRQAPGQGLEWMGWMNPNSGNTGYAQKFQGRVTMTRDTSTSTVYMELSSLRSEDTAVYYCARDSSGWYDVFDYWGQGTLVTVSSGGGGSGGGGSGGGGSGGGGSDIQMTQSPSSLSASVGDRVTITCRASQGIRNDLGWYQQKPGKAPKLLIYAASTLQSGVPSRFSGSGSGTDFTLTISSLQPEDFATYYCQQSYGAPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 149 - DNA, 2325 bp, synthetic construct
  misc_feature 1..2325: note = DB280 VHVL x TSC456 scFv-Fc-scFv TRI131
  source 1..2325: mol_type = other DNA
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacagcctcaacttatactatatgcactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcgagcctcgattgtagtggtggtagctgctactccgaatatgatgcttttgatatctggggccaagggaccacggtcaccgtctcctcaggcggcggcggcagcggcggcggcggcagcggcggcggaggctccggcggcggcggcagcgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 150 - protein, 754 aa, synthetic construct
  REGION 1..754: note = DB280 VHVL x TSC456 scFv-Fc-scFv TRI131
QVQLVQSGAEVKKPGASVKVSCKASGYSLNLYYMHWVRQAPGQGLEWMGWMNPNSGNTGYAQKFQGRVTMTRDTSTSTVYMELSSLRSEDTAVYYCASLDCSGGSCYSEYDAFDIWGQGTTVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 151 - DNA, 2304 bp, synthetic construct
  misc_feature 1..2304: note = DB331 VHVL x TSC456 scFv-Fc-scFv TRI132
  source 1..2304: mol_type = other DNA
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggatacaccttcaccagctactatatgcactgggtgcgtcaggcccctggacaagggcttgagtggatgggatggatgaaccctaacagtggtaacacaggctatgcacagaagttccagggccgtgtcaccatgacccgcgacacgtccacgagcacagtctacatggagctgagcagcctgcgttctgaggacacggccgtgtattactgtgcaacagatctcgcgggggaagccttgttcgacccctggggccagggcaccctggtcaccgtctcctcaggcggcggcggcagcggcggcggcggcagcggcggcggaggctccggcggcggcggcagcgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 152 - protein, 747 aa, synthetic construct
  REGION 1..747: note = DB331 VHVL x TSC456 scFv-Fc-scFv TRI132
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCATDLAGEALFDPWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 153 - DNA, 2298 bp, synthetic construct
  misc_feature 1..2298: note = DB415 VHVL x TSC456 scFv-Fc-scFv TRI138
  source 1..2298: mol_type = other DNA
atggaagcaccagcgcagcttctcttcctcctgctactctggctcccagataccaccggtgaggtgcagctggtggagtctgggggaggcttggtacagcctggggggtccctgcgcctctcctgtgcagcctctggaatcaccttcagtagttatggcatgcattgggtccgccaggctccagggaaggggctggagtgggtctcaggtattagttggaatagtggtaacagagtctatgtggactctgtgaagggccggttcaccatctcccgcgacaattccaagaacacgctgtatctgcaaatgaacagcctgcgcgccgaggacacggccgtatattactgtgcgagagatactaatgatgcttttgatatctggggccaagggaccacggtcaccgtctcctcaggtggaggcggttcaggcggaggtggatccggcggtggcggctccggtggcggcggatctgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactggagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcatga

SEQ ID NO: 154 - protein, 745 aa, synthetic construct
  REGION 1..745: note = DB415 VHVL x TSC456 scFv-Fc-scFv TRI138
EVQLVESGGGLVQPGGSLRLSCAASGITFSSYGMHWVRQAPGKGLEWVSGISWNSGNRVYVDSVKGRFTISRDNSKNTLYLQMNSLRAEDTAVYYCARDTNDAFDIWGQGTTVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 155 - DNA, 2304 bp, synthetic construct
  misc_feature 1..2304: note = DB435 VHVL x TSC456 scFv-Fc-scFv TRI139
  source 1..2304: mol_type = other DNA
atggaagcaccagcgcagcttctcttcctcctgctactctggctcccagataccaccggtcaggtgcagctggtgcagtctggggctgaggtgaagaagcctggggcctcagtgaaggtttcctgcaaggcatctggaggcaccttcagcagctatgctatcagctgggtgcgtcaggcccctggacaagggcttgagtggatgggctggatcacccctcacaatggtaacataaagtatgcacgggagttccagggccgtgtcaccatgacccgcgacacgtccacgagcacagtctacatggagctgagcagcctgcgttctgaggacacggccgtgtattactgtgcgaaagatctgaactggaacgcagcctttgactactggggccaggggaccctggtcaccgtctcctcaggtggaggcggttcaggcggaggtggatccggcggtggcggctccggtggcggcggatctgacatccagatgacccagtctccatcctccctgtctgcatctgtaggagacagagtcaccatcacttgccgggcaagtcagagcattagcagctatctgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggaggtaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactggagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcatga

SEQ ID NO: 156 - protein, 747 aa, synthetic construct
  REGION 1..747: note = DB435 VHVL x TSC456 scFv-Fc-scFv TRI139
QVQLVQSGAEVKKPGASVKVSCKASGGTFSSYAISWVRQAPGQGLEWMGWITPHNGNIKYAREFQGRVTMTRDTSTSTVYMELSSLRSEDTAVYYCAKDLNWNAAFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 157 - protein, 108 aa, synthetic construct
  REGION 1..108: note = TSC455 variable light domain
DIQMTQSPSTLSASVGDRVTMTCSASSSVSYMNWYQQKPGKAPKRWIYDSSKLASGVPSRFSGSGSGTEYTLTISSLQPDDFATYYCQQWSRNPPTFGGGTKVEIKRS

SEQ ID NO: 158 - protein, 108 aa, synthetic construct
  REGION 1..108: note = TSC456 variable light domain
DIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 159 - protein, 121 aa, synthetic construct
  REGION 1..121: note = TSC455 and TSC456 variable heavy domain
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSS

SEQ ID NO: 160 - protein, 107 aa, synthetic construct
  REGION 1..107: note = DRA222 variable light domain
DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITS

SEQ ID NO: 161 - protein, 121 aa, synthetic construct
  REGION 1..121: note = DRA222 variable heavy domain
QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS

SEQ ID NO: 162 - protein, 10 aa, synthetic construct
  REGION 1..10: note = Cris7 and DRA222 VL CDR1 (Kabat)
SASSSVSYMN

SEQ ID NO: 163 - protein, 7 aa, synthetic construct
  REGION 1..7: note = Cris7 and DRA222 VL CDR2 (Kabat)
DSSKLAS

SEQ ID NO: 164 - protein, 9 aa, synthetic construct
  REGION 1..9: note = Cris7 and DRA222 VL CDR3 (Kabat)
QQWSRNPPT

SEQ ID NO: 165 - protein, 5 aa, synthetic construct
  REGION 1..5: note = Cris7 and DRA222 VH CDR1 (Kabat)
RSTMH

SEQ ID NO: 166 - protein, 16 aa, synthetic construct
  REGION 1..16: note = Cris7 and DRA222 VH CDR2 (Kabat)
YINPSSAYTNYNQKFK

SEQ ID NO: 167 - protein, 11 aa, synthetic construct
  REGION 1..11: note = Cris7 and DRA222 VH CDR3 (Kabat)
QVHYDYNGFPY

SEQ ID NO: 168 - protein, 7 aa, synthetic construct
  REGION 1..7: note = Cris7 and DRA222 VL CDR1 (IMGT)
ASSSVSY

SEQ ID NO: 169 - skipped

SEQ ID NO: 170 - protein, 9 aa, synthetic construct
  REGION 1..9: note = Cris7 and DRA222 VL CDR3 (IMGT)
QQWSRNPPT

SEQ ID NO: 171 - protein, 8 aa, synthetic construct
  REGION 1..8: note = Cris7 and DRA222 VH CDR1 (IMGT)
GYTFTRST

SEQ ID NO: 172 - protein, 8 aa, synthetic construct
  REGION 1..8: note = Cris7 and DRA222 VH CDR2 (IMGT)
INPSSAYT

SEQ ID NO: 173 - protein, 9 aa, synthetic construct
  REGION 1..9: note = Cris7 and DRA222 VH CDR3 (IMGT)
QQWSRNPPT

SEQ ID NO: 174 - protein, 5 aa, synthetic construct
  REGION 1..5: note = I2C VH CDR1 (Kabat)
KYAMN

SEQ ID NO: 175 - protein, 19 aa, synthetic construct
  REGION 1..19: note = I2C VH CDR2 (Kabat)
RIRSKYNNYATYYADSVKD

SEQ ID NO: 176 - protein, 14 aa, synthetic construct
  REGION 1..14: note = I2C VH CDR3 (Kabat)
HGNFGNSYISYWAY

SEQ ID NO: 177 - skipped

SEQ ID NO: 178 - protein, 9 aa, synthetic construct
  REGION 1..9: note = I2C VL CDR3 (IMGT)
VLWYSNRWV

SEQ ID NO: 179 - protein, 8 aa, synthetic construct
  REGION 1..8: note = I2C VH CDR1 (IMGT)
GFTFNKYA

SEQ ID NO: 180 - protein, 10 aa, synthetic construct
  REGION 1..10: note = I2C VH CDR2 (IMGT)
IRSKYNNYAT

SEQ ID NO: 181 - protein, 16 aa, synthetic construct
  REGION 1..16: note = I2C VH CDR3 (IMGT)
VRHGNFGNSYISYWAY

SEQ ID NO: 182 - protein, 10 aa, synthetic construct
  REGION 1..10: note = HuM291 VL CDR1 (Kabat)
SASSSVSYMN

SEQ ID NO: 183 - protein, 7 aa, synthetic construct
  REGION 1..7: note = HuM291 VL CDR2 (Kabat)
DTSKLAS

SEQ ID NO: 184 - protein, 9 aa, synthetic construct
  REGION 1..9: note = HuM291 VL CDR3 (Kabat)
QQWSSNPPT

SEQ ID NO: 185 - protein, 5 aa, synthetic construct
  REGION 1..5: note = HuM291 VH CDR1 (Kabat)
SYTMH

SEQ ID NO: 186 - protein, 17 aa, synthetic construct
  REGION 1..17: note = HuM291 VH CDR2 (Kabat)
YINPRSGYTHYNQKLKD

SEQ ID NO: 187 - protein, 11 aa, synthetic construct
  REGION 1..11: note = HuM291 VH CDR3 (Kabat)
SAYYDYDGFAY

SEQ ID NO: 188 - protein, 7 aa, synthetic construct
  REGION 1..7: note = HuM291 VL CDR1 (IMGT)
ASSSVSY

SEQ ID NO: 189 - skipped

SEQ ID NO: 190 - protein, 9 aa, synthetic construct
  REGION 1..9: note = HuM291 VL CDR3 (IMGT)
QQWSSNPPT

SEQ ID NO: 191 - protein, 8 aa, synthetic construct
  REGION 1..8: note = HuM291 VH CDR1 (IMGT)
GYTFISYT

SEQ ID NO: 192 - protein, 8 aa, synthetic construct
  REGION 1..8: note = HuM291 VH CDR2 (IMGT)
INPRSGYT

SEQ ID NO: 193 - protein, 13 aa, synthetic construct
  REGION 1..13: note = HuM291 VH CDR3 (IMGT)
ARSAYYDYDGFAY

SEQ ID NO: 194 - DNA, 339 bp, Mus sp.
  source 1..339: mol_type = other DNA
gacatcatgatgtcccagtccccctcctccctggccgtgtccgtgggcgagaagttcaccatgacctgcaagtcctcccagtccctgttcttcggctccacccagaagaactacctggcctggtaccagcagaagcccggccagtcccccaagctgctgatctactgggcctccacccgggagtccggcgtgcccgaccggttcaccggctccggctccggcaccgacttcaccctggccatctcctccgtgatgcccgaggacctggccgtgtactactgccagcagtactacaactacccctggaccttcggcggcggcaccaagctggagatcaag

SEQ ID NO: 195 - protein, 113 aa, Mus sp.
DIMMSQSPSSLAVSVGEKFTMTCKSSQSLFFGSTQKNYLAWYQQKPGQSPKLLIYWASTRESGVPDRFTGSGSGTDFTLAISSVMPEDLAVYYCQQYYNYPWTFGGGTKLEIK

SEQ ID NO: 196 - DNA, 345 bp, Mus sp.
  source 1..345: mol_type = other DNA
cagctgcaggagtccggccccggcctggtgaagccctcccagtccctgtccctgacctgctccgtgaccgactactccatcacctccggctactactggaactggattcggcagttccccggcaacaagctggagtggatgggctacatctcctacgacggctccaacaactacaacccctccctgaagaaccggatctccatcacccgggacacctccaagaaccagttcttcctgaagctgtcctccgtgaccaccgaggacaccgccacctactactgctcccggggcgagggcttctacttcgactcctggggccagggcaccaccctgaccgtgtcctcg

SEQ ID NO: 197 - protein, 116 aa, Mus sp.
VQLQESGPGLVKPSQSLSLTCSVTDYSITSGYYWNWIRQFPGNKLEWMGYISYDGSNNYNPSLKNRISITRDTSKNQFFLKLSSVTTEDTATYYCSRGEGFYFDSWGQGTTLTVSS

SEQ ID NO: 198 - protein, 20 aa, synthetic construct
  REGION 1..20: note = secretory signal sequence
MEAPAQLLFLLLLWLPDTTG

SEQ ID NO: 199 - DNA, 1074 bp, synthetic construct
  misc_feature 1..1074: note = human CD123 ectodomain, with Avi-3xFLAG-His affinity tag TRI032
  source 1..1074: mol_type = other DNA
atggaagcaccagcgcagcttctcttcctcctgctactctggctcccagataccaccggtaaggaggaccccaacccccccatcaccaacctgaggatgaaggccaaggcccagcagctgacctgggacctgaacaggaacgtgacagacatcgaatgcgtgaaggatgccgactacagcatgcccgccgtgaacaactcctactgccagttcggcgccatcagcctgtgcgaggtgacaaactacaccgtgagagtggccaacccccccttcagcacctggatcctgtttcccgagaacagcggcaaaccctgggctggcgctgagaacctgacctgctggatccacgacgtggactttctgtcctgcagctgggctgtgggacccggagctcctgccgatgtgcagtacgacctgtacctgaatgtggccaacagaagacagcagtacgagtgcctgcattacaagaccgacgcccagggaaccaggatcggctgcaggtttgatgacatcagcaggctgtcctccggcagccagtccagccacatcctggtgagaggcagatccgccgccttcggcattccctgcacagacaagttcgtcgtcttcagccagatcgagattctgaccccccccaacatgaccgccaagtgtaacaagacccacagcttcatgcactggaagatgaggagccacttcaacaggaagttcaggtacgagctccagatccagaagaggatgcagcccgtgatcaccgagcaggtgagggacaggacatccttccagctgctgaatcccggcacatacaccgtgcagatcagggccagggaaagggtgtacgagttcctgtccgcctggagcaccccccagaggttcgagtgtgaccaggaggagggagccaataccagggcctggagatcctcgagtctcaacgatatttttgaagcccaaaaaattgagtggcatgaagattacaaggacgatgacgacaaagactataaggacgacgacgataaggattacaaggatgacgatgataagcaccatcatcatcaccatcaccaccaccactga

SEQ ID NO: 200 - protein, 337 aa, synthetic construct
  REGION 1..337: note = human CD123 ectodomain, with Avi-3xFLAG-His affinity tag TRI032
KEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRSSSLNDIFEAQKIEWHEDYKDDDDKDYKDDDDKDYKDDDDKHHHHHHHHHH

SEQ ID NO: 201 - DNA, 996 bp, Homo sapiens
  source 1..996: mol_type = other DNA
atggtcctcctttggctcacgctgctcctgatcgccctgccctgtctcctgcaaacgaaggaaggtgggaagccttgggcaggtgcggagaatctgacctgctggattcatgacgtggatttcttgagctgcagctgggcggtaggcccgggggcccccgcggacgtccagtacgacctgtacttgaacgttgccaacaggcgtcaacagtacgagtgtcttcactacaaaacggatgctcagggaacacgtatcgggtgtcgtttcgatgacatctctcgactctccagcggttctcaaagttcccacatcctggtgcggggcaggagcgcagccttcggtatcccctgcacagataagtttgtcgtcttttcacagattgagatattaactccacccaacatgactgcaaagtgtaataagacacattcctttatgcactggaaaatgagaagtcatttcaatcgcaaatttcgctatgagcttcagatacaaaagagaatgcagcctgtaatcacagaacaggtcagagacagaacctccttccagctactcaatcctggaacgtacacagtacaaataagagcccgggaaagagtgtatgaattcttgagcgcctggagcaccccccagcgcttcgagtgcgaccaggaggagggcgcaaacacacgtgcctggcggacgtcgctgctgatcgcgctggggacgctgctggccctggtctgtgtcttcgtgatctgcagaaggtatctggtgatgcagagactctttccccgcatccctcacatgaaagaccccatcggtgacagcttccaaaacgacaagctggtggtctgggaggcgggcaaagccggcctggaggagtgtctggtgactgaagtacaggtcgtgcagaaaactacgcgtacgcggccgctcgagcagaaactcatctcagaagaggatctggcagcaaatgatatcctggattacaaggatgacgacgataaggtttaa

SEQ ID NO: 202 - protein, 310 aa, Homo sapiens
GGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKTTRTRPLEQKLISEEDLAANDILDYKDDDDKV

SEQ ID NO: 203 - DNA, 1140 bp, Homo sapiens
  source 1..1140: mol_type = other DNA
atggaagcccccgcccagctgctcttcctgctgctcctgtggctgcctgacaccaccggcaaggaagaccccaatgcccccatcaggaacctgagaatgaaggagaaggcccagcagctcatgtgggatctgaacaggaacgtgaccgacgtggagtgtatcaagggcaccgactactccatgcccgccatgaataacagctattgccagttcggcgccatcagcctgtgcgaggtcaccaactacaccgtgagagtggccagcccccccttctccacctggattctgttccctgagaacagcggcacccctagggctggcgctgagaatctgacatgctgggtccatgacgtggacttcctgagctgcagctgggtggtgggacctgctgctcccgctgacgtgcagtacgatctgtatctgaacaaccccaactcccacgagcagtacaggtgcctgcactacaagacagacgctagaggcacccagatcggctgcaggttcgatgatatcgcccctctgagcaggggatcccagagctcccatatcctggtgaggggcaggtccgccgctgtgagcattccttgcaccgacaagttcgtcttcttcagccagatcgagaggctgaccccccctaacatgacaggcgagtgcaacgagacccacagcttcatgcactggaagatgaagagccatttcaacaggaaattcaggtacgaactgaggattcagaagagaatgcagcccgtgaggacagagcaggtgagggatacaaccagcttccagctgcccaatcctggcacctataccgtgcagatcagggctagagagaccgtgtacgagtttctgtccgcctggagcaccccccagaggtttgaatgtgaccaggaggagggagcctccagcagggcttggagaaccagcctcctcatcgccctgggcacactgctggctctgctgtgtgtgttcctgatctgcagaaggtacctggtgatgcagaggctcttccctaggattccccacatgaaggaccccatcggcgacaccttccagcaggacaaactggtggtgtgggaagccggaaaggccggcctggaggaatgcctcgtgtccgaggtgcaggtggtggagaagacctaa

SEQ ID NO: 204 - protein, 359 aa, Homo sapiens
KEDPNAPIRNLRMKEKAQQLMWDLNRNVTDVECIKGTDYSMPAMNNSYCQFGAISLCEVTNYTVRVASPPFSTWILFPENSGTPRAGAENLTCWVHDVDFLSCSWVVGPAAPADVQYDLYLNNPNSHEQYRCLHYKTDARGTQIGCRFDDIAPLSRGSQSSHILVRGRSAAVSIPCTDKFVFFSQIERLTPPNMTGECNETHSFMHWKMKSHFNRKFRYELRIQKRMQPVRTEQVRDTTSFQLPNPGTYTVQIRARETVYEFLSAWSTPQRFECDQEEGASSRAWRTSLLIALGTLLALLCVFLICRRYLVMQRLFPRIPHMKDPIGDTFQQDKLVVWEAGKAGLEECLVSEVQVVEKT

SEQ ID NO: 205 - DNA, 1713 bp, Homo sapiens
  source 1..1713: mol_type = other DNA
gtcaggttcatggttacgaagctgctgaccccaggatcccagcccgtgggagagaagggggtctctgacagcccccacccctccccactgccagatccttattgggtctgagtttcaggggtggggccccagctggaggttataaaacagctcaatcggggagtacaaccttcggtttctcttcggggaaagctgctttcagcgcacacgggaagatatcagaaacatcctaggatcaggacaccccagatcttctcaactggaaccacgaaggctgtttcttccacacagtactttgatctccatttaagcaggcacctctgtcctgcgttccggagctgcgttcccgatggtcctcctttggctcacgctgctcctgatcgccctgccctgtctcctgcaaacgaaggaagatccaaacccaccaatcacgaacctaaggatgaaagcaaaggctcagcagttgacctgggaccttaacagaaatgtgaccgatatcgagtgtgttaaagacgccgactattctatgccggcagtgaacaatagctattgccagtttggagcaatttccttatgtgaagtgaccaactacaccgtccgagtggccaacccaccattctccacgtggatcctcttccctgagaacagtgggaagccttgggcaggtgcggagaatctgacctgctggattcatgacgtggatttcttgagctgcagctgggcggtaggcccgggggcccccgcggacgtccagtacgacctgtacttgaacgttgccaacaggcgtcaacagtacgagtgtcttcactacaaaacggatgctcagggaacacgtatcgggtgtcgtttcgatgacatctctcgactctccagcggttctcaaagttcccacatcctggtgcggggcaggagcgcagccttcggtatcccctgcacagataagtttgtcgtcttttcacagattgagatattaactccacccaacatgactgcaaagtgtaataagacacattcctttatgcactggaaaatgagaagtcatttcaatcgcaaatttcgctatgagcttcagatacaaaagagaatgcagcctgtaatcacagaacaggtcagagacagaacctccttccagctactcaatcctggaacgtacacagtacaaataagagcccgggaaagagtgtatgaattcttgagcgcctggagcaccccccagcgcttcgagtgcgaccaggaggagggcgcaaacacacgtgcctggcggacgtcgctgctgatcgcgctggggacgctgctggccctggtctgtgtcttcgtgatctgcagaaggtatctggtgatgcagagactctttccccgcatccctcacatgaaagaccccatcggtgacagcttccaaaacgacaagctggtggtctgggaggcgggcaaagccggcctggaggagtgtctggtgactgaagtacaggtcgtgcagaaaacttgagactggggttcagggcttgtgggggtctgcctcaatctccctggccgggccaggcgcctgcacagactggctgctggacctgcgcacgcagcccaggaatggacattcctaacgggtggtgggcatgggagatgcctgtgtaatttcgtccgaagctgccaggaagaagaacagaactttgtgtgtttatttcatgataaagtgatttttttttttttaacccaaaa

SEQ ID NO: 206 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 207 - DNA, 1479 bp, Homo sapiens
  source 1..1479: mol_type = other DNA
gtcaggttcatggttacgaagctgctgaccccaggatcccagcccgtgggagagaagggggtctctgacagcccccacccctccccactgccagatccttattgggtctgagtttcaggggtggggccccagctggaggttataaaacagctcaatcggggagtacaaccttcggtttctcttcggggaaagctgctttcagcgcacacgggaagatatcagaaacatcctaggatcaggacaccccagatcttctcaactggaaccacgaaggctgtttcttccacacagtactttgatctccatttaagcaggcacctctgtcctgcgttccggagctgcgttcccgatggtcctcctttggctcacgctgctcctgatcgccctgccctgtctcctgcaaacgaaggaaggtgggaagccttgggcaggtgcggagaatctgacctgctggattcatgacgtggatttcttgagctgcagctgggcggtaggcccgggggcccccgcggacgtccagtacgacctgtacttgaacgttgccaacaggcgtcaacagtacgagtgtcttcactacaaaacggatgctcagggaacacgtatcgggtgtcgtttcgatgacatctctcgactctccagcggttctcaaagttcccacatcctggtgcggggcaggagcgcagccttcggtatcccctgcacagataagtttgtcgtcttttcacagattgagatattaactccacccaacatgactgcaaagtgtaataagacacattcctttatgcactggaaaatgagaagtcatttcaatcgcaaatttcgctatgagcttcagatacaaaagagaatgcagcctgtaatcacagaacaggtcagagacagaacctccttccagctactcaatcctggaacgtacacagtacaaataagagcccgggaaagagtgtatgaattcttgagcgcctggagcaccccccagcgcttcgagtgcgaccaggaggagggcgcaaacacacgtgcctggcggacgtcgctgctgatcgcgctggggacgctgctggccctggtctgtgtcttcgtgatctgcagaaggtatctggtgatgcagagactctttccccgcatccctcacatgaaagaccccatcggtgacagcttccaaaacgacaagctggtggtctgggaggcgggcaaagccggcctggaggagtgtctggtgactgaagtacaggtcgtgcagaaaacttgagactggggttcagggcttgtgggggtctgcctcaatctccctggccgggccaggcgcctgcacagactggctgctggacctgcgcacgcagcccaggaatggacattcctaacgggtggtgggcatgggagatgcctgtgtaatttcgtccgaagctgccaggaagaagaacagaactttgtgtgtttatttcatgataaagtgatttttttttttttaacccaaaa

SEQ ID NO: 208 - protein, 300 aa, Homo sapiens
MVLLWLTLLLIALPCLLQTKEGGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 209 - protein, 107 aa, synthetic construct
  REGION 1..107: note = CRIS-7 monoclonal antibody VL amino acid sequence
QVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQITR

SEQ ID NO: 210 - protein, 121 aa, synthetic construct
  REGION 1..121: note = CRIS-7 monoclonal antibody VH amino acid sequence
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSA

SEQ ID NO: 211 - protein, 106 aa, synthetic construct [sequence omitted]

SEQ ID NO: 212 - protein, 120 aa, synthetic construct [sequence omitted]

SEQ ID NO: 213 - protein, 15 aa, synthetic construct
  REGION 1..15: note = peptide linker
GGGGSGGGGSGGGGS

SEQ ID NO: 214 - protein, 25 aa, synthetic construct
  REGION 1..25: note = peptide linker
  VARIANT 6..25: note = Residues at positions 6-25 may be absent
GGGGSGGGGSGGGGSGGGGSGGGGS

SEQ ID NO: 215 - protein, 15 aa, synthetic construct
  REGION 1..15: note = sss(s)-hIgG1 hinge
EPKSSDKTHTSPPSS

SEQ ID NO: 216 - protein, 15 aa, synthetic construct
  REGION 1..15: note = csc(s)-hIgG1 hinge
EPKSCDKTHTSPPCS

SEQ ID NO: 217 - protein, 15 aa, synthetic construct
  REGION 1..15: note = ssc(s)-hIgG1 hinge
EPKSSDKTHTSPPCS

SEQ ID NO: 218 - protein, 15 aa, synthetic construct
  REGION 1..15: note = scc(s)-hIgG1 hinge
EPKSSDKTHTCPPCS

SEQ ID NO: 219 - protein, 15 aa, synthetic construct
  REGION 1..15: note = css(s)-hIgG1 hinge
EPKSCDKTHTSPPSS

SEQ ID NO: 220 - protein, 15 aa, synthetic construct
  REGION 1..15: note = scs(s)-hIgG1 hinge
EPKSSDKTHTCPPSS

SEQ ID NO: 221 - protein, 15 aa, synthetic construct
  REGION 1..15: note = ccc(s)-hIgG1 hinge
EPKSCDKTHTSPPCS

SEQ ID NO: 222 - protein, 15 aa, synthetic construct
  REGION 1..15: note = ccc(p)-hIgG1 hinge
EPKSCDKTHTSPPCP

SEQ ID NO: 223 - protein, 15 aa, synthetic construct
  REGION 1..15: note = sss(p)-hIgG1 hinge
EPKSSDKTHTSPPSP

SEQ ID NO: 224 - protein, 15 aa, synthetic construct
  REGION 1..15: note = csc(p)-hIgG1 hinge
EPKSCDKTHTSPPCP

SEQ ID NO: 225 - protein, 15 aa, synthetic construct
  REGION 1..15: note = ssc(p)-hIgG1 hinge
EPKSSDKTHTSPPCP

SEQ ID NO: 226 - protein, 15 aa, synthetic construct
  REGION 1..15: note = scc(p)-hIgG1 hinge
EPKSSDKTHTCPPCP

SEQ ID NO: 227 - protein, 15 aa, synthetic construct
  REGION 1..15: note = css(p)-hIgG1 hinge
EPKSCDKTHTSPPSP

SEQ ID NO: 228 - protein, 15 aa, synthetic construct
  REGION 1..15: note = scs(p)-hIgG1 hinge
EPKSSDKTHTCPPSP

SEQ ID NO: 229 - protein, 6 aa, synthetic construct
  REGION 1..6: note = Scppcp linker
SCPPCP

SEQ ID NO: 230 - protein, 20 aa, synthetic construct
  REGION 1..20: note = STD1 linker
NYGGGGSGGGGSGGGGSGNS

SEQ ID NO: 231 - protein, 38 aa, synthetic construct
  REGION 1..38: note = STD2 linker
NYGGGGSGGGGSGGGGSGNYGGGGSGGGGSGGGGSGNS

SEQ ID NO: 232 - skipped

SEQ ID NO: 233 - protein, 8 aa, synthetic construct
  REGION 1..8: note = H2 linker
GGGGSGNS

SEQ ID NO: 234 - protein, 10 aa, synthetic construct
  REGION 1..10: note = H3 linker
NYGGGGSGNS

SEQ ID NO: 235 - protein, 13 aa, synthetic construct
  REGION 1..13: note = H4 linker
GGGGSGGGGSGNS

SEQ ID NO: 236 - protein, 15 aa, synthetic construct
  REGION 1..15: note = H5 linker
NYGGGGSGGGGSGNS

SEQ ID NO: 237 - protein, 18 aa, synthetic construct
  REGION 1..18: note = H6 linker
GGGGSGGGGSGGGGSGNS

SEQ ID NO: 238 - protein, 8 aa, synthetic construct
  REGION 1..8: note = H7 linker
GCPPCPNS

SEQ ID NO: 239 - protein, 15 aa, synthetic construct
  REGION 1..15: note = (G4S)3 linker
GGGGSGGGGSGGGGS

SEQ ID NO: 240 - protein, 16 aa, synthetic construct
  REGION 1..16: note = H105 linker
SGGGGSGGGGSGGGGS

SEQ ID NO: 241 - protein, 20 aa, synthetic construct
  REGION 1..20: note = (G4S)4 linker
GGGGSGGGGSGGGGSGGGGS

SEQ ID NO: 242 - protein, 21 aa, synthetic construct
  REGION 1..21: note = H75 (NKG2A quadruple mutant) linker
QRHNNSSLNTGTQMAGHSPNS

SEQ ID NO: 243 - protein, 16 aa, synthetic construct
  REGION 1..16: note = H83 (NKG2A derived) linker
SSLNTGTQMAGHSPNS

SEQ ID NO: 244 - protein, 18 aa, synthetic construct
  REGION 1..18: note = H106 (NKG2A derived) linker
QRHNNSSLNTGTQMAGHS

SEQ ID NO: 245 - protein, 14 aa, synthetic construct
  REGION 1..14: note = H81 (NKG2D derived) linker
EVQIPLTESYSPNS

SEQ ID NO: 246 - protein, 20 aa, synthetic construct
  REGION 1..20: note = H91 (NKG2D derived) linker
NSLANQEVQIPLTESYSPNS

SEQ ID NO: 247 - protein, 19 aa, synthetic construct
  REGION 1..19: note = H94 linker
SGGGGSGGGGSGGGGSPNS

SEQ ID NO: 248 - protein, 19 aa, synthetic construct
  REGION 1..19: note = H111 linker
SGGGGSGGGGSGGGGSPGS

SEQ ID NO: 249 - protein, 15 aa, synthetic construct
  REGION 1..15: note = H16 linker
LSVKADFLTPSIGNS

SEQ ID NO: 250 - protein, 20 aa, synthetic construct
  REGION 1..20: note = H17 linker
LSVKADFLTPSISCPPCPNS

SEQ ID NO: 251 - protein, 15 aa, synthetic construct
  REGION 1..15: note = H18 linker
LSVLANFSQPEIGNS

SEQ ID NO: 252 - protein, 20 aa, synthetic construct
  REGION 1..20: note = H19 linker
LSVLANFSQPEISCPPCPNS

SEQ ID NO: 253 - protein, 15 aa, synthetic construct
  REGION 1..15: note = H20 linker
LKIQERVSKPKISNS

SEQ ID NO: 254 - protein, 20 aa, synthetic construct
  REGION 1..20: note = H21 linker
LKIQERVSKPKISCPPCPNS

SEQ ID NO: 255 - protein, 15 aa, synthetic construct
  REGION 1..15: note = H22 linker
LNVSERPFPPHIQNS

SEQ ID NO: 256 - protein, 21 aa, synthetic construct
  REGION 1..21: note = H23 linker
LDVSERPFPPHIQSCPPCPNS

SEQ ID NO: 257 - protein, 15 aa, synthetic construct
  REGION 1..15: note = H24 linker
REQLAEVTLSLKANS

SEQ ID NO: 258 - protein, 20 aa, synthetic construct
  REGION 1..20: note = H25 linker
REQLAEVTLSLKACPPCPNS

SEQ ID NO: 259 - protein, 15 aa, synthetic construct
  REGION 1..15: note = H26 linker
RIHQMNSELSVLANS

SEQ ID NO: 260 - protein, 20 aa, synthetic construct
  REGION 1..20: note = H27 linker
RIHQMNSELSVLACPPCPNS

SEQ ID NO: 261 - protein, 15 aa, synthetic construct
  REGION 1..15: note = H28 linker
DTKGKNVLEKIFSNS

SEQ ID NO: 262 - protein, 15 aa, synthetic construct
  REGION 1..15: note = H30 linker
LPPETQESQEVTLNS

SEQ ID NO: 263 - protein, 15 aa, synthetic construct
  REGION 1..15: note = H32 linker
RIHLNVSERPFPPNS

SEQ ID NO: 264 - protein, 20 aa, synthetic construct
  REGION 1..20: note = H33 linker
RIHLNVSERPFPPCPPCPNS

SEQ ID NO: 265 - protein, 13 aa, synthetic construct
  REGION 1..13: note = H36 linker
GCPPCPGGGGSNS

SEQ ID NO: 266 - protein, 9 aa, synthetic construct
  REGION 1..9: note = H40 linker
GCPPCPANS

SEQ ID NO: 267 - protein, 9 aa, synthetic construct
  REGION 1..9: note = H41 linker
GCPPCPANS

SEQ ID NO: 268 - protein, 8 aa, synthetic construct
  REGION 1..8: note = H42 linker
GCPPCPNS

SEQ ID NO: 269 - protein, 13 aa, synthetic construct
  REGION 1..13: note = H44 linker
GGGASCPPCPGNS

SEQ ID NO: 270 - protein, 13 aa, synthetic construct
  REGION 1..13: note = H45 linker
GGGASCPPCAGNS

SEQ ID NO: 271 - protein, 12 aa, synthetic construct
  REGION 1..12: note = H46 linker
GGGASCPPCANS

SEQ ID NO: 272 - protein, 15 aa, synthetic construct
  REGION 1..15: note = H47 linker
LSVKADFLTPSIGNS

SEQ ID NO: 273 - protein, 11 aa, synthetic construct
  REGION 1..11: note = H48 linker
ADFLTPSIGNS

SEQ ID NO: 274 - protein, 15 aa, synthetic construct
  REGION 1..15: note = H50 linker
LSVLANFSQPEIGNS

SEQ ID NO: 275 - protein, 15 aa, synthetic construct
  REGION 1..15: note = H51 linker
LSVLANFSQPEIGNS

SEQ ID NO: 276 - protein, 11 aa, synthetic construct
  REGION 1..11: note = H52 linker
SQPEIVPISNS

SEQ ID NO: 277 - protein, 16 aa, synthetic construct
  REGION 1..16: note = H53 linker
SQPEIVPISCPPCPNS

SEQ ID NO: 278 - protein, 19 aa, synthetic construct
  REGION 1..19: note = H54 linker
SVLANFSQPEISCPPCPNS

SEQ ID NO: 279 - protein, 15 aa, synthetic construct
  REGION 1..15: note = H55 linker
RIHQMNSELSVLANS

SEQ ID NO: 280 - protein, 12 aa, synthetic construct
  REGION 1..12: note = H56 linker
QMNSELSVLANS

SEQ ID NO: 281 - protein, 10 aa, synthetic construct
  REGION 1..10: note = H57 linker
VSERPFPPNS

SEQ ID NO: 282 - protein, 15 aa, synthetic construct
  REGION 1..15: note = H58 linker
KPFFTCGSADTCPNS

SEQ ID NO: 283 - protein, 15 aa, synthetic construct
  REGION 1..15: note = H59 linker
KPFFTCGSADTCPNS

SEQ ID NO: 284 - protein, 15 aa, synthetic construct
  REGION 1..15: note = H60 linker
QYNCPGQYTFSMPNS

SEQ ID NO: 285 - protein, 21 aa, synthetic construct
  REGION 1..21: note = H61 linker
EPAFTPGPNIELQKDSDCPNS

SEQ ID NO: 286 - protein, 21 aa, synthetic construct
  REGION 1..21: note = H62 linker
QRHNNSSLNTRTQKARHCPNS

SEQ ID NO: 287 - protein, 20 aa, synthetic construct
  REGION 1..20: note = H63 linker
NSLFNQEVQIPLTESYCPNS

SEQ ID NO: 288 - protein, 17 aa, synthetic construct
  REGION 1..17: note = H114 linker
GGGGSGGGGSGGGGSPS

SEQ ID NO: 289 - protein, 14 aa, synthetic construct
  REGION 1..14: note = CD3-binding domain LCDR1
GSSTGAVTSGYYPN

SEQ ID NO: 290 - protein, 14 aa, synthetic construct
  REGION 1..14: note = CD3-binding domain LCDR1
RSSTGAVTSGYYPN

SEQ ID NO: 291 - protein, 14 aa, synthetic construct
  REGION 1..14: note = CD3-binding domain LCDR1
GSSTGAVTSGNYPN

SEQ ID NO: 292 - protein, 7 aa, synthetic construct
  REGION 1..7: note = CD3-binding domain LCDR2
GTKFLAP

SEQ ID NO: 293 - protein, 7 aa, synthetic construct
  REGION 1..7: note = CD3-binding domain LCDR2
ATDMRPS

SEQ ID NO: 294 - protein, 7 aa, synthetic construct
  REGION 1..7: note = CD3-binding domain LCDR2
GTKFLAP

SEQ ID NO: 295 - protein, 9 aa, synthetic construct
  REGION 1..9: note = CD3-binding domain LCDR3
ALWYSNRWV

SEQ ID NO: 296 - protein, 9 aa, synthetic construct
  REGION 1..9: note = CD3-binding domain LCDR3
ALWYSNRWV

SEQ ID NO: 297 - protein, 9 aa, synthetic construct
  REGION 1..9: note = CD3-binding domain LCDR3
VLWYSNRWV

SEQ ID NO: 298 - protein, 5 aa, synthetic construct
  REGION 1..5: note = CD3-binding domain HCDR1
IYAMN

SEQ ID NO: 299 - protein, 5 aa, synthetic construct
  REGION 1..5: note = CD3-binding domain HCDR1
KYAMN

SEQ ID NO: 300 - protein, 5 aa, synthetic construct
  REGION 1..5: note = CD3-binding domain HCDR1
SYAMN

SEQ ID NO: 301 - protein, 19 aa, synthetic construct
  REGION 1..19: note = CD3-binding domain HCDR2
RIRSKYNNYATYYADSVKS

SEQ ID NO: 302 - protein, 19 aa, synthetic construct
  REGION 1..19: note = CD3-binding domain HCDR2
RIRSKYNNYATYYADSVKD

SEQ ID NO: 303 - protein, 19 aa, synthetic construct
  REGION 1..19: note = CD3-binding domain HCDR2
RIRSKYNNYATYYADSVKG

SEQ ID NO: 304 - protein, 14 aa, synthetic construct
  REGION 1..14: note = CD3-binding domain HCDR3
HGNFGNSYVSFFAY

SEQ ID NO: 305 - protein, 14 aa, synthetic construct
  REGION 1..14: note = CD3-binding domain HCDR3
HGNFGNSYISYWAY

SEQ ID NO: 306 - protein, 14 aa, synthetic construct
  REGION 1..14: note = CD3-binding domain HCDR3
HGNFGNSYLSFWAY

SEQ ID NO: 307 - protein, 14 aa, synthetic construct
  REGION 1..14: note = I2C VL CDR1 (Kabat)
GSSTGAVTSGNYPN

SEQ ID NO: 308 - protein, 7 aa, synthetic construct
  REGION 1..7: note = I2C VL CDR2 (Kabat)
GTKFLAP

SEQ ID NO: 309 - protein, 9 aa, synthetic construct
  REGION 1..9: note = I2C VL CDR3 (Kabat)
VLWYSNRWV

SEQ ID NO: 310 - protein, 9 aa, synthetic construct
  REGION 1..9: note = I2C VL CDR1 (IMGT)
TGAVTSGNY

SEQ ID NO: 311 - protein, 251 aa, synthetic construct
  REGION 1..251: note = TSC455 (anti-CD3) TSC394 F87Y scFv
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTEYTLTISSLQPDDFATYYCQQWSRNPPTFGGGTKVEIKRSSS

SEQ ID NO: 312 - protein, 251 aa, synthetic construct
  REGION 1..251: note = TSC456 (anti-CD3) TSC394 E86D F87Y scFv
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRSSS

SEQ ID NO: 313 - protein, 247 aa, synthetic construct
  REGION 1..247: note = DRA222 (anti-CD3) scFv
QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITSSS

SEQ ID NO: 314 - protein, 35 aa, synthetic construct
  REGION 1..35: note = peptide linker
  VARIANT 6..35: note = Residues at positions 6-35 may be absent
GGGGSGGGGSGGGGSGGGGSGGGGSGGGGSGGGGS

SEQ ID NO: 315 - protein, 5 aa, synthetic construct
  REGION 1..5: note = peptide linker
GGGGS

SEQ ID NO: 316 - protein, 22 aa, synthetic construct
  REGION 1..22: note = peptide linker
GGGGSGGGGSGGGGSGGGGSPS

(19) United States
(12) Patent Application Publication
(10) Pub. No.: US 2024/0343801 A1
(43) Pub. Date: Oct. 17, 2024

(54) CD123 BINDING PROTEINS AND RELATED COMPOSITIONS AND METHODS

(71) Applicant: APTEVO RESEARCH AND DEVELOPMENT LLC, Seattle, WA (US)

(72) Inventors: Gabriela HERNANDEZ-HOYOS, Seattle, WA (US); Elaine T. SEWELL, Seattle, WA (US); Catherine J. MCMAHAN, Seattle, WA (US); David BIENVENUE, Seattle, WA (US); John W. BLANKENSHIP, Seattle, WA (US); Danielle MITCHELL, Seattle, WA (US); Peter PAVLIK, Seattle, WA (US)

(21) Appl. No.: 18/442,779

(22) Filed: Feb. 15, 2024

Related U.S. Application Data
(63) Continuation of application No. 17/557,440, filed on Dec. 21, 2021, now Pat. No. 11,939,392.
Application No. 17/557,440 is a continuation of application No. 16/335,561, filed on Mar. 21, 2019, now Pat. No. 11,242,400.
(60) Provisional application No. 62/466,192, filed on Mar. 2, 2017.
(60) Provisional application No. 62/397,736, filed on Sep. 21, 2016.

(51) Int. Cl. C07K 16/28; A61K 39/00; A61P 35/00; A61P 35/02; C07K 16/00; C07K 16/30; C12N 15/62

(52) U.S. Cl. CPC C07K 16/2809; A61P 35/00; A61P 35/02; C07K 16/2866; C07K 16/2896; C07K 16/3061; C12N 15/62; A61K 2039/505; A61K 2039/545; C07K 16/005; C07K 2317/24; C07K 2317/31; C07K 2317/33; C07K 2317/524; C07K 2317/526; C07K 2317/53; C07K 2317/565; C07K 2317/622; C07K 2317/73; C07K 2317/732; C07K 2317/734; C07K 2317/75; C07K 2317/90; C07K 2317/92; C07K 2317/94

(57) ABSTRACT

The present disclosure relates to protein molecules that specifically bind to CD123, which may have at least one humanized or human CD123-binding domain. Such molecules are useful for the treatment of cancer. The protein molecule binding to CD123 may have a second binding domain that binds to another target. In one embodiment, multi-specific polypeptide molecules bind both CD123-expressing cells and the T-cell receptor complex on T-cells to induce target-dependent T-cell cytotoxicity, activation, and proliferation. The disclosure also provides pharmaceutical compositions comprising the CD123-binding polypeptide molecules, nucleic acid molecules encoding these polypeptides and methods of making these molecules.

This application is a continuation application of U.S. application Ser. No. 17/557,440, filed Dec. 21, 2021 which is a continuation application of U.S. application Ser. No. 16/335,561, filed Mar. 21, 2019, now U.S. Pat. No. 11,242,400 issued Feb. 8, 2022, which is a U.S. national stage application of International Application No. PCT/US2017/052808, filed Sep. 21, 2017, which claims priority to and benefit of U.S. Provisional Patent Application No. 62/397,736, filed on Sep. 21, 2016, and U.S. Provisional Patent Application No. 62/466,192, filed on Mar. 2, 2017. The contents of each of these applications are herein incorporated by reference in their entirety.

### REFERENCE TO AN ELECTRONIC SEQUENCE LISTING

The contents of the electronic sequence listing (APVO_054_07US_SeqList_ST26.xml; Size: 439,447 bytes; and Date of Creation: Feb. 15, 2024) are herein incorporated by reference in its entirety.

### FIELD OF THE DISCLOSURE

The present disclosure relates to molecules that specifically bind to CD123, which may have at least one humanized CD123-binding domain. These molecules are useful for the characterization or treatment of disorders characterized by overexpression of CD123, such as cancer. A protein therapeutic binding to CD123 may be a monospecific protein therapeutic or a multi-specific protein therapeutic. A multi-specific protein therapeutic may bind both CD123-expressing cells and the T-cell receptor complex on T-cells to induce target-dependent T-cell cytotoxicity, activation and proliferation.

### BACKGROUND OF THE DISCLOSURE

CD123 is also known as the alpha chain of the human interleukin-3 (IL-3) receptor. CD123 is a type I transmembrane glycoprotein and is a member of the cytokine receptor superfamily. The interleukin-3 receptor is a heterodimer formed by CD123 and the beta chain (CD131). IL-3 binds to CD123, and signal transduction is provided by CD131. IL-3 regulates the function and production of hematopoietic and immune cells and stimulates endothelial cell proliferation (Testa et al., Biomark Res. 2:4 (2014)).

CD123 is overexpressed in many hematologic malignancies, including a subset of acute myeloid leukemia (AML), B-lymphoid leukemia, blastic plasmocytoid dendritic neoplasms (BPDCN) and hairy cell leukemia. Id. While most AML patients respond well to initial therapies, the majority of AML patients are ultimately diagnosed with relapsed or refractory disease (Ramos et al., J. Clin. Med. 4:665-695 (2015)). There is a need for molecules targeting CD123 with increased efficiency and potency and reduced adverse effects and that may be used to treat disorders associated with dysregulation of CD123.

### SUMMARY OF THE DISCLOSURE

In some embodiments, the disclosure encompasses a recombinant polypeptide comprising a CD123-binding domain, wherein the CD123-binding domain comprises (i) an immunoglobulin light chain variable region comprising LCDR1, LCDR2, and LCDR3, and (ii) an immunoglobulin heavy chain variable region comprising HCDR1, HCDR2, and HCDR3, wherein the LCDR1 comprises an amino acid sequence as set forth in SEQ ID NO:6 or a sequence that differs from SEQ ID NO:6 by at least one amino acid substitution; the LCDR2 comprises an amino acid sequence as set forth in SEQ ID NO:8 or a sequence that differs from SEQ ID NO:8 by at least one amino acid substitution; the LCDR3 comprises an amino acid sequence as set forth in SEQ ID NO:10 or a sequence that differs from SEQ ID NO:10 by at least one amino acid substitution; the HCDR1 comprises an amino acid sequence as set forth in SEQ ID NO:12 or a sequence that differs from SEQ ID NO:12 by at least one amino acid substitution; the HCDR2 comprises an amino acid sequence as set forth in SEQ ID NO:14 or a sequence that differs from SEQ ID NO:14 by at least one amino acid substitution; and the HCDR3 comprises an amino acid sequence as set forth in SEQ ID NO:16 or a sequence that differs from SEQ ID NO:16 by at least one amino acid substitution. For instance, the disclosure encompasses a recombinant polypeptide comprising a CD123-binding domain, wherein the CD123-binding domain comprises (i) an immunoglobulin light chain variable region comprising LCDR1, LCDR2, and LCDR3, and (ii) an immunoglobulin heavy chain variable region comprising HCDR1, HCDR2, and HCDR3, wherein the LCDR1 comprises an amino acid sequence as set forth in SEQ ID NO:6 or a sequence that differs from SEQ ID NO:6 by one or two amino acid substitutions; the LCDR2 comprises an amino acid sequence as set forth in SEQ ID NO:8 or a sequence that differs from SEQ ID NO:8 by one or two amino acid substitutions; the LCDR3 comprises an amino acid sequence as set forth in SEQ ID NO:10 or a sequence that differs from SEQ ID NO:10 by one or two amino acid substitutions; the HCDR1 comprises an amino acid sequence as set forth in SEQ ID NO:12 or a sequence that differs from SEQ ID NO:12 by one or two amino acid substitutions; the HCDR2 comprises an amino acid sequence as set forth in SEQ ID NO:14 or a sequence that differs from SEQ ID NO:14 by one or two amino acid substitutions; and the HCDR3 comprises an amino acid sequence as set forth in SEQ ID NO:16 or a sequence that differs from SEQ ID NO:16 by one or two amino acid substitutions. The invention includes a recombinant polypeptide with a CD-123 binding domain that comprises (a) the LCDR1 amino acid sequence as set forth in SEQ ID NO:6; (b) the LCDR2 amino acid sequence as set forth in SEQ ID NO:8; (c) the LCDR3 amino acid sequence as set forth in SEQ ID NO:10; (d) the HCDR1 amino acid sequence as set forth in SEQ ID NO:12; (e) the HCDR2 amino acid sequence as set forth in SEQ ID NO:14; and (f) the HCDR3 amino acid sequence as set forth in SEQ ID NO:16. A recombinant polypeptide that binds CD123 may comprises (i) an immunoglobulin light chain variable region comprising an amino acid sequence that is at least 88%, at least 90%, at least 92%, at least 95%, at least 97%, at least 98% or at least 99% identical to the amino acid sequence set forth in SEQ ID NO:2; and (ii) an immunoglobulin heavy chain variable region comprising an amino acid sequence that is at least 85%, at least 90%, at least 92%, at least 95%, at least 97%, at least 98% or at least 99% identical to the amino acid sequence set forth in SEQ ID NO:4. For instance, the invention includes a recombinant polypeptide of claim 1, wherein the CD123-binding domain comprises: (i) the immunoglobulin light chain variable region comprising the amino acid sequence of SEQ ID NO:2; and (ii) the immunoglobulin heavy chain variable region comprising the amino acid sequence of SEQ ID NO:4.

In another embodiment, the invention includes a recombinant CD123-binding polypeptide comprising a CD123-binding domain, wherein the binding domain comprises an immunoglobulin light chain variable region comprising LCDR1, LCDR2, LCDR3 and an immunoglobulin heavy chain variable region comprising HCDR1, HCDR2 and HCDR3, and wherein (i) (a) the HCDR1 comprises an amino acid sequence as set forth in SEQ ID NO:28 or a sequence that differs from SEQ ID NO:28 by at least one amino acid substitution; (b) the HCDR2 comprises an amino acid sequence as set forth in SEQ ID NO:30 or a sequence that differs from SEQ ID NO:30 by at least one amino acid substitution; and (c) the HCDR3 comprises an amino acid sequence as set forth in SEQ ID NO:32 or a sequence that differs from SEQ ID NO:32 by at least one amino acid substitution; or (ii) (a) the HCDR1 comprises an amino acid sequence as set forth in SEQ ID NO:36 or a sequence that differs from SEQ ID NO:36 by at least one amino acid substitution; (b) the HCDR2 comprises an amino acid sequence as set forth in SEQ ID NO:38 or a sequence that differs from SEQ ID NO:38 by at least one amino acid substitution; and (c) the HCDR3 comprises an amino acid sequence as set forth in SEQ ID NO:40 or a sequence that differs from SEQ ID NO:40 by at least one amino acid substitution; or (iii)(a) the HCDR1 comprises an amino acid sequence as set forth in SEQ ID NO:44 or a sequence that differs from SEQ ID NO:44 by at least one amino acid substitution;(b) the HCDR2 comprises an amino acid sequence as set forth in SEQ ID NO:46 or a sequence that differs from SEQ ID NO:46 by at least one amino acid substitution; and (c) the HCDR3 comprises an amino acid sequence as set forth in SEQ ID NO:48 or a sequence that differs from SEQ ID NO:48 by at least one amino acid substitution; or (iv) (a) the HCDR1 comprises an amino acid sequence as set forth in SEQ ID NO:100 or a sequence that differs from SEQ ID NO:100 by at least one amino acid substitution; (b) the HCDR2 comprises an amino acid sequence as set forth in SEQ ID NO:102 or a sequence that differs from SEQ ID NO:102 by at least one amino acid substitution; and (c) the HCDR3 comprises an amino acid sequence as set forth in SEQ ID NO:104 or a sequence that differs from SEQ ID NO:104 by at least one amino acid substitution; or (v)(a) the HCDR1 comprises an amino acid sequence as set forth in SEQ ID NO:108 or a sequence that differs from SEQ ID NO:108 by at least one amino acid substitution;(b) the HCDR2 comprises an amino acid sequence as set forth in SEQ ID NO:110 or a sequence that differs from SEQ ID NO: 110 by at least one amino acid substitution; and (c) the HCDR3 comprises an amino acid sequence as set forth in SEQ ID NO: 112 or a sequence that differs from SEQ ID NO: 112 by at least one amino acid substitution; or (vi)(a) the HCDR1 comprises an amino acid sequence as set forth in SEQ ID NO: 116 or a sequence that differs from SEQ ID NO: 116 by at least one amino acid substitution;(b) the HCDR2 comprises an amino acid sequence as set forth in SEQ ID NO:118 or a sequence that differs from SEQ ID NO:118 by at least one amino acid substitution; and (c) the HCDR3 comprises an amino acid sequence as set forth in SEQ ID NO:120 or a sequence that differs from SEQ ID NO:120 by at least one amino acid substitution; or (vii)(a) the HCDR1 comprises an amino acid sequence as set forth in SEQ ID NO:124 or a sequence that differs from SEQ ID NO:124 by at least one amino acid substitution; (b) the HCDR2 comprises an amino acid sequence as set forth in SEQ ID NO:126 or a sequence that differs from SEQ ID NO:126 by at least one amino acid substitution; and (c) the HCDR3 comprises an amino acid sequence as set forth in SEQ ID NO:128 or a sequence that differs from SEQ ID NO:128 by at least one amino acid substitution. The invention includes recombinant polypeptides comprising a heavy chain variable region as defined by (i)-(vii) and a light chain variable region comprising (a) the LCDR1 comprises an amino acid sequence as set forth in SEQ ID NO:22 or a sequence that differs from SEQ ID NO:22 by at least one amino acid substitution; (b) the LCDR2 comprises an amino acid sequence as set forth in SEQ ID NO:24 or a sequence that differs from SEQ ID NO:24 by at least one amino acid substitution; and (c) the LCDR3 comprises an amino acid sequence as set forth in SEQ ID NO:26 or a sequence that differs from SEQ ID NO:26 by at least one amino acid substitution. For instance, the invention includes a recombinant polypeptide comprising a light chain variable region comprises an amino acid sequence that is at least 88%, at least 90%, at least 92%, at least 95%, at least 97%, at least 98% or at least 99% identical to the amino acid sequence set forth in SEQ ID NO:18, and a heavy chain variable region comprises an amino acid sequence that is at least 85%, at least 90%, at least 92%, at least 95%, at least 97%, at least 98% or at least 99% identical to the amino acid sequence set forth in SEQ ID NO:20, 34, 42, 98, 106, 114, or 122.

In certain embodiments, a recombinant CD123-binding polypeptide comprises (a) the LCDR1 has an amino acid sequence set forth in SEQ ID NO:54 or a sequence that differs from SEQ ID NO:54 by at least one amino acid substitution; (b) the LCDR2 has an amino acid sequence set forth in SEQ ID NO:56 or a sequence that differs from SEQ ID NO:56 by at least one amino acid substitution; (c) the LCDR3 has an amino acid sequence set forth in SEQ ID NO:58 or a sequence that differs from SEQ ID NO:58 by at least one amino acid substitution; (d) the HCDR1 has an amino acid sequence set forth in SEQ ID NO:60 or a sequence that differs from SEQ ID NO:60 by at least one amino acid substitution; (e) the HCDR2 has an amino acid sequence set forth in SEQ ID NO:61 or a sequence that differs from SEQ ID NO:61 by at least one amino acid substitution; and (f) the HCDR3 has an amino acid sequence set forth in SEQ ID NO:62 or a sequence that differs from SEQ ID NO:62 by at least one amino acid substitution. For instance, the invention includes a recombinant polypeptide comprising a CD123-binding domain, wherein (i) the immunoglobulin light chain variable region comprises an amino acid sequence that is at least 88%, at least 90%, at least 92%, at least 95%, at least 97%, at least 98% or at least 99% identical to the amino acid sequence set forth in SEQ ID NO:50; and (ii) the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:52.

In another embodiment, the recombinant polypeptide comprising a CD123-binding domain, comprises (a) the LCDR1 comprises an amino acid sequence as set forth in SEQ ID NO:70 or a sequence that differs from SEQ ID NO:70 by at least one amino acid substitution; (b) the LCDR2 comprises an amino acid sequence as set forth in SEQ ID NO:72 or a sequence that differs from SEQ ID NO:72 by at least one amino acid substitution; (c) the LCDR3 comprises an amino acid sequence as set forth in SEQ ID NO:74 or a sequence that differs from SEQ ID NO:74 by at least one amino acid substitution; (d) the HCDR1 comprises an amino acid sequence as set forth in SEQ ID NO:76 or a sequence that differs from SEQ ID NO:76 by at least one amino acid substitution; (e) the HCDR2 comprises an amino acid sequence as set forth in SEQ ID NO:78 or a sequence that differs from SEQ ID NO:78 by at least one amino acid substitution; and (f) the HCDR3 comprises an amino acid sequence as set forth in SEQ ID NO:80 or a sequence that differs from SEQ ID NO:80 by at least one amino acid substitution. For instance, the invention includes a recombinant polypeptide comprising the variable regions set forth in SEQ ID Nos: 66 and 68.

In certain embodiments, the recombinant polypeptide comprises (a) the LCDR1 comprises an amino acid sequence as set forth in SEQ ID NO:86 or a sequence that differs from SEQ ID NO:86 by at least one amino acid substitution; (b) the LCDR2 comprises an amino acid sequence as set forth in SEQ ID NO:88 or a sequence that differs from SEQ ID NO:88 by at least one amino acid substitution; (c) the LCDR3 comprises an amino acid sequence as set forth in SEQ ID NO:90 or a sequence that differs from SEQ ID NO:90 by at least one amino acid substitution; (d) the HCDR1 comprises an amino acid sequence as set forth in SEQ ID NO:92 or a sequence that differs from SEQ ID NO:92 by at least one amino acid substitution; (e) the HCDR2 comprises an amino acid sequence as set forth in SEQ ID NO:94 or a sequence that differs from SEQ ID NO:94 by at least one amino acid substitution; and (f) the HCDR3 comprises an amino acid sequence as set forth in SEQ ID NO:96 or a sequence that differs from SEQ ID NO:96 by at least one amino acid substitution. For instance, the invention includes a polypeptide comprising the variable domains as set forth in SEQ ID NO:82 and 84.

(ii) the immunoglobulin heavy chain variable region comprising an amino acid sequence that is at least 85%, at least 90%, at least 92%, at least 95%, at least 97%, at least 98% or at least 99% identical to the amino acid sequence set forth in SEQ ID NO:84.

The polypeptides of the invention bind to human CD123 with specificity. In certain embodiments, the polypeptides bind to non-human primate CD123. In further embodiments, the polypeptides bind to cynomolgous monkey CD123. The invention includes a CD123-binding polypeptide comprising a human CD123-binding domain and a CD123-binding polypeptide comprising a humanized CD123-binding domain. In one embodiment, the CD123-binding domain is a single chain variable fragment (scFv).

The recombinant polypeptides of the invention include bi-specific polypeptides comprising a second binding domain. In one embodiment, the second binding domain binds a T-cell, CD3, CD3ε or a T-cell receptor (TCR) complex or a component of a T-cell receptor complex with specificity. For instance, the invention includes recombinant CD123-binding polypeptides comprising a CD3 binding scFv. The invention includes combining any one of the CD123-binding scFvs disclosed with a disclosed CD3 binding scFv. In one embodiment, the polypeptide comprises, from (i) the CD123-binding domain, (ii) a hinge region, (iii) an immunoglobulin constant region, (iv) a carboxyl-terminus linker, and (v) the second binding domain. For instance, the invention includes a recombinant polypeptide comprising, in order from amino to carboxyl terminus, (i) the CD123-binding domain, (ii) the hinge region, (iii) the immunoglobulin constant region, (iv) the carboxyl-terminus linker, and (v) the second binding domain.

In one embodiment of the invention, the bi-specific polypeptides comprising a CD123 binding domain and a CD3 binding domain comprise a modified immunoglobulin constant region engineered to exhibit no to minimal antibody-dependent cell-mediated cytotoxicity (ADCC) activity and/or complement-dependent cytotoxicity (CDC) activity. In one embodiment of the invention, the CD3 binding domain is derived from a monoclonal antibody selected from CRIS-7, HuM291 and I2C.

In certain embodiments of the invention, the CD123-binding polypeptide comprises (i) an amino acid sequence at least about 93% identical, at least about 95% identical, at least about 97% identical, at least about 98% identical or at least about 99% identical to the amino acid sequence of SEQ ID NO:130; (ii) an amino acid sequence at least about 93% identical, at least about 95% identical, at least about 97% identical, at least about 98% identical or at least about 99% identical to the amino acid sequence of SEQ ID NO:132; (iii) an amino acid sequence at least about 93% identical, at least about 95% identical, at least about 97% identical, at least about 98% identical or at least about 99% identical to the amino acid sequence of SEQ ID NO:134; (iv) an amino acid sequence at least about 93% identical, at least about 95% identical, at least about 97% identical, at least about 98% identical or at least about 99% identical to the amino acid sequence of SEQ ID NO:136; (v) an amino acid sequence at least about 93% identical, at least about 95% identical, at least about 97% identical, at least about 98% identical or at least about 99% identical to the amino acid sequence of SEQ ID NO:138; (vi) an amino acid sequence at least about 93% identical, at least about 95% identical, at least about 97% identical, at least about 98% identical or at least about 99% identical to the amino acid sequence of SEQ ID NO:140; (vii) an amino acid sequence at least about 93% identical, at least about 95% identical, at least about 97% identical, at least about 98% identical or at least about 99% identical to the amino acid sequence of SEQ ID NO:142; (viii) an amino acid sequence at least about 93% identical, at least about 95% identical, at least about 97% identical, at least about 98% identical or at least about 99% identical to the amino acid sequence of SEQ ID NO:144; (iv) an amino acid sequence at least about 93% identical, at least about 95% identical, at least about 97% identical, at least about 98% identical or at least about 99% identical to the amino acid sequence of SEQ ID NO:146; (x) amino acid sequence at least about 93% identical, at least about 95% identical, at least about 97% identical, at least about 98% identical or at least about 99% identical to the amino acid sequence of SEQ ID NO:148; (xi) an amino acid sequence at least about 93% identical, at least about 95% identical, at least about 97% identical, at least about 98% identical or at least about 99% identical to the amino acid sequence of SEQ ID NO:150; (xii) an amino acid sequence at least about 93% identical, at least about 95% identical, at least about 97% identical, at least about 98% identical or at least about 99% identical to the amino acid sequence of SEQ ID NO:152; (xiii) an amino acid sequence at least about 93% identical, at least about 95% identical, at least about 97% identical, at least about 98% identical or at least about 99% identical to the amino acid sequence of SEQ ID NO:154; or (xiv) an amino acid sequence at least about 93% identical, at least about 95% identical, at least about 97% identical, at least about 98% identical or at least about 99% identical to the amino acid sequence of SEQ ID NO:156.

In one embodiment of the invention, the recombinant polypeptide induces redirected T-cell cytotoxicity (RTCC). In certain embodiments, the recombinant polypeptide induces T-cell activation or T-cell proliferation. In certain embodiments, the polypeptide induces T-cell-dependent lysis of CD123-expressing cells.

In certain embodiments of the invention, the bi-specific polypeptide comprising a CD123-binding domain and a CD3-binding domain when bound to a CD3 protein on a T cell induces reduced cytokine release from said T cell as compared to an OKT3 antibody control. In certain embodiments of the invention, the bi-specific polypeptide comprising a CD123-binding domain and a CRIS-7 derived CD3-binding domain induces reduced cytokine release from said T cell as compared to a bi-specific comprising an CD3-binding domain derived from OKT3 or I2C. In certain embodiments of the invention, the bi-specific polypeptide comprising a CD123-binding domain (e.g., a CD123-binding domain comprising an amino acid sequence at least 93%, at least 95%, at least 96%, at least 97%, at least 98%, at least 99% or 100% identical to SEQ ID NO: 2 and/or SEQ ID NO:4) and a CRIS-7 derived CD3-binding domain and in the scFv-Fc-scFv format induces reduced cytokine release in a non-human primate or human as compared to a bi-specific polypeptide comprising a CD123-binding domain and I2C derived CD3-binding domain in an scFv-scFv or diabody format.

In certain embodiments of the invention, the bispecific polypeptide comprising a CD123-binding domain and a CD3-binding domain (for instance, a recombinant polypeptide comprising an amino acid sequence at least 93%, at least 95%, at least 96%, at least 97%, at least 98%, at least 99% or 100% identical to SEQ ID NO:130 or SEQ ID NO:132) induces reduced cytokine release in a non-human primate or human as compared to MGD006 or TRI168. In one embodiment of the invention, a recombinant polypeptide comprising an amino acid sequence at least 93%, at least 95%, at least 96%, at least 97%, at least 98%, at least 99% or 100% identical to SEQ ID NO:130 or SEQ ID NO:132 induces reduced levels of IFNγ, IL-2, TNFα and/or IL-10 as compared to MGD006 or TRI168.

The disclosure encompasses an isolated nucleic acid molecule encoding a CD123-binding polypeptide described herein or a portion of said CD123-binding polypeptide. The isolated nucleic acid molecule may comprise a nucleotide sequence set forth in SEQ ID NO:1, SEQ ID NO:3, SEQ ID NO:17, SEQ ID NO:19, SEQ ID NO:33, SEQ ID NO:41, SEQ ID NO:49, SEQ ID NO:51, SEQ ID NO:65, SEQ ID NO:67, SEQ ID NO:81, SEQ ID NO:83, SEQ ID NO:97, SEQ ID NO:105, SEQ ID NO:113, SEQ ID NO:121, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, or SEQ ID NO:155.

The disclosure relates to an expression vector comprising a nucleic acid segment encoding a CD123-binding polypeptide described herein, wherein the nucleic acid segment is operatively linked to regulatory sequences suitable for expression of the nucleic acid segment in a host cell. The nucleic acid segment of the expression vector may comprise a nucleotide sequence set forth in SEQ ID NO:1, SEQ ID NO:3, SEQ ID NO:17, SEQ ID NO:19, SEQ ID NO:33, SEQ ID NO:41, SEQ ID NO:49, SEQ ID NO:51, SEQ ID NO:65, SEQ ID NO:67, SEQ ID NO:81, SEQ ID NO:83, SEQ ID NO:97, SEQ ID NO:105, SEQ ID NO:113, SEQ ID NO:121, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, or SEQ ID NO:155.

The disclosure includes a recombinant host cell comprising an expression vector described herein.

The disclosure relates to a method for producing a CD123-binding polypeptide, the method comprising culturing a recombinant host cell comprising an expression vector described herein under conditions whereby the nucleic acid segment of the vector is expressed, thereby producing the CD123-binding polypeptide. The method may further comprise recovering the CD123-binding polypeptide.

In some embodiments, the disclosure relates to a pharmaceutical composition comprising a CD123-binding polypeptide described herein and a pharmaceutically acceptable carrier, diluent, or excipient. The pharmaceutical composition may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form. The pharmaceutical composition formulated as an oral unit dosage form may be selected from the group consisting of: tablets, pills, pellets, capsules, powders, lozenges, granules, solutions, suspensions, emulsions, syrups, elixirs, sustained-release formulations, aerosols, and sprays.

The disclosure also relates to a method for inducing redirected T-cell cytotoxicity (RTCC) against a cell expressing CD123, the method comprising: contacting said CD123-expressing cell with a CD123-binding polypeptide described herein, wherein the second binding domain specifically binds a T-cell, CD3, CD3ε or a T-cell receptor (TCR) complex or a component of a T-cell receptor complex; and wherein said contacting is under conditions whereby RTCC against the CD123-expressing cell is induced.

The disclosure encompasses a method for inducing T-cell dependent lysis of a cell expressing CD123, the method comprising: contacting said CD123-expressing cell with a CD123-binding polypeptide or CD123-binding protein described herein, wherein the second binding domain specifically binds a T-cell, CD3, CD3ε or a T-cell receptor (TCR) complex or a component of a T-cell receptor complex; and wherein said contacting is under conditions whereby T-cell dependent lysis of the CD123-expressing cell is induced.

The disclosure encompasses a method for treating a disorder (e.g., cancer) in a subject, wherein said disorder is characterized by overexpression of CD123, the method comprising administering to the subject a therapeutically effective amount of a CD123-binding polypeptide described herein. The disclosure also relates to a CD123-binding polypeptide described herein for the manufacture of a medicament for treatment of a disorder (e.g., cancer) in a subject, wherein said disorder is characterized by overexpression of CD123. The disclosure includes a CD123-binding polypeptide described herein for use in treating a disorder (e.g., cancer) in a subject, wherein said disorder is characterized by overexpression of CD123. The cancer treated by the CD123-binding polypeptides described herein may be acute myeloid leukemia (AML), B-lymphoid leukemia, blastic plasmocytoid dendritic neoplasm (BPDCN), or hairy cell leukemia.

These and other embodiments and/or other aspects of the disclosure will become evident upon reference to the following detailed description of the disclosure and the attached drawings.

### BRIEF DESCRIPTION OF THE DRAWINGS

FIG. 1A-FIG. 1D show the binding of 13 different bispecific anti-CD123×anti-CD3ε molecules (TRI123, TRI125, TRI126, TRI127, TRI128, TRI129, TRI130, TRI131, TRI132, TRI134, TRI137, TRI138 and TRI139) in three independent experiments to the CD123 (+) Molm-13 human tumor cell line.

FIG. 2A-FIG. 2C show the binding of 13 different bispecific anti-CD123×anti-CD3ε molecules (TRI123, TRI125, TRI126, TRI127, TRI128, TRI129, TRI130, TRI131, TRI132, TRI134, TRI137, TRI138 and TRI139) in two independent experiments to CHO cells stably expressing cynomolgus CD123 protein.

FIG. 3A-FIG. 3C show the results from chromium-51 release assays with the Molm-13 cell line measured at 4 hours using 13 different bispecific anti-CD123×anti-CD3ε molecules (TRI123, TRI125, TRI126, TRI127, TRI128, TRI129, TRI130, TRI131, TRI132, TRI134, TRI137, TRI138 and TRI139) in two independent experiments. All of the bispecific anti-CD123×anti-CD3ε molecules showed efficient target cell lysis at 4 hours ranging between 24-48% maximum specific lysis.

FIG. 4A-FIG. 4D show an induction of T-cell proliferation at low concentrations (10 pM).

FIG. 5A-FIG. 5D show target dependent activation of CD4⁺ and CD8⁺ T-cells in the presence of Molm-13 cells.

FIG. 6 shows an example of SPR analysis of TRI-130 at different ECD concentrations.

FIG. 7 shows concentration versus time curves for TRI129 and TRI130 bispecific molecules.

FIG. 8 shows reduction in tumor volume in a mouse tumor model treated with TRI129.

FIG. 9 shows reduction in tumor volume in a mouse tumor model treated with TRI130.

FIG. 10 shows that treatment of a mouse tumor model with TRI129 or TRI130 results in increase in survival.

FIG. 11 is an illustration of a recombinant CD123-binding polypeptide capable of RTCC in the CD123-binding domain—hinge domain—immunoglobulin constant domain—CD3 binding domain configuration.

FIG. 12 shows the results from a chromium-51 release assay with the Molm-13, KG-1a and Daudi cell lines measured at 4 hours using the TRI130 bispecific anti-CD123×anti-CD3ε molecule. This assay measured the cytotoxicity of TRI130 incubated with CD123-positive or C123-negative tumor cell lines and purified human T-cells.

FIG. 13 shows the WinNonlin® non-compartmental (NCA) estimates and fit of half-life (HL) for treatment groups of cynomolgus monkeys treated with TRI130, as described in Example 14.

FIG. 14 shows a graph depicting the change in lymphocyte population over time in cynomolgus monkeys treated with TRI130, as described in Example 14.

FIG. 15 shows a graph depicting the change in basophil population over time in cynomolgus monkeys treated with TRI130, as described in Example 14.

FIG. 16 shows a graph depicting the tumor burden as measured by bioluminescence levels over time in a disseminated xenograft mouse model of acute myeloid leukemia (AML), as described in Example 15.

FIG. 17 shows bioluminescent images of tumor burden in mice at day 14.

FIG. 18 shows the results of assays measuring TRI130- and TRI168-induced T-cell activation with CD123⁺ Molm-13 target cells, as described in Example 17.

FIG. 19 shows the results of assays measuring TRI130- and TRI168-induced T-cell cytotoxicity of CD123⁺ Molm-13 target cells, as described in Example 17.

FIG. 20 shows the results of assays measuring TRI130- and TRI168-induced T-cell cytokine release with CD123⁺ Molm-13 target cells, as described in Example 17.

FIG. 21 shows the results of assays measuring TRI130- and TRI168-induced T-cell cytokine release in peripheral blood mononuclear cells (PBMC) cultures, as described in Example 17.

FIG. 22 shows the results of assays measuring cytotoxicity of TRI130 incubated with AML cell samples from AML subjects, as described in Example 18.

### DETAILED DESCRIPTION OF THE DISCLOSURE

The disclosure provides binding domains that specifically bind to CD123 (also known as interleukin-3 receptor alpha chain) and binding molecules (e.g. polypeptides and proteins) that specifically bind to CD123. These binding molecules may bind specifically to CD123 and to another target. Administration of a therapeutically effective amount of a CD123-binding polypeptide or protein to a patient in need thereof is useful for treatment of certain disorders associated with the over-expression of CD123, including certain cancers. In one embodiment, a CD123-binding polypeptide or protein binds both a target cell over-expressing CD123 and a T-cell, thereby “cross-linking” the target cell over-expressing CD123 and the T-cell. The binding of both domains to their targets elicits potent target-dependent redirected T-cell cytotoxicity (RTCC) (e.g., induces target-dependent T-cell cytotoxicity, T-cell activation and/or T-cell proliferation). The CD123-binding therapeutics of the disclosure offer various advantages in treating patients, for example, effective binding to CD123, efficient induction of RTCC activity, reduced levels of cytokine release and/or a lower risk of adverse events (e.g., toxicity). In certain aspects, the CD123-binding proteins bind to CD123 more effectively in certain formats (e.g., scFv compared to parent antibody) and/or certain orientations (e.g., VL-VH compared to VH-VL), leading to higher potency and improved utility in treating disorders associated with over-expression of CD123.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. In addition, it should be understood that the polypeptides comprising the various combinations of the components (e.g., domains or regions) and substituents described herein, are disclosed by the present application to the same extent as if each polypeptide was set forth individually. Thus, selection of particular components of individual polypeptides is within the scope of the present disclosure.

As used herein, the term “binding domain” or “binding region” refers to the domain, region, portion, or site of a protein, polypeptide, oligopeptide, or peptide or antibody or binding domain derived from an antibody that possesses the ability to specifically recognize and bind to a target molecule, such as an antigen, ligand, receptor, substrate, or inhibitor (e.g., CD123, CD3). Exemplary binding domains include single-chain antibody variable regions (e.g., domain antibodies, sFv, scFv, scFab), receptor ectodomains, and ligands (e.g., cytokines, chemokines). In certain embodiments, the binding domain comprises or consists of an antigen binding site (e.g., comprising a variable heavy chain sequence and variable light chain sequence or three light chain complementary determining regions (CDRs) and three heavy chain CDRs from an antibody placed into alternative framework regions (FRs) (e.g., human FRs optionally comprising one or more amino acid substitutions). A variety of assays are known for identifying binding domains of the present disclosure that specifically bind a particular target, including Western blot, ELISA, phage display library screening, and BIACORE® interaction analysis. As used herein, a CD123-binding polypeptide can have a “first binding domain” and, optionally, a “second binding domain.” In certain embodiments, the “first binding domain” is a CD123-binding domain and the format is an antibody or antibody-like protein or domain. In certain embodiments comprising both the first and second binding domains, the second binding domain is a T-cell binding domain such as a scFv derived from a mouse monoclonal antibody (e.g., CRIS-7) or phage display (e.g., I2C) that binds to a T-cell surface antigen (e.g., CD3). In other embodiments, the second binding domain is a second CD123-binding domain. In yet other embodiments, the second binding domain is a binding domain other than a CD123-binding domain or a T-cell binding domain.

“Cytokine release” or “cytokine storm” or “infusion reaction” refers to the release of cytokines from T-cells. When cytokines are released into the circulation, systemic symptoms such as fever, nausea, chills, hypotension, tachycardia, asthenia, headache, rash, scratchy throat, and dyspnea can result. Some patients may experience severe, life-threatening reactions that result from massive release of cytokines. “Reduced” cytokine release refers to the to the reduction in the release of at least one cytokine (e.g., IFNγ, IL-2, TNFα and/or IL-10) following administration of a recombinant polypeptide of the invention as compared to the OKT-3 antibody or other CD3 binding bispecific molecule. Reduced cytokine release can be measured using in vitro assays or in vivo.

A binding domain or protein “specifically binds” a target if it binds the target with an affinity or Kₐ (i.e., an equilibrium association constant of a particular binding interaction with units of 1/M) equal to or greater than 10⁵ M⁻¹, while not significantly binding other components present in a test sample. Binding domains can be classified as “high affinity” binding domains and “low affinity” binding domains. “High affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding domains refer to those binding domains with a Kₐ of up to 10⁷ M⁻¹, up to 10⁶ M⁻¹, up to 10⁵ M⁻¹. Alternatively, affinity can be defined as an equilibrium dissociation constant (K_{d}) of a particular binding interaction with units of M (e.g., 10⁻⁵ M to 10⁻¹³ M). Affinities of binding domain polypeptides and single chain polypeptides according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

“CD3” is known in the art as a multi-protein complex of six chains (see, e.g., Abbas and Lichtman, 2003; Janeway et al., p. 172 and 178, 1999), which are subunits of the T-cell receptor complex. In mammals, the CD3 subunits of the T-cell receptor complex are a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T-cell receptor chains. The intracellular tails of the CD3γ, CD3δ, and CD3ε chains each contain a single conserved motif known as an immunoreceptor tyrosine-based activation motif or ITAM, whereas each CD3ζ chain has three. It is believed the ITAMs are important for the signaling capacity of a TCR complex. CD3ζ as used in the present disclosure can be from various animal species, including human, monkey, mouse, rat, or other mammals.

As used herein, a “conservative substitution” is recognized in the art as a substitution of one amino acid for another amino acid that has similar properties. Exemplary conservative substitutions are well-known in the art (see, e.g., WO 97/09433, page 10, published Mar. 13, 1997; Lehninger, Biochemistry, Second Edition; Worth Publishers, Inc. NY:NY (1975), pp. 71-77; Lewin, Genes IV, Oxford University Press, NY and Cell Press, Cambridge, MA (1990), p. 8). In certain embodiments, a conservative substitution includes a leucine to serine substitution.

As used herein, the term “derivative” refers to a modification of one or more amino acid residues of a peptide by chemical or biological means, either with or without an enzyme, e.g., by glycosylation, alkylation, acylation, ester formation, or amide formation.

As used herein, a polypeptide or amino acid sequence “derived from” a designated polypeptide or protein refers to the origin of the polypeptide. In certain embodiments, the polypeptide or amino acid sequence which is derived from a particular sequence (sometimes referred to as the “starting” or “parent” or “parental” sequence) has an amino acid sequence that is essentially identical to the starting sequence or a portion thereof, wherein the portion consists of at least 10-20 amino acids, at least 20-30 amino acids, or at least 30-50 amino acids, or at least 50-150 amino acids, or which is otherwise identifiable to one of ordinary skill in the art as having its origin in the starting sequence. For example, a binding domain can be derived from an antibody, e.g., a Fab, F(ab′)2, Fab′, scFv, single domain antibody (sdAb), etc.

Polypeptides derived from another polypeptide can have one or more mutations relative to the starting polypeptide, e.g., one or more amino acid residues which have been substituted with another amino acid residue or which has one or more amino acid residue insertions or deletions. The polypeptide can comprise an amino acid sequence which is not naturally occurring. Such variations necessarily have less than 100% sequence identity or similarity with the starting polypeptide. In one embodiment, the variant will have an amino acid sequence from about 60% to less than 100% amino acid sequence identity or similarity with the amino acid sequence of the starting polypeptide. In another embodiment, the variant will have an amino acid sequence from about 75% to less than 100%, from about 80% to less than 100%, from about 85% to less than 100%, from about 90% to less than 100%, from about 95% to less than 100% amino acid sequence identity or similarity with the amino acid sequence of the starting polypeptide.

As used herein, unless otherwise provided, a position of an amino acid residue in a variable region of an immunoglobulin molecule is numbered according to the IMGT numbering convention (Brochet, X, et al, Nucl. Acids Res. (2008) 36, W503-508) and a position of an amino acid residue in a constant region of an immunoglobulin molecule is numbered according to EU nomenclature (Ward et al., 1995 Therap. Immunol. 2:77-94). Other numbering conventions are known in the art (e.g., the Kabat numbering convention (Kabat, Sequences of Proteins of Immunological Interest, 5^{th} ed. Bethesda, MD: Public Health Service, National Institutes of Health (1991)).

As used herein, the term “dimer” refers to a biological entity that consists of two subunits associated with each other via one or more forms of intramolecular forces, including covalent bonds (e.g., disulfide bonds) and other interactions (e.g., electrostatic interactions, salt bridges, hydrogen bonding, and hydrophobic interactions), and is stable under appropriate conditions (e.g., under physiological conditions, in an aqueous solution suitable for expressing, purifying, and/or storing recombinant proteins, or under conditions for non-denaturing and/or non-reducing electrophoresis). A “heterodimer” or “heterodimeric protein,” as used herein, refers to a dimer formed from two different polypeptides. A heterodimer does not include an antibody formed from four polypeptides (i.e., two light chains and two heavy chains). A “homodimer” or “homodimeric protein,” as used herein, refers to a dimer formed from two identical polypeptides. The recombinant polypeptides of the invention exist primarily in a dimerized form. All disclosure of the polypeptide, including characteristics and activities (such as binding and RTCC) should be understood to include the polypeptide in its dimer form as well as other multimeric forms.

In some embodiments, a CD123-binding polypeptide comprises, in order from amino-terminus to carboxyl-terminus or in order from carboxyl-terminus to amino-terminus, (i) the CD123-binding domain, (ii) a hinge region, (iii) an immunoglobulin constant region, (iv) a carboxyl-terminus linker (or an amino-terminus linker), and (v) a second binding domain. As used herein and depending on context, a “hinge region” or a “hinge” refers to a polypeptide region between a binding domain (e.g., a CD123-binding domain) and an immunoglobulin constant region. As used herein and depending on context, a “linker” may refer to (1) a polypeptide region between V_{H} and V_{L} regions in a single-chain Fv (scFv) or (2) a polypeptide region between an immunoglobulin constant region and a second binding domain in a CD123-binding polypeptide comprising two binding domains. A polypeptide region between an immunoglobulin constant region and a second binding domain in a CD123-binding polypeptide comprising two binding domains may also be referred to as a “carboxyl-terminus linker” or an “amino-terminus linker.” Non-limiting examples of carboxyl-terminus and amino-terminus linkers include flexible linkers comprising glycine-serine (e.g., (Gly₄Ser)) repeats (SEQ ID NO: 315), and linkers derived from (a) an interdomain region of a transmembrane protein (e.g., a type I transmembrane protein); (b) a stalk region of a type II C-lectin; or (c) an immunoglobulin hinge. Non-limiting examples of hinges and linkers are provided in Tables 1 and 2. In some embodiments, a “linker” provides a spacer function compatible with interaction of the two sub-binding domains so that the resulting polypeptide retains a specific binding affinity to the same target molecule as an antibody that comprises the same light and heavy chain variable regions. In certain embodiments, a linker is comprised of five to about 35 amino acids, for instance, about 15 to about 25 amino acids. In some embodiments, a linker is comprised of at least 5 amino acids, at least 7 amino acids or at least 9 amino acids.

A “wild-type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody. In certain embodiments, a wild type immunoglobulin hinge region sequence is human, and can comprise a human IgG hinge region.

An “altered wild-type immunoglobulin hinge region” or “altered immunoglobulin hinge region” refers to (a) a wild type immunoglobulin hinge region with up to 30% amino acid changes (e.g., up to 25%, 20%, 15%, 10%, or 5% amino acid substitutions or deletions), or (b) a portion of a wild type immunoglobulin hinge region that has a length of about 5 amino acids (e.g., about 5, 6, 7, 8, 9, 10, 11, 12, 13, 14, 15, 16, 17, 18, 19, or 20 amino acids) up to about 120 amino acids (for instance, having a length of about 10 to about 40 amino acids or about 15 to about 30 amino acids or about 15 to about 20 amino acids or about 20 to about 25 amino acids), has up to about 30% amino acid changes (e.g., up to about 25%, 20%, 15%, 10%, 5%, 4%, 3%, 2%, or 1% amino acid substitutions or deletions or a combination thereof), and has an IgG core hinge region as disclosed in US 2013/0129723 and US 2013/0095097.

As used herein, the term “humanized” refers to a process of making an antibody or immunoglobulin binding proteins and polypeptides derived from a non-human species (e.g., mouse or rat) less immunogenic to humans, while still retaining antigen-binding properties of the original antibody, using genetic engineering techniques. In some embodiments, the binding domain(s) of an antibody or immunoglobulin binding proteins and polypeptides (e.g., light and heavy chain variable regions, Fab, scFv) are humanized. Non-human binding domains can be humanized using techniques known as CDR grafting (Jones et al., Nature 321:522 (1986)) and variants thereof, including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., “Derivation of therapeutically active humanized and veneered anti-CD18 antibodies.” In: Metcalf BW, Dalton B J, eds. Cellular adhesion: molecular definition to therapeutic potential. New York: Plenum Press, 1994: 291-312). If derived from a non-human source, other regions of the antibody or immunoglobulin binding proteins and polypeptides, such as the hinge region and constant region domains, can also be humanized.

An “immunoglobulin dimerization domain” or “immunoglobulin heterodimerization domain”, as used herein, refers to an immunoglobulin domain of a polypeptide chain that preferentially interacts or associates with a different immunoglobulin domain of a second polypeptide chain, wherein the interaction of the different immunoglobulin heterodimerization domains substantially contributes to or efficiently promotes heterodimerization of the first and second polypeptide chains (i.e., the formation of a dimer between two different polypeptide chains, which is also referred to as a “heterodimer”). The interactions between immunoglobulin heterodimerization domains “substantially contributes to or efficiently promotes” the heterodimerization of first and second polypeptide chains if there is a statistically significant reduction in the dimerization between the first and second polypeptide chains in the absence of the immunoglobulin heterodimerization domain of the first polypeptide chain and/or the immunoglobulin heterodimerization domain of the second polypeptide chain. In certain embodiments, when the first and second polypeptide chains are co-expressed, at least 60%, at least about 60% to about 70%, at least about 70% to about 80%, at least 80% to about 90%, 91%, 92%, 93%, 94%, 95%, 96%, 97%, 98%, or 99% of the first and second polypeptide chains form heterodimers with each other. Representative immunoglobulin heterodimerization domains include an immunoglobulin CH1 domain, an immunoglobulin CL domain (e.g., Cκ or Cλ isotypes), or derivatives thereof, including wild type immunoglobulin CH1 and CL domains and altered (or mutated) immunoglobulin CH1 and CL domains, as provided therein.

An “immunoglobulin constant region” or “constant region” is a term defined herein to refer to a peptide or polypeptide sequence that corresponds to or is derived from part or all of one or more constant region domains. In certain embodiments, the immunoglobulin constant region corresponds to or is derived from part or all of one or more constant region domains, but not all constant region domains of a source antibody. In certain embodiments, the constant region comprises IgG CH2 and CH3 domains, e.g., IgG1 CH2 and CH3 domains. In certain embodiments, the constant region does not comprise a CH1 domain. In certain embodiments, the constant region domains making up the constant region are human. In some embodiments (for example, in certain variations of a CD123-binding polypeptide or protein comprising a second binding domain that specifically binds CD3 or another T-cell surface antigen), the constant region domains of a fusion protein of this disclosure lack or have minimal effector functions of antibody-dependent cell-mediated cytotoxicity (ADCC) and complement activation and complement-dependent cytotoxicity (CDC), while retaining the ability to bind some Fc receptors (such as FcRn, the neonatal Fc receptor) and retaining a relatively long half-life in vivo. In other variations, a fusion protein of this disclosure includes constant domains that retain such effector function of one or both of ADCC and CDC. In certain embodiments, a binding domain of this disclosure is fused to a human IgG1 constant region, wherein the IgG1 constant region has one or more of the following amino acids mutated: leucine at position 234 (L234), leucine at position 235 (L235), glycine at position 237 (G237), glutamate at position 318 (E318), lysine at position 320 (K320), lysine at position 322 (K322), or any combination thereof (numbering according to EU). For example, any one or more of these amino acids can be changed to alanine. In a further embodiment, an IgG1 Fc domain has each of L234, L235, G237, E318, K320, and K322 (according to EU numbering) mutated to an alanine (i.e., L234A, L235A, G237A, E318A, K320A, and K322A, respectively), and optionally an N297A mutation as well (i.e., essentially eliminating glycosylation of the CH2 domain). In another embodiment, the IgG1 Fc domain has each of L234A, L235A, G237A and K322A mutations. For instance, the invention includes a recombinant polypeptide comprising a CD123 binding domain or scFv with an amino acid sequence at least 93%, at least 95%, at least 97%, at least 98%, at least 99% or 100% identical to SEQ ID NO:130; an IgG1 domain comprising the mutations L234A, L235A, G237A and K322A; and a CD3 binding domain. The invention includes a recombinant polypeptide comprising a CD123 binding domain comprising an amino acid sequence at least 93%, at least 95%, at least 97%, at least 98%, at least 99% or 100% identical to SEQ ID NO:130; an IgG1 domain comprising the mutations L234A, L235A, G237A and K322A; and a CD3 binding domain comprising an amino acid sequence at least 93%, at least 95%, at least 97%, at least 98%, at least 99% or 100% identical to SEQ ID NO:192 or SEQ ID NO:193.

“Fc region” or “Fc domain” refers to a polypeptide sequence corresponding to or derived from the portion of a source antibody that is responsible for binding to antibody receptors on cells and the C1q component of complement. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc fragment consists of the disulfide-linked heavy chain hinge regions, CH2, and CH3 domains. However, more recently the term has been applied to a single chain consisting of CH3, CH2, and at least a portion of the hinge sufficient to form a disulfide-linked dimer with a second such chain. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc includes variants of naturally occurring sequences.

In some embodiments, a CD123-binding protein comprises a protein scaffold as generally disclosed in, for example, in US Patent Application Publication Nos. 2003/0133939, 2003/0118592, and 2005/0136049. A CD123-binding protein may comprise, in order from amino-terminus to carboxyl-terminus: a first binding domain, a hinge region, and an immunoglobulin constant region. In other embodiments, a CD123-binding protein comprises a protein scaffold as generally disclosed in, for example, in US Patent Application Publication No. 2009/0148447. A CD123-binding protein may comprise, in order from amino-terminus to carboxyl-terminus: an immunoglobulin constant region, a hinge region and a first binding domain.

CD123-binding polypeptides and proteins disclosed herein may incorporate a multi-specific binding protein scaffold. Multi-specific binding proteins and polypeptides using scaffolds are disclosed, for instance, in PCT Application Publication No. WO 2007/146968, U.S. Patent Application Publication No. 2006/0051844, PCT Application Publication No. WO 2010/040105, PCT Application Publication No. WO 2010/003108, U.S. Pat. Nos. 7,166,707 and 8,409,577, which are each incorporated herein by reference in their entirety. A CD123-binding protein may comprise two binding domains (the domains can be designed to specifically bind the same or different targets), a hinge region, a linker (e.g., a carboxyl-terminus or an amino-terminus linker), and an immunoglobulin constant region. A CD123-binding protein may be a homodimeric protein comprising two identical, disulfide-bonded polypeptides.

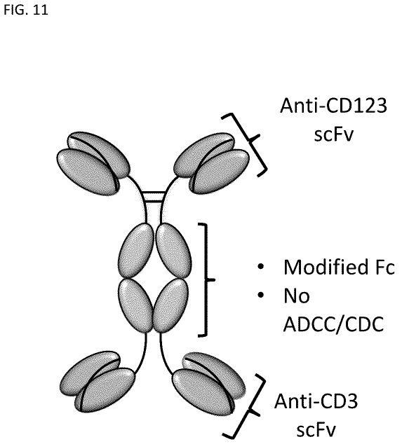

In one embodiment of the invention, the CD123-binding protein comprises, in order from amino-terminus to carboxyl terminus, a first binding domain, a hinge region, an immunoglobulin constant region and a second binding domain. FIG. 11 illustrates a CD123-binding protein in this configuration.

As used herein, the term “junction amino acids” or “junction amino acid residues” refers to one or more (e.g., about 2-10) amino acid residues between two adjacent regions or domains of a polypeptide, such as between a hinge and an adjacent immunoglobulin constant region or between a hinge and an adjacent binding domain or between a peptide linker and an adjacent immunoglobulin variable domain or an adjacent immunoglobulin constant region. Junction amino acids can result from the construct design of a polypeptide (e.g., amino acid residues resulting from the use of a restriction enzyme site during the construction of a nucleic acid molecule encoding a polypeptide).

As used herein, the term “patient in need” or “subject in need” refers to a patient at risk of, or suffering from, a disease, disorder or condition that is amenable to treatment or amelioration with a CD123-binding protein or polypeptide or a composition thereof provided herein. A patient in need may, for instance, be a patient diagnosed with a disease associated with the expression of CD123 such as acute myeloid leukemia (AML), B-lymphoid leukemia, blastic plasmocytoid dendritic neoplasms (BPDCN), hairy cell leukemia (HCL), myelodysplastic syndrome (MDS), acute lymphoblastic leukemia (ALL), refractory anemia with excess blasts (RAEB), chronic myeloid leukemia and Hodgkin's lymphoma.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

As used herein, the term “promoter” refers to a region of DNA involved in binding RNA polymerase to initiate transcription.

As used herein, the terms “nucleic acid,” “nucleic acid molecule,” or “polynucleotide” refer to deoxyribonucleotides or ribonucleotides and polymers thereof in either single- or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene. As used herein, the terms “nucleic acid,” “nucleic acid molecule,” or “polynucleotide” are intended to include DNA molecules (e.g., cDNA or genomic DNA), RNA molecules (e.g., mRNA), analogs of the DNA or RNA generated using nucleotide analogs, and derivatives, fragments and homologs thereof.

The term “expression” refers to the biosynthesis of a product encoded by a nucleic acid. For example, in the case of nucleic acid segment encoding a polypeptide of interest, expression involves transcription of the nucleic acid segment into mRNA and the translation of mRNA into one or more polypeptides.

The terms “expression unit” and “expression cassette” are used interchangeably herein and denote a nucleic acid segment encoding a polypeptide of interest and capable of providing expression of the nucleic acid segment in a host cell. An expression unit typically comprises a transcription promoter, an open reading frame encoding the polypeptide of interest, and a transcription terminator, all in operable configuration. In addition to a transcriptional promoter and terminator, an expression unit can further include other nucleic acid segments such as, e.g., an enhancer or a polyadenylation signal.

The term “expression vector,” as used herein, refers to a nucleic acid molecule, linear or circular, comprising one or more expression units. In addition to one or more expression units, an expression vector can also include additional nucleic acid segments such as, for example, one or more origins of replication or one or more selectable markers. Expression vectors are generally derived from plasmid or viral DNA, or can contain elements of both.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage “sequence identity” is calculated by determining the number of positions at which the identical nucleic acid base or amino acid residue occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. The comparison window for nucleic acid sequences can be, for instance, at least 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option. Two nucleotide or amino acid sequences are considered to have “substantially similar sequence identity” or “substantial sequence identity” if the two sequences have at least 80%, at least 85%, at least 90%, at least 95%, at least 96%, at least 97%, at least 98%, or at least 99% sequence identity relative to each other.

As used herein, a “polypeptide” or “polypeptide chain” is a single, linear and contiguous arrangement of covalently linked amino acids. Polypeptides can have or form one or more intrachain disulfide bonds. With regard to polypeptides as described herein, reference to amino acid residues corresponding to those specified by SEQ ID NO includes post-translational modifications of such residues.

As used herein, “CD123-binding protein” may be used interchangeably with “CD123-binding polypeptide,” “polypeptide,” and “recombinant polypeptide.” Such molecules specifically bind to CD123 (e.g., human CD123), also known as Cluster of Differentiation 123, Interleukin-3 receptor alpha chain, and IL3RA. CD123 is a type I transmembrane glycoprotein, with an extracellular domain comprising a predicted Ig-like domain and two FnIII domains. The CD123-binding proteins of the disclosure bind to the extracellular domain of CD123. The term “CD123” may refer to any isoform of CD123. Exemplary human CD123 nucleotide and amino acid sequences are provided in SEQ ID NOs:205 and 206 and SEQ ID NOs:207 and 208, respectively. CD123 associates with the beta chain of the interleukin-3 receptor to form the receptor.

A “protein” is a macromolecule comprising one or more polypeptide chains. A protein can also comprise non-peptidic components, such as carbohydrate groups. Carbohydrates and other non-peptidic substituents can be added to a protein by the cell in which the protein is produced, and will vary with the type of cell. Proteins are defined herein in terms of their amino acid backbone structures; substituents such as carbohydrate groups are generally not specified, but may be present nonetheless. A protein may be an antibody or an antigen-binding fragment of an antibody. In some embodiments, a protein may also be an scFv-Fc-scFv molecule, scFv-scFv dimer, or a diabody.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl-terminus of the reference sequence, but is not necessarily at the carboxyl-terminus of the complete polypeptide.

“T-cell receptor” (TCR) is a molecule found on the surface of T-cells that, along with CD3, is generally responsible for recognizing antigens bound to major histocompatibility complex (MHC) molecules. It consists of a disulfide-linked heterodimer of the highly variable α and β chains in most T-cells. In other T-cells, an alternative receptor made up of variable γ and δ chains is expressed. Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see Abbas and Lichtman, Cellular and Molecular Immunology (5th Ed.), Editor: Saunders, Philadelphia, 2003; Janeway et al., Immunobiology: The Immune System in Health and Disease, 4^{th} Ed., Current Biology Publications, p148, 149, and 172, 1999). TCR as used in the present disclosure can be from various animal species, including human, mouse, rat, or other mammals.

“TCR complex,” as used herein, refers to a complex formed by the association of CD3 chains with other TCR chains. For example, a TCR complex can be composed of a CD3γ chain, a CD3δ chain, two CD3ε chains, a homodimer of CD3ζ chains, a TCRα chain, and a TCRβ chain. Alternatively, a TCR complex can be composed of a CD3γ chain, a CD3δ chain, two CD3ε chains, a homodimer of CD3ζ chains, a TCRγ chain, and a TCRδ chain.

“A component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3E, or a sub-TCR complex of TCRα, TCRβ, CD3γ, CD3δ, and two CD3ε chains).

“Antibody-dependent cell-mediated cytotoxicity” and “ADCC,” as used herein, refer to a cell-mediated process in which nonspecific cytotoxic cells that express FcγRs (e.g., monocytic cells such as Natural Killer (NK) cells and macrophages) recognize bound antibody (or other protein capable of binding FcγRs) on a target cell and subsequently cause lysis of the target cell. In principle, any effector cell with an activating FcγR can be triggered to mediate ADCC. The primary cells for mediating ADCC are NK cells, which express only FcγRIII, whereas monocytes, depending on their state of activation, localization, or differentiation, can express FcγRI, FcγRII, and FcγRIII. For a review of FcγR expression on hematopoietic cells, see, e.g., Ravetch et al., 1991, Annu. Rev. Immunol., 9:457-92.

The term “having ADCC activity,” as used herein in reference to a polypeptide or protein, means that the polypeptide or protein (for example, one comprising an immunoglobulin hinge region and an immunoglobulin constant region having CH2 and CH3 domains, such as derived from IgG (e.g., IgG1)), is capable of mediating antibody-dependent cell-mediated cytotoxicity (ADCC) through binding of a cytolytic Fc receptor (e.g., FcγRIII) on a cytolytic immune effector cell expressing the Fc receptor (e.g., an NK cell).

“Complement-dependent cytotoxicity” and “CDC,” as used herein, refer to a process in which components in normal serum (“complement”), together with an antibody or other C1q-complement-binding protein bound to a target antigen, exhibit lysis of a target cell expressing the target antigen. Complement consists of a group of serum proteins that act in concert and in an orderly sequence to exert their effect.

The terms “classical complement pathway” and “classical complement system,” as used herein, are synonymous and refer to a particular pathway for the activation of complement. The classical pathway requires antigen-antibody complexes for initiation and involves the activation, in an orderly fashion, of nine major protein components designated C1 through C9. For several steps in the activation process, the product is an enzyme that catalyzes the subsequent step. This cascade provides amplification and activation of large amounts of complement by a relatively small initial signal.

The term “having CDC activity,” as used herein in reference to a polypeptide or protein, means that the polypeptide or protein (for example, one comprising an immunoglobulin hinge region and an immunoglobulin constant region having CH2 and CH3 domains, such as derived from IgG (e.g., IgG1)) is capable of mediating complement-dependent cytotoxicity (CDC) through binding of C1q complement protein and activation of the classical complement system. In one embodiment of the invention, the recombinant polypeptide has been modified to abate CDC activity.

“Redirected T-cell cytotoxicity” and “RTCC,” as used herein, refer to a T-cell-mediated process in which a cytotoxic T-cell is recruited to a target cell using a multi-specific protein that is capable of specifically binding both the cytotoxic T-cell and the target cell, and whereby a target-dependent cytotoxic T-cell response is elicited against the target cell. Polypeptides and proteins comprising anti-CD123 and anti-CD3 binding domains, as disclosed herein, are capable of RTCC.

As used herein, the term “treatment,” “treating,” or “ameliorating” refers to either a therapeutic treatment or prophylactic/preventative treatment. A treatment is therapeutic if at least one symptom of disease in an individual receiving treatment improves or a treatment can delay worsening of a progressive disease in an individual, or prevent onset of additional associated diseases.

As used herein, the term “therapeutically effective amount (or dose)” or “effective amount (or dose)” of a specific binding molecule or compound refers to that amount of the compound sufficient to result in amelioration of one or more symptoms of the disease being treated in a statistically significant manner or a statistically significant improvement in organ function. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially or simultaneously (in the same formulation or concurrently in separate formulations).

As used herein, the term “transformation,” “transfection,” and “transduction” refer to the transfer of nucleic acid (i.e., a nucleotide polymer) into a cell. As used herein, the term “genetic transformation” refers to the transfer and incorporation of DNA, especially recombinant DNA, into a cell. The transferred nucleic acid can be introduced into a cell via an expression vector.

As used herein, the term “variant” or “variants” refers to a nucleic acid or polypeptide differing from a reference nucleic acid or polypeptide, but retaining essential properties thereof. Generally, variants are overall closely similar, and, in many regions, identical to the reference nucleic acid or polypeptide. For instance, a variant may exhibit at least about 70%, at least about 80%, at least about 90%, at least about 95%, at least about 96%, at least about 97%, at least about 98% or at least about 99% sequence identity compared to the active portion or full length reference nucleic acid or polypeptide.

The terms “light chain variable region” (also referred to as “light chain variable domain” or “VL” or V_{L}) and “heavy chain variable region” (also referred to as “heavy chain variable domain” or “VH” or V_{H}) refer to the variable binding region from an antibody light and heavy chain, respectively. The variable binding regions are made up of discrete, well-defined sub-regions known as “complementarity determining regions” (CDRs) and “framework regions” (FRs), generally comprising in order FR1-CDR1-FR2-CDR2-FR3-CDR3-FR4 from amino-terminus to carboxyl-terminus. In one embodiment, the FRs are humanized. The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). A “Fab” (fragment antigen binding) is the part of an antibody that binds to antigens and includes the variable region and CH1 domain of the heavy chain linked to the light chain via an inter-chain disulfide bond.

The present disclosure describes binding domains that specifically bind CD123 (e.g., human CD123), as well as polypeptides and proteins comprising these binding domains. In some embodiments, the CD123-binding proteins and polypeptides comprise a second binding domain, which may bind to CD123 or to a different target. The polypeptides and proteins comprising binding domains of this disclosure can further comprise immunoglobulin constant regions, linker peptides, hinge regions, immunoglobulin dimerization/heterodimerization domains, junctional amino acids, tags, etc. These components of the disclosed polypeptides and proteins are described in further detail below.

Additionally, the CD123-binding polypeptides and proteins disclosed herein can be in the form of an antibody or a fusion protein of any of a variety of different formats (e.g., the fusion protein can be in the form of a CD123-binding bispecific or multi-specific molecule). Non-limiting examples of bispecific molecules include a scFv-Fc-scFv molecule. Some bispecific molecules comprise or consist of an anti-CD123 scFv linked to a second binding domain scFv and do not include other sequences such as an immunoglobulin constant region. In other embodiments, a CD123-binding protein is a diabody.

A CD123-binding protein in accordance with the present disclosure generally includes at least one CD123-binding polypeptide chain comprising (a) a CD123-binding domain as set forth herein. In certain variations, the CD123-binding polypeptide further includes (b) a hinge region carboxyl-terminal to the CD123-binding domain, and (c) an immunoglobulin constant region. In further variations, the CD123-binding polypeptide further includes (d) a carboxyl-terminus linker carboxyl-terminal to the immunoglobulin constant region, and (e) a second binding domain carboxyl-terminal to the carboxyl-terminus linker.

In yet other variations, the CD123-binding polypeptide comprises (b) a hinge region amino-terminal to the CD123-binding domain, and (c) an immunoglobulin sub-region amino-terminal to the hinge region.

In some embodiments, recombinant polypeptides are capable of homodimerization, typically through disulfide bonding, via the immunoglobulin constant region and/or hinge region (e.g., via an immunoglobulin constant region comprising IgG CH2 and CH3 domains and an IgG hinge region). Thus, in certain embodiments of the present disclosure, two identical single chain CD123-binding polypeptides homodimerize to form a dimeric CD123-binding protein. An example of a homodimer of the invention is provided in FIG. 11.

In other embodiments, a CD123-binding polypeptide includes a heterodimerization domain that is capable of heterodimerization with a different heterodimerization domain in a second, non-identical polypeptide chain. In certain variations, the second polypeptide chain for heterodimerization includes a second binding domain. Accordingly, in certain embodiments of the present disclosure, two non-identical polypeptide chains, one comprising the CD123-binding domain and the second optionally comprising a second binding domain, dimerize to form a heterodimeric CD123-binding protein. Examples of types of heterodimers include those described in U.S. Patent Application Publication No. 2013/0095097 and US 2013/0129723.

In some embodiments, a CD123-binding domain, protein or polypeptide is conjugated to a drug or a toxic moiety.

CD123-binding polypeptides, proteins, and their various components used in the therapeutics of the present disclosure are further described below.

As indicated above, the disclosure relates to binding domains that specifically bind CD123. In some variations, the CD123-binding domain is capable of competing for binding to CD123 with an antibody having V_{L} and V_{H} regions having amino acid sequences as shown in SEQ ID NO:194 and SEQ ID NO:196, respectively (e.g., 12F1). The murine anti-CD123 antibody 12F1 is described in, for example, U.S. Patent Application Publication No. 2013/041739 and Kuo et al, (2012) Protein Eng Design Select, p 1-9.

The CD123-binding domain may comprise sequences shown in Table 3, and some relevant SEQ ID NOs are summarized in Table 6. In certain embodiments, the CD123-binding domain comprises (i) an immunoglobulin light chain variable region (VL) comprising CDRs LCDR1, LCDR2, and LCDR3, and (ii) an immunoglobulin heavy chain variable region (VH) comprising CDRs HCDR1, HCDR2, and HCDR3 with HCDR1 comprising an amino acid sequence as set forth in SEQ ID NO:12, with HCDR2 comprising an amino acid sequence as set forth in SEQ ID NO:14 and with HCDR3 comprising an amino acid sequence as set forth in SEQ ID NO:16. In certain embodiments, the CD123-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some such embodiments, (i) the LCDR1 has an amino acid sequence set forth in SEQ ID NO:6 or a sequence that differs from SEQ ID NO:6 by at least one amino acid substitution; (ii) the LCDR2 has an amino acid sequence set forth in SEQ ID NO:8 or a sequence that differs from SEQ ID NO:8 by at least one amino acid substitution; (iii) the LCDR3 has an amino acid sequence set forth in SEQ ID NO:10 or a sequence that differs from SEQ ID NO:10 by at least one amino acid substitution; (iv) the HCDR1 has an amino acid sequence set forth in SEQ ID NO:12 or a sequence that differs from SEQ ID NO:12 by at least one amino acid substitution; (v) the HCDR2 has an amino acid sequence set forth in SEQ ID NO:14 or a sequence that differs from SEQ ID NO:14 by at least one amino acid substitution; and (vi) the HCDR3 has an amino acid sequence set forth in SEQ ID NO:16 or a sequence that differs from SEQ ID NO:16 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In certain embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody. For instance, the invention includes a recombinant polypeptide comprising (i) the LCDR1 has an amino acid sequence set forth in SEQ ID NO:6 or a sequence that differs from SEQ ID NO:6 by one or two amino acid substitutions; (ii) the LCDR2 has an amino acid sequence set forth in SEQ ID NO:8 or a sequence that differs from SEQ ID NO:8 by one or two amino acid substitutions; (iii) the LCDR3 has an amino acid sequence set forth in SEQ ID NO:10 or a sequence that differs from SEQ ID NO:10 by one or two amino acid substitutions; (iv) the HCDR1 has an amino acid sequence set forth in SEQ ID NO:12 or a sequence that differs from SEQ ID NO:12 by one or two amino acid substitutions; (v) the HCDR2 has an amino acid sequence set forth in SEQ ID NO:14 or a sequence that differs from SEQ ID NO:14 by one or two amino acid substitutions; and (vi) the HCDR3 has an amino acid sequence set forth in SEQ ID NO:16 or a sequence that differs from SEQ ID NO:16 by one or two amino acid substitutions. The amino acid substitution described above may be a conservative or a non-conservative amino acid substitution.

In related embodiments, a recombinant polypeptide of the invention comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:2) or to a heavy chain variable region (V_{H}) (e.g., SEQ ID NO:4), or both. In one embodiment, the CD123-binding domain of the recombinant polypeptide is an scfv comprising a variable heavy chain comprising SEQ ID NO:4 and a variable light chain comprising SEQ ID NO:2 in the VHVL orientation. In another embodiment, the CD123-binding domain of the recombinant polypeptide is an scFv comprising a variable light chain comprising SEQ ID NO:2 and a variable heavy chain comprising SEQ ID NO:4 in the VLVH orientation. For instance, in certain embodiments, the polypeptide of the invention comprises an amino acid sequence of SEQ ID NO:130 or SEQ ID NO:132. The invention includes a recombinant polypeptide that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of SEQ ID NO:130 or SEQ ID NO:132.

In certain embodiments, the CD123-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some such embodiments, (i) the LCDR1 has an amino acid sequence set forth in SEQ ID NO:22 or a sequence that differs from SEQ ID NO:22 by at least one amino acid substitution; (ii) the LCDR2 has an amino acid sequence set forth in SEQ ID NO:24 or a sequence that differs from SEQ ID NO:24 by at least one amino acid substitution; (iii) the LCDR3 has an amino acid sequence set forth in SEQ ID NO:26 or a sequence that differs from SEQ ID NO:26 by at least one amino acid substitution; (iv) the HCDR1 has an amino acid sequence set forth in SEQ ID NO:28 or a sequence that differs from SEQ ID NO:28 by at least one amino acid substitution; (v) the HCDR2 has an amino acid sequence set forth in SEQ ID NO:30 or a sequence that differs from SEQ ID NO:30 by at least one amino acid substitution; and (vi) the HCDR3 has an amino acid sequence set forth in SEQ ID NO:32 or a sequence that differs from SEQ ID NO:32 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In certain embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody.

In related embodiments, a CD123-binding domain comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:18) or to a heavy chain variable region (V_{H}) (e.g., SEQ ID NO:20), or both.

In certain embodiments, the CD123-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some such embodiments, (i) the LCDR1 has an amino acid sequence set forth in SEQ ID NO:22 or a sequence that differs from SEQ ID NO:22 by at least one amino acid substitution; (ii) the LCDR2 has an amino acid sequence set forth in SEQ ID NO:24 or a sequence that differs from SEQ ID NO:24 by at least one amino acid substitution; (iii) the LCDR3 has an amino acid sequence set forth in SEQ ID NO:26 or a sequence that differs from SEQ ID NO:26 by at least one amino acid substitution; (iv) the HCDR1 has an amino acid sequence set forth in SEQ ID NO:36 or a sequence that differs from SEQ ID NO:36 by at least one amino acid substitution; (v) the HCDR2 has an amino acid sequence set forth in SEQ ID NO:38 or a sequence that differs from SEQ ID NO:38 by at least one amino acid substitution; and (vi) the HCDR3 has an amino acid sequence set forth in SEQ ID NO:40 or a sequence that differs from SEQ ID NO:40 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In certain embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody.

In related embodiments, a CD123-binding domain comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:18) or to a heavy chain variable region (V_{H}) (e.g., SEQ ID NO:34), or both.

In certain embodiments, the CD123-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some such embodiments, (i) the LCDR1 has an amino acid sequence set forth in SEQ ID NO:22 or a sequence that differs from SEQ ID NO:22 by at least one amino acid substitution; (ii) the LCDR2 has an amino acid sequence set forth in SEQ ID NO:24 or a sequence that differs from SEQ ID NO:24 by at least one amino acid substitution; (iii) the LCDR3 has an amino acid sequence set forth in SEQ ID NO:26 or a sequence that differs from SEQ ID NO:26 by at least one amino acid substitution; (iv) the HCDR1 has an amino acid sequence set forth in SEQ ID NO:44 or a sequence that differs from SEQ ID NO:44 by at least one amino acid substitution; (v) the HCDR2 has an amino acid sequence set forth in SEQ ID NO:46 or a sequence that differs from SEQ ID NO:46 by at least one amino acid substitution; and (vi) the HCDR3 has an amino acid sequence set forth in SEQ ID NO:48 or a sequence that differs from SEQ ID NO:48 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In certain embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody.

In related embodiments, a CD123-binding domain comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:18) or to a heavy chain variable region (V_{H}) (e.g., SEQ ID NO:42), or both.

In certain embodiments, the CD123-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some such embodiments, (i) the LCDR1 has an amino acid sequence set forth in SEQ ID NO:22 or a sequence that differs from SEQ ID NO:22 by at least one amino acid substitution; (ii) the LCDR2 has an amino acid sequence set forth in SEQ ID NO:24 or a sequence that differs from SEQ ID NO:24 by at least one amino acid substitution; (iii) the LCDR3 has an amino acid sequence set forth in SEQ ID NO:26 or a sequence that differs from SEQ ID NO:26 by at least one amino acid substitution; (iv) the HCDR1 has an amino acid sequence set forth in SEQ ID NO:100 or a sequence that differs from SEQ ID NO:100 by at least one amino acid substitution; (v) the HCDR2 has an amino acid sequence set forth in SEQ ID NO:102 or a sequence that differs from SEQ ID NO:102 by at least one amino acid substitution; and (vi) the HCDR3 has an amino acid sequence set forth in SEQ ID NO:104 or a sequence that differs from SEQ ID NO:104 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In certain embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody.

In related embodiments, a CD123-binding domain comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:18) or to a heavy chain variable region (V_{H}) (e.g., SEQ ID NO:98), or both.

In certain embodiments, the CD123-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some such embodiments, (i) the LCDR1 has an amino acid sequence set forth in SEQ ID NO:22 or a sequence that differs from SEQ ID NO:22 by at least one amino acid substitution; (ii) the LCDR2 has an amino acid sequence set forth in SEQ ID NO:24 or a sequence that differs from SEQ ID NO:24 by at least one amino acid substitution; (iii) the LCDR3 has an amino acid sequence set forth in SEQ ID NO:26 or a sequence that differs from SEQ ID NO:26 by at least one amino acid substitution; (iv) the HCDR1 has an amino acid sequence set forth in SEQ ID NO:116 or a sequence that differs from SEQ ID NO:116 by at least one amino acid substitution; (v) the HCDR2 has an amino acid sequence set forth in SEQ ID NO:118 or a sequence that differs from SEQ ID NO:118 by at least one amino acid substitution; and (vi) the HCDR3 has an amino acid sequence set forth in SEQ ID NO:120 or a sequence that differs from SEQ ID NO:120 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In certain embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody.

In related embodiments, a CD123-binding domain comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:18) or to a heavy chain variable region (V_{H}) (e.g., SEQ ID NO: 114), or both.

In certain embodiments, the CD123-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some such embodiments, (i) the LCDR1 has an amino acid sequence set forth in SEQ ID NO:22 or a sequence that differs from SEQ ID NO:22 by at least one amino acid substitution; (ii) the LCDR2 has an amino acid sequence set forth in SEQ ID NO:24 or a sequence that differs from SEQ ID NO:24 by at least one amino acid substitution; (iii) the LCDR3 has an amino acid sequence set forth in SEQ ID NO:26 or a sequence that differs from SEQ ID NO:26 by at least one amino acid substitution; (iv) the HCDR1 has an amino acid sequence set forth in SEQ ID NO:124 or a sequence that differs from SEQ ID NO:124 by at least one amino acid substitution; (v) the HCDR2 has an amino acid sequence set forth in SEQ ID NO:126 or a sequence that differs from SEQ ID NO:126 by at least one amino acid substitution; and (vi) the HCDR3 has an amino acid sequence set forth in SEQ ID NO:128 or a sequence that differs from SEQ ID NO:128 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In certain embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody.

In related embodiments, a CD123-binding domain comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:18) or to a heavy chain variable region (V_{H}) (e.g., SEQ ID NO:122), or both.

In certain embodiments, the CD123-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some such embodiments, (i) the LCDR1 has an amino acid sequence set forth in SEQ ID NO:54 or a sequence that differs from SEQ ID NO:54 by at least one amino acid substitution; (ii) the LCDR2 has an amino acid sequence set forth in SEQ ID NO:56 or a sequence that differs from SEQ ID NO:56 by at least one amino acid substitution; (iii) the LCDR3 has an amino acid sequence set forth in SEQ ID NO:58 or a sequence that differs from SEQ ID NO:58 by at least one amino acid substitution; (iv) the HCDR1 has an amino acid sequence set forth in SEQ ID NO:60 or a sequence that differs from SEQ ID NO:60 by at least one amino acid substitution; (v) the HCDR2 has an amino acid sequence set forth in SEQ ID NO:62 or a sequence that differs from SEQ ID NO:62 by at least one amino acid substitution; and (vi) the HCDR3 has an amino acid sequence set forth in SEQ ID NO:64 or a sequence that differs from SEQ ID NO:64 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In certain embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody.

In related embodiments, a CD123-binding domain comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:50) or to a heavy chain variable region (V_{H}) (e.g., SEQ ID NO:52), or both.

In certain embodiments, the CD123-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some such embodiments, (i) the LCDR1 has an amino acid sequence set forth in SEQ ID NO:70 or a sequence that differs from SEQ ID NO:70 by at least one amino acid substitution; (ii) the LCDR2 has an amino acid sequence set forth in SEQ ID NO:72 or a sequence that differs from SEQ ID NO:72 by at least one amino acid substitution; (iii) the LCDR3 has an amino acid sequence set forth in SEQ ID NO:74 or a sequence that differs from SEQ ID NO:74 by at least one amino acid substitution; (iv) the HCDR1 has an amino acid sequence set forth in SEQ ID NO:76 or a sequence that differs from SEQ ID NO:76 by at least one amino acid substitution; (v) the HCDR2 has an amino acid sequence set forth in SEQ ID NO:78 or a sequence that differs from SEQ ID NO:78 by at least one amino acid substitution; and (vi) the HCDR3 has an amino acid sequence set forth in SEQ ID NO:80 or a sequence that differs from SEQ ID NO:80 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In certain embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody.

In related embodiments, a CD123-binding domain comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:66) or to a heavy chain variable region (V_{H}) (e.g., SEQ ID NO:68), or both.

In certain embodiments, the CD123-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some such embodiments, (i) the LCDR1 has an amino acid sequence set forth in SEQ ID NO:86 or a sequence that differs from SEQ ID NO:86 by at least one amino acid substitution; (ii) the LCDR2 has an amino acid sequence set forth in SEQ ID NO:88 or a sequence that differs from SEQ ID NO:88 by at least one amino acid substitution; (iii) the LCDR3 has an amino acid sequence set forth in SEQ ID NO:90 or a sequence that differs from SEQ ID NO:90 by at least one amino acid substitution; (iv) the HCDR1 has an amino acid sequence set forth in SEQ ID NO:92 or a sequence that differs from SEQ ID NO:92 by at least one amino acid substitution; (v) the HCDR2 has an amino acid sequence set forth in SEQ ID NO:94 or a sequence that differs from SEQ ID NO:94 by at least one amino acid substitution; and (vi) the HCDR3 has an amino acid sequence set forth in SEQ ID NO:96 or a sequence that differs from SEQ ID NO:96 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In certain embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody.

In related embodiments, a CD123-binding domain comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:82) or to a heavy chain variable region (V_{H}) (e.g., SEQ ID NO:84), or both.

In certain embodiments, a CD123-binding domain comprises humanized immunoglobulin V_{L} and/or V_{H} regions. Techniques for humanizing immunoglobulin V_{L} and V_{H} regions are known in the art and are discussed, for example, in U.S. Patent Application Publication No. 2006/0153837. In certain embodiments, a CD123-binding domain comprises human immunoglobulin V_{L} and/or V_{H} regions.

“Humanization” is expected to result in an antibody that is less immunogenic, with complete retention of the antigen-binding properties of the original molecule. In order to retain all of the antigen-binding properties of the original antibody, the structure of its antigen binding site should be reproduced in the “humanized” version. This can be achieved by grafting only the nonhuman CDRs onto human variable framework domains and constant regions, with or without retention of critical framework residues (Jones et al., Nature 321:522 (1986); Verhoeyen et al., Science 239:1539 (1988)) or by recombining the entire nonhuman variable domains (to preserve ligand-binding properties), but “cloaking” them with a human-like surface through judicious replacement of exposed residues (to reduce antigenicity) (Padlan, Molec. Immunol. 28:489 (1991)).

Essentially, humanization by CDR grafting involves recombining only the CDRs of a non-human antibody onto a human variable region framework and a human constant region. Theoretically, this should substantially reduce or eliminate immunogenicity (except if allotypic or idiotypic differences exist). However, it has been reported that some framework residues of the original antibody also may need to be preserved (Reichmann et al., Nature, 332:323 (1988); Queen et al., Proc. Natl. Acad. Sci. USA, 86:10, 029 (1989)).

The framework residues that need to be preserved are amenable to identification through computer modeling. Alternatively, critical framework residues can potentially be identified by comparing known antigen-binding site structures (Padlan, Molec. Immunol., 31(3):169-217 (1994), incorporated herein by reference).

The residues that potentially affect antigen binding fall into several groups. The first group comprises residues that are contiguous with the antigen site surface, which could therefore make direct contact with antigens. These residues include the amino-terminal residues and those adjacent to the CDRs. The second group includes residues that could alter the structure or relative alignment of the CDRs, either by contacting the CDRs or another peptide chain in the antibody. The third group comprises amino acids with buried side chains that could influence the structural integrity of the variable domains. The residues in these groups are usually found in the same positions (Padlan, 1994, supra) although their positions as identified may differ depending on the numbering system (see Kabat et al., “Sequences of proteins of immunological interest, 5th ed., Pub. No. 91-3242, U.S. Dept. Health & Human Services, NIH, Bethesda, Md., 1991).

Knowledge about humanized antibodies in the art is applicable to the polypeptides according to the disclosure, even if these polypeptides are not antibodies.

In some embodiments, the disclosure relates to CD123-binding domains wherein (i) the immunoglobulin light chain variable region comprises an amino acid sequence that is at least 88%, at least 90%, at least 92%, at least 95%, at least 97%, at least 98% or at least 99% identical to the amino acid sequence set forth in SEQ ID NO:2 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:4; (ii) the immunoglobulin light chain variable region comprises an amino acid sequence that is at least 88%, at least 90%, at least 92%, at least 95%, at least 97%, at least 98% or at least 99% identical to the amino acid sequence set forth in SEQ ID NO:18 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:20; (iii) the immunoglobulin light chain variable region comprises an amino acid sequence that is at least 88%, at least 90%, at least 92%, at least 95%, at least 97%, at least 98% or at least 99% identical to the amino acid sequence set forth in SEQ ID NO:18 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:34; (iv) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:18 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:42; (v) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:50 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:52; (vi) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:66 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:68; (vii) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:82 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:84; (viii) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:18 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:122; (ix) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:18 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:98; (x) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:18 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:106; or (xi) the immunoglobulin light chain variable region comprises an amino acid sequence that is at least 85%, at least 90%, at least 92%, at least 95%, at least 97%, at least 98% or at least 99% identical to the amino acid sequence set forth in SEQ ID NO:18 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO: 114.

In further embodiments, each CDR comprises no more than one, two, or three substitutions, insertions or deletions, as compared to that from a monoclonal antibody or fragment or derivative thereof that specifically binds to a target of interest (e.g., CD123).

In certain embodiments, a CD123-binding protein can comprise one or more additional binding domains (e.g., second binding domain) that bind a target other than CD123. These other binding domains can comprise, for example, a particular cytokine or a molecule that targets the binding domain polypeptide to a particular cell type, a toxin, an additional cell receptor, an antibody, etc.

In certain embodiments, a CD123-binding molecule or protein can comprise a T-cell binding domain for recruitment of T-cells to target cells expressing CD123. In certain embodiments, a CD123-binding protein as described herein can comprise (i) a binding domain that specifically binds a TCR complex or a component thereof (e.g., TCRα, TCRβ, CD3γ, CD3δ, and CD3ε) and (ii) another binding domain that specifically binds to CD123. A CD123-binding protein can utilize essentially any binding domain that binds a T-cell, e.g., an antibody derived binding domain. Exemplary anti-CD3 antibodies from which the CD3 binding domain can be derived include the CRIS-7 monoclonal antibody (Reinherz, E. L. et al. (eds.), Leukocyte typing II., Springer Verlag, New York, (1986); V_{L} and V_{H} amino acid sequences respectively shown in SEQ ID NO:209 (QVVLTQSPAIMSAFPGEKVTMTCSASSSVSYMNWYQQKSGTSPKRWIYDSSKLASGVPARFS GSGSGTSYSLTISSMETEDAATYYCQQWSRNPPTFGGGTKLQITR) and SEQ ID NO:210 (QVQLQQSGAELARPGASVKMSCKASGYTFTRSTMHWVKQRPGQGLEWIGYINP SSAYTNYNQKFKDKATLTADKSSSTAYMQLSSLTSEDSAVYYCASPQVHYDYNGFPYWGQGT LVTVSA)); HuM291 (Chau et al. (2001) Transplantation 71:941-950; V_{L} and V_{H} amino acid sequences respectively shown in SEQ ID NO:211(DIQMTQSPSSLSASVGDRVTITCSASSSVSYMNWYQQKPGKAPKRLIYDTSKLASGVP SRFSGSGSGTDFTLTISSLQPEDFATYYCQQWSSNPPTFGGGTKVEIK) and SEQ ID NO:212 (QVQLVQSGAEVKKPGASVKVSCKASGYTFISYTMHWVRQAPGQGLEWMGYINPRSGYTHYN QKLKDKATLTADKSASTAYMELSSLRSEDTAVYYCARSAYYDYDGFAYWGQGTLVTVSS)); BC3 monoclonal antibody (Anasetti et al. (1990) J. Exp. Med. 172:1691); OKT3 monoclonal antibody (Ortho multicenter Transplant Study Group (1985) N. Engl. J. Med. 313:337) and derivatives thereof such as OKT3 ala-ala (also referred to as OKT3 AA-FL or OKT3 FL), a humanized, Fc variant with alanine substitutions at positions 234 and 235 (Herold et al. (2003) J. Clin. Invest. 11:409); visilizumab (Carpenter et al. (2002) Blood 99:2712), G19-4 monoclonal antibody (Ledbetter et al., 1986, J. Immunol. 136:3945), 145-2C11 monoclonal antibody (Hirsch et al. (1988) J. Immunol. 140: 3766) and I2C monoclonal antibody (see, e.g., US 2011/0293619 and US20120244162). For example, a CD3 binding domain may comprise a CD3 binding domain disclosed in U.S. Patent Application Publication No. 2012/0244162, including a CD3 binding domain comprising a VL region selected from SEQ ID NO: 17, 21, 35, 39, 53, 57, 71, 75, 89, 83, 107, 111, 125, 129, 143, 147, 161, 165, 179 and 183 of US 2012/0244162 and/or a VH region selected from SEQ ID NO:15, 19, 33, 37, 51, 55, 69, 73, 87, 91, 105, 109, 123, 127, 141, 145, 159, 163, 177 and 181 of US 2012/0244162. In some embodiments, a CD3 binding domain comprises an amino acid sequence selected from SEQ ID NO: 23, 25, 41, 43, 59, 61, 77, 79, 95, 97, 113, 115, 131, 133, 149, 151, 167, 169, 185, and 187 of US 2012/0244162. In some embodiments, a CD3 binding domain is one described in WO2004/106380, WO2005/040220A1, US 2014/0099318 or derived from a CD3 binding domain thereof. An exemplary anti-TCR antibody is the BMA031 monoclonal antibody (Borst et al. (1990) Human Immunology 29:175-188). The CD3 binding domain may be derived from any of the antibodies or sequences described in WO 2013/158856 (incorporated herein by reference in its entirety). In certain variations, the second binding domain of a CD123-binding polypeptide described herein comprises: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 has the amino acid sequences set forth in SEQ ID NOs:162, 163 and 164, respectively, and the HCDR1, HCDR2, and HCDR3 has the amino acid sequences set forth in SEQ ID NOs: 165, 166 and 167, respectively; or (b) the LCDR1, LCDR2 and LCDR3 has the amino acid sequences set forth in SEQ ID NO:168, SEQ ID NO:169, and SEQ ID NO:170, respectively, and the HCDR1, HCDR2, and HCDR3 has the amino acid sequences set forth in SEQ ID NO: 171, SEQ ID NO:172, and SEQ ID NO:173, respectively. In other aspects, the second binding domain of a CD123-binding polypeptide described herein comprises: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 has the amino acid sequences set forth in SEQ ID NOs: 171, 172 and 173, respectively, and the HCDR1, HCDR2, and HCDR3 has the amino acid sequences set forth in SEQ ID NOs: 174, 175 and 176, respectively; or (b) the LCDR1, LCDR2 and LCDR3 has the amino acid sequences set forth in SEQ ID NOs: 176, 177 and 178, respectively, and the HCDR1, HCDR2, and HCDR3 has the amino acid sequences set forth in SEQ ID NOs: 179, 180 and 181, respectively. In certain embodiments, the second binding domain of a CD123-binding polypeptide described herein comprises: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 has the amino acid sequences set forth in SEQ ID NOs: 182, 183 and 184, respectively, and the HCDR1, HCDR2, and HCDR3 has the amino acid sequences set forth in SEQ ID NOs: 185, 186 and 187, respectively; or (b) the LCDR1, LCDR2 and LCDR3 has the amino acid sequences set forth in SEQ ID NOs: 188, 189 and 190, respectively, and the HCDR1, HCDR2, and HCDR3 has the amino acid sequences set forth in SEQ ID NOs: 191, 192 and 193, respectively. In some aspects, the second binding domains comprising the CDR sequences recited in this paragraph are humanized.

In some embodiments of a CD123-binding protein comprising a second binding domain that specifically binds CD3ε, the second binding domain competes for binding to CD3ε with the CRIS-7, HuM291 or I2C monoclonal antibody. In certain variations, the CD3-binding domain comprises an immunoglobulin light chain variable region (V_{L}) and an immunoglobulin heavy chain variable region (V_{H}) derived from the CRIS-7, HuM291 or I2C monoclonal antibody (e.g., the V_{L} and V_{H} of the second binding domain can be humanized variable regions comprising, respectively, the light chain CDRs and the heavy chain CDRs of the monoclonal antibody). A second binding domain may comprise the light chain variable region, the heavy chain variable region, or both, of the DRA222, TSC455, or TSC456 CD3-binding domains. The amino acid sequences of DRA222, TSC455, and TSC456 are provided in Table 3. The DRA222 binding domains are also described in WO 2013/158856. TSC455 may also be referred to as TSC394 F87Y. TSC455 may also be referred to as TSC394 E86D F87Y or TSC394 DY. In some embodiments, the second binding domain specifically binds CD3 and comprises an immunoglobulin light chain variable region and an immunoglobulin heavy chain variable region; wherein the immunoglobulin light chain variable region comprises an amino acid sequence that is at least about 93% identical, at least about 95% identical, at least about 97% identical, at least about 98% identical or at least about 99% identical to the amino acid sequence in SEQ ID NO:157; or at least about 94% identical, at least about 95% identical, at least about 97% identical, at least about 98% identical or at least about 99% identical to the amino acid sequence in SEQ ID NO:158; and wherein the immunoglobulin heavy chain variable region comprises an amino acid sequence that is at least about 82% identical, at least about 85% identical, at least about 87% identical, at least about 90% identical, at least about 92% identical, at least about 95% identical, at least about 97% identical, at least about 98% identical or at least about 99% identical to the amino acid sequence in SEQ ID NO:159. In some embodiments, a CD123-binding polypeptide or protein further comprising a CD3-binding domain may have a low level of high molecular weight aggregates produced during recombinant expression of the polypeptide or protein. A CD123-binding polypeptide or protein further comprising a CD3-binding domain may exhibit a relatively long stability in human serum, depending on the CD3-binding domain present in the polypeptide or protein.

In certain variations, the second binding domain of a CD123-binding polypeptide described herein is a CD3-binding domain and comprises one or more of the CD3-binding sequences (e.g., CDRs or variable regions) disclosed in US 2013/0129730, US 2011/0293619, U.S. Pat. No. 7,635,472, WO 2010/037836, WO 2004/106381, or WO 2011/121110; each incorporated herein by reference in its entirety. In some embodiments, a CD3-binding domain comprises one or more of the following sequences:

| LCDR1 | LCDR2 | LCDR3 |
|---|---|---|
| GSSTGAV | GTKFLAP | ALWYSNR |
| TSGYYPN | (SEQ ID | WV |
| (SEQ ID | NO: 292) | (SEQ ID |
| NO: 289) | | NO: 295) |
| RSSTGAV | ATDMRPS | ALWYSNR |
| TSGYYPN | (SEQ ID | WV |
| (SEQ ID | NO: 293) | (SEQ ID |
| NO: 290) | | NO: 296) |
| GSSTGAV | GTKFLAP | VLWYSNR |
| TSGNYPN | (SEQ ID | WV |
| (SEQ ID | NO: 294) | (SEQ ID |
| NO: 291) | | NO: 297) |

In various embodiments, a CD3-binding domain comprises one or more of the following sequences:

| HCDR1 | HCDR2 | HCDR3 |
|---|---|---|
| IYAMN | RIRSKYN | HGNFGNS |
| (SEQ ID | NYATYYA | YVSFFAY |
| NO: 298) | DSVKS | (SEQ ID |
| | (SEQ ID | NO: 304) |
| | NO: 301) | |
| KYAMN | RIRSKYN | HGNFGNS |
| (SEQ ID | NYATYYA | YISYWAY |
| NO: 299) | DSVKD | (SEQ ID |
| | (SEQ ID | NO: 305) |
| | NO: 302) | |
| SYAMN | RIRSKYN | HGNFGNS |
| (SEQ ID | NYATYYA | YLSFWAY |
| NO: 300) | DSVKG | (SEQ ID |
| | (SEQ ID | NO: 306) |
| | NO: 303) | |

In certain embodiments, the CD123-binding polypeptide used in the methods and compositions described herein is a bispecific single chain molecule comprising a CD123 binding domain and a CD3 binding domain. In some embodiments, a CD123- and/or a CD3-binding domain is derived from an antibody and comprises a variable heavy chain (VH) and a variable light chain (VL). For example, an scFv comprises a VH and VL. These binding domains and variable chains may be arranged in any order that still retains some binding to the target(s). For example, the variable domains may be arranged in the order such as VH CD123-VL CD123-VH CD3-VL CD3; VL CD123-VH CD123-VH CD3-VL CD3; VH CD123-VL CD123-VL CD3-VH CD3; VL CD123-VH CD123-VL CD3-VH CD3; VH CD3-VL CD3-VH CD123-VL CD123; VL CD3-VH CD3-VL CD123-VH CD123; VH CD3-VL CD3-VL CD123-VH CD123; or VL CD3-VH CD3-VH CD123-VL CD123. The pairs of VH regions and VL regions in the binding domain binding to CD3 may be in the format of a single chain antibody (scFv). The VH and VL regions may be arranged in the order VH-VL or VL-VH. In some embodiments, the scFv may bind to CD123 more effectively than the antibody comprising the same VH and VL region sequences in the same orientation. In certain embodiments, the scFv may bind more effectively to CD123 in the VL-VH orientation than in the VH-VL orientation, or vice versa (see, e.g., Example 2). The VH-region may be positioned N-terminally to a linker sequence. The VL region may be positioned C-terminally to the linker sequence. The domain arrangement in the CD3 binding domain of the bispecific single chain molecule may be VH-VL, with said CD3 binding domain located C-terminally to the CD123-binding domain. A bispecific molecule may comprise an scFv binding to CD123 linked to an scFv binding to CD3. These scFvs may be linked with a short peptide. In some embodiments, bispecific single chain molecules do not comprise a hinge region or a constant region (see, for example, US 2013/0295121, WO 2010/037836, WO 2004/106381 and WO 2011/121110; each incorporated herein by reference in its entirety).

In some embodiments, a binding domain is a single-chain Fv fragment (scFv) that comprises V_{H} and V_{L} regions specific for a target of interest. In certain embodiments, the V_{H} and V_{L} regions are human or humanized.

In some variations, a binding domain is a single-chain Fv (scFv) comprising immunoglobulin V_{L} and V_{H} regions joined by a peptide linker. The use of peptide linkers for joining V_{L} and V_{H} regions is well-known in the art, and a large number of publications exist within this particular field. In some embodiments, a peptide linker is a 15mer consisting of three repeats of a Gly-Gly-Gly-Gly-Ser amino acid sequence ((Gly₄Ser)₃) (SEQ ID NO:213). Other linkers have been used, and phage display technology, as well as selective infective phage technology, has been used to diversify and select appropriate linker sequences (Tang et al., J. Biol. Chem. 271, 15682-15686, 1996; Hennecke et al., Protein Eng. 11, 405-410, 1998). In certain embodiments, the V_{L} and V_{H} regions are joined by a peptide linker having an amino acid sequence comprising the formula (Gly₄Ser)ₙ, wherein n=1-5 (SEQ ID NO:214). Other suitable linkers can be obtained by optimizing a simple linker (e.g., (Gly₄Ser)ₙ) (SEQ ID NO: 315) through random mutagenesis.

In some embodiments, a CD123-binding polypeptide comprises, in order from amino-terminus to carboxyl-terminus (or in order from carboxyl-terminus to amino-terminus), (i) the CD123-binding domain, (ii) a hinge region, (iii) an immunoglobulin constant region, (iv) a carboxyl-terminus linker (or an amino-terminus linker), and (v) the second binding domain. As used herein in the context of a polypeptide construct comprising a first binding domain and a second binding domain, a “hinge region” or a “hinge” refers to a polypeptide region between the first binding domain and the Fc region. A “carboxyl-terminus linker” or “an amino-terminus linker” refers to a polypeptide region between the Fc region and the second binding domain. In some embodiments, a carboxyl-terminus (or an amino-terminus linker) linker comprises or consists of SEQ ID NO:248. In certain embodiments, a hinge is a wild-type human immunoglobulin hinge region. In certain other embodiments, one or more amino acid residues can be added at the amino- or carboxyl-terminus of a wild type immunoglobulin hinge region as part of a fusion protein construct design. For example, additional junction amino acid residues at the hinge amino-terminus can be “RT,” “RSS,” “TG,” or “T,” or at the hinge carboxyl-terminus can be “SG”, or a hinge deletion can be combined with an addition, such as ΔP with “SG” added at the carboxyl-terminus.

In certain embodiments, a hinge, a carboxyl-terminus linker, or an amino-terminus linker is an altered immunoglobulin hinge in which one or more cysteine residues in a wild type immunoglobulin hinge region is substituted with one or more other amino acid residues (e.g., serine or alanine).

Exemplary altered immunoglobulin hinges, carboxyl-terminus linkers, and amino-terminus linkers include an immunoglobulin human IgG1 hinge region having one, two or three cysteine residues found in a wild type human IgG1 hinge substituted by one, two or three different amino acid residues (e.g., serine or alanine). An altered immunoglobulin hinge can additionally have a proline substituted with another amino acid (e.g., serine or alanine). For example, the above-described altered human IgG1 hinge can additionally have a proline located carboxyl-terminal to the three cysteines of wild type human IgG1 hinge region substituted by another amino acid residue (e.g., serine, alanine). In one embodiment, the prolines of the core hinge region are not substituted.

In certain embodiments, a hinge, a carboxyl-terminus linker, or an amino-terminus linker polypeptide comprises or is a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to a wild type immunoglobulin hinge region, such as a wild type human IgG1 hinge, a wild type human IgG2 hinge, or a wild type human IgG4 hinge.

In further embodiments, a hinge, a carboxyl-terminus linker, or an amino-terminus linker present in a CD123-binding polypeptide can be a hinge that is not based on or derived from an immunoglobulin hinge (i.e., not a wild-type immunoglobulin hinge or an altered immunoglobulin hinge). Examples for such hinges and carboxyl-terminus linkers include peptides of about five to about 150 amino acids derived from an interdomain region of a transmembrane protein or stalk region of a type II C-lectin, for instance, peptides of about eight to 25 amino acids and peptides of about seven to 18 amino acids. In another embodiment, a carboxyl-terminus linker or amino-terminus linker comprises a (Gly₄Ser) repeat (SEQ ID NO: 315) such as (Gly₄Ser)₃PS (SEQ ID NO: 316).

In certain embodiments, hinge, carboxyl-terminus linker, and amino-terminal linker sequences have about 5 to 150 amino acids, 5 to 10 amino acids, 10 to 20 amino acids, 20 to 30 amino acids, 30 to 40 amino acids, 40 to 50 amino acids, 50 to 60 amino acids, 5 to 60 amino acids, 5 to 40 amino acids, 8 to 20 amino acids, or 10 to 15 amino acids. The hinge or linker can be primarily flexible, but can also provide more rigid characteristics or can contain primarily α-helical structure with minimal β-sheet structure. The lengths or the sequences of the hinges and linkers can affect the binding affinities of the binding domains to which the hinges are directly or indirectly (via another region or domain, such as an heterodimerization domain) connected as well as one or more activities of the Fc region portions to which the hinges or linkers are directly or indirectly connected.

In certain embodiments, hinge, carboxyl-terminus linker, and amino-terminal linker sequences are stable in plasma and serum and are resistant to proteolytic cleavage. The first lysine in the IgG1 upper hinge region can be mutated to minimize proteolytic cleavage, for instance, the lysine can be substituted with methionine, threonine, alanine or glycine, or is deleted.

In some embodiments of the disclosure, a CD123-binding polypeptide is capable of forming a heterodimer with a second polypeptide chain and comprises a hinge region (a) immediately amino-terminal to an immunoglobulin constant region (e.g., amino-terminal to a CH2 domain wherein the immungobloubolin constant region includes CH2 and CH3 domains, or amino-terminal to a CH3 domain wherein the immunoglobulin sub-regions includes CH3 and CH4 domains), (b) interposed between and connecting a binding domain (e.g., scFv) and a immunoglobulin heterodimerization domain, (c) interposed between and connecting a immunoglobulin heterodimerization domain and an immunoglobulin constant region (e.g., wherein the immunoglobulin constant region includes CH2 and CH3 domains or CH3 and CH4 domains), (d) interposed between and connecting an immunoglobulin constant region and a binding domain, (e) at the amino-terminus of a polypeptide chain, or (f) at the carboxyl-terminus of a polypeptide chain. A polypeptide chain comprising a hinge region as described herein will be capable of associating with a different polypeptide chain to form a heterodimeric protein provided herein, and the heterodimer formed will contain a binding domain that retains its target specificity or its specific target binding affinity.

Some exemplary hinge, carboxyl-terminus linker, and amino-terminus linker sequences suitable for use in accordance with the present disclosure are shown in the Tables 1 and 2 below. Additional exemplary hinge and linker regions are set forth in SEQ ID NOs: 241-244, 601, 78, 763-791, 228, 379-434, 618-749 of US 2013/0129723 (said sequences incorporated by reference herein).

TABLE 1
Exemplary hinges and linkers

| | | SEQ ID |
|---|---|---|
| Name | Amino Acid Sequence | NO |
| sss(s)-hIgG1 | EPKSSDKTHTSPPSS | SEQ ID |
| hinge | | NO: 215 |
| csc(s)-hIgG1 | EPKSCDKTHTSPPCS | SEQ ID |
| hinge | | NO: 216 |
| ssc(s)-hIgG1 | EPKSSDKTHTSPPCS | SEQ ID |
| hinge | | NO: 217 |
| scc(s)-hIgG1 | EPKSSDKTHTCPPCS | SEQ ID |
| hinge | | NO: 218 |
| css(s)-hIgG1 | EPKSCDKTHTSPPSS | SEQ ID |
| hinge | | NO: 219 |
| scs(s)-hIgG1 | EPKSSDKTHTCPPSS | SEQ ID |
| hinge | | NO: 220 |
| ccc(s)-hIgG1 | EPKSCDKTHTSPPCS | SEQ ID |
| hinge | | NO: 221 |
| ccc(p)-hIgG1 | EPKSCDKTHTSPPCP | SEQ ID |
| hinge | | NO: 222 |
| sss(p)-hIgG1 | EPKSSDKTHTSPPSP | SEQ ID |
| hinge | | NO: 223 |
| csc(p)-hIgG1 | EPKSCDKTHTSPPCP | SEQ ID |
| hinge | | NO: 224 |
| ssc(p)-hIgG1 | EPKSSDKTHTSPPCP | SEQ ID |
| hinge | | NO: 225 |
| scc(p)-hIgG1 | EPKSSDKTHTCPPCP | SEQ ID |
| hinge | | NO: 226 |
| css(p)-hIgG1 | EPKSCDKTHTSPPSP | SEQ ID |
| hinge | | NO: 227 |
| scs(p)-hIgG1 | EPKSSDKTHTCPPSP | SEQ ID |
| hinge | | NO: 228 |
| Scppcp | SCPPCP | SEQ ID |
| | | NO: 229 |
| STD1 | NYGGGGSGGGGSGGGGSG | SEQ ID |
| | NS | NO: 230 |
| STD2 | NYGGGGSGGGGSGGGGS | SEQ ID |
| | GNYGGGGSGGGGSGGGGS | NO: 231 |
| | GNS | |
| H1 | NS | SEQ ID |
| | | NO: 232 |
| H2 | GGGGSGNS | SEQ ID |
| | | NO: 233 |
| H3 | NYGGGGSGNS | SEQ ID |
| | | NO: 234 |
| H4 | GGGGSGGGGSGNS | SEQ ID |
| | | NO: 235 |
| H5 | NYGGGGSGGGGSGNS | SEQ ID |
| | | NO: 236 |
| H6 | GGGGSGGGGSGGGGSGNS | SEQ ID |
| | | NO: 237 |
| H7 | GCPPCPNS | SEQ ID |
| | | NO: 238 |
| (G₄S)₃ | GGGGSGGGGSGGGGS | SEQ ID |
| | | NO: 239 |
| H105 | SGGGGSGGGGSGGGGS | SEQ ID |
| | | NO: 240 |
| (G₄S)₄ | GGGGSGGGGSGGGGSGGG | SEQ ID |
| | GS | NO: 241 |
| H75 (NKG2A | QRHNNSSLNTGTQMAGHS | SEQ ID |
| quadruple | PNS | NO: 242 |
| mutant) | | |
| H83 (NKG2A | SSLNTGTQMAGHSPNS | SEQ ID |
| derived) | | NO: 243 |
| H106 (NKG2A | QRHNNSSLNTGTQMAGHS | SEQ ID |
| derived) | | NO: 244 |
| H81 (NKG2D | EVQIPLTESYSPNS | SEQ ID |
| derived) | | NO: 245 |
| H91 (NKG2D | NSLANQEVQIPLTESYSP | SEQ ID |
| derived) | NS | NO: 246 |
| H94 | SGGGGSGGGGSGGGGSPNS | SEQ ID |
| | | NO: 247 |
| H111 | SGGGGSGGGGSGGGGSPGS | SEQ ID |
| | | NO: 248 |
| H114 | GGGGSGGGGSGGGGSPS | SEQ ID |
| | | NO: 288 |

TABLE 2
Exemplary hinges and linkers (derived
from H7 hinge, stalk region of a type II C-
lectin, or interdomain region of a type I
transmembrane protein)

| | | Molecule and/or | |
|---|---|---|---|
| | Amino Acid | hinge from | SEQ ID |
| Name | Sequence | which derived | NO |
| H16 | LSVKADFLTP | CD80 | SEQ ID |
| | SIGNS | | NO: 249 |
| H17 | LSVKADFLTP | CD80 + H7 | SEQ ID |
| | SISCPPCPNS | | NO: 250 |
| H18 | LSVLANFSQP | CD86 | SEQ ID |
| | EIGNS | | NO: 251 |
| H19 | LSVLANFSQP | CD86 + H7 | SEQ ID |
| | EISCPPCPNS | | NO: 252 |
| H20 | LKIQERVSKP | CD2 | SEQ ID |
| | KISNS | | NO: 253 |
| H21 | LKIQERVSKP | CD2 + H7 | SEQ ID |
| | KISCPPCPNS | | NO: 254 |
| H22 | LNVSERPFPP | CD22 | SEQ ID |
| | HIQNS | | NO: 255 |
| H23 | LDVSERPFPP | CD22 + H7 | SEQ ID |
| | HIQSCPPCPN | | NO: 256 |
| | S | | |
| H24 | REQLAEVTLS | CD80 | SEQ ID |
| | LKANS | | NO: 257 |
| H25 | REQLAEVTLS | CD80 + H7 | SEQ ID |
| | LKACPPCPNS | | NO: 258 |
| H26 | RIHQMNSELS | CD86 | SEQ ID |
| | VLANS | | NO: 259 |
| H27 | RIHQMNSELS | CD86 + H7 | SEQ ID |
| | VLACPPCPNS | | NO: 260 |
| H28 | DTKGKNVLEK | CD2 | SEQ ID |
| | IFSNS | | NO: 261 |
| H30 | LPPETQESQE | CD22 | SEQ ID |
| | VTLNS | | NO: 262 |
| H32 | RIHLNVSERP | CD22 | SEQ ID |
| | FPPNS | | NO: 263 |
| H33 | RIHLNVSERP | CD22 + H7 | SEQ ID |
| | FPPCPPCPNS | | NO: 264 |
| H36 | GCPPCPGGGG | H7 | SEQ ID |
| | SNS | | NO: 265 |
| H40 | GCPPCPANS | H7 | SEQ ID |
| | | | NO: 266 |
| H41 | GCPPCPANS | H7 | SEQ ID |
| | | | NO: 267 |
| H42 | GCPPCPNS | H7 | SEQ ID |
| | | | NO: 268 |
| H44 | GGGASCPPCP | H7 | SEQ ID |
| | GNS | | NO: 269 |
| H45 | GGGASCPPCA | H7 | SEQ ID |
| | GNS | | NO: 270 |
| H46 | GGGASCPPCA | H7 | SEQ ID |
| | NS | | NO: 271 |
| H47 | LSVKADFLTP | CD80 | SEQ ID |
| | SIGNS | | NO: 272 |
| H48 | ADFLTPSIGN | CD80 | SEQ ID |
| | S | | NO: 273 |
| H50 | LSVLANFSQP | CD86 | SEQ ID |
| | EIGNS | | NO: 274 |
| H51 | LSVLANFSQP | CD86 | SEQ ID |
| | EIGNS | | NO: 275 |
| H52 | SQPEIVPISN | CD86 | SEQ ID |
| | S | | NO: 276 |
| H53 | SQPEIVPISC | CD86 + H7 | SEQ ID |
| | PPCPNS | | NO: 277 |
| H54 | SVLANFSQPE | CD86 + H7 | SEQ ID |
| | ISCPPCPNS | | NO: 278 |
| H55 | RIHQMNSELS | CD86 | SEQ ID |
| | VLANS | | NO: 279 |
| H56 | QMNSELSVLA | CD86 | SEQ ID |
| | NS | | NO: 280 |
| H57 | VSERPFPPNS | CD22 | SEQ ID |
| | | | NO: 281 |
| H58 | KPFFTCGSAD | CD72 | SEQ ID |
| | TCPNS | | NO: 282 |
| H59 | KPFFTCGSAD | CD72 | SEQ ID |
| | TCPNS | | NO: 283 |
| H60 | QYNCPGQYTF | CD69 | SEQ ID |
| | SMPNS | | NO: 284 |
| H61 | EPAFTPGPNI | CD94 | SEQ ID |
| | ELQKDSDCPN | | NO: 285 |
| | S | | |
| H62 | QRHNNSSLNT | NKG2A | SEQ ID |
| | RTQKARHCPN | | NO: 286 |
| | S | | |
| H63 | NSLFNQEVQI | NKG2D | SEQ ID |
| | PLTESYCPNS | | NO: 287 |

As indicated herein, in certain embodiments, polypeptides of the present disclosure comprise an immunoglobulin constant region (also referred to as a constant region) in a polypeptide chain. The inclusion of an immunoglobulin constant region slows clearance of the homodimeric and heterodimeric proteins formed from two CD123-binding polypeptide chains from circulation after administration to a subject. By mutations or other alterations, an immunoglobulin constant region further enables relatively easy modulation of dimeric polypeptide effector functions (e.g., ADCC, ADCP, CDC, complement fixation, and binding to Fc receptors), which can either be increased or decreased depending on the disease being treated, as known in the art and described herein. In certain embodiments, an immunoglobulin constant region of one or both of the polypeptide chains of the polypeptide homodimers and heterodimers of the present disclosure will be capable of mediating one or more of these effector functions In other embodiments, one or more of these effector functions are reduced or absent in an immunoglobulin constant region of one or both of the polypeptide chains of the polypeptide homodimers and heterodimers of the present disclosure, as compared to a corresponding wild-type immunoglobulin constant region. For example, for dimeric CD123-binding polypeptides designed to elicit RTCC, such as, e.g., via the inclusion of a CD3-binding domain, an immunoglobulin constant region may have reduced or no effector function relative to a corresponding wild-type immunoglobulin constant region.

An immunoglobulin constant region present in polypeptides of the present disclosure can comprise or is derived from part or all of: a CH2 domain, a CH3 domain, a CH4 domain, or any combination thereof. For example, an immunoglobulin constant region can comprise a CH2 domain, a CH3 domain, both CH2 and CH3 domains, both CH3 and CH4 domains, two CH3 domains, a CH4 domain, two CH4 domains, and a CH2 domain and part of a CH3 domain.

A CH2 domain that can form an immunoglobulin constant region of a polypeptide of the present disclosure can be a wild type immunoglobulin CH2 domain or an altered immunoglobulin CH2 domain thereof from certain immunoglobulin classes or subclasses (e.g., IgG1, IgG2, IgG3, IgG4, IgA1, IgA2, or IgD) and from various species (including human, mouse, rat, and other mammals).

In certain embodiments, a CH2 domain is a wild type human immunoglobulin CH2 domain, such as wild type CH2 domains of human IgG1, IgG2, IgG3, IgG4, IgA1, IgA2, or IgD, as set forth in SEQ ID NOS: 115, 199-201 and 195-197, respectively, of US 2013/0129723 (said sequences incorporated by reference herein). In certain embodiments, the CH2 domain is a wild type human IgG1 CH2 domain as set forth in SEQ ID NO:115 of US 2013/0129723 (said sequence incorporated by reference herein).

In certain embodiments, a CH2 domain is an altered immunoglobulin CH2 region (e.g., an altered human IgG1 CH2 domain) that comprises an amino acid substitution at the asparagine of position 297 (e.g., asparagine to alanine). Such an amino acid substitution reduces or eliminates glycosylation at this site and abrogates efficient Fc binding to FcγR and C1q. The sequence of an altered human IgG1 CH2 domain with an Asn to Ala substitution at position 297 is set forth in SEQ ID NO:324 of US 2013/0129723 (said sequence incorporated by reference herein).

In certain embodiments, a CH2 domain is an altered immunoglobulin CH2 region (e.g., an altered human IgG1 CH2 domain) that comprises at least one substitution or deletion at positions 234 to 238. For example, an immunoglobulin CH2 region can comprise a substitution at position 234, 235, 236, 237 or 238, positions 234 and 235, positions 234 and 236, positions 234 and 237, positions 234 and 238, positions 234-236, positions 234, 235 and 237, positions 234, 236 and 238, positions 234, 235, 237, and 238, positions 236-238, or any other combination of two, three, four, or five amino acids at positions 234-238. In addition or alternatively, an altered CH2 region can comprise one or more (e.g., two, three, four or five) amino acid deletions at positions 234-238, for instance, at one of position 236 or position 237 while the other position is substituted. The above-noted mutation(s) decrease or eliminate the antibody-dependent cell-mediated cytotoxicity (ADCC) activity or Fc receptor-binding capability of a polypeptide heterodimer that comprises the altered CH2 domain. In certain embodiments, the amino acid residues at one or more of positions 234-238 has been replaced with one or more alanine residues. In further embodiments, only one of the amino acid residues at positions 234-238 have been deleted while one or more of the remaining amino acids at positions 234-238 can be substituted with another amino acid (e.g., alanine or serine).

In certain other embodiments, a CH2 domain is an altered immunoglobulin CH2 region (e.g., an altered human IgG1 CH2 domain) that comprises one or more amino acid substitutions at positions 253, 310, 318, 320, 322, and 331. For example, an immunoglobulin CH2 region can comprise a substitution at position 253, 310, 318, 320, 322, or 331, positions 318 and 320, positions 318 and 322, positions 318, 320 and 322, or any other combination of two, three, four, five or six amino acids at positions 253, 310, 318, 320, 322, and 331. The above-noted mutation(s) decrease or eliminate the complement-dependent cytotoxicity (CDC) of a polypeptide heterodimer that comprises the altered CH2 domain.

In certain other embodiments, in addition to the amino acid substitution at position 297, an altered CH2 region (e.g., an altered human IgG1 CH2 domain) can further comprise one or more (e.g., two, three, four, or five) additional substitutions at positions 234-238. For example, an immunoglobulin CH2 region can comprise a substitution at positions 234 and 297, positions 234, 235, and 297, positions 234, 236 and 297, positions 234-236 and 297, positions 234, 235, 237 and 297, positions 234, 236, 238 and 297, positions 234, 235, 237, 238 and 297, positions 236-238 and 297, or any combination of two, three, four, or five amino acids at positions 234-238 in addition to position 297. In addition or alternatively, an altered CH2 region can comprise one or more (e.g., two, three, four or five) amino acid deletions at positions 234-238, such as at position 236 or position 237. The additional mutation(s) decreases or eliminates the antibody-dependent cell-mediated cytotoxicity (ADCC) activity or Fc receptor-binding capability of a polypeptide heterodimer that comprises the altered CH2 domain. In certain embodiments, the amino acid residues at one or more of positions 234-238 have been replaced with one or more alanine residues. In further embodiments, only one of the amino acid residues at positions 234-238 has been deleted while one or more of the remaining amino acids at positions 234-238 can be substituted with another amino acid (e.g., alanine or serine).

In certain embodiments, in addition to one or more (e.g., 2, 3, 4, or 5) amino acid substitutions at positions 234-238, a mutated CH2 region (e.g., an altered human IgG1 CH2 domain) in a fusion protein of the present disclosure can contain one or more (e.g., 2, 3, 4, 5, or 6) additional amino acid substitutions (e.g., substituted with alanine) at one or more positions involved in complement fixation (e.g., at positions I253, H310, E318, K320, K322, or P331). Examples of mutated immunoglobulin CH2 regions include human IgG1, IgG2, IgG4 and mouse IgG2a CH2 regions with alanine substitutions at positions 234, 235, 237 (if present), 318, 320 and 322. An exemplary mutated immunoglobulin CH2 region is mouse IGHG2c CH2 region with alanine substitutions at L234, L235, G237, E318, K320, and K322.

In still further embodiments, in addition to the amino acid substitution at position 297 and the additional deletion(s) or substitution(s) at positions 234-238, an altered CH2 region (e.g., an altered human IgG1 CH2 domain) can further comprise one or more (e.g., two, three, four, five, or six) additional substitutions at positions 253, 310, 318, 320, 322, and 331. For example, an immunoglobulin CH2 region can comprise a (1) substitution at position 297, (2) one or more substitutions or deletions or a combination thereof at positions 234-238, and one or more (e.g., 2, 3, 4, 5, or 6) amino acid substitutions at positions I253, H310, E318, K320, K322, and P331, such as one, two, three substitutions at positions E318, K320 and K322. The amino acids at the above-noted positions can be substituted by alanine or serine.

In certain embodiments, an immunoglobulin CH2 region polypeptide comprises: (i) an amino acid substitution at the asparagines of position 297 and one amino acid substitution at position 234, 235, 236 or 237; (ii) an amino acid substitution at the asparagine of position 297 and amino acid substitutions at two of positions 234-237; (iii) an amino acid substitution at the asparagine of position 297 and amino acid substitutions at three of positions 234-237; (iv) an amino acid substitution at the asparagine of position 297, amino acid substitutions at positions 234, 235 and 237, and an amino acid deletion at position 236; (v) amino acid substitutions at three of positions 234-237 and amino acid substitutions at positions 318, 320 and 322; or (vi) amino acid substitutions at three of positions 234-237, an amino acid deletion at position 236, and amino acid substitutions at positions 318, 320 and 322.

Exemplary altered immunoglobulin CH2 regions with amino acid substitutions at the asparagine of position 297 include: human IgG1 CH2 region with alanine substitutions at L234, L235, G237 and N297 and a deletion at G236 (SEQ ID NO:325 of US 2013/0129723, said sequence incorporated by reference herein), human IgG2 CH2 region with alanine substitutions at V234, G236, and N297 (SEQ ID NO:326 of US 2013/0129723, said sequence incorporated by reference herein), human IgG4 CH2 region with alanine substitutions at F234, L235, G237 and N297 and a deletion of G236 (SEQ ID NO:322 of US 2013/0129723, said sequence incorporated by reference herein), human IgG4 CH2 region with alanine substitutions at F234 and N297 (SEQ ID NO:343 of US 2013/0129723, said sequence incorporated by reference herein), human IgG4 CH2 region with alanine substitutions at L235 and N297 (SEQ ID NO:344 of US 2013/0129723, said sequence incorporated by reference herein), human IgG4 CH2 region with alanine substitutions at G236 and N297 (SEQ ID NO:345 of US 2013/0129723, said sequence incorporated by reference herein), and human IgG4 CH2 region with alanine substitutions at G237 and N297 (SEQ ID NO:346 of US 2013/0129723, said sequence incorporated by reference herein).

In certain embodiments, in addition to the amino acid substitutions described above, an altered CH2 region (e.g., an altered human IgG1 CH2 domain) can contain one or more additional amino acid substitutions at one or more positions other than the above-noted positions. Such amino acid substitutions can be conservative or non-conservative amino acid substitutions. For example, in certain embodiments, P233 can be changed to E233 in an altered IgG2 CH2 region (see, e.g., SEQ ID NO:326 of US 2013/0129723, said sequence incorporated by reference herein). In addition or alternatively, in certain embodiments, the altered CH2 region can contain one or more amino acid insertions, deletions, or both. The insertion(s), deletion(s) or substitution(s) can be anywhere in an immunoglobulin CH2 region, such as at the N- or C-terminus of a wild type immunoglobulin CH2 region resulting from linking the CH2 region with another region (e.g., a binding domain or an immunoglobulin heterodimerization domain) via a hinge.

In certain embodiments, an altered CH2 region in a polypeptide of the present disclosure comprises or is a sequence that is at least 90%, at least 91%, at least 92%, at least 93%, at least 94%, at least 95%, at least 96%, at least 97%, at least 98%, at least 99% identical to a wild type immunoglobulin CH2 region, such as the CH2 region of wild type human IgG1, IgG2, or IgG4, or mouse IgG2a (e.g., IGHG2c).

An altered immunoglobulin CH2 region in a CD123-binding polypeptide of the present disclosure can be derived from a CH2 region of various immunoglobulin isotypes, such as IgG1, IgG2, IgG3, IgG4, IgA1, IgA2, and IgD, from various species (including human, mouse, rat, and other mammals). In certain embodiments, an altered immunoglobulin CH2 region in a fusion protein of the present disclosure can be derived from a CH2 region of human IgG1, IgG2 or IgG4, or mouse IgG2a (e.g., IGHG2c), whose sequences are set forth in SEQ ID NOS:115, 199, 201, and 320 of US 2013/0129723 (said sequences incorporated by reference herein).

In certain embodiments, an altered CH2 domain is a human IgG1 CH2 domain with alanine substitutions at positions 235, 318, 320, and 322 (i.e., a human IgG1 CH2 domain with L235A, E318A, K320A and K322A substitutions) (SEQ ID NO:595 of US 2013/0129723, said sequence incorporated by reference herein), and optionally an N297 mutation (e.g., to alanine). In certain other embodiments, an altered CH2 domain is a human IgG1 CH2 domain with alanine substitutions at positions 234, 235, 237, 318, 320 and 322 (i.e., a human IgG1 CH2 domain with L234A, L235A, G237A, E318A, K320A and K322A substitutions) (SEQ ID NO:596 of US 2013/0129723, said sequence incorporated by reference herein), and optionally an N297 mutation (e.g., to alanine).

In certain embodiments, an altered CH2 domain is an altered human IgG1 CH2 domain with mutations known in the art that enhance immunological activities such as ADCC, ADCP, CDC, complement fixation, Fc receptor binding, or any combination thereof.

The CH3 domain that can form an immunoglobulin constant region of a polypeptide of the present disclosure can be a wild type immunoglobulin CH3 domain or an altered immunoglobulin CH3 domain thereof from certain immunoglobulin classes or subclasses (e.g., IgG1, IgG2, IgG3, IgG4, IgA1, IgA2, IgD, IgE, IgM) of various species (including human, mouse, rat, and other mammals). In certain embodiments, a CH3 domain is a wild type human immunoglobulin CH3 domain, such as wild type CH3 domains of human IgG1, IgG2, IgG3, IgG4, IgA1, IgA2, IgD, IgE, or IgM as set forth in SEQ ID NOS:116, 208-210, 204-207, and 212, respectively of US 2013/0129723 (said sequences incorporated by reference herein). In certain embodiments, the CH3 domain is a wild type human IgG1 CH3 domain as set forth in SEQ ID NO:116 of US 2013/0129723 (said sequence incorporated by reference herein). In certain embodiments, a CH3 domain is an altered human immunoglobulin CH3 domain, such as an altered CH3 domain based on or derived from a wild-type CH3 domain of human IgG1, IgG2, IgG3, IgG4, IgA1, IgA2, IgD, IgE, or IgM antibodies. For example, an altered CH3 domain can be a human IgG1 CH3 domain with one or two mutations at positions H433 and N434 (positions are numbered according to EU numbering). The mutations in such positions can be involved in complement fixation. In certain other embodiments, an altered CH3 domain can be a human IgG1 CH3 domain but with one or two amino acid substitutions at position F405 or Y407. The amino acids at such positions are involved in interacting with another CH3 domain. In certain embodiments, an altered CH3 domain can be an altered human IgG1 CH3 domain with its last lysine deleted. The sequence of this altered CH3 domain is set forth in SEQ ID NO:761 of US 2013/0129723 (said sequence incorporated by reference herein).

In certain embodiments, CD123-binding polypeptides forming a polypeptide heterodimer comprise a CH3 pair that comprises so called “knobs-into-holes” mutations (see, Marvin and Zhu, Acta Pharmacologica Sinica 26:649-58, 2005; Ridgway et al., Protein Engineering 9:617-21, 1996). More specifically, mutations can be introduced into each of the two CH3 domains of each polypeptide chain so that the steric complementarity required for CH3/CH3 association obligates these two CH3 domains to pair with each other. For example, a CH3 domain in one single chain polypeptide of a polypeptide heterodimer can contain a T366W mutation (a “knob” mutation, which substitutes a small amino acid with a larger one), and a CH3 domain in the other single chain polypeptide of the polypeptide heterodimer can contain a Y407A mutation (a “hole” mutation, which substitutes a large amino acid with a smaller one). Other exemplary knobs-into-holes mutations include (1) a T366Y mutation in one CH3 domain and a Y407T in the other CH3 domain, and (2) a T366W mutation in one CH3 domain and T366S, L368A and Y407V mutations in the other CH3 domain.

The CH4 domain that can form an immunoglobulin constant region of CD123-binding polypeptides of the present disclosure can be a wild type immunoglobulin CH4 domain or an altered immunoglobulin CH4 domain thereof from IgE or IgM molecules. In certain embodiments, the CH4 domain is a wild type human immunoglobulin CH4 domain, such as wild type CH4 domains of human IgE and IgM molecules as set forth in SEQ ID NOS:213 and 214, respectively, of US 2013/0129723 (said sequences incorporated by reference herein). In certain embodiments, a CH4 domain is an altered human immunoglobulin CH4 domain, such as an altered CH4 domain based on or derived from a CH4 domain of human IgE or IgM molecules, which have mutations that increase or decrease an immunological activity known to be associated with an IgE or IgM Fc region.

In certain embodiments, an immunoglobulin constant region of CD123-binding polypeptides of the present disclosure comprises a combination of CH2, CH3 or CH4 domains (i.e., more than one constant region domain selected from CH2, CH3 and CH4). For example, the immunoglobulin constant region can comprise CH2 and CH3 domains or CH3 and CH4 domains. In certain other embodiments, the immunoglobulin constant region can comprise two CH3 domains and no CH2 or CH4 domains (i.e., only two or more CH3). The multiple constant region domains that form an immunoglobulin constant region can be based on or derived from the same immunoglobulin molecule, or the same class or subclass immunoglobulin molecules. In certain embodiments, the immunoglobulin constant region is an IgG CH2CH3 (e.g., IgG1 CH2CH3, IgG2 CH2CH3, and IgG4 CH2CH3) and can be a human (e.g., human IgG1, IgG2, and IgG4) CH2CH3. For example, in certain embodiments, the immunoglobulin constant region comprises (1) wild type human IgG1 CH2 and CH3 domains, (2) human IgG1 CH2 with N297A substitution (i.e., CH2(N297A)) and wild type human IgG1 CH3, or (3) human IgG1 CH2(N297A) and an altered human IgG1 CH3 with the last lysine deleted.

Alternatively, the multiple constant region domains can be based on or derived from different immunoglobulin molecules, or different classes or subclasses immunoglobulin molecules. For example, in certain embodiments, an immunoglobulin constant region comprises both human IgM CH3 domain and human IgG1 CH3 domain. The multiple constant region domains that form an immunoglobulin constant region can be directly linked together or can be linked to each other via one or more (e.g., about 2-10) amino acids.

Exemplary immunoglobulin constant regions are set forth in SEQ ID NOS:305-309, 321, 323, 341, 342, and 762 of US 2013/0129723 (said sequences incorporated by reference herein).

In certain embodiments, the immunoglobulin constant regions of both CD123-binding polypeptides of a polypeptide dimer are identical to each other. In certain other embodiments, the immunoglobulin constant region of one polypeptide chain of a dimeric protein is different from the immunoglobulin constant region of the other polypeptide chain of the dimer. For example, one immunoglobulin constant region of a heterodimeric protein can contain a CH3 domain with a “knob” mutation, whereas the other immunoglobulin constant region of the heterodimeric protein can contain a CH3 domain with a “hole” mutation.

The disclosure relates to CD123-binding proteins and polypeptides that may comprise any of the sequences shown in Table 3. In some embodiments, CD123-binding proteins and polypeptides may comprise a signal sequence. Sequences for various cloned sequences and components are also presented in Table 3. Amino acid sequences given for polypeptide constructs do not include the human or rabbit immunoglobulin leader sequences. CDR sequences and amino acid substitution positions shown are those defined using the IMGT criteria (Brochet, X, et al, Nucl. Acids Res. (2008) 36, W503-508). Thus, the first residue in FR1 of a heavy or light chain variable domain or region is considered to be position 1.

TABLE 3
Binding Polypeptide Sequences and Components

| | | | SEQ ID NOs: |
|---|---|---|---|
| | Nucleotide | Amino Acid | nucleotide |
| Name | Sequence | Sequence | (amino acid) |
| OMT1 | gacatcgtgatgacccagtctccagactccctggctgtgtctctg | divmtqspdslavslg | SEQ ID NO: 1 |
| variable | ggcgagagggccaccatcaactgcaagtccagccacagtgtttta | eratincksshsvlys | (SEQ ID NO: 2) |
| light | tacagctccaacaataagaactacttagcttggtaccagcagaaa | snnknylawyqqkpgq | |
| chain | ccaggacagcctcctaagctgctcatttactgggcatctacccgg | ppklliywastresgv | |
| domain | gaatccggggtccctgaccgattcagtggcagcgggtctgggaca | pdrfsgsgsgtdftlt | |
| | gatttcactctcaccatcagcagcctgcaggctgaagatgtggca | isslqaedvavyycqq | |
| | gtttattactgtcagcaatattatagtactcctccgaccactttc | yystppttfgggtkve | |
| | ggcggagggaccaaggtggagatcaaa | ik | |
| OMT1 | gaggtgcagctgttggagtctgggggaggcttggtacagcctggg | evqllesggglvqpgg | SEQ ID NO: 3 |
| variable | gggtccctgagactctcctgtgcagcctctggattcacctttagc | slrlscaasgftfssy | (SEQ ID NO: 4) |
| heavy | agctatggcatgagctgggtccgccaggctccagggaaggggctg | gmswvrqapgkglegv | |
| chain | gagggggtctcagctattagtggtagtggtggtagcacatactac | saisgsggstyyadsv | |
| domain | gcagactccgtgaagggccggttcaccatctccagagacaattcc | kgrftisrdnskntly | |
| | aagaacacgctgtatctgcaaatgaacagcctgagagccgaggac | lqmnslraedtavyyc | |
| | acggccgtatattactgtgcgaaagaaaagttacgatattttgac | akeklryfdwlsdafd | |
| | tggttatccgatgcttttgatatctggggccaagggacaatggtc | iwgqgtmvtvss | |
| | accgtctcttca | | |
| OMT1 | cacagtgttttatacagctccaacaataagaactac | HSVLYSSNNKNY | SEQ ID NO: 5 |
| CDR L1 | | | (SEQ ID NO: 6) |
| OMT1 | tgggcatct | WAS | SEQ ID NO: 7 |
| CDR L2 | | | (SEQ ID NO: 8) |
| OMT1 | cagcaatattatagtactcctccgaccact | QQYYSTPPTT | SEQ ID NO: 9 |
| CDR L3 | | | (SEQ ID NO: 10) |
| OMT1 | ggattcacctttagcagctatggc | GFTFSSYG | SEQ ID NO: 11 |
| CDR H1 | | | (SEQ ID NO: 12) |
| OMT1 | attagtggtagtggtggtagcaca | ISGSGGST | SEQ ID NO: 13 |
| CDR H2 | | | (SEQ ID NO: 14) |
| OMT1 | gcgaaagaaaagttacgatattttgactggttatccgatgctttt | AKEKLRYFDWLSDAFD | SEQ ID NO: 15 |
| CDR H3 | gatatc | I | (SEQ ID NO: 16) |
| DB8 | gacatccagatgacccagtctccatcctccctgtctgcatctgta | diqmtqspsslsasvg | SEQ ID NO: 17 |
| variable | ggagacagagtcaccatcacttgccgggcaagtcagagcattagc | drvtitcrasqsissy | (SEQ ID NO: 18) |
| light | agctatctgaattggtatcagcagaaaccagggaaagcccctaag | lnwyqqkpgkapklli | |
| chain | ctcctgatctatgctgcatccagtttgcaaagtggggtcccatca | yaasslqsgvpsrfsg | |
| domain | aggttcagtggcagtggatctgggacagatttcactctcaccatc | sgsgtdftltisslqp | |
| | agcagtctgcaacctgaagattttgcaacttactactgtcaacag | edfatyycqqsystpl | |
| | agttacagtacccctctcactttcggcggaggtaccaaggtggag | tfgggtkveik | |
| | atcaaa | | |
| DB8 | caggtgcagctggtgcagtctggggctgaggtgaagaagcctggg | qvqlvqsgaevkkpga | SEQ ID NO: 19 |
| variable | gcctcagtgaaggtttcctgcaaggcatctggatacatcttcacc | svkvsckasgyiftdy | (SEQ ID NO: 20) |
| heavy | gactactatatgcactgggtgcgtcaggcccctggacaagggctt | ymhwvrqapgqglewm | |
| chain | gagtggatgggatggatgagccctaacagtggtaacacaggctat | gwmspnsgntgyaqkf | |
| domain | gcacagaagttccagggccgtgtcaccatgacccgcgacacgtcc | qgrvtmtrdtststvy | |
| | acgagcacagtctacatggagctgagcagcctgcgttctgaggac | melssirsedtavyyc | |
| | acggccgtgtattactgtgcgagagatgcggcggattacggtgac | ardaadygdyvafdiw | |
| | tacgttgcttttgatatctggggccaagggacaatggtcaccgtc | gqgtmvtvss | |
| | tcttca | | |
| DB8 | cagagcattagcagctat | QSISSY | SEQ ID NO: 21 |
| CDR L1 | | | (SEQ ID NO: 22) |
| DB8 | gctgcatcc | AAS | SEQ ID NO: 23 |
| CDR L2 | | | (SEQ ID NO: 24) |
| DB8 | caacagagttacagtacccctctcact | QQSYSTPLT | SEQ ID NO: 25 |
| CDR L3 | | | (SEQ ID NO: 26) |
| DB8 | ggatacatcttcaccgactactat | GYIFTDYY | SEQ ID NO: 27 |
| CDR H1 | | | (SEQ ID NO: 28) |
| DB8 | atgagccctaacagtggtaacaca | MSPNSGNT | SEQ ID NO: 29 |
| CDR H2 | | | (SEQ ID NO: 30) |
| DB8 | gcgagagatgcggcggattacggtgactacgttgcttttgatatc | ARDAADYGDYVAFDI | SEQ ID NO: 31 |
| CDR H3 | | | (SEQ ID NO: 32) |
| DB60 | gacatccagatgacccagtctccatcctccctgtctgcatctgta | diqmtqspsslsasvg | SEQ ID NO: 17 |
| variable | ggagacagagtcaccatcacttgccgggcaagtcagagcattagc | drvtitcrasqsissy | (SEQ ID NO: 18) |
| light | agctatctgaattggtatcagcagaaaccagggaaagcccctaag | lnwyqqkpgkapklli | |
| chain | ctcctgatctatgctgcatccagtttgcaaagtggggtcccatca | yaasslqsgvpsrfsg | |
| domain | aggttcagtggcagtggatctgggacagatttcactctcaccatc | sgsgtdftltisslqp | |
| | agcagtctgcaacctgaagattttgcaacttactactgtcaacag | edfatyycqqsystpl | |
| | agttacagtacccctctcactttcggcggaggtaccaaggtggag | tfgggtkveik | |
| | atcaaa | | |
| DB60 | caggtgcagctggtgcagtctggggctgaggtgaagaagcctggg | qvqlvqsgaevkkpga | SEQ ID NO: 33 |
| variable | gcctc | s | (SEQ ID NO: 34) |
| heavy | agtgaaggtttcctgcaaggcatctggatacaccttcaccagcta | vkvsckasgytftsyy | |
| chain | ctatat | m | |
| domain | gcactgggtgcgtcaggcccctggacaagggcttgagtggatggg | hwvrqapgqglewmg | |
| | gtgg | | |
| | atcaaccctaacagtggtgacacaagctatgcacagaagttccag | winpnsgdtsyaqkfq | |
| | ggcc | g | |
| | gtgtcaccatgacccgcgacacgtccacgagcacagtctacatgg | rvtmtrdtststvyme | |
| | agctg | ls | |
| | agcagcctgcgttctgaggacacggccgtgtattactgtgcgcag | slrsedtavyycaqds | |
| | gatag | sg | |
| | tagtggttccggggcttttgatatctggggccaagggacaatggt | sgafdiwgqgtmvtvs | |
| | caccgt | s | |
| | ctcttca | | |
| DB60 | cagagcattagcagctat | QSISSY | SEQ ID NO: 21 |
| CDR L1 | | | (SEQ ID NO: 22) |
| DB60 | gctgcatcc | AAS | SEQ ID NO: 23 |
| CDR L2 | | | (SEQ ID NO: 24) |
| DB60 | caacagagttacagtacccctctcact | QQSYSTPLT | SEQ ID NO: 25 |
| CDR L3 | | | (SEQ ID NO: 26) |
| DB60 | ggatacaccttcaccagctactat | GYTFTSYY | SEQ ID NO: 35 |
| CDR H1 | | | (SEQ ID NO: 36) |
| DB60 | atcaaccctaacagtggtgacaca | INPNSGDT | SEQ ID NO: 37 |
| CDR H2 | | | (SEQ ID NO: 38) |
| DB60 | gcgcaggatagtagtggttccggggcttttgatatc | AQDSSGSGAFDI | SEQ ID NO: 39 |
| CDR H3 | | | (SEQ ID NO: 40) |
| DB65 | gacatccagatgacccagtctccatcctccctgtctgcatctgta | diqmtqspsslsasvg | SEQ ID NO: 17 |
| variable | ggagacagagtcaccatcacttgccgggcaagtcagagcattagc | drvtitcrasqsissy | (SEQ ID NO: 18) |
| light | agctatctgaattggtatcagcagaaaccagggaaagcccctaag | lnwyqqkpgkapklli | |
| chain | ctcctgatctatgctgcatccagtttgcaaagtggggtcccatca | yaasslqsgvpsrfsg | |
| domain | aggttcagtggcagtggatctgggacagatttcactctcaccatc | sgsgtdftltisslqp | |
| | agcagtctgcaacctgaagattttgcaacttactactgtcaacag | edfatyycqqsystpl | |
| | agttacagtacccctctcactttcggcggaggtaccaaggtggag | tfgggtkveik | |
| | atcaaa | | |
| DB65 | caggtgcagctggtgcagtctggggctgaggtgaagaagcctggg | qvqlvqsgaevkkpga | SEQ ID NO: 41 |
| variable | gcctcagtgaaggtttcctgcaaggcatctggatacaccttcacc | svkvsckasgytftgy | (SEQ ID NO: 42) |
| heavy | ggctactatatgcactgggtgcgtcaggcccctggacaagggctt | ymhwvrqapgqglewm | |
| chain | gagtggatgggatggatgaaccctaacagtggtaacacaggctat | gwmnpnsgntgyaqkf | |
| domain | gcacagaagttccagggccgtgtcaccatgacccgcgacacgtcc | qgrvtmtrdtststvy | |
| | acgagcacagtctacatggagctgagcagcctgcgttctgaggac | melsslrsedtavyyc | |
| | acggccgtgtattactgtgcgaaagaggaaccgatttttggagtg | akeepifgvvmdafdi | |
| | gttatggatgcttttgatatctggggccaagggacaatggtcacc | wgqgtmvtvss | |
| | gtctcctca | | |
| DB65 | cagagcattagcagctat | QSISSY | SEQ ID NO: 21 |
| CDR L1 | | | (SEQ ID NO: 22) |
| DB65 | gctgcatcc | AAS | SEQ ID NO: 23 |
| CDR L2 | | | (SEQ ID NO: 24) |
| DB65 | caacagagttacagtacccctctcact | QQSYSTPLT | SEQ ID NO: 25 |
| CDR L3 | | | (SEQ ID NO: 26) |
| DB65 | ggatacaccttcaccggctactat | GYTFTGYY | SEQ ID NO: 43 |
| CDR H1 | | | (SEQ ID NO: 44) |
| DB65 | atgaaccctaacagtggtaacaca | MNPNSGNT | SEQ ID NO: 45 |
| CDR H2 | | | (SEQ ID NO: 46) |
| DB65 | gcgaaagaggaaccgatttttggagtggttatggatgcttttgat | AKEEPIFGVVMDAFDI | SEQ ID NO: 47 |
| CDR H3 | atc | | (SEQ ID NO: 48) |
| DB82 | gacatccagatgacccagtctccatcctccctgtctgcatctgta | diqmtqspsslsasvg | SEQ ID NO: 49 |
| variable | ggagaccgcgtcaccatcacttgccgggcaagtcagaccataaac | drvtitcrasqtinny | (SEQ ID NO: 50) |
| light | aactatttgaactggtatcagcagaaaccagggaaagcccctaag | lnwyqqkpgkapklli | |
| chain | ctcctgatctattctgcatctactttgcaaagtggggtcccatca | ysastlqsgvpsrfsg | |
| domain | cgtttcagtggcagtggatctgggacagatttcactctcaccatc | sgsgtdftltisslqp | |
| | agcagtctgcaacctgaagattttgcaacttactactgtcaccag | edfatyychqsytspl | |
| | agttacacttcacctctcactttcggcggaggtaccaaggtggag | tfgggtkveik | |
| | atcaaa | | |
| DB82 | gaggtgcagctggtggagtctgggggaggcttggtacagcctggg | evqlvesggglvqpgg | SEQ ID NO: 51 |
| variable | gggtccctgcgcctctcctgtgcagcctctggattcacctttagc | slriscaasgftfssy | (SEQ ID NO: 52) |
| heavy | agctatgccatgagctgggtccgccaggctccagggaaggggctg | amswvrqapgkglewv | |
| chain | gagtgggtctcagttattagtgccaatagtgctggtctaggccat | svisansaglghadsv | |
| domain | gcggactctgtgaagggccggttcaccatctcccgcgacaattcc | kgrftisrdnskntly | |
| | aagaacacgctgtatctgcaaatgaacagcctgcgcgccgaggac | lqmnslraedtavyyc | |
| | acggccgtatattactgtgcgagagtgggctatagcagctcggct | arvgysssadafdiwg | |
| | gatgcttttgatatctggggccaagggacaatggtcaccgtctcc | qgtmvtvss | |
| | tcg | | |
| DB82 | cagaccataaacaactat | QTINNY | SEQ ID NO: 53 |
| CDR L1 | | | (SEQ ID NO: 54) |
| DB82 | tctgcatct | SAS | SEQ ID NO: 55 |
| CDR L2 | | | (SEQ ID NO: 56) |
| DB82 | caccagagttacacttcacctctcact | HQSYTSPLT | SEQ ID NO: 57 |
| CDR L3 | | | (SEQ ID NO: 58) |
| DB82 | ggattcacctttagcagctatgcc | GFTFSSYA | SEQ ID NO: 59 |
| CDR H1 | | | (SEQ ID NO: 60) |
| DB82 | attagtgccaatagtgctggtcta | ISANSAGL | SEQ ID NO: 61 |
| CDR H2 | | | (SEQ ID NO: 62) |
| DB82 | gcgagagtgggctatagcagctcggctgatgcttttgatatc | ARVGYSSSADAFDI | SEQ ID NO: 63 |
| CDR H3 | | | (SEQ ID NO: 64) |
| DB83 | gatgttgtgatgactcagtctccactctccctgcccgtcacccct | dvvmtqsplslpvtpg | SEQ ID NO: 65 |
| variable | ggagagccggcctccatctcctgcaggtctagtcagagcctcctg | epasiscrssqsllhs | (SEQ ID NO: 66) |
| light | catagtaatggagacaactatttggattggtacctgcagaagcca | ngdnyldwylqkpgqs | |
| chain | gggcagtctccacagctcctgatctatttgggttctaatcgggcc | pqlliylgsnrasgvp | |
| domain | tccggggtccctgaccgtttcagtggcagtggatcaggcacagat | drfsgsgsgtdftlki | |
| | tttacactgaaaatcagccgtgtggaggctgaggatgttggggtt | srveaedvgvyycmqa | |
| | tattactgcatgcaagctacacactggccactcactttcggccct | thwpltfgpgtkvdik | |
| | ggtaccaaagtggatatcaaa | | |
| DB83 | caggtgcagctggtgcagtctggggctgaggtgaagaagcctggg | qvqlvqsgaevkkpga | SEQ ID NO: 67 |
| variable | gcctcagtgaaggtttcctgcaaggcatctggatacaccttcact | svkvsckasgytftsy | (SEQ ID NO: 68) |
| heavy | agctatgctatgcattgggtgcgtcaggcccctggacaagggctt | amhwvrqapgqglewm | |
| chain | gagtggatgggacttgttgatcctgaagatggtgaaacaatatat | glvdpedgetiyaekf | |
| domain | gcagagaagttccagggccgtgtcaccatgacccgcgacacgtcc | qgrvtmtrdtststvy | |
| | acgagcacagtctacatggagctgagcagcctgcgttctgaggac | melsslrsedtavyyc | |
| | acggccgtgtattactgtgcgagacgaacgtattactatgatagt | arrtyyydssgsryaf | |
| | agtggttcccgttatgcttttgatatctggggccaagggaccacg | diwgqgttv | |
| | gtcaccgtctcttca | tvss | |
| DB83 | cagagcctcctgcatagtaatggagacaactat | QSLLHSNGDNY | SEQ ID NO: 69 |
| CDR L1 | | | (SEQ ID NO: 70) |
| DB83 | ttgggttct | LGS | SEQ ID NO: 71 |
| CDR L2 | | | (SEQ ID NO: 72) |
| DB83 | atgcaagctacacactggccactcact | MQATHWPLT | SEQ ID NO: 73 |
| CDR L3 | | | (SEQ ID NO: 74) |
| DB83 | ggatacaccttcactagctatgct | GYTFTSYA | SEQ ID NO: 75 |
| CDR H1 | | | (SEQ ID NO: 76) |
| DB83 | gttgatcctgaagatggtgaaaca | VDPEDGET | SEQ ID NO: 77 |
| CDR H2 | | | (SEQ ID NO: 78) |
| DB83 | gcgagacgaacgtattactatgatagtagtggttcccgttatgct | ARRTYYYDSSGSRYAF | SEQ ID NO: 79 |
| CDR H3 | tttgatatc | DI | (SEQ ID NO: 80) |
| DB86 | gacatccagatgacccagtctccatcctccctgtctgcatctgta | diqmtqspsslsasvg | SEQ ID NO: 81 |
| variable | ggagaccgcgtcaccatcacttgccgggcaagtcagggcatcaga | drvtitcrasqgirnd | (SEQ ID NO: 82) |
| light | aatgatttaggttggtatcagcagaaaccagggaaagcccctaag | lgwyqqkpgkapklli | |
| chain | ctcctgatctatgctgcatccactttgcaatcaggggtcccatca | yaastlqsgvpsrfsg | |
| domain | cgtttcagtggcagtggatctgggacagatttcactctcaccatc | sgsgtdftltisslqp | |
| | agcagtctgcaacctgaagattttgcaacttactactgtcaacag | edfatyycqqsygapl | |
| | agttacggtgcccccctcactttcggcggaggtaccaaggtggag | tfgggtkveik | |
| | atcaaa | | |
| DB86 | caggtgcagctggtgcagtctggggctgaggtgaagaagcctggg | qvqlvqsgaevkkpga | SEQ ID NO: 83 |
| variable | gcctcagtgaaggtttcctgcaaggcatctggatatatgttcagt | svkvsckasgymfsgh | (SEQ ID NO: 84) |
| heavy | ggccattctgcacactgggtgcgtcaggcccctggacaagggctt | sahwvrqapgqglewm | |
| chain | gagtggatgggatggatgaaccctaacagtggtaacacaggctat | gwmnpnsgntgyaqkf | |
| domain | gcacagaagttccagggccgtgtcaccatgacccgcgacacgtcc | qgrvtmtrdtststvy | |
| | acgagcacagtctacatggagctgagcagcctgcgttctgaggac | melsslrsedtavyyc | |
| | acggccgtgtattactgtgcgagagatagcagtggctggtacgat | ardssgwydvfdywgq | |
| | gtctttgactactggggccaggggaccctggtcaccgtctcctca | gtlvtvss | |
| DB86 | cagggcatcagaaatgat | QGIRND | SEQ ID NO: 85 |
| CDR L1 | | | (SEQ ID NO: 86) |
| DB86 | gctgcatcc | AAS | SEQ ID NO: 87 |
| CDR L2 | | | (SEQ ID NO: 88) |
| DB86 | caacagagttacggtgcccccctc | QQSYGAPLT | SEQ ID NO: 89 |
| CDR L3 | | | (SEQ ID NO: 90) |
| DB86 | ggatatatgttcagtggccattct | GYMFSGHS | SEQ ID NO: 91 |
| CDR H1 | | | (SEQ ID NO: 92) |
| DB86 | atgaaccctaacagtggtaacaca | MNPNSGNT | SEQ ID NO: 93 |
| CDR H2 | | | (SEQ ID NO: 94) |
| DB86 | gcgagagatagcagtggctggtacgatgtctttgactac | ARDSSGWYDVFDY | SEQ ID NO: 95 |
| CDR H3 | | | (SEQ ID NO: 96) |
| DB280 | gacatccagatgacccagtctccatcctccctgtctgcatctgta | diqmtqspsslsasvg | SEQ ID NO: 17 |
| variable | ggagacagagtcaccatcacttgccgggcaagtcagagcattagc | drvtitcrasqsissy | (SEQ ID NO: 18) |
| light | agctatctgaattggtatcagcagaaaccagggaaagcccctaag | lnwyqqkpgkapklli | |
| chain | ctcctgatctatgctgcatccagtttgcaaagtggggtcccatca | yaasslqsgvpsrfsg | |
| domain | aggttcagtggcagtggatctgggacagatttcactctcaccatc | sgsgtdftltisslqp | |
| | agcagtctgcaacctgaagattttgcaacttactactgtcaacag | edfatyycqqsystpl | |
| | agttacagtacccctctcactttcggcggaggtaccaaggtggag | tfgggtkveik | |
| | atcaaa | | |
| DB280 | caggtgcagctggtgcagtctggggctgaggtgaagaagcctggg | qvqlvqsgaevkkpga | SEQ ID NO: 97 |
| variable | gcctcagtgaaggtttcctgcaaggcatctggatacagcctcaac | svkvsckasgyslnly | (SEQ ID NO: 98) |
| heavy | ttatactatatgcactgggtgcgtcaggcccctggacaagggctt | ymhwvrqapgqglewm | |
| chain | gagtggatgggatggatgaaccctaacagtggtaacacaggctat | gwmnpnsgntgyaqkf | |
| domain | gcacagaagttccagggccgtgtcaccatgacccgcgacacgtcc | qgrvtmtrdtststvy | |
| | acgagcacagtctacatggagctgagcagcctgcgttctgaggac | melsslrsedtavyyc | |
| | acggccgtgtattactgtgcgagcctcgattgtagtggtggtagc | asldcsggscyseyda | |
| | tgctactccgaatatgatgcttttgatatctggggccaagggacc | fdiwgqgttvtvss | |
| | acggtcaccgtctcctca | | |
| DB280 | cagagcattagcagctat | QSISSY | SEQ ID NO: 21 |
| CDR L1 | | | (SEQ ID NO: 22) |
| DB280 | gctgcatcc | AAS | SEQ ID NO: 23 |
| CDR L2 | | | (SEQ ID NO: 24) |
| DB280 | caacagagttacagtacccctctcact | QQSYSTPLT | SEQ ID NO: 25 |
| CDR L3 | | | (SEQ ID NO: 26) |
| DB280 | ggatacagcctcaacttatactat | GYSLNLYY | SEQ ID NO: 99 |
| CDR H1 | | | (SEQ ID |
| | | | NO: 100) |
| DB280 | atgaaccctaacagtggtaacaca | MNPNSGNT | SEQ ID NO: 101 |
| CDR H2 | | | (SEQ ID |
| | | | NO: 102) |
| DB280 | gcgagcctcgattgtagtggtggtagctgctactccgaatatgat | ASLDCSGGSCYSEYDA | SEQ ID NO: 103 |
| CDR H3 | gcttttgatatc | FDI | (SEQ ID |
| | | | NO: 104) |
| DB331 | gacatccagatgacccagtctccatcctccctgtctgcatctgta | diqmtqspsslsasvg | SEQ ID NO: 17 |
| variable | ggagacagagtcaccatcacttgccgggcaagtcagagcattagc | drvtitcrasqsissy | (SEQ ID NO: 18) |
| light | agctatctgaattggtatcagcagaaaccagggaaagcccctaag | lnwyqqkpgkapklli | |
| chain | ctcctgatctatgctgcatccagtttgcaaagtggggtcccatca | yaasslqsgvpsrfsg | |
| domain | aggttcagtggcagtggatctgggacagatttcactctcaccatc | sgsgtdftltisslqp | |
| | agcagtctgcaacctgaagattttgcaacttactactgtcaacag | edfatyycqqsystpl | |
| | agttacagtacccctctcactttcggcggaggtaccaaggtggag | tfgggtkveik | |
| | atcaaa | | |
| DB331 | caggtgcagctggtgcagtctggggctgaggtgaagaagcctggg | qvqlvqsgaevkkpga | SEQ ID NO: 105 |
| variable | gcctcagtgaaggtttcctgcaaggcatctggatacaccttcacc | svkvsckasgytftsy | (SEQ ID |
| heavy | agctactatatgcactgggtgcgtcaggcccctggacaagggctt | ymhwvrqapgqglewm | NO: 106) |
| chain | gagtggatgggatggatgaaccctaacagtggtaacacaggctat | gwmnpnsgntgyaqkf | |
| domain | gcacagaagttccagggccgtgtcaccatgacccgcgacacgtcc | qgrvtmtrdtststvy | |
| | acgagcacagtctacatggagctgagcagcctgcgttctgaggac | melsslrsedtavyyc | |
| | acggccgtgtattactgtgcaacagatctcgcgggggaagccttg | atdlageaifdpwgqg | |
| | ttcgacccctggggccagggcaccctggtcaccgtctcctca | tlvtvss | |
| DB331 | cagagcattagcagctat | QSISSY | SEQ ID NO: 21 |
| CDR L1 | | | (SEQ ID NO: 22) |
| DB331 | gctgcatcc | AAS | SEQ ID NO: 23 |
| CDR L2 | | | (SEQ ID NO: 24) |
| DB331 | caacagagttacagtacccctctcact | QQSYSTPLT | SEQ ID NO: 25 |
| CDR L3 | | | (SEQ ID NO: 26) |
| DB331 | ggatacaccttcaccagctactat | GYTFTSYY | SEQ ID NO: 107 |
| CDR H1 | | | (SEQ ID |
| | | | NO: 108) |
| DB331 | atgaaccctaacagtggtaacaca | MNPNSGNT | SEQ ID NO: 109 |
| CDR H2 | | | (SEQ ID |
| | | | NO: 110) |
| DB331 | gcaacagatctcgcgggggaagccttgttcgacccc | ATDLAGEALFDP | SEQ ID NO: 111 |
| CDR H3 | | | (SEQ ID |
| | | | NO: 112) |
| DB415 | gacatccagatgacccagtctccatcctccctgtctgcatctgta | diqmtqspsslsasvg | SEQ ID NO: 17 |
| variable | ggagacagagtcaccatcacttgccgggcaagtcagagcattagc | drvtitcrasqsissy | (SEQ ID NO: 18) |
| light | agctatctgaattggtatcagcagaaaccagggaaagcccctaag | lnwyqqkpgkapklli | |
| chain | ctcctgatctatgctgcatccagtttgcaaagtggggtcccatca | yaasslqsgvpsrfsg | |
| domain | aggttcagtggcagtggatctgggacagatttcactctcaccatc | sgsgtdftltisslqp | |
| | agcagtctgcaacctgaagattttgcaacttactactgtcaacag | edfatyycqqsystpl | |
| | agttacagtacccctctcactttcggcggaggtaccaaggtggag | tfgggtkveik | |
| | atcaaa | | |
| DB415 | gaggtgcagctggtggagtctgggggaggcttggtacagcctggg | evqlvesggglvqpgg | SEQ ID NO: 113 |
| variable | gggtccctgcgcctctcctgtgcagcctctggaatcaccttcagt | slriscaasgitfssy | (SEQ ID |
| heavy | agttatggcatgcattgggtccgccaggctccagggaaggggctg | gmhwvrqapgkglewv | NO: 114) |
| chain | gagtgggtctcaggtattagttggaatagtggtaacagagtctat | sgiswnsgnrvyvdsv | |
| domain | gtggactctgtgaagggccggttcaccatctcccgcgacaattcc | kgrftisrdnskntly | |
| | aagaacacgctgtatctgcaaatgaacagcctgcgcgccgaggac | lqmnslraedtavyyc | |
| | acggccgtatattactgtgcgagagatactaatgatgcttttgat | ardtndafdiwgqgtt | |
| | atctggggccaagggaccacggtcaccgtctcctca | vtvss | |
| DB415 | cagagcattagcagctat | QSISSY | SEQ ID NO: 21 |
| CDR L1 | | | (SEQ ID NO: 22) |
| DB415 | gctgcatcc | AAS | SEQ ID NO: 23 |
| CDR L2 | | | (SEQ ID NO: 24) |
| DB415 | caacagagttacagtacccctctcact | QQSYSTPLT | SEQ ID NO: 25 |
| CDR L3 | | | (SEQ ID NO: 26) |
| DB415 | ggaatcaccttcagtagttatggc | GITFSSYG | SEQ ID NO: 115 |
| CDR H1 | | | (SEQ ID |
| | | | NO: 116) |
| DB415 | attagttggaatagtggtaacaga | ISWNSGNR | SEQ ID NO: 117 |
| CDR H2 | | | (SEQ ID |
| | | | NO: 118) |
| DB415 | gcgagagatactaatgatgcttttgatatc | ARDTNDAFDI | SEQ ID NO: 119 |
| CDR H3 | | | (SEQ ID |
| | | | NO: 120) |
| DB435 | gacatccagatgacccagtctccatcctccctgtctgcatctgta | diqmtqspsslsasvg | SEQ ID NO: 17 |
| variable | ggagacagagtcaccatcacttgccgggcaagtcagagcattagc | drvtitcrasqsissy | (SEQ ID NO: 18) |
| light | agctatctgaattggtatcagcagaaaccagggaaagcccctaag | lnwyqqkpgkapklli | |
| chain | ctcctgatctatgctgcatccagtttgcaaagtggggtcccatca | yaasslqsgvpsrfsg | |
| domain | aggttcagtggcagtggatctgggacagatttcactctcaccatc | sgsgtdftltisslqp | |
| | agcagtctgcaacctgaagattttgcaacttactactgtcaacag | edfatyycqqsystpl | |
| | agttacagtacccctctcactttcggcggaggtaccaaggtggag | tfgggtkveik | |
| | atcaaa | | |
| DB435 | caggtgcagctggtgcagtctggggctgaggtgaagaagcctggg | qvqlvqsgaevkkpga | SEQ ID NO: 121 |
| variable | gcctcagtgaaggtttcctgcaaggcatctggaggcaccttcagc | svkvsckasggtfssy | (SEQ ID |
| heavy | agctatgctatcagctgggtgcgtcaggcccctggacaagggctt | aiswvrqapgqglewm | NO: 122) |
| chain | gagtggatgggctggatcacccctcacaatggtaacataaagtat | gwitphngnikyaref | |
| domain | gcacgggagttccagggccgtgtcaccatgacccgcgacacgtcc | qgrvtmtrdtststvy | |
| | acgagcacagtctacatggagctgagcagcctgcgttctgaggac | melsslrsedtavyyc | |
| | acggccgtgtattactgtgcgaaagatctgaactggaacgcagcc | akdlnwnaafdywgqg | |
| | tttgactactggggccaggggaccctggtcaccgtctcctca | tlvtvss | |
| DB435 | cagagcattagcagctat | QSISSY | SEQ ID NO: 21 |
| CDR L1 | | | (SEQ ID NO: 22) |
| DB435 | gctgcatcc | AAS | SEQ ID NO: 233 |
| CDR L2 | | | (SEQ ID NO: 24) |
| DB435 | caacagagttacagtacccctctcact | QQSYSTPLT | SEQ ID NO: 25 |
| CDR L3 | | | (SEQ ID NO: 26) |
| DB435 | ggaggcaccttcagcagctatgct | GGTFSSYA | SEQ ID NO: 123 |
| CDR H1 | | | (SEQ ID |
| | | | NO: 124) |
| DB435 | atcacccctcacaatggtaacata | ITPHNGNI | SEQ ID NO: 125 |
| CDR H2 | | | (SEQ ID |
| | | | NO: 126) |
| DB435 | gcgaaagatctgaactggaacgcagcctttgactac | AKDLNWNAAFDY | SEQ ID NO: 127 |
| CDR H3 | | | (SEQ ID |
| | | | NO: 128) |
| OMT1 | atggaagcaccagcgcagcttctcttcctcctgctactctggctc | evqllesggglvqpgg | SEQ ID NO: 129 |
| VHVL x | ccagataccaccggtgaggtgcagctgttggagtctgggggaggc | slrlscaasgftfssy | (SEQ ID |
| TSC456 | ttggtacagcctggggggtccctgagactctcctgtgcagcctct | gmswvrqapgkglegv | NO: 130) |
| scFv-Fc- | ggattcacctttagcagctatggcatgagctgggtccgccaggct | saisgsggstyyadsv | |
| scFv | ccagggaaggggctggagggggtctcagctattagtggtagtggt | kgrftisrdnskntly | |
| TRI129 | ggtagcacatactacgcagactccgtgaagggccggttcaccatc | lqmnslraedtavyyc | |
| | tccagagacaattccaagaacacgctgtatctgcaaatgaacagc | akeklryfdwlsdafd | |
| | ctgagagccgaggacacggccgtatattactgtgcgaaagaaaag | iwgqgtmvtvssgggg | |
| | ttacgatattttgactggttatccgatgcttttgatatctggggc | sggggsggggsggggs | |
| | caagggacaatggtcaccgtctcttcaggtggaggcggttcaggc | divmtqspdslavslg | |
| | ggaggtggatccggcggtggcggctccggtggcggcggatctgac | eratincksshsvlys | |
| | atcgtgatgacccagtctccagactccctggctgtgtctctgggc | snnknylawyqqkpgq | |
| | gagagggccaccatcaactgcaagtccagccacagtgttttatac | ppklliywastresgv | |
| | agctccaacaataagaactacttagcttggtaccagcagaaacca | pdrfsgsgsgtdftlt | |
| | ggacagcctcctaagctgctcatttactgggcatctacccgggaa | isslqaedvavyycqq | |
| | tccggggtccctgaccgattcagtggcagcgggtctgggacagat | yystppttfgggtkve | |
| | ttcactctcaccatcagcagcctgcaggctgaagatgtggcagtt | iksssepkssdkthtc | |
| | tattactgtcagcaatattatagtactcctccgaccactttcggc | ppcpapeaagapsvfl | |
| | ggagggaccaaggtggagatcaaatcctcgagtgagcccaaatct | fppkpkdtlmisrtpe | |
| | tctgacaaaactcacacatgcccaccgtgcccagcacctgaagcc | vtcvvvdvshedpevk | |
| | gcgggtgcaccgtcagtcttcctcttccccccaaaacccaaggac | fnwyvdgvevhnaktk | |
| | accctcatgatctcccggacccctgaggtcacatgcgtggtggtg | preeqynstyrvvsvl | |
| | gacgtgagccacgaagaccctgaggtcaagttcaactggtacgtg | tvlhqdwingkeykca | |
| | gacggcgtggaggtgcataatgccaagacaaagccgcgggaggag | vsnkalpapiektisk | |
| | cagtacaacagcacgtaccgtgtggtcagcgtcctcaccgtcctg | akgqprepqvytipps | |
| | caccaggactggctgaatggcaaggaatacaagtgcgcggtctcc | rdeltknqvsltclvk | |
| | aacaaagccctcccagcccccatcgagaaaaccatctccaaagcc | gfypsdiavewesngq | |
| | aaagggcagccccgagaaccacaggtgtacaccctgcccccatcc | pennykttppvldsdg | |
| | cgggatgagctgaccaagaaccaggtcagcctgacctgcctggtc | sfflyskltvdksrwq | |
| | aaaggcttctatccaagcgacatcgccgtggagtgggagagcaat | qgnvfscsvmhealhn | |
| | gggcagccggagaacaactacaagaccacgcctcccgtgctggac | hytqkslslspgsggg | |
| | tccgacggctccttcttcctctacagcaagctcaccgtggacaag | gsggggsggggspsqv | |
| | agcaggtggcagcaggggaacgtcttctcatgctccgtgatgcat | qlvqsgpevkkpgssv | |
| | gaggctctgcacaaccactacacgcagaagagcctctccctgtct | kvsckasgytfsrstm | |
| | ccgggttccggaggagggggttcaggtgggggaggttctggcggc | hwvrqapgqglewigy | |
| | gggggaagcccttcacaggtgcaactggtgcagagtggacccgag | inpssaytnynqkfkd | |
| | gttaaaaaaccagggtcctccgttaaggttagctgcaaagcctct | rvtitadkststayme | |
| | ggctacacattttccaggagtacaatgcactgggtgaggcaggct | lsslrsedtavyycar | |
| | cctggacagggactcgagtggatcgggtatatcaacccatctagc | pqvhydyngfpywgqg | |
| | gcctataccaattacaaccaaaagtttaaggaccgagttaccatt | tlvtvssggggsgggg | |
| | accgctgacaaatccaccagtacagcttatatggagctgtcatct | sggggsggggsdiqmt | |
| | cttaggtccgaggacactgctgtttattactgcgctcgtcctcag | qspstlsasvgdrvtm | |
| | gttcactatgactataatggttttccctactggggtcagggaacc | tcsasssvsymnwyqq | |
| | ctggtgactgtctcttctggcggtggaggcagcggtgggggtggg | kpgkapkrwiydsskl | |
| | tctggaggcggtggcagtggcggcggaggctctgatattcagatg | asgvpsrfsgsgsgtd | |
| | actcagtctcctagcactctcagcgccagcgtgggggatcgtgtg | ytltisslqpddfaty | |
| | acaatgacttgctccgctagcagtagtgtgtcttacatgaattgg | ycqqwsrnpptfgggt | |
| | tatcagcagaagcccgggaaagcacctaagcgctggatctatgac | kveikrs | |
| | tcttccaagctggcaagtggtgtcccctcacggttctctggctca | | |
| | ggttctggtactgactatactttgactatctcctccctccagccc | | |
| | gatgatttcgctacctattattgtcagcagtggagccgtaaccca | | |
| | cccactttcggaggcggtaccaaagtggagatcaagaggtcataa | | |
| OMT1 | atggaagcaccagcgcagcttctcttcctcctgctactctggctc | divmtqspdslavslg | SEQ ID NO: 131 |
| VLVH x | ccagataccaccggtgacatcgtgatgacccagtctccagactcc | er | (SEQ ID |
| TSC456 | ctggctgtgtctctgggcgagagggccaccatcaactgcaagtcc | atincksshsvlyssn | NO: 132) |
| scFv- | agccacagtgttttatacagctccaacaataagaactacttagct | nk | |
| Fc- | tggtaccagcagaaaccaggacagcctcctaagctgctcatttac | nylawyqqkpgqppkl | |
| scFv | tgggcatctacccgggaatccggggtccctgaccgattcagtggc | l | |
| TRI130 | agcgggtctgggacagatttcactctcaccatcagcagcctgcag | iywastresgvpdrfs | |
| | gctgaagatgtggcagtttattactgtcagcaatattatagtact | gs | |
| | cctccgaccactttcggcggagggaccaaggtggagatcaaaggt | gsgtdftltisslqae | |
| | ggaggcggttcaggcggaggtggatccggcggtggcggctccggt | dva | |
| | ggcggcggatctgaggtgcagctgttggagtctgggggaggcttg | vyycqqyystppttfg | |
| | gtacagcctggggggtccctgagactctcctgtgcagcctctgga | gg | |
| | ttcacctttagcagctatggcatgagctgggtccgccaggctcca | tkveikggggsggggs | |
| | gggaaggggctggagggggtctcagctattagtggtagtggtggt | gg | |
| | agcacatactacgcagactccgtgaagggccggttcaccatctcc | ggsggggsevqllesg | |
| | agagacaattccaagaacacgctgtatctgcaaatgaacagcctg | gg | |
| | agagccgaggacacggccgtatattactgtgcgaaagaaaagtta | lvqpggslrlscaasg | |
| | cgatattttgactggttatccgatgcttttgatatctggggccaa | ftfs | |
| | gggacaatggtcaccgtctcctcgagtgagcccaaatcttctgac | sygmswvrqapgkgle | |
| | aaaactcacacatgcccaccgtgcccagcacctgaagccgcgggt | gvsaisgsggstyyad | |
| | gcaccgtcagtcttcctcttccccccaaaacccaaggacaccctc | sv | |
| | atgatctcccggacccctgaggtcacatgcgtggtggtggacgtg | kgrftisrdnskntly | |
| | agccacgaagaccctgaggtcaagttcaactggtacgtggacggc | lqm | |
| | gtggaggtgcataatgccaagacaaagccgcgggaggagcagtac | nslraedtavyycake | |
| | aacagcacgtaccgtgtggtcagcgtcctcaccgtcctgcaccag | kl | |
| | gactggctgaatggcaaggaatacaagtgcgcggtctccaacaaa | ryfdwlsdafdiwgqg | |
| | gccctcccagcccccatcgagaaaaccatctccaaagccaaaggg | t | |
| | cagccccgagaaccacaggtgtacaccctgcccccatcccgggat | mvtvsssepkssdkth | |
| | gagctgaccaagaaccaggtcagcctgacctgcctggtcaaaggc | t | |
| | ttctatccaagcgacatcgccgtggagtgggagagcaatgggcag | cppcpapeaagapsvf | |
| | ccggagaacaactacaagaccacgcctcccgtgctggactccgac | l | |
| | ggctccttcttcctctacagcaagctcaccgtggacaagagcagg | fppkpkdtlmisrtpe | |
| | tggcagcaggggaacgtcttctcatgctccgtgatgcatgaggct | vt | |
| | ctgcacaaccactacacgcagaagagcctctccctgtctccgggt | cvvvdvshedpevkfn | |
| | tccggaggagggggttcaggtgggggaggttctggcggcggggga | wyvdgvevhnaktkpr | |
| | agcccttcacaggtgcaactggtgcagagtggacccgaggttaaa | eeqynstyrwsvltvl | |
| | aaaccagggtcctccgttaaggttagctgcaaagcctctggctac | h | |
| | acattttccaggagtacaatgcactgggtgaggcaggctcctgga | qdwingkeykcavsnk | |
| | cagggactcgagtggatcgggtatatcaacccatctagcgcctat | alpapiektiskakgq | |
| | accaattacaaccaaaagtttaaggaccgagttaccattaccgct | pr | |
| | gacaaatccaccagtacagcttatatggagctgtcatctcttagg | epqvytippsrdeltk | |
| | tccgaggacactgctgtttattactgcgctcgtcctcaggttcac | nq | |
| | tatgactataatggttttccctactggggtcagggaaccctggtg | vsltclvkgfypsdia | |
| | actgtctcttctggcggtggaggcagcggtgggggtgggtctgga | ve | |
| | ggcggtggcagtggcggcggaggctctgatattcagatgactcag | wesngqpennykttpp | |
| | tctcctagcactctcagcgccagcgtgggggatcgtgtgacaatg | vldsdgsfflyskltv | |
| | acttgctccgctagcagtagtgtgtcttacatgaattggtatcag | dksr | |
| | cagaagcccgggaaagcacctaagcgctggatctatgactcttcc | wqqgnvfscsvmheal | |
| | aagctggcaagtggtgtcccctcacggttctctggctcaggttct | hnhytqkslslspgsg | |
| | ggtactgactatactttgactatctcctccctccagcccgatgat | gg | |
| | ttcgctacctattattgtcagcagtggagccgtaacccacccact | gsggggsggggspsqv | |
| | ttcggaggcggtaccaaagtggagatcaagaggtcataa | q | |
| | | lvqsgpevkkpgssvk | |
| | | v | |
| | | sckasgytfsrstmhw | |
| | | v | |
| | | rqapgqglewigyinp | |
| | | s | |
| | | saytnynqkfkdrvti | |
| | | ta | |
| | | dkststaymelsslrs | |
| | | ed | |
| | | tavyycarpqvhydyn | |
| | | g | |
| | | fpywgqgtlvtvssgg | |
| | | g | |
| | | gsggggsggggsgggg | |
| | | s | |
| | | diqmtqspstlsasvg | |
| | | dr | |
| | | vtmtcsasssvsymnw | |
| | | yqqkpgkapkrwiyds | |
| | | sklasgvpsrfsgsgs | |
| | | gtd | |
| | | ytltisslqpddfaty | |
| | | ycq | |
| | | qwsrnpptfgggtkve | |
| | | ikrs | |
| DB8 VHVL x | atggaagcaccagcgcagcttctcttcctcctgctactctggctc | qvqlvqsgaevkkpga | SEQ ID NO: 133 |
| TSC456 | ccagataccaccggtcaggtgcagctggtgcagtctggggctgag | svkvsckasgyiftdy | (SEQ ID |
| scFv-Fc- | gtgaagaagcctggggcctcagtgaaggtttcctgcaaggcatct | ymhwvrqapgqglewm | NO: 134) |
| scFv | ggatacatcttcaccgactactatatgcactgggtgcgtcaggcc | gwmspnsgntgyaqkf | |
| TRI123 | cctggacaagggcttgagtggatgggatggatgagccctaacagt | qgrvtmtrdtststvy | |
| | ggtaacacaggctatgcacagaagttccagggccgtgtcaccatg | melssirsedtavyyc | |
| | acccgcgacacgtccacgagcacagtctacatggagctgagcagc | ardaadygdyvafdiw | |
| | ctgcgttctgaggacacggccgtgtattactgtgcgagagatgcg | gqgtmvtvssggggsg | |
| | gcggattacggtgactacgttgcttttgatatctggggccaaggg | gggsggggsggggsdi | |
| | acaatggtcaccgtctcttcaggcggcggcggcagcggcggcggc | qmtqspsslsasvgdr | |
| | ggcagcggcggcggaggctccggcggcggcggcagcgacatccag | vtitcrasqsissyln | |
| | atgacccagtctccatcctccctgtctgcatctgtaggagacaga | wyqqkpgkapklliya | |
| | gtcaccatcacttgccgggcaagtcagagcattagcagctatctg | asslqsgvpsrfsgsg | |
| | aattggtatcagcagaaaccagggaaagcccctaagctcctgatc | sgtdftltisslqped | |
| | tatgctgcatccagtttgcaaagtggggtcccatcaaggttcagt | fatyycqqsystpltf | |
| | ggcagtggatctgggacagatttcactctcaccatcagcagtctg | gggtkveiksssepks | |
| | caacctgaagattttgcaacttactactgtcaacagagttacagt | sdkthtcppcpapeaa | |
| | acccctctcactttcggcggaggtaccaaggtggagatcaaatcc | gapsvflfppkpkdtl | |
| | tcgagtgagcccaaatcttctgacaaaactcacacatgcccaccg | misrtpevtcvwdvsh | |
| | tgcccagcacctgaagccgcgggtgcaccgtcagtcttcctcttc | edpevkfnwyvdgvev | |
| | cccccaaaacccaaggacaccctcatgatctcccggacccctgag | hnaktkpreeqynsty | |
| | gtcacatgcgtggtggtggacgtgagccacgaagaccctgaggtc | rvvsvltvlhqdwing | |
| | aagttcaactggtacgtggacggcgtggaggtgcataatgccaag | keykcavsnkalpapi | |
| | acaaagccgcgggaggagcagtacaacagcacgtaccgtgtggtc | ektiskakgqprepqv | |
| | agcgtcctcaccgtcctgcaccaggactggctgaatggcaaggaa | ytippsrdeltknqvs | |
| | tacaagtgcgcggtctccaacaaagccctcccagcccccatcgag | ltclvkgfypsdiave | |
| | aaaaccatctccaaagccaaagggcagccccgagaaccacaggtg | wesngqpennykttpp | |
| | tacaccctgcccccatcccgggatgagctgaccaagaaccaggtc | vldsdgsfflyskltv | |
| | agcctgacctgcctggtcaaaggcttctatccaagcgacatcgcc | dksrwqqgnvfscsvm | |
| | gtggagtgggagagcaatgggcagccggagaacaactacaagacc | healhnhytqkslsls | |
| | acgcctcccgtgctggactccgacggctccttcttcctctacagc | pgsggggsggggsggg | |
| | aagctcaccgtggacaagagcaggtggcagcaggggaacgtcttc | gspsqvqlvqsgpevk | |
| | tcatgctccgtgatgcatgaggctctgcacaaccactacacgcag | kpgssvkvsckasgyt | |
| | aagagcctctccctgtctccgggttccggaggagggggttcaggt | fsrstmhwvrqapgqg | |
| | gggggaggttctggcggcgggggaagcccttcacaggtgcaactg | lewigyinpssaytny | |
| | gtgcagagtggacccgaggttaaaaaaccagggtcctccgttaag | nqkfkdrvtitadkst | |
| | gttagctgcaaagcctctggctacacattttccaggagtacaatg | staymelsslrsedta | |
| | cactgggtgaggcaggctcctggacagggactcgagtggatcggg | vyycarpqvhydyngf | |
| | tatatcaacccatctagcgcctataccaattacaaccaaaagttt | pywgqgtlvtvssggg | |
| | aaggaccgagttaccattaccgctgacaaatccaccagtacagct | gsggggsggggsgggg | |
| | tatatggagctgtcatctcttaggtccgaggacactgctgtttat | sdiqmtqspstlsasv | |
| | tactgcgctcgtcctcaggttcactatgactataatggttttccc | gdrvtmtcsasssvsy | |
| | tactggggtcagggaaccctggtgactgtctcttctggcggtgga | mnwyqqkpgkapkrwi | |
| | ggcagcggtgggggtgggtctggaggcggtggcagtggcggcgga | ydssklasgvpsrfsg | |
| | ggctctgatattcagatgactcagtctcctagcactctcagcgcc | sgsgtdytltisslqp | |
| | agcgtgggggatcgtgtgacaatgacttgctccgctagcagtagt | ddfatyycqqwsrnpp | |
| | gtgtcttacatgaattggtatcagcagaagcccgggaaagcacct | tfgggtkveikrs | |
| | aagcgctggatctatgactcttccaagctggcaagtggtgtcccc | | |
| | tcacggttctctggctcaggttctggtactgactatactttgact | | |
| | atctcctccctccagcccgatgatttcgctacctattattgtcag | | |
| | cagtggagccgtaacccacccactttcggaggcggtaccaaagtg | | |
| | gagatcaagaggtcataa | | |
| DB8 VLVH x | atggaagcaccagcgcagcttctcttcctcctgctactctggctc | diqmtqspsslsasvg | SEQ ID NO: 135 |
| TSC456 | ccagataccaccggtgacatccagatgacccagtctccatcctcc | drvtitcrasqsissy | (SEQ ID |
| scFv-Fc- | ctgtctgcatctgtaggagacagagtcaccatcacttgccgggca | lnwyqqkpgkapklli | NO: 136) |
| scFv | agtcagagcattagcagctatctgaattggtatcagcagaaacca | yaasslqsgvpsrfsg | |
| TRI124 | gggaaagcccctaagctcctgatctatgctgcatccagtttgcaa | sgsgtdftltisslqp | |
| | agtggggtcccatcaaggttcagtggcagtggatctgggacagat | edfatyycqqsystpl | |
| | ttcactctcaccatcagcagtctgcaacctgaagattttgcaact | tfgggtkveikggggs | |
| | tactactgtcaacagagttacagtacccctctcactttcggcgga | ggggsggggsggggsq | |
| | ggtaccaaggtggagatcaaaggcggcggcggcagcggcggcggc | vqlvqsgaevkkpgas | |
| | ggcagcggcggcggaggctccggcggcggcggcagccaggtgcag | vkvsckasgyiftdyy | |
| | ctggtgcagtctggggctgaggtgaagaagcctggggcctcagtg | mhwvrqapgqglewmg | |
| | aaggtttcctgcaaggcatctggatacatcttcaccgactactat | wmspnsgntgyaqkfq | |
| | atgcactgggtgcgtcaggcccctggacaagggcttgagtggatg | grvtmtrdtststvym | |
| | ggatggatgagccctaacagtggtaacacaggctatgcacagaag | elssirsedtavyyca | |
| | ttccagggccgtgtcaccatgacccgcgacacgtccacgagcaca | rdaadygdyvafdiwg | |
| | gtctacatggagctgagcagcctgcgttctgaggacacggccgtg | qgtmvtvsssepkssd | |
| | tattactgtgcgagagatgcggcggattacggtgactacgttgct | kthtcppcpapeaaga | |
| | tttgatatctggggccaagggacaatggtcaccgtctcctcgagt | psvflfppkpkdtlmi | |
| | gagcccaaatcttctgacaaaactcacacatgcccaccgtgccca | srtpevtcvwdvshed | |
| | gcacctgaagccgcgggtgcaccgtcagtcttcctcttcccccca | pevkfnwyvdgvevhn | |
| | aaacccaaggacaccctcatgatctcccggacccctgaggtcaca | aktkpreeqynstyrv | |
| | tgcgtggtggtggacgtgagccacgaagaccctgaggtcaagttc | vsvltvlhqdwlngke | |
| | aactggtacgtggacggcgtggaggtgcataatgccaagacaaag | ykcavsnkalpapiek | |
| | ccgcgggaggagcagtacaacagcacgtaccgtgtggtcagcgtc | tiskakgqprepqvyt | |
| | ctcaccgtcctgcaccaggactggctgaatggcaaggaatacaag | ippsrdeltknqvslt | |
| | tgcgcggtctccaacaaagccctcccagcccccatcgagaaaacc | clvkgfypsdiavewe | |
| | atctccaaagccaaagggcagccccgagaaccacaggtgtacacc | sngqpennykttppvl | |
| | ctgcccccatcccgggatgagctgaccaagaaccaggtcagcctg | dsdgsfflyskltvdk | |
| | acctgcctggtcaaaggcttctatccaagcgacatcgccgtggag | srwqqgnvfscsvmhe | |
| | tgggagagcaatgggcagccggagaacaactacaagaccacgcct | alhnhytqkslslspg | |
| | cccgtgctggactccgacggctccttcttcctctacagcaagctc | sggggsggggsggggs | |
| | accgtggacaagagcaggtggcagcaggggaacgtcttctcatgc | psqvqlvqsgpevkkp | |
| | tccgtgatgcatgaggctctgcacaaccactacacgcagaagagc | gssvkvsckasgytfs | |
| | ctctccctgtctccgggttccggaggagggggttcaggtggggga | rstmhwvrqapgqgle | |
| | ggttctggcggcgggggaagcccttcacaggtgcaactggtgcag | wigyinpssaytnynq | |
| | agtggacccgaggttaaaaaaccagggtcctccgttaaggttagc | kfkdrvtitadkstst | |
| | tgcaaagcctctggctacacattttccaggagtacaatgcactgg | aymelsslrsedtavy | |
| | gtgaggcaggctcctggacagggactcgagtggatcgggtatatc | ycarpqvhydyngfpy | |
| | aacccatctagcgcctataccaattacaaccaaaagtttaaggac | wgqgtlvtvssggggs | |
| | cgagttaccattaccgctgacaaatccaccagtacagcttatatg | ggggsggggsggggsd | |
| | gagctgtcatctcttaggtccgaggacactgctgtttattactgc | iqmtqspstlsasvgd | |
| | gctcgtcctcaggttcactatgactataatggttttccctactgg | rvtmtcsasssvsymn | |
| | ggtcagggaaccctggtgactgtctcttctggcggtggaggcagc | wyqqkpgkapkrwiyd | |
| | ggtgggggtgggtctggaggcggtggcagtggcggcggaggctct | ssklasgvpsrfsgsg | |
| | gatattcagatgactcagtctcctagcactctcagcgccagcgtg | sgtdytltisslqpdd | |
| | ggggatcgtgtgacaatgacttgctccgctagcagtagtgtgtct | fatyycqqwsrnpptf | |
| | tacatgaattggtatcagcagaagcccgggaaagcacctaagcgc | gggtkveikrs | |
| | tggatctatgactcttccaagctggcaagtggtgtcccctcacgg | | |
| | ttctctggctcaggttctggtactgactatactttgactatctcc | | |
| | tccctccagcccgatgatttcgctacctattattgtcagcagtgg | | |
| | agccgtaacccacccactttcggaggcggtaccaaagtggagatc | | |
| | aagaggtcataa | | |
| DB8 VHVL x | atggaagcaccagcgcagcttctcttcctcctgctactctg | qvqlvqsgaevkkpga | SEQ ID NO: 137 |
| TSC456 | gctcccagataccaccggtcaggtgcagctggtgcagtctggggc | svkvsckasgyiftdy | (SEQ ID |
| scFv-Fc- | tgaggtgaagaagcctggggcctcagtgaaggtttcctgcaaggc | ymhwvrqapgqglewm | NO: 138) |
| scFv | atctggatacatcttcaccgactactatatgcactgggtgcgtca | gwmspnsgntgyaqkf | |
| TRI137 | ggcccctggacaagggcttgagtggatgggatggatgagccctaa | qgrvtmtrdtststvy | |
| | cagtggtaacacaggctatgcacagaagttccagggccgtgtcac | melssirsedtavyyc | |
| | catgacccgcgacacgtccacgagcacagtctacatggagctgag | ardaadygdyvafdiw | |
| | cagcctgcgttctgaggacacggccgtgtattactgtgcgagaga | gqgtmvtvssggggsg | |
| | tgcggcggattacggtgactacgttgcttttgatatctggggcca | gggsggggsggggsdi | |
| | agggacaatggtcaccgtctcttcaggtggaggcggttcaggcgg | qmtqspsslsasvgdr | |
| | aggtggatccggcggtggcggctccggtggcggcggatctgacat | vtitcrasqsissyln | |
| | ccagatgacccagtctccatcctccctgtctgcatctgtaggaga | wyqqkpgkapklliya | |
| | cagagtcaccatcacttgccgggcaagtcagagcattagcagcta | asslqsgvpsrfsgsg | |
| | tctgaattggtatcagcagaaaccagggaaagcccctaagctcct | sgtdftltisslqped | |
| | gatctatgctgcatccagtttgcaaagtggggtcccatcaaggtt | fatyycqqsystpltf | |
| | cagtggcagtggatctgggacagatttcactctcaccatcagcag | gggtkveiksssepks | |
| | tctgcaacctgaagattttgcaacttactactgtcaacagagtta | sdkthtcppcpapeaa | |
| | cagtacccctctcactttcggcggaggtaccaaggtggagatcaa | gapsvflfppkpkdtl | |
| | atcctcgagtgagcccaaatcttctgacaaaactcacacatgccc | misrtpevtcvvvdvs | |
| | accgtgcccagcacctgaagccgcgggtgcaccgtcagtcttcct | hedpevkfnwyvdgve | |
| | cttccccccaaaacccaaggacaccctcatgatctcccggacccc | vhnaktkpreeqynst | |
| | tgaggtcacatgcgtggtggtggacgtgagccacgaagaccctga | yrvvsvltvlhqdwin | |
| | ggtcaagttcaactggtacgtggacggcgtggaggtgcataatgc | gkeykcavsnkalpap | |
| | caagacaaagccgcgggaggagcagtacaacagcacgtaccgtgt | iektiskakgqprepq | |
| | ggtcagcgtcctcaccgtcctgcaccaggactggctgaatggcaa | vytippsrdeltknqv | |
| | ggaatacaagtgcgcggtctccaacaaagccctcccagcccccat | sltclvkgfypsdiav | |
| | cgagaaaaccatctccaaagccaaagggcagccccgagaaccaca | ewesngqpennykttp | |
| | ggtgtacaccctgcccccatcccgggatgagctgaccaagaacca | pvldsdgsfflysklt | |
| | ggtcagcctgacctgcctggtcaaaggcttctatccaagcgacat | vdksrwqqgnvfscsv | |
| | cgccgtggagtgggagagcaatgggcagccggagaacaactacaa | mhealhnhytqkslsl | |
| | gaccacgcctcccgtgctggactccgacggctccttcttcctcta | spgsggggsggggsgg | |
| | cagcaagctcaccgtggacaagagcaggtggcagcaggggaacgt | ggspsqvqlvqsgpev | |
| | cttctcatgctccgtgatgcatgaggctctgcacaaccactacac | kkpgssvkvsckasgy | |
| | gcagaagagcctctccctgtctccgggttccggaggagggggttc | tfsrstmhwvrqapgq | |
| | aggtgggggaggttctggcggcgggggaagcccttcacaggtgca | glewigyinpssaytn | |
| | actggtgcagagtggacccgaggttaaaaaaccagggtcctccgt | ynqkfkdrvtitadks | |
| | taaggttagctgcaaagcctctggctacacattttccaggagtac | tstaymelsslrsedt | |
| | aatgcactgggtgaggcaggctcctggacagggactcgagtggat | avyycarpqvhydyng | |
| | cgggtatatcaacccatctagcgcctataccaattacaaccaaaa | fpywgqgtlvtvssgg | |
| | gtttaaggaccgagttaccattaccgctgacaaatccaccagtac | ggsggggsggggsggg | |
| | agcttatatggagctgtcatctcttaggtccgaggacactgctgt | gsdiqmtqspstlsas | |
| | ttattactgcgctcgtcctcaggttcactatgactataatggttt | vgdrvtmtcsasssvs | |
| | tccctactggggtcagggaaccctggtgactgtctcttctggcgg | ymnwyqqkpgkapkrw | |
| | tggaggcagcggtgggggtgggtctggaggcggtggcagtggcgg | iydssklasgvpsrfs | |
| | cggaggctctgatattcagatgactcagtctcctagcactctcag | gsgsgtdytltisslq | |
| | cgccagcgtgggggatcgtgtgacaatgacttgctccgctagcag | pddfatyycqqwsrnp | |
| | tagtgtgtcttacatgaattggtatcagcagaagcccgggaaagc | ptfgggtkveikrs | |
| | acctaagcgctggatctatgactcttccaagctggcaagtggtgt | | |
| | cccctcacggttctctggctcaggttctggtactgactatacttt | | |
| | gactatctcctccctccagcccgatgatttcgctacctattattg | | |
| | tcagcagtggagccgtaacccacccactttcggaggcggtaccaa | | |
| | agtggagatcaagaggtcatga | | |
| DB60 VHVL | atggaagcaccagcgcagcttctcttcctcctgctactctggctc | qvqlvqsgaevkkpga | SEQ ID NO: 139 |
| x TSC456 | ccagataccaccggtcaggtgcagctggtgcagtctggggctgag | svkvsckasgytftsy | (SEQ ID |
| scFv-Fc- | gtgaagaagcctggggcctcagtgaaggtttcctgcaaggcatct | ymhwvrqapgqglewm | NO: 140) |
| scFv | ggatacaccttcaccagctactatatgcactgggtgcgtcaggcc | gwinpnsgdtsyaqkf | |
| TRI125 | cctggacaagggcttgagtggatggggtggatcaaccctaacagt | qgrvtmtrdtststvy | |
| | ggtgacacaagctatgcacagaagttccagggccgtgtcaccatg | melsslrsedtavyyc | |
| | acccgcgacacgtccacgagcacagtctacatggagctgagcagc | aqdssgsgafdiwgqg | |
| | ctgcgttctgaggacacggccgtgtattactgtgcgcaggatagt | tmvtvssggggsgggg | |
| | agtggttccggggcttttgatatctggggccaagggacaatggtc | sggggsggggsdiqmt | |
| | accgtctcttcaggcggcggcggcagcggcggcggcggcagcggc | qspsslsasvgdrvti | |
| | ggcggaggctccggcggcggcggcagcgacatccagatgacccag | tcrasqsissylnwyq | |
| | tctccatcctccctgtctgcatctgtaggagacagagtcaccatc | qkpgkapklliyaass | |
| | acttgccgggcaagtcagagcattagcagctatctgaattggtat | lqsgvpsrfsgsgsgt | |
| | cagcagaaaccagggaaagcccctaagctcctgatctatgctgca | dftltisslqpedfat | |
| | tccagtttgcaaagtggggtcccatcaaggttcagtggcagtgga | yycqqsystpltfggg | |
| | tctgggacagatttcactctcaccatcagcagtctgcaacctgaa | tkveiksssepkssdk | |
| | gattttgcaacttactactgtcaacagagttacagtacccctctc | thtcppcpapeaagap | |
| | actttcggcggaggtaccaaggtggagatcaaatcctcgagtgag | svfifppkpkdtlmis | |
| | cccaaatcttctgacaaaactcacacatgcccaccgtgcccagca | rtpevtcvvvdvshed | |
| | cctgaagccgcgggtgcaccgtcagtcttcctcttccccccaaaa | pevkfnwyvdgvevhn | |
| | cccaaggacaccctcatgatctcccggacccctgaggtcacatgc | aktkpreeqynstyrv | |
| | gtggtggtggacgtgagccacgaagaccctgaggtcaagttcaac | vsvltvlhqdwlngke | |
| | tggtacgtggacggcgtggaggtgcataatgccaagacaaagccg | ykcavsnkalpapiek | |
| | cgggaggagcagtacaacagcacgtaccgtgtggtcagcgtcctc | tiskakgqprepqvyt | |
| | accgtcctgcaccaggactggctgaatggcaaggaatacaagtgc | lppsrdeltknqvslt | |
| | gcggtctccaacaaagccctcccagcccccatcgagaaaaccatc | clvkgfypsdiavewe | |
| | tccaaagccaaagggcagccccgagaaccacaggtgtacaccctg | sngqpennykttppvl | |
| | cccccatcccgggatgagctgaccaagaaccaggtcagcctgacc | dsdgsfflyskltvdk | |
| | tgcctggtcaaaggcttctatccaagcgacatcgccgtggagtgg | srwqqgnvfscsvmhe | |
| | gagagcaatgggcagccggagaacaactacaagaccacgcctccc | alhnhytqkslslspg | |
| | gtgctggactccgacggctccttcttcctctacagcaagctcacc | sggggsggggsggggs | |
| | gtggacaagagcaggtggcagcaggggaacgtcttctcatgctcc | psqvqlvqsgpevkkp | |
| | gtgatgcatgaggctctgcacaaccactacacgcagaagagcctc | gssvkvsckasgytfs | |
| | tccctgtctccgggttccggaggagggggttcaggtgggggaggt | rstmhwvrqapgqgle | |
| | tctggcggcgggggaagcccttcacaggtgcaactggtgcagagt | wigyinpssaytnynq | |
| | ggacccgaggttaaaaaaccagggtcctccgttaaggttagctgc | kfkdrvtitadkstst | |
| | aaagcctctggctacacattttccaggagtacaatgcactgggtg | aymelsslrsedtavy | |
| | aggcaggctcctggacagggactcgagtggatcgggtatatcaac | ycarpqvhydyngfpy | |
| | ccatctagcgcctataccaattacaaccaaaagtttaaggaccga | wgqgtlvtvssggggs | |
| | gttaccattaccgctgacaaatccaccagtacagcttatatggag | ggggsggggsggggsd | |
| | ctgtcatctcttaggtccgaggacactgctgtttattactgcgct | iqmtqspstlsasvgd | |
| | cgtcctcaggttcactatgactataatggttttccctactggggt | rvtmtcsasssvsymn | |
| | cagggaaccctggtgactgtctcttctggcggtggaggcagcggt | wyqqkpgkapkrwiyd | |
| | gggggtgggtctggaggcggtggcagtggcggcggaggctctgat | ssklasgvpsrfsgsg | |
| | attcagatgactcagtctcctagcactctcagcgccagcgtgggg | sgtdytltisslqpdd | |
| | gatcgtgtgacaatgacttgctccgctagcagtagtgtgtcttac | fatyycqqwsrnpptf | |
| | atgaattggtatcagcagaagcccgggaaagcacctaagcgctgg | gggtkveikrs | |
| | atctatgactcttccaagctggcaagtggtgtcccctcacggttc | | |
| | tctggctcaggttctggtactgactatactttgactatctcctcc | | |
| | ctccagcccgatgatttcgctacctattattgtcagcagtggagc | | |
| | cgtaacccacccactttcggaggcggtaccaaagtggagatcaag | | |
| | aggtcataa | | |
| DB65 VHVL | atggaagcaccagcgcagcttctcttcctcctgctactctggctc | qvqlvqsgaevkkpga | SEQ ID NO: 141 |
| x TSC456 | ccagataccaccggtcaggtgcagctggtgcagtctggggctgag | svkvsckasgytftgy | (SEQ ID |
| scFv-Fc- | gtgaagaagcctggggcctcagtgaaggtttcctgcaaggcatct | ymhwvrqapgqglewm | NO: 142) |
| scFv | ggatacaccttcaccggctactatatgcactgggtgcgtcaggcc | gwmnpnsgntgyaqkf | |
| TRI126 | cctggacaagggcttgagtggatgggatggatgaaccctaacagt | qgrvtmtrdtststvy | |
| | ggtaacacaggctatgcacagaagttccagggccgtgtcaccatg | melsslrsedtavyyc | |
| | acccgcgacacgtccacgagcacagtctacatggagctgagcagc | akeepifgvvmdafdi | |
| | ctgcgttctgaggacacggccgtgtattactgtgcgaaagaggaa | wgqgtmvtvssggggs | |
| | ccgatttttggagtggttatggatgcttttgatatctggggccaa | ggggsggggsggggsd | |
| | gggacaatggtcaccgtctcctcaggcggcggcggcagcggcggc | iqmtqspsslsasvgd | |
| | ggcggcagcggcggcggaggctccggcggcggcggcagcgacatc | rvtitcrasqsissyl | |
| | cagatgacccagtctccatcctccctgtctgcatctgtaggagac | nwyqqkpgkapklliy | |
| | agagtcaccatcacttgccgggcaagtcagagcattagcagctat | aasslqsgvpsrfsgs | |
| | ctgaattggtatcagcagaaaccagggaaagcccctaagctcctg | gsgtdftltisslqpe | |
| | atctatgctgcatccagtttgcaaagtggggtcccatcaaggttc | dfatyycqqsystplt | |
| | agtggcagtggatctgggacagatttcactctcaccatcagcagt | fgggtkveiksssepk | |
| | ctgcaacctgaagattttgcaacttactactgtcaacagagttac | ssdkthtcppcpapea | |
| | agtacccctctcactttcggcggaggtaccaaggtggagatcaaa | agapsvflfppkpkdt | |
| | tcctcgagtgagcccaaatcttctgacaaaactcacacatgccca | lmisrtpevtcvvvdv | |
| | ccgtgcccagcacctgaagccgcgggtgcaccgtcagtcttcctc | shedpevkfnwyvdgv | |
| | ttccccccaaaacccaaggacaccctcatgatctcccggacccct | evhnaktkpreeqyns | |
| | gaggtcacatgcgtggtggtggacgtgagccacgaagaccctgag | tyrvvsvltvlhqdwi | |
| | gtcaagttcaactggtacgtggacggcgtggaggtgcataatgcc | ngkeykcavsnkalpa | |
| | aagacaaagccgcgggaggagcagtacaacagcacgtaccgtgtg | piektiskakgqprep | |
| | gtcagcgtcctcaccgtcctgcaccaggactggctgaatggcaag | qvytlppsrdeltknq | |
| | gaatacaagtgcgcggtctccaacaaagccctcccagcccccatc | vsltclvkgfypsdia | |
| | gagaaaaccatctccaaagccaaagggcagccccgagaaccacag | vewesngqpennyktt | |
| | gtgtacaccctgcccccatcccgggatgagctgaccaagaaccag | ppvldsdgsfflyskl | |
| | gtcagcctgacctgcctggtcaaaggcttctatccaagcgacatc | tvdksrwqqgnvfscs | |
| | gccgtggagtgggagagcaatgggcagccggagaacaactacaag | vmhealhnhytqksls | |
| | accacgcctcccgtgctggactccgacggctccttcttcctctac | lspgsggggsggggsg | |
| | agcaagctcaccgtggacaagagcaggtggcagcaggggaacgtc | gggspsqvqlvqsgpe | |
| | ttctcatgctccgtgatgcatgaggctctgcacaaccactacacg | vkkpgssvkvsckasg | |
| | cagaagagcctctccctgtctccgggttccggaggagggggttca | ytfsrstmhwvrqapg | |
| | ggtgggggaggttctggcggcgggggaagcccttcacaggtgcaa | qglewigyinpssayt | |
| | ctggtgcagagtggacccgaggttaaaaaaccagggtcctccgtt | nynqkfkdrvtitadk | |
| | aaggttagctgcaaagcctctggctacacattttccaggagtaca | ststaymelsslrsed | |
| | atgcactgggtgaggcaggctcctggacagggactcgagtggatc | tavyycarpqvhydyn | |
| | gggtatatcaacccatctagcgcctataccaattacaaccaaaag | gfpywgqgtlvtvssg | |
| | tttaaggaccgagttaccattaccgctgacaaatccaccagtaca | gggsggggsggggsgg | |
| | gcttatatggagctgtcatctcttaggtccgaggacactgctgtt | ggsdiqmtqspstlsa | |
| | tattactgcgctcgtcctcaggttcactatgactataatggtttt | svgdrvtmtcsasssv | |
| | ccctactggggtcagggaaccctggtgactgtctcttctggcggt | symnwyqqkpgkapkr | |
| | ggaggcagcggtgggggtgggtctggaggcggtggcagtggcggc | wiydssklasgvpsrf | |
| | ggaggctctgatattcagatgactcagtctcctagcactctcagc | sgsgsgtdytltissl | |
| | gccagcgtgggggatcgtgtgacaatgacttgctccgctagcagt | qpddfatyycqqwsrn | |
| | agtgtgtcttacatgaattggtatcagcagaagcccgggaaagca | pptfgggtkveikrs | |
| | cctaagcgctggatctatgactcttccaagctggcaagtggtgtc | | |
| | ccctcacggttctctggctcaggttctggtactgactatactttg | | |
| | actatctcctccctccagcccgatgatttcgctacctattattgt | | |
| | cagcagtggagccgtaacccacccactttcggaggcggtaccaaa | | |
| | gtggagatcaagaggtcataa | | |
| DB82 VLVH | atggaagcaccagcgcagcttctcttcctcctgctactctggctc | diqmtqspsslsasvg | SEQ ID NO: 143 |
| x TSC456 | ccagataccaccggtgacatccagatgacccagtctccatcctcc | drvtitcrasqtinny | (SEQ ID |
| scFv-Fc- | ctgtctgcatctgtaggagaccgcgtcaccatcacttgccgggca | lnwyqqkpgkapklli | NO: 144) |
| scFv | agtcagaccataaacaactatttgaactggtatcagcagaaacca | ysastlqsgvpsrfsg | |
| TRI127 | gggaaagcccctaagctcctgatctattctgcatctactttgcaa | sgsgtdftltisslqp | |
| | agtggggtcccatcacgtttcagtggcagtggatctgggacagat | edfatyychqsytspl | |
| | ttcactctcaccatcagcagtctgcaacctgaagattttgcaact | tfgggtkveikggggs | |
| | tactactgtcaccagagttacacttcacctctcactttcggcgga | ggggsggggsggggse | |
| | ggtaccaaggtggagatcaaaggcggcggcggcagcggcggcggc | vqlvesggglvqpggs | |
| | ggcagcggcggcggaggctccggcggcggcggcagcgaggtgcag | lrlscaasgftfssya | |
| | ctggtggagtctgggggaggcttggtacagcctggggggtccctg | mswvrqapgkglewvs | |
| | cgcctctcctgtgcagcctctggattcacctttagcagctatgcc | visansaglghadsvk | |
| | atgagctgggtccgccaggctccagggaaggggctggagtgggtc | grftisrdnskntlyl | |
| | tcagttattagtgccaatagtgctggtctaggccatgcggactct | qmnslraedtavyyca | |
| | gtgaagggccggttcaccatctcccgcgacaattccaagaacacg | rvgysssadafdiwgq | |
| | ctgtatctgcaaatgaacagcctgcgcgccgaggacacggccgta | gtmvtvsssepkssdk | |
| | tattactgtgcgagagtgggctatagcagctcggctgatgctttt | thtcppcpapeaagap | |
| | gatatctggggccaagggacaatggtcaccgtctcctcgagtgag | svfifppkpkdtlmis | |
| | cccaaatcttctgacaaaactcacacatgcccaccgtgcccagca | rtpevtcvvvdvshed | |
| | cctgaagccgcgggtgcaccgtcagtcttcctcttccccccaaaa | pevkfnwyvdgvevhn | |
| | cccaaggacaccctcatgatctcccggacccctgaggtcacatgc | aktkpreeqynstyrv | |
| | gtggtggtggacgtgagccacgaagaccctgaggtcaagttcaac | vsvltvlhqdwlngke | |
| | tggtacgtggacggcgtggaggtgcataatgccaagacaaagccg | ykcavsnkalpapiek | |
| | cgggaggagcagtacaacagcacgtaccgtgtggtcagcgtcctc | tiskakgqprepqvyt | |
| | accgtcctgcaccaggactggctgaatggcaaggaatacaagtgc | lppsrdeltknqvslt | |
| | gcggtctccaacaaagccctcccagcccccatcgagaaaaccatc | clvkgfypsdiavewe | |
| | tccaaagccaaagggcagccccgagaaccacaggtgtacaccctg | sngqpennykttppvl | |
| | cccccatcccgggatgagctgaccaagaaccaggtcagcctgacc | dsdgsfflyskltvdk | |
| | tgcctggtcaaaggcttctatccaagcgacatcgccgtggagtgg | srwqqgnvfscsvmhe | |
| | gagagcaatgggcagccggagaacaactacaagaccacgcctccc | alhnhytqkslslspg | |
| | gtgctggactccgacggctccttcttcctctacagcaagctcacc | sggggsggggsggggs | |
| | gtggacaagagcaggtggcagcaggggaacgtcttctcatgctcc | psqvqlvqsgpevkkp | |
| | gtgatgcatgaggctctgcacaaccactacacgcagaagagcctc | gssvkvsckasgytfs | |
| | tccctgtctccgggttccggaggagggggttcaggtgggggaggt | rstmhwvrqapgqgle | |
| | tctggcggcgggggaagcccttcacaggtgcaactggtgcagagt | wigyinpssaytnynq | |
| | ggacccgaggttaaaaaaccagggtcctccgttaaggttagctgc | kfkdrvtitadkstst | |
| | aaagcctctggctacacattttccaggagtacaatgcactgggtg | aymelsslrsedtavy | |
| | aggcaggctcctggacagggactcgagtggatcgggtatatcaac | ycarpqvhydyngfpy | |
| | ccatctagcgcctataccaattacaaccaaaagtttaaggaccga | wgqgtlvtvssggggs | |
| | gttaccattaccgctgacaaatccaccagtacagcttatatggag | ggggsggggsggggsd | |
| | ctgtcatctcttaggtccgaggacactgctgtttattactgcgct | iqmtqspstlsasvgd | |
| | cgtcctcaggttcactatgactataatggttttccctactggggt | rvtmtcsasssvsymn | |
| | cagggaaccctggtgactgtctcttctggcggtggaggcagcggt | wyqqkpgkapkrwiyd | |
| | gggggtgggtctggaggcggtggcagtggcggcggaggctctgat | ssklasgvpsrfsgsg | |
| | attcagatgactcagtctcctagcactctcagcgccagcgtgggg | sgtdytltisslqpdd | |
| | gatcgtgtgacaatgacttgctccgctagcagtagtgtgtcttac | fatyycqqwsrnpptf | |
| | atgaattggtatcagcagaagcccgggaaagcacctaagcgctgg | gggtkveikrs | |
| | atctatgactcttccaagctggcaagtggtgtcccctcacggttc | | |
| | tctggctcaggttctggtactgactatactttgactatctcctcc | | |
| | ctccagcccgatgatttcgctacctattattgtcagcagtggagc | | |
| | cgtaacccacccactttcggaggcggtaccaaagtggagatcaag | | |
| | aggtcataa | | |
| DB83 VHVL | atggaagcaccagcgcagcttctcttcctcctgctactctggctc | qvqlvqsgaevkkpga | SEQ ID NO: 145 |
| x TSC456 | ccagataccaccggtcaggtgcagctggtgcagtctggggctgag | svkvsckasgytftsy | (SEQ ID |
| scFv-Fc- | gtgaagaagcctggggcctcagtgaaggtttcctgcaaggcatct | amhwvrqapgqglewm | NO: 146) |
| scFv | ggatacaccttcactagctatgctatgcattgggtgcgtcaggcc | glvdpedgetiyaekf | |
| TRI134 | cctggacaagggcttgagtggatgggacttgttgatcctgaagat | qgrvtmtrdtststvy | |
| | ggtgaaacaatatatgcagagaagttccagggccgtgtcaccatg | melsslrsedtavyyc | |
| | acccgcgacacgtccacgagcacagtctacatggagctgagcagc | arrtyyydssgsryaf | |
| | ctgcgttctgaggacacggccgtgtattactgtgcgagacgaacg | diwgqgttvtvssggg | |
| | tattactatgatagtagtggttcccgttatgcttttgatatctgg | gsggggsggggsgggg | |
| | ggccaagggaccacggtcaccgtctcttcaggcggcggcggcagc | sdwmtqsplslpvtpg | |
| | ggcggcggcggcagcggcggcggaggctccggcggcggcggcagc | epasiscrssqsllhs | |
| | gatgttgtgatgactcagtctccactctccctgcccgtcacccct | ngdnyldwylqkpgqs | |
| | ggagagccggcctccatctcctgcaggtctagtcagagcctcctg | pqlliylgsnrasgvp | |
| | catagtaatggagacaactatttggattggtacctgcagaagcca | drfsgsgsgtdftlki | |
| | gggcagtctccacagctcctgatctatttgggttctaatcgggcc | srveaedvgvyycmqa | |
| | tccggggtccctgaccgtttcagtggcagtggatcaggcacagat | thwpltfgpgtkvdik | |
| | tttacactgaaaatcagccgtgtggaggctgaggatgttggggtt | sssepkssdkthtcpp | |
| | tattactgcatgcaagctacacactggccactcactttcggccct | cpapeaagapsvflfp | |
| | ggtaccaaagtggatatcaaatcctcgagtgagcccaaatcttct | pkpkdtlmisrtpevt | |
| | gacaaaactcacacatgcccaccgtgcccagcacctgaagccgcg | cvvvdvshedpevkfn | |
| | ggtgcaccgtcagtcttcctcttccccccaaaacccaaggacacc | wyvdgvevhnaktkpr | |
| | ctcatgatctcccggacccctgaggtcacatgcgtggtggtggac | eeqynstyrvvsvltv | |
| | gtgagccacgaagaccctgaggtcaagttcaactggtacgtggac | lhqdwlngkeykcavs | |
| | ggcgtggaggtgcataatgccaagacaaagccgcgggaggagcag | nkalpapiektiskak | |
| | tacaacagcacgtaccgtgtggtcagcgtcctcaccgtcctgcac | gqprepqvytlppsrd | |
| | caggactggctgaatggcaaggaatacaagtgcgcggtctccaac | eltknqvsltclvkgf | |
| | aaagccctcccagcccccatcgagaaaaccatctccaaagccaaa | ypsdiavewesngqpe | |
| | gggcagccccgagaaccacaggtgtacaccctgcccccatcccgg | nnykttppvldsdgsf | |
| | gatgagctgaccaagaaccaggtcagcctgacctgcctggtcaaa | flyskltvdksrwqqg | |
| | ggcttctatccaagcgacatcgccgtggagtgggagagcaatggg | nvfscsvmhealhnhy | |
| | cagccggagaacaactacaagaccacgcctcccgtgctggactcc | tqkslslspgsggggs | |
| | gacggctccttcttcctctacagcaagctcaccgtggacaagagc | ggggsggggspsqvql | |
| | aggtggcagcaggggaacgtcttctcatgctccgtgatgcatgag | vqsgpevkkpgssvkv | |
| | gctctgcacaaccactacacgcagaagagcctctccctgtctccg | sckasgytfsrstmhw | |
| | ggttccggaggagggggttcaggtgggggaggttctggcggcggg | vrqapgqglewigyin | |
| | ggaagcccttcacaggtgcaactggtgcagagtggacccgaggtt | pssaytnynqkfkdrv | |
| | aaaaaaccagggtcctccgttaaggttagctgcaaagcctctggc | titadkststaymels | |
| | tacacattttccaggagtacaatgcactgggtgaggcaggctcct | slrsedtavyycarpq | |
| | ggacagggactcgagtggatcgggtatatcaacccatctagcgcc | vhydyngfpywgqgtl | |
| | tataccaattacaaccaaaagtttaaggaccgagttaccattacc | vtvssggggsggggsg | |
| | gctgacaaatccaccagtacagcttatatggagctgtcatctctt | gggsggggsdiqmtqs | |
| | aggtccgaggacactgctgtttattactgcgctcgtcctcaggtt | pstlsasvgdrvtmtc | |
| | cactatgactataatggttttccctactggggtcagggaaccctg | sasssvsymnwyqqkp | |
| | gtgactgtctcttctggcggtggaggcagcggtgggggtgggtct | gkapkrwiydssklas | |
| | ggaggcggtggcagtggcggcggaggctctgatattcagatgact | gvpsrfsgsgsgtdyt | |
| | cagtctcctagcactctcagcgccagcgtgggggatcgtgtgaca | ltisslqpddfatyyc | |
| | atgacttgctccgctagcagtagtgtgtcttacatgaattggtat | qqwsrnpptfgggtkv | |
| | cagcagaagcccgggaaagcacctaagcgctggatctatgactct | eikrs | |
| | tccaagctggcaagtggtgtcccctcacggttctctggctcaggt | | |
| | tctggtactgactatactttgactatctcctccctccagcccgat | | |
| | gatttcgctacctattattgtcagcagtggagccgtaacccaccc | | |
| | actttcggaggcggtaccaaagtggagatcaagaggtcataa | | |
| DB86 VHVL | atggaagcaccagcgcagcttctcttcctcctgctactctggctc | qvqlvqsgaevkkpga | SEQ ID NO: 147 |
| x TSC456 | ccagataccaccggtcaggtgcagctggtgcagtctggggctgag | svkvsckasgymfsgh | (SEQ ID |
| scFv-Fc- | gtgaagaagcctggggcctcagtgaaggtttcctgcaaggcatct | sahwvrqapgqglewm | NO: 148) |
| scFv | ggatatatgttcagtggccattctgcacactgggtgcgtcaggcc | gwmnpnsgntgyaqkf | |
| TRI128 | cctggacaagggcttgagtggatgggatggatgaaccctaacagt | qgrvtmtrdtststvy | |
| | ggtaacacaggctatgcacagaagttccagggccgtgtcaccatg | melsslrsedtavyyc | |
| | acccgcgacacgtccacgagcacagtctacatggagctgagcagc | ardssgwydvfdywgq | |
| | ctgcgttctgaggacacggccgtgtattactgtgcgagagatagc | gtlvtvssggggsggg | |
| | agtggctggtacgatgtctttgactactggggccaggggaccctg | gsggggsggggsdiqm | |
| | gtcaccgtctcctcaggtggaggcggttcaggcggaggtggatcc | tqspsslsasvgdrvt | |
| | ggcggtggcggctccggtggcggcggatctgacatccagatgacc | itcrasqgirndlgwy | |
| | cagtctccatcctccctgtctgcatctgtaggagaccgcgtcacc | qqkpgkapklliyaas | |
| | atcacttgccgggcaagtcagggcatcagaaatgatttaggttgg | tlqsgvpsrfsgsgsg | |
| | tatcagcagaaaccagggaaagcccctaagctcctgatctatgct | tdftltisslqpedfa | |
| | gcatccactttgcaatcaggggtcccatcacgtttcagtggcagt | tyycqqsygapltfgg | |
| | ggatctgggacagatttcactctcaccatcagcagtctgcaacct | gtkveiksssepkssd | |
| | gaagattttgcaacttactactgtcaacagagttacggtgccccc | kthtcppcpapeaaga | |
| | ctcactttcggcggaggtaccaaggtggagatcaaatcctcgagt | psvflfppkpkdtlmi | |
| | gagcccaaatcttctgacaaaactcacacatgcccaccgtgccca | srtpevtcvvvdvshe | |
| | gcacctgaagccgcgggtgcaccgtcagtcttcctcttcccccca | dpevkfnwyvdgvevh | |
| | aaacccaaggacaccctcatgatctcccggacccctgaggtcaca | naktkpreeqynstyr | |
| | tgcgtggtggtggacgtgagccacgaagaccctgaggtcaagttc | vvsvltvlhqdwlngk | |
| | aactggtacgtggacggcgtggaggtgcataatgccaagacaaag | eykcavsnkalpapie | |
| | ccgcgggaggagcagtacaacagcacgtaccgtgtggtcagcgtc | ktiskakgqprepqvy | |
| | ctcaccgtcctgcaccaggactggctgaatggcaaggaatacaag | tlppsrdeltknqvsl | |
| | tgcgcggtctccaacaaagccctcccagcccccatcgagaaaacc | tcivkgfypsdiavew | |
| | atctccaaagccaaagggcagccccgagaaccacaggtgtacacc | esngqpennykttppv | |
| | ctgcccccatcccgggatgagctgaccaagaaccaggtcagcctg | ldsdgsfflyskltvd | |
| | acctgcctggtcaaaggcttctatccaagcgacatcgccgtggag | ksrwqqgnvfscsvmh | |
| | tgggagagcaatgggcagccggagaacaactacaagaccacgcct | ealhnhytqkslslsp | |
| | cccgtgctggactccgacggctccttcttcctctacagcaagctc | gsggggsggggsgggg | |
| | accgtggacaagagcaggtggcagcaggggaacgtcttctcatgc | spsqvqlvqsgpevkk | |
| | tccgtgatgcatgaggctctgcacaaccactacacgcagaagagc | pgssvkvsckasgytf | |
| | ctctccctgtctccgggttccggaggagggggttcaggtggggga | srstmhwvrqapgqgl | |
| | ggttctggcggcgggggaagcccttcacaggtgcaactggtgcag | ewigyinpssaytnyn | |
| | agtggacccgaggttaaaaaaccagggtcctccgttaaggttagc | qkfkdrvtitadksts | |
| | tgcaaagcctctggctacacattttccaggagtacaatgcactgg | taymelsslrsedtav | |
| | gtgaggcaggctcctggacagggactcgagtggatcgggtatatc | yycarpqvhydyngfp | |
| | aacccatctagcgcctataccaattacaaccaaaagtttaaggac | ywgqgtlvtvssgggg | |
| | cgagttaccattaccgctgacaaatccaccagtacagcttatatg | sggggsggggsggggs | |
| | gagctgtcatctcttaggtccgaggacactgctgtttattactgc | diqmtqspstlsasvg | |
| | gctcgtcctcaggttcactatgactataatggttttccctactgg | drvtmtcsasssvsym | |
| | ggtcagggaaccctggtgactgtctcttctggcggtggaggcagc | nwyqqkpgkapkrwiy | |
| | ggtgggggtgggtctggaggcggtggcagtggcggcggaggctct | dssklasgvpsrfsgs | |
| | gatattcagatgactcagtctcctagcactctcagcgccagcgtg | gsgtdytltisslqpd | |
| | ggggatcgtgtgacaatgacttgctccgctagcagtagtgtgtct | dfatyycqqwsrnppt | |
| | tacatgaattggtatcagcagaagcccgggaaagcacctaagcgc | fgggtkveikrs | |
| | tggatctatgactcttccaagctggcaagtggtgtcccctcacgg | | |
| | ttctctggctcaggttctggtactgactatactttgactatctcc | | |
| | tccctccagcccgatgatttcgctacctattattgtcagcagtgg | | |
| | agccgtaacccacccactttcggaggcggtaccaaagtggagatc | | |
| | aagaggtcataa | | |
| DB280 | atggaagcaccagcgcagcttctcttcctcctgctactctggctc | qvqlvqsgaevkkpga | SEQ ID NO: 149 |
| VHVL x | ccagataccaccggtcaggtgcagctggtgcagtctggggctgag | svkvsckasgyslnly | (SEQ ID |
| TSC456 | gtgaagaagcctggggcctcagtgaaggtttcctgcaaggcatct | ymhwvrqapgqglewm | NO: 150) |
| scFv-Fc- | ggatacagcctcaacttatactatatgcactgggtgcgtcaggcc | gwmnpnsgntgyaqkf | |
| scFv | cctggacaagggcttgagtggatgggatggatgaaccctaacagt | qgrvtmtrdtststvy | |
| TRI131 | ggtaacacaggctatgcacagaagttccagggccgtgtcaccatg | melsslrsedtavyyc | |
| | acccgcgacacgtccacgagcacagtctacatggagctgagcagc | asldcsggscyseyda | |
| | ctgcgttctgaggacacggccgtgtattactgtgcgagcctcgat | fdiwgqgttvtvssgg | |
| | tgtagtggtggtagctgctactccgaatatgatgcttttgatatc | ggsggggsggggsggg | |
| | tggggccaagggaccacggtcaccgtctcctcaggcggcggcggc | gsdiqmtqspsslsas | |
| | agcggcggcggcggcagcggcggcggaggctccggcggcggcggc | vgdrvtitcrasqsis | |
| | agcgacatccagatgacccagtctccatcctccctgtctgcatct | sylnwyqqkpgkapkl | |
| | gtaggagacagagtcaccatcacttgccgggcaagtcagagcatt | liyaasslqsgvpsrf | |
| | agcagctatctgaattggtatcagcagaaaccagggaaagcccct | sgsgsgtdftltissl | |
| | aagctcctgatctatgctgcatccagtttgcaaagtggggtccca | qpedfatyycqqsyst | |
| | tcaaggttcagtggcagtggatctgggacagatttcactctcacc | pltfgggtkveiksss | |
| | atcagcagtctgcaacctgaagattttgcaacttactactgtcaa | epkssdkthtcppcpa | |
| | cagagttacagtacccctctcactttcggcggaggtaccaaggtg | peaagapsvflfppkp | |
| | gagatcaaatcctcgagtgagcccaaatcttctgacaaaactcac | kdtlmisrtpevtcvv | |
| | acatgcccaccgtgcccagcacctgaagccgcgggtgcaccgtca | vdvshedpevkfnwyv | |
| | gtcttcctcttccccccaaaacccaaggacaccctcatgatctcc | dgvevhnaktkpreeq | |
| | cggacccctgaggtcacatgcgtggtggtggacgtgagccacgaa | ynstyrvvsvltvlhq | |
| | gaccctgaggtcaagttcaactggtacgtggacggcgtggaggtg | dwingkeykcavsnka | |
| | cataatgccaagacaaagccgcgggaggagcagtacaacagcacg | lpapiektiskakgqp | |
| | taccgtgtggtcagcgtcctcaccgtcctgcaccaggactggctg | repqvytlppsrdelt | |
| | aatggcaaggaatacaagtgcgcggtctccaacaaagccctccca | knqvsltclvkgfyps | |
| | gcccccatcgagaaaaccatctccaaagccaaagggcagccccga | diavewesngqpenny | |
| | gaaccacaggtgtacaccctgcccccatcccgggatgagctgacc | kttppvldsdgsffly | |
| | aagaaccaggtcagcctgacctgcctggtcaaaggcttctatcca | skltvdksrwqqgnvf | |
| | agcgacatcgccgtggagtgggagagcaatgggcagccggagaac | scsvmhealhnhytqk | |
| | aactacaagaccacgcctcccgtgctggactccgacggctccttc | slslspgsggggsggg | |
| | ttcctctacagcaagctcaccgtggacaagagcaggtggcagcag | gsggggspsqvqlvqs | |
| | gggaacgtcttctcatgctccgtgatgcatgaggctctgcacaac | gpevkkpgssvkvsck | |
| | cactacacgcagaagagcctctccctgtctccgggttccggagga | asgytfsrstmhwvrq | |
| | gggggttcaggtgggggaggttctggcggcgggggaagcccttca | apgqglewigyinpss | |
| | caggtgcaactggtgcagagtggacccgaggttaaaaaaccaggg | aytnynqkfkdrvtit | |
| | tcctccgttaaggttagctgcaaagcctctggctacacattttcc | adkststaymelsslr | |
| | aggagtacaatgcactgggtgaggcaggctcctggacagggactc | sedtavyycarpqvhy | |
| | gagtggatcgggtatatcaacccatctagcgcctataccaattac | dyngfpywgqgtlvtv | |
| | aaccaaaagtttaaggaccgagttaccattaccgctgacaaatcc | ssggggsggggsgggg | |
| | accagtacagcttatatggagctgtcatctcttaggtccgaggac | sggggsdiqmtqspst | |
| | actgctgtttattactgcgctcgtcctcaggttcactatgactat | lsasvgdrvtmtcsas | |
| | aatggttttccctactggggtcagggaaccctggtgactgtctct | ssvsymnwyqqkpgka | |
| | tctggcggtggaggcagcggtgggggtgggtctggaggcggtggc | pkrwiydssklasgvp | |
| | agtggcggcggaggctctgatattcagatgactcagtctcctagc | srfsgsgsgtdytlti | |
| | actctcagcgccagcgtgggggatcgtgtgacaatgacttgctcc | sslqpddfatyycqqw | |
| | gctagcagtagtgtgtcttacatgaattggtatcagcagaagccc | srnpptfgggtkveik | |
| | gggaaagcacctaagcgctggatctatgactcttccaagctggca | rs | |
| | agtggtgtcccctcacggttctctggctcaggttctggtactgac | | |
| | tatactttgactatctcctccctccagcccgatgatttcgctacc | | |
| | tattattgtcagcagtggagccgtaacccacccactttcggaggc | | |
| | ggtaccaaagtggagatcaagaggtcataa | | |
| DB331 | atggaagcaccagcgcagcttctcttcctcctgctactctggctc | qvqlvqsgaevkkpga | SEQ ID NO: 151 |
| VHVL x | ccagataccaccggtcaggtgcagctggtgcagtctggggctgag | svkvsckasgytftsy | (SEQ ID |
| TSC456 | gtgaagaagcctggggcctcagtgaaggtttcctgcaaggcatct | ymhwvrqapgqglewm | NO: 152) |
| scFv-Fc- | ggatacaccttcaccagctactatatgcactgggtgcgtcaggcc | gwmnpnsgntgyaqkf | |
| scFv | cctggacaagggcttgagtggatgggatggatgaaccctaacagt | qgrvtmtrdtststvy | |
| TRI132 | ggtaacacaggctatgcacagaagttccagggccgtgtcaccatg | melsslrsedtavyyc | |
| | acccgcgacacgtccacgagcacagtctacatggagctgagcagc | atdlagealfdpwgqg | |
| | ctgcgttctgaggacacggccgtgtattactgtgcaacagatctc | tlvtvssggggsgggg | |
| | gcgggggaagccttgttcgacccctggggccagggcaccctggtc | sggggsggggsdiqmt | |
| | accgtctcctcaggcggcggcggcagcggcggcggcggcagcggc | qspsslsasvgdrvti | |
| | ggcggaggctccggcggcggcggcagcgacatccagatgacccag | tcrasqsissylnwyq | |
| | tctccatcctccctgtctgcatctgtaggagacagagtcaccatc | qkpgkapklliyaass | |
| | acttgccgggcaagtcagagcattagcagctatctgaattggtat | lqsgvpsrfsgsgsgt | |
| | cagcagaaaccagggaaagcccctaagctcctgatctatgctgca | dftltisslqpedfat | |
| | tccagtttgcaaagtggggtcccatcaaggttcagtggcagtgga | yycqqsystpltfggg | |
| | tctgggacagatttcactctcaccatcagcagtctgcaacctgaa | tkveiksssepkssdk | |
| | gattttgcaacttactactgtcaacagagttacagtacccctctc | thtcppcpapeaagap | |
| | actttcggcggaggtaccaaggtggagatcaaatcctcgagtgag | svflfppkpkdtlmis | |
| | cccaaatcttctgacaaaactcacacatgcccaccgtgcccagca | rtpevtcvvvdvshed | |
| | cctgaagccgcgggtgcaccgtcagtcttcctcttccccccaaaa | pevkfnwyvdgvevhn | |
| | cccaaggacaccctcatgatctcccggacccctgaggtcacatgc | aktkpreeqynstyrv | |
| | gtggtggtggacgtgagccacgaagaccctgaggtcaagttcaac | vsvltvlhqdwlngke | |
| | tggtacgtggacggcgtggaggtgcataatgccaagacaaagccg | ykcavsnkalpapiek | |
| | cgggaggagcagtacaacagcacgtaccgtgtggtcagcgtcctc | tiskakgqprepqvyt | |
| | accgtcctgcaccaggactggctgaatggcaaggaatacaagtgc | lppsrdeltknqvslt | |
| | gcggtctccaacaaagccctcccagcccccatcgagaaaaccatc | clvkgfypsdiavewe | |
| | tccaaagccaaagggcagccccgagaaccacaggtgtacaccctg | sngqpennykttppvl | |
| | cccccatcccgggatgagctgaccaagaaccaggtcagcctgacc | dsdgsfflyskltvdk | |
| | tgcctggtcaaaggcttctatccaagcgacatcgccgtggagtgg | srwqqgnvfscsvmhe | |
| | gagagcaatgggcagccggagaacaactacaagaccacgcctccc | alhnhytqkslslspg | |
| | gtgctggactccgacggctccttcttcctctacagcaagctcacc | sggggsggggsggggs | |
| | gtggacaagagcaggtggcagcaggggaacgtcttctcatgctcc | psqvqlvqsgpevkkp | |
| | gtgatgcatgaggctctgcacaaccactacacgcagaagagcctc | gssvkvsckasgytfs | |
| | tccctgtctccgggttccggaggagggggttcaggtgggggaggt | rstmhwvrqapgqgle | |
| | tctggcggcgggggaagcccttcacaggtgcaactggtgcagagt | wigyinpssaytnynq | |
| | ggacccgaggttaaaaaaccagggtcctccgttaaggttagctgc | kfkdrvtitadkstst | |
| | aaagcctctggctacacattttccaggagtacaatgcactgggtg | aymelsslrsedtavy | |
| | aggcaggctcctggacagggactcgagtggatcgggtatatcaac | ycarpqvhydyngfpy | |
| | ccatctagcgcctataccaattacaaccaaaagtttaaggaccga | wgqgtlvtvssggggs | |
| | gttaccattaccgctgacaaatccaccagtacagcttatatggag | ggggsggggsggggsd | |
| | ctgtcatctcttaggtccgaggacactgctgtttattactgcgct | iqmtqspstlsasvgd | |
| | cgtcctcaggttcactatgactataatggttttccctactggggt | rvtmtcsasssvsymn | |
| | cagggaaccctggtgactgtctcttctggcggtggaggcagcggt | wyqqkpgkapkrwiyd | |
| | gggggtgggtctggaggcggtggcagtggcggcggaggctctgat | ssklasgvpsrfsgsg | |
| | attcagatgactcagtctcctagcactctcagcgccagcgtgggg | sgtdytltisslqpdd | |
| | gatcgtgtgacaatgacttgctccgctagcagtagtgtgtcttac | fatyycqqwsrnpptf | |
| | atgaattggtatcagcagaagcccgggaaagcacctaagcgctgg | gggtkveikrs | |
| | atctatgactcttccaagctggcaagtggtgtcccctcacggttc | | |
| | tctggctcaggttctggtactgactatactttgactatctcctcc | | |
| | ctccagcccgatgatttcgctacctattattgtcagcagtggagc | | |
| | cgtaacccacccactttcggaggcggtaccaaagtggagatcaag | | |
| | aggtcataa | | |
| DB415 | atggaagcaccagcgcagcttctcttcctcctgctactctggctc | evqlvesggglvqpgg | SEQ ID NO: 153 |
| VHVL x | ccagataccaccggtgaggtgcagctggtggagtctgggggaggc | slriscaasgitfssy | (SEQ ID |
| TSC456 | ttggtacagcctggggggtccctgcgcctctcctgtgcagcctct | gmhwvrqapgkglewv | NO: 154) |
| scFv-Fc- | ggaatcaccttcagtagttatggcatgcattgggtccgccaggct | sgiswnsgnrvyvdsv | |
| scFv | ccagggaaggggctggagtgggtctcaggtattagttggaatagt | kgrftisrdnskntly | |
| TRI138 | ggtaacagagtctatgtggactctgtgaagggccggttcaccatc | lqmnslraedtavyyc | |
| | tcccgcgacaattccaagaacacgctgtatctgcaaatgaacagc | ardtndafdiwgqgtt | |
| | ctgcgcgccgaggacacggccgtatattactgtgcgagagatact | vtvssggggsggggsg | |
| | aatgatgcttttgatatctggggccaagggaccacggtcaccgtc | gggsggggsdiqmtqs | |
| | tcctcaggtggaggcggttcaggcggaggtggatccggcggtggc | psslsasvgdrvtitc | |
| | ggctccggtggcggcggatctgacatccagatgacccagtctcca | rasqsissylnwyqqk | |
| | tcctccctgtctgcatctgtaggagacagagtcaccatcacttgc | pgkapklliyaasslq | |
| | cgggcaagtcagagcattagcagctatctgaattggtatcagcag | sgvpsrfsgsgsgtdf | |
| | aaaccagggaaagcccctaagctcctgatctatgctgcatccagt | tltisslqpedfatyy | |
| | ttgcaaagtggggtcccatcaaggttcagtggcagtggatctggg | cqqsystpltfgggtk | |
| | acagatttcactctcaccatcagcagtctgcaacctgaagatttt | veiksssepkssdkth | |
| | gcaacttactactgtcaacagagttacagtacccctctcactttc | tcppcpapeaagapsv | |
| | ggcggaggtaccaaggtggagatcaaatcctcgagtgagcccaaa | flfppkpkdtlmisrt | |
| | tcttctgacaaaactcacacatgcccaccgtgcccagcacctgaa | pevtcvvvdvshedpe | |
| | gccgcgggtgcaccgtcagtcttcctcttccccccaaaacccaag | vkfnwyvdgvevhnak | |
| | gacaccctcatgatctcccggacccctgaggtcacatgcgtggtg | tkpreeqynstyrvvs | |
| | gtggacgtgagccacgaagaccctgaggtcaagttcaactggtac | vltvlhqdwlngkeyk | |
| | gtggacggcgtggaggtgcataatgccaagacaaagccgcgggag | cavsnkalpapiekti | |
| | gagcagtacaacagcacgtaccgtgtggtcagcgtcctcaccgtc | skakgqprepqvytlp | |
| | ctgcaccaggactggctgaatggcaaggaatacaagtgcgcggtc | psrdeltknqvsltcl | |
| | tccaacaaagccctcccagcccccatcgagaaaaccatctccaaa | vkgfypsdiavewesn | |
| | gccaaagggcagccccgagaaccacaggtgtacaccctgccccca | gqpennykttppvlds | |
| | tcccgggatgagctgaccaagaaccaggtcagcctgacctgcctg | dgsfflyskltvdksr | |
| | gtcaaaggcttctatccaagcgacatcgccgtggagtgggagagc | wqqgnvfscsvmheal | |
| | aatgggcagccggagaacaactacaagaccacgcctcccgtgctg | hnhytqkslslspgsg | |
| | gactccgacggctccttcttcctctacagcaagctcaccgtggac | gggsggggsggggsps | |
| | aagagcaggtggcagcaggggaacgtcttctcatgctccgtgatg | qvqlvqsgpevkkpgs | |
| | catgaggctctgcacaaccactacacgcagaagagcctctccctg | svkvsckasgytfsrs | |
| | tctccgggttccggaggagggggttcaggtgggggaggttctggc | tmhwvrqapgqglewi | |
| | ggcgggggaagcccttcacaggtgcaactggtgcagagtggaccc | gyinpssaytnynqkf | |
| | gaggttaaaaaaccagggtcctccgttaaggttagctgcaaagcc | kdrvtitadkststay | |
| | tctggctacacattttccaggagtacaatgcactgggtgaggcag | melsslrsedtavyyc | |
| | gctcctggacagggactggagtggatcgggtatatcaacccatct | arpqvhydyngfpywg | |
| | agcgcctataccaattacaaccaaaagtttaaggaccgagttacc | qgtlvtvssggggsgg | |
| | attaccgctgacaaatccaccagtacagcttatatggagctgtca | ggsggggsggggsdiq | |
| | tctcttaggtccgaggacactgctgtttattactgcgctcgtcct | mtqspstlsasvgdrv | |
| | caggttcactatgactataatggttttccctactggggtcaggga | tmtcsasssvsymnwy | |
| | accctggtgactgtctcttctggcggtggaggcagcggtgggggt | qqkpgkapkrwiydss | |
| | gggtctggaggcggtggcagtggcggcggaggctctgatattcag | klasgvpsrfsgsgsg | |
| | atgactcagtctcctagcactctcagcgccagcgtgggggatcgt | tdytltisslqpddfa | |
| | gtgacaatgacttgctccgctagcagtagtgtgtcttacatgaat | tyycqqwsrnpptfgg | |
| | tggtatcagcagaagcccgggaaagcacctaagcgctggatctat | gtkveikrs | |
| | gactcttccaagctggcaagtggtgtcccctcacggttctctggc | | |
| | tcaggttctggtactgactatactttgactatctcctccctccag | | |
| | cccgatgatttcgctacctattattgtcagcagtggagccgtaac | | |
| | ccacccactttcggaggcggtaccaaagtggagatcaagaggtca | | |
| | tga | | |
| DB435 | atggaagcaccagcgcagcttctcttcctcctgctactctggctc | qvqlvqsgaevkkpga | SEQ ID NO: 155 |
| VHVL x | ccagataccaccggtcaggtgcagctggtgcagtctggggctgag | svkvsckasggtfssy | (SEQ ID |
| TSC456 | gtgaagaagcctggggcctcagtgaaggtttcctgcaaggcatct | aiswvrqapgqglewm | NO: 156) |
| scFv-Fc- | ggaggcaccttcagcagctatgctatcagctgggtgcgtcaggcc | gwitphngnikyaref | |
| scFv | cctggacaagggcttgagtggatgggctggatcacccctcacaat | qgrvtmtrdtststvy | |
| TRI139 | ggtaacataaagtatgcacgggagttccagggccgtgtcaccatg | melsslrsedtavyyc | |
| | acccgcgacacgtccacgagcacagtctacatggagctgagcagc | akdlnwnaafdywgqg | |
| | ctgcgttctgaggacacggccgtgtattactgtgcgaaagatctg | tlvtvssggggsgggg | |
| | aactggaacgcagcctttgactactggggccaggggaccctggtc | sggggsggggsdiqmt | |
| | accgtctcctcaggtggaggcggttcaggcggaggtggatccggc | qspsslsasvgdrvti | |
| | ggtggcggctccggtggcggcggatctgacatccagatgacccag | tcrasqsissylnwyq | |
| | tctccatcctccctgtctgcatctgtaggagacagagtcaccatc | qkpgkapklliyaass | |
| | acttgccgggcaagtcagagcattagcagctatctgaattggtat | lqsgvpsrfsgsgsgt | |
| | cagcagaaaccagggaaagcccctaagctcctgatctatgctgca | dftltisslqpedfat | |
| | tccagtttgcaaagtggggtcccatcaaggttcagtggcagtgga | yycqqsystpltfggg | |
| | tctgggacagatttcactctcaccatcagcagtctgcaacctgaa | tkveiksssepkssdk | |
| | gattttgcaacttactactgtcaacagagttacagtacccctctc | thtcppcpapeaagap | |
| | actttcggcggaggtaccaaggtggagatcaaatcctcgagtgag | svfifppkpkdtlmis | |
| | cccaaatcttctgacaaaactcacacatgcccaccgtgcccagca | rtpevtcvvvdvshed | |
| | cctgaagccgcgggtgcaccgtcagtcttcctcttccccccaaaa | pevkfnwyvdgvevhn | |
| | cccaaggacaccctcatgatctcccggacccctgaggtcacatgc | aktkpreeqynstyrv | |
| | gtggtggtggacgtgagccacgaagaccctgaggtcaagttcaac | vsvltvlhqdwlngke | |
| | tggtacgtggacggcgtggaggtgcataatgccaagacaaagccg | ykcavsnkalpapiek | |
| | cgggaggagcagtacaacagcacgtaccgtgtggtcagcgtcctc | tiskakgqprepqvyt | |
| | accgtcctgcaccaggactggctgaatggcaaggaatacaagtgc | lppsrdeltknqvslt | |
| | gcggtctccaacaaagccctcccagcccccatcgagaaaaccatc | clvkgfypsdiavewe | |
| | tccaaagccaaagggcagccccgagaaccacaggtgtacaccctg | sngqpennykttppvl | |
| | cccccatcccgggatgagctgaccaagaaccaggtcagcctgacc | dsdgsfflyskltvdk | |
| | tgcctggtcaaaggcttctatccaagcgacatcgccgtggagtgg | srwqqgnvfscsvmhe | |
| | gagagcaatgggcagccggagaacaactacaagaccacgcctccc | alhnhytqkslslspg | |
| | gtgctggactccgacggctccttcttcctctacagcaagctcacc | sggggsggggsggggs | |
| | gtggacaagagcaggtggcagcaggggaacgtcttctcatgctcc | psqvqlvqsgpevkkp | |
| | gtgatgcatgaggctctgcacaaccactacacgcagaagagcctc | gssvkvsckasgytfs | |
| | tccctgtctccgggttccggaggagggggttcaggtgggggaggt | rstmhwvrqapgqgle | |
| | tctggcggcgggggaagcccttcacaggtgcaactggtgcagagt | wigyinpssaytnynq | |
| | ggacccgaggttaaaaaaccagggtcctccgttaaggttagctgc | kfkdrvtitadkstst | |
| | aaagcctctggctacacattttccaggagtacaatgcactgggtg | aymelsslrsedtavy | |
| | aggcaggctcctggacagggactggagtggatcgggtatatcaac | ycarpqvhydyngfpy | |
| | ccatctagcgcctataccaattacaaccaaaagtttaaggaccga | wgqgtlvtvssggggs | |
| | gttaccattaccgctgacaaatccaccagtacagcttatatggag | ggggsggggsggggsd | |
| | ctgtcatctcttaggtccgaggacactgctgtttattactgcgct | iqmtqspstlsasvgd | |
| | cgtcctcaggttcactatgactataatggttttccctactggggt | rvtmtcsasssvsymn | |
| | cagggaaccctggtgactgtctcttctggcggtggaggcagcggt | wyqqkpgkapkrwiyd | |
| | gggggtgggtctggaggcggtggcagtggcggcggaggctctgat | ssklasgvpsrfsgsg | |
| | attcagatgactcagtctcctagcactctcagcgccagcgtgggg | sgtdytltisslqpdd | |
| | gatcgtgtgacaatgacttgctccgctagcagtagtgtgtcttac | fatyycqqwsrnpptf | |
| | atgaattggtatcagcagaagcccgggaaagcacctaagcgctgg | gggtkveikrs | |
| | atctatgactcttccaagctggcaagtggtgtcccctcacggttc | | |
| | tctggctcaggttctggtactgactatactttgactatctcctcc | | |
| | ctccagcccgatgatttcgctacctattattgtcagcagtggagc | | |
| | cgtaacccacccactttcggaggcggtaccaaagtggagatcaag | | |
| | aggtcatga | | |
| Cris7 and | | RSTMH | (SEQ ID |
| DRA222 VH | | | NO: 165) |
| CDR1 | | | |
| (Kabat) | | | |
| Cris7 and | | YINPSSAYTNYNQKF | (SEQ ID |
| DRA222 VH | | K | NO: 166) |
| CDR2 | | | |
| (Kabat) | | | |
| Cris7 and | | QVHYDYNGFPY | (SEQ ID |
| DRA222 VH | | | NO: 167) |
| CDR3 | | | |
| (Kabat) | | | |
| Cris7 and | | SASSSVSYMN | (SEQ ID |
| DRA222 VL | | | NO: 162) |
| CDR1 | | | |
| (Kabat) | | | |
| Cris7 and | | DSSKLAS | (SEQ ID |
| DRA222 VL | | | NO: 163) |
| CDR2 | | | |
| (Kabat) | | | |
| Cris7 and | | QQWSRNPPT | (SEQ ID |
| DRA222 VL | | | NO: 164) |
| CDR3 | | | |
| (Kabat) | | | |
| Cris7 and | | GYTFTRST | (SEQ ID |
| DRA222 VH | | | NO: 171) |
| CDR1 | | | |
| (IMGT) | | | |
| Cris7 and | | INPSSAYT | (SEQ ID |
| DRA222 VH | | | NO: 172) |
| CDR2 | | | |
| (IMGT) | | | |
| Cris7 and | | QQWSRNPPT | (SEQ ID |
| DRA222 VH | | | NO: 173) |
| CDR3 | | | |
| (IMGT) | | | |
| Cris7 and | | ASSSVSY | (SEQ ID |
| DRA222 VL | | | NO: 168) |
| CDR1 | | | |
| (IMGT) | | | |
| Cris7 and | | DSS | (SEQ ID |
| DRA222 VL | | | NO: 169) |
| CDR2 | | | |
| (IMGT) | | | |
| Cris7 and | | QQWSRNPPT | (SEQ ID |
| DRA222 VL | | | NO: 170) |
| CDR3 | | | |
| (IMGT) | | | |
| I2C VH | | KYAMN | (SEQ ID |
| CDR1 | | | NO: 174) |
| (Kabat) | | | |
| I2C VH | | RIRSKYNNYATYYAD | (SEQ ID |
| CDR2 | | SVKD | NO: 175) |
| (Kabat) | | | |
| I2C VH | | HGNFGNSYISYWAY | (SEQ ID |
| CDR3 | | | NO: 176) |
| (Kabat) | | | |
| I2C VL | | GSSTGAVTSGNYPN | (SEQ ID |
| CDR1 | | | NO: 307) |
| (Kabat) | | | |
| I2C VL | | GTKFLAP | (SEQ ID |
| CDR2 | | | NO: 308) |
| (Kabat) | | | |
| I2C VL | | VLWYSNRWV | (SEQ ID |
| CDR3 | | | NO: 309) |
| (Kabat) | | | |
| I2C VH | | GFTFNKYA | (SEQ ID |
| CDR1 | | | NO: 179) |
| (IMGT) | | | |
| I2C VH | | IRSKYNNYAT | (SEQ ID |
| CDR2 | | | NO: 180) |
| (IMGT) | | | |
| I2C VH | | VRHGNFGNSYISYW | (SEQ ID |
| CDR3 | | AY | NO: 181) |
| (IMGT) | | | |
| I2C VL | | TGAVTSGNY | (SEQ ID |
| CDR1 | | | NO: 310) |
| (IMGT) | | | |
| I2C VL | | GTK | (SEQ ID |
| CDR2 | | | NO: 177) |
| (IMGT) | | | |
| I2C VL | | VLWYSNRWV | (SEQ ID |
| CDR3 | | | NO: 178) |
| (IMGT) | | | |
| HuM291 | | SYTMH | (SEQ ID |
| VH CDR1 | | | NO: 185) |
| (Kabat) | | | |
| HuM291 | | YINPRSGYTHYNQKL | (SEQ ID |
| VH CDR2 | | KD | NO: 186) |
| (Kabat) | | | |
| HuM291 | | SAYYDYDGFAY | (SEQ ID |
| VH CDR3 | | | NO: 187) |
| (Kabat) | | | |
| HuM291 VL | | SASSSVSYMN | (SEQ ID |
| CDR1 | | | NO: 182) |
| (Kabat) | | | |
| HuM291 VL | | DTSKLAS | (SEQ ID |
| CDR2 | | | NO: 183) |
| (Kabat) | | | |
| HuM291 VL | | QQWSSNPPT | (SEQ ID |
| CDR3 | | | NO: 184) |
| (Kabat) | | | |
| HuM291 | | GYTFISYT | (SEQ ID |
| VH CDR1 | | | NO: 191) |
| (IMGT) | | | |
| HuM291 | | INPRSGYT | (SEQ ID |
| VH CDR2 | | | NO: 192) |
| (IMGT) | | | |
| HuM291 | | ARSAYYDYDGFAY | (SEQ ID |
| VH CDR3 | | | NO: 193) |
| (IMGT) | | | |
| HuM291 VL | | ASSSVSY | (SEQ ID |
| CDR1 | | | NO: 188) |
| (IMGT) | | | |
| HuM291 VL | | DTS | (SEQ ID |
| CDR2 | | | NO: 189) |
| (IMGT) | | | |
| HuM291 VL | | QQWSSNPPT | (SEQ ID |
| CDR3 | | | NO: 190) |
| (IMGT) | | | |
| TSC455 | | QVQLVQSGPEVKKP | (SEQ ID |
| (anti-CD3) | | GSSVKVSCKASGYTF | NO: 311) |
| TSC394 | | SRSTMHWVRQAPG | |
| F87Y scFv | | QGLEWIGYINPSSAY | |
| | | TNYNQKFKDRVTIT | |
| | | ADKSTSTAYMELSSL | |
| | | RSEDTAVYYCARPQ | |
| | | VHYDYNGFPYWGQ | |
| | | GTLVTVSSGGGGSG | |
| | | GGGSGGGGSGGGG | |
| | | SDIQMTQSPSTLSAS | |
| | | VGDRVTMTCSASSS | |
| | | VSYMNWYQQKPG | |
| | | KAPKRWIYDSSKLAS | |
| | | GVPSRFSGSGSGTEY | |
| | | TLTISSLQPDDFATYY | |
| | | CQQWSRNPPTFGG | |
| | | GTKVEIKRSSS | |
| TSC456 | | QVQLVQSGPEVKKP | (SEQ ID |
| (anti-CD3) | | GSSVKVSCKASGYTF | NO: 312) |
| TSC394 | | SRSTMHWVRQAPG | |
| E86D F87Y | | QGLEWIGYINPSSAY | |
| scFv | | TNYNQKFKDRVTIT | |
| | | ADKSTSTAYMELSSL | |
| | | RSEDTAVYYCARPQ | |
| | | VHYDYNGFPYWGQ | |
| | | GTLVTVSSGGGGSG | |
| | | GGGSGGGGSGGGG | |
| | | SDIQMTQSPSTLSAS | |
| | | VGDRVTMTCSASSS | |
| | | VSYMNWYQQKPG | |
| | | KAPKRWIYDSSKLAS | |
| | | GVPSRFSGSGSGTD | |
| | | YTLTISSLQPDDFATY | |
| | | YCQQWSRNPPTFG | |
| | | GGTKVEIKRSSS | |
| TSC455 and | | QVQLVQSGPEVKKP | (SEQ ID |
| TSC456 | | GSSVKVSCKASGYTF | NO: 159) |
| variable | | SRSTMHWVRQAPG | |
| heavy | | QGLEWIGYINPSSAY | |
| domain | | TNYNQKFKDRVTIT | |
| | | ADKSTSTAYMELSSL | |
| | | RSEDTAVYYCARPQ | |
| | | VHYDYNGFPYWGQ | |
| | | GTLVTVSS | |
| TSC455 | | DIQMTQSPSTLSAS | (SEQ ID |
| variable | | VGDRVTMTCSASSS | NO: 157) |
| light | | VSYMNWYQQKPG | |
| domain | | KAPKRWIYDSSKLAS | |
| | | GVPSRFSGSGSGTEY | |
| | | TLTISSLQPDDFATYY | |
| | | CQQWSRNPPTFGG | |
| | | GTKVEIKRS | |
| TSC456 | | DIQMTQSPSTLSAS | (SEQ ID |
| variable | | VGDRVTMTCSASSS | NO: 158) |
| light | | VSYMNWYQQKPG | |
| domain | | KAPKRWIYDSSKLAS | |
| | | GVPSRFSGSGSGTD | |
| | | YTLTISSLQPDDFATY | |
| | | YCQQWSRNPPTFG | |
| | | GGTKVEIKRS | |
| DRA222 | | QVQLVESGGGVVQ | (SEQ ID |
| (anti-CD3) | | PGRSLRLSCKASGYT | NO: 313) |
| scFv | | FTRSTMHWVRQAP | |
| | | GQGLEWIGYINPSS | |
| | | AYTNYNQKFKDRFTI | |
| | | SADKSKSTAFLQMD | |
| | | SLRPEDTGVYFCARP | |
| | | QVHYDYNGFPYWG | |
| | | QGTPVTVSSGGGGS | |
| | | GGGGSGGGGSAQD | |
| | | IQMTQSPSSLSASV | |
| | | GDRVTMTCSASSSV | |
| | | SYMNWYQQKPGK | |
| | | APKRWIYDSSKLAS | |
| | | GVPARFSGSGSGTD | |
| | | YTLTISSLQPEDFATY | |
| | | YCQQWSRNPPTFG | |
| | | GGTKLQITSSS | |
| DRA222 | | QVQLVESGGGVVQ | (SEQ ID |
| variable | | PGRSLRLSCKASGYT | NO: 161) |
| heavy | | FTRSTMHWVRQAP | |
| domain | | GQGLEWIGYINPSS | |
| | | AYTNYNQKFKDRFTI | |
| | | SADKSKSTAFLQMD | |
| | | SLRPEDTGVYFCARP | |
| | | QVHYDYNGFPYWG | |
| | | QGTPVTVSS | |
| DRA222 | | DIQMTQSPSSLSAS | (SEQ ID |
| variable | | VGDRVTMTCSASSS | NO: 160) |
| light | | VSYMNWYQQKPG | |
| domain | | KAPKRWIYDSSKLAS | |
| | | GVPARFSGSGSGTD | |
| | | YTLTISSLQPEDFATY | |
| | | YCQQWSRNPPTFG | |
| | | GGTKLQITS | |

CD123-binding proteins may comprise any of the CD123-binding domains described above. In some aspects, CD123-binding proteins comprise humanized V_{H} or V_{L} amino acid sequences, or both.

The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:2 or SEQ ID NO:4. The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:2 and SEQ ID NO:4. The polypeptides may comprise an amino acid sequence that is at least about 95%, at least about 97% identical, at least about 99% or 100% identical to the amino acid sequence set forth in SEQ ID NO:2 or SEQ ID NO:4. In some embodiments, the polypeptide comprises an amino acid sequence that is that is at least about 95%, at least about 97% identical, or at least about 99% identical to the amino acid sequence set forth in SEQ ID NO:130 or SEQ ID NO:132. In some embodiments, the polypeptide comprises an amino acid sequence that is that is at least about 95%, at least about 97% identical, or at least about 99% identical to the scFv portion of the amino acid sequence set forth in SEQ ID NO:130 or SEQ ID NO:132. In certain embodiments, the polypeptide comprises or consists of an amino acid sequence set forth in SEQ ID NO:2, SEQ ID NO:4, SEQ ID NO:130, or SEQ ID NO:132.

The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 or SEQ ID NO:20. The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 and SEQ ID NO:20. The polypeptides may comprise an amino acid sequence that is at least about 95%, at least about 97% identical, at least about 99% or 100% identical to the amino acid sequence set forth in SEQ ID NO:18 or SEQ ID NO:20. In some embodiments, the polypeptide comprises an amino acid sequence that is that is at least about 95%, at least about 97% identical, or at least about 99% identical to the amino acid sequence set forth in SEQ ID NO:134, SEQ ID NO:136, or SEQ ID NO:138. In some embodiments, the polypeptide comprises an amino acid sequence that is that is at least about 95%, at least about 97% identical, or at least about 99% identical to the scFv portion of the amino acid sequence set forth in SEQ ID NO:134, SEQ ID NO:136, or SEQ ID NO:138. In certain embodiments, the polypeptide comprises or consists of an amino acid sequence set forth in SEQ ID NO:18, SEQ ID NO:20, SEQ ID NO:134, SEQ ID NO:136, or SEQ ID NO:138.

The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 or SEQ ID NO:34. The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 and SEQ ID NO:34. The polypeptides may comprise an amino acid sequence that is at least about 95%, at least about 97% identical, at least about 99% or 100% identical to the amino acid sequence set forth in SEQ ID NO:18 or SEQ ID NO:34. In some embodiments, the polypeptide comprises an amino acid sequence that is that is at least about 95%, at least about 97% identical, or at least about 99% identical to the amino acid sequence set forth in SEQ ID NO:140. In some embodiments, the polypeptide comprises an amino acid sequence that is that is at least about 95%, at least about 97% identical, or at least about 99% identical to the scFv portion of the amino acid sequence set forth in SEQ ID NO:140. In certain embodiments, the polypeptide comprises or consists of an amino acid sequence set forth in SEQ ID NO:18, SEQ ID NO:34, or SEQ ID NO:140.

The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 or SEQ ID NO:42. The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 and SEQ ID NO:42. The polypeptides may comprise an amino acid sequence that is at least about 95%, at least about 97% identical, at least about 99% or 100% identical to the amino acid sequence set forth in SEQ ID NO:18 or SEQ ID NO:42. In some embodiments, the polypeptide comprises an amino acid sequence that is that is at least about 95%, at least about 97% identical, or at least about 99% identical to the amino acid sequence set forth in SEQ ID NO:142. In some embodiments, the polypeptide comprises an amino acid sequence that is that is at least about 95%, at least about 97% identical, or at least about 99% identical to the scFv portion of the amino acid sequence set forth in SEQ ID NO:142. In certain embodiments, the polypeptide comprises or consists of an amino acid sequence set forth in SEQ ID NO:18, SEQ ID NO:42, or SEQ ID NO:142.

The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:50 or SEQ ID NO:52. The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:50 and SEQ ID NO:52. The polypeptides may comprise an amino acid sequence that is at least about 95%, at least about 97% identical, at least about 99% or 100% identical to the amino acid sequence set forth in SEQ ID NO:50 or SEQ ID NO:52. In some embodiments, the polypeptide comprises an amino acid sequence that is that is at least about 95%, at least about 97% identical, or at least about 99% identical to the amino acid sequence set forth in SEQ ID NO:144. In some embodiments, the polypeptide comprises an amino acid sequence that is that is at least about 95%, at least about 97% identical, or at least about 99% identical to the scFv portion of the amino acid sequence set forth in SEQ ID NO:144. In certain embodiments, the polypeptide comprises or consists of an amino acid sequence set forth in SEQ ID NO:50, SEQ ID NO:52, or SEQ ID NO:144.

The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:66 or SEQ ID NO:68. The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:66 and SEQ ID NO:68. The polypeptides may comprise an amino acid sequence that is at least about 95%, at least about 97% identical, at least about 99% or 100% identical to the amino acid sequence set forth in SEQ ID NO:66 or SEQ ID NO:68. In some embodiments, the polypeptide comprises an amino acid sequence that is that is at least about 95%, at least about 97% identical, or at least about 99% identical to the amino acid sequence set forth in SEQ ID NO:146. In some embodiments, the polypeptide comprises an amino acid sequence that is that is at least about 95%, at least about 97% identical, or at least about 99% identical to the scFv portion of the amino acid sequence set forth in SEQ ID NO:146. In certain embodiments, the polypeptide comprises or consists of an amino acid sequence set forth in SEQ ID NO:66, SEQ ID NO:68, or SEQ ID NO:146.

The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:82 or SEQ ID NO:84. The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:82 and SEQ ID NO:84. The polypeptides may comprise an amino acid sequence that is at least about 95%, at least about 97% identical, at least about 99% or 100% identical to the amino acid sequence set forth in SEQ ID NO:82 or SEQ ID NO:84. In some embodiments, the polypeptide comprises an amino acid sequence that is that is at least about 95%, at least about 97% identical, or at least about 99% identical to the amino acid sequence set forth in SEQ ID NO:148. In some embodiments, the polypeptide comprises an amino acid sequence that is that is at least about 95%, at least about 97% identical, or at least about 99% identical to the scFv portion of the amino acid sequence set forth in SEQ ID NO:148. In certain embodiments, the polypeptide comprises or consists of an amino acid sequence set forth in SEQ ID NO:82, SEQ ID NO:84, or SEQ ID NO:148.

The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 or SEQ ID NO:98. The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 and SEQ ID NO:98. The polypeptides may comprise an amino acid sequence that is at least about 95%, at least about 97% identical, at least about 99% or 100% identical to the amino acid sequence set forth in SEQ ID NO:18 or SEQ ID NO:150. In some embodiments, the polypeptide comprises an amino acid sequence that is that is at least about 95%, at least about 97% identical, or at least about 99% identical to the amino acid sequence set forth in SEQ ID NO:150. In some embodiments, the polypeptide comprises an amino acid sequence that is that is at least about 95%, at least about 97% identical, or at least about 99% identical to the scFv portion of the amino acid sequence set forth in SEQ ID NO:140. In certain embodiments, the polypeptide comprises or consists of an amino acid sequence set forth in SEQ ID NO:18, SEQ ID NO:98, or SEQ ID NO:150.

The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 or SEQ ID NO:106. The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 and SEQ ID NO:106. The polypeptides may comprise an amino acid sequence that is at least about 95%, at least about 97% identical, at least about 99% or 100% identical to the amino acid sequence set forth in SEQ ID NO:18 or SEQ ID NO:106. In some embodiments, the polypeptide comprises an amino acid sequence that is that is at least about 95%, at least about 97% identical, or at least about 99% identical to the amino acid sequence set forth in SEQ ID NO:152. In some embodiments, the polypeptide comprises an amino acid sequence that is that is at least about 95%, at least about 97% identical, or at least about 99% identical to the scFv portion of the amino acid sequence set forth in SEQ ID NO:152. In certain embodiments, the polypeptide comprises or consists of an amino acid sequence set forth in SEQ ID NO:18, SEQ ID NO:106, or SEQ ID NO:152.

The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 or SEQ ID NO:114. The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 and SEQ ID NO:114. The polypeptides may comprise an amino acid sequence that is at least about 95%, at least about 97% identical, at least about 99% or 100% identical to the amino acid sequence set forth in SEQ ID NO:18 or SEQ ID NO:114. In some embodiments, the polypeptide comprises an amino acid sequence that is that is at least about 95%, at least about 97% identical, or at least about 99% identical to the amino acid sequence set forth in SEQ ID NO:154. In some embodiments, the polypeptide comprises an amino acid sequence that is that is at least about 95%, at least about 97% identical, or at least about 99% identical to the scFv portion of the amino acid sequence set forth in SEQ ID NO:154. In certain embodiments, the polypeptide comprises or consists of an amino acid sequence set forth in SEQ ID NO:18, SEQ ID NO:114, or SEQ ID NO:154.

The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 or SEQ ID NO:122. The polypeptides may comprise an amino acid sequence that is at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the amino acid sequence as set forth in SEQ ID NO:18 and SEQ ID NO:122. The polypeptides may comprise an amino acid sequence that is at least about 95%, at least about 97% identical, at least about 99% or 100% identical to the amino acid sequence set forth in SEQ ID NO:18 or SEQ ID NO:122. In some embodiments, the polypeptide comprises an amino acid sequence that is that is at least about 95%, at least about 97% identical, or at least about 99% identical to the amino acid sequence set forth in SEQ ID NO:156. In some embodiments, the polypeptide comprises an amino acid sequence that is that is at least about 95%, at least about 97% identical, or at least about 99% identical to the scFv portion of the amino acid sequence set forth in SEQ ID NO:156. In certain embodiments, the polypeptide comprises or consists of an amino acid sequence set forth in SEQ ID NO:18, SEQ ID NO:122, or SEQ ID NO:156.

The polypeptides disclosed herein may have improved characteristics compared to other CD123-binding domains or polypeptides. For example, a CD123-binding domain or polypeptide may exhibit a reduced isoelectric point compared to the isoelectric point of a different CD123-binding domain or polypeptide. “Isoelectric point” or “pl” is the pH at which net charge is zero. The isoelectric point of a protein may be measured by any suitable method, e.g., analytical capillary isoelectric focusing chromatography.

A CD123-binding domain or protein disclosed herein may bind to CD123 (e.g., human CD123) with a higher affinity than the parent antibody.

In one embodiment of the invention, the recombinant polypeptide comprises, in order from amino to carboxyl terminus, (i) a human or humanized CD123-binding domain, (ii) a hinge region, (iii) an immunoglobulin constant region, (iv) a carboxyl-terminus linker, and (v) a human or humanized second binding domain that specifically binds a T-cell, CD3, CD3ε or a T-cell receptor (TCR) complex or a component of a T-cell receptor complex, wherein the human or humanized CD123-binding domain comprises an amino acid sequence at least about 80%, at least about 90%, at least about 95%, at least about 96%, at least about 97%, at least about 98%, at least about 99% or at least about 99.5% identical to SEQ ID NO:2 and SEQ ID NO:4 and wherein the human or humanized second binding domain that specifically binds a T-cell, CD3, CD3ε or a T-cell receptor (TCR) complex or a component of a T-cell receptor complex comprises an amino acid sequence at least about 80%, at least about 90%, at least about 95%, at least about 96%, at least about 97%, at least about 98%, at least about 99% or at least about 99.5% identical to SEQ ID NO:311 or SEQ ID NO:312.

In one embodiment of the invention, the recombinant polypeptide does not contain a CD123-binding domain derived from murine antibody 12F1 (SEQ ID Nos:195 and 197). For instance, in one embodiment, the CD123-binding domain does not comprise a heavy chain or light chain variable domain that is significantly identical to a heavy chain or light chain variable domain of 12F1 (e.g., is not 95% identical or greater to 12F1 heavy and light chain variable domains) or does not contain all six CDRs as contained in murine 12F1. In one embodiment of the invention, the CD123-binding domain does not compete for binding to CD123 with murine antibody 12F1 (SEQ ID NOs:195 and 197). In one embodiment of the invention, the recombinant polypeptide is cross-reactive to cynomolgous CD123 whereas antibody 12F1 (and humanized derivatives thereof) is not cross-reactive to cynomolgous CD123.

TABLE 4
12F1 Murine Antibody Sequences

| | | | SEQ ID Nos: |
|---|---|---|---|
| | | | nucleotide |
| Name | Nucleotide Sequence | Amino Acid Sequence | (amino acid) |
| 12F1 | gacatcatgatgtcccagtccccctcctccctg | dimmsqspsslavsvgekftmtckssqs | SEQ ID NO: 194 |
| murine | gccgtgtccgtgggcgagaagttcaccatgac | lffgstqknylawyqqkpgqspklliywa | (SEQ ID NO: 195) |
| variable | ctgcaagtcctcccagtccctgttcttcggctcc | stresgvpdrftgsgsgtdftlaissvmpe | |
| light | acccagaagaactacctggcctggtaccagca | dlavyycqqyynypwtfgggtkleik | |
| chain | gaagcccggccagtcccccaagctgctgatct | | |
| domain | actgggcctccacccgggagtccggcgtgccc | | |
| | gaccggttcaccggctccggctccggcaccga | | |
| | cttcaccctggccatctcctccgtgatgcccga | | |
| | ggacctggccgtgtactactgccagcagtacta | | |
| | caactacccctggaccttcggcggcggcacca | | |
| | agctggagatcaag | | |
| 12F1 | cagctgcaggagtccggccccggcct | vqlqesgpglvkpsqslsltcsvtd | SEQ ID NO: 196 |
| murine | ggtgaagccctcccagtccctgtccctg | ysitsgyywnwirqfpgnklew | (SEQ ID NO: 197) |
| variable | acctgctccgtgaccgactactccatca | mgyisydgsnnynpslknrisitr | |
| heavy | cctccggctactactggaactggattcg | dtsknqfflklssvttedtatyycsr | |
| chain | gcagttccccggcaacaagctggagtg | gegfyfdswgqgttltvss | |
| domain | gatgggctacatctcctacgacggctcc | | |
| | aacaactacaacccctccctgaagaac | | |
| | cggatctccatcacccgggacacctcc | | |
| | aagaaccagttcttcctgaagctgtcctc | | |
| | cgtgaccaccgaggacaccgccacct | | |
| | actactgctcccggggcgagggcttct | | |
| | acttcgactcctggggccagggcacca | | |
| | ccctgaccgtgtcctcg | | |

In one embodiment, the polypeptide of the invention (including in dimer form) binds to human CD123 and non-human primate (NHP) CD123 with specificity. In another embodiment of the invention, the polypeptide binds to cynomolgus monkey CD123.

The disclosure also includes nucleic acids (e.g., DNA or RNA) encoding CD123-binding domains and polypeptides described herein. Nucleic acids of the disclosure include nucleic acids having a region that is substantially identical to a polynucleotide as listed in Table 3, infra. In certain embodiments, a nucleic acid in accordance with the present disclosure has at least 80%, typically at least about 90%, and more typically at least about 95% or at least about 98% identity to a polypeptide-encoding polynucleotide as listed in Table 3. Nucleic acids of the disclosure also include complementary nucleic acids. In some instances, the sequences will be fully complementary (no mismatches) when aligned. In other instances, there can be up to about a 20% mismatch in the sequences. In some embodiments of the disclosure are provided nucleic acids encoding both first and second polypeptide chains of a heterodimeric CD123-binding protein of the disclosure. The nucleic acid sequences provided herein can be exploited using codon optimization, degenerate sequence, silent mutations, and other DNA techniques to optimize expression in a particular host, and the present disclosure encompasses such sequence modifications.

The invention includes a recombinant polypeptide encoded by a nucleic acid comprising at nucleic acid sequence at least about 80%, at least about 85%, at least about 90%, at least about 95%, at least about 96%, at least about 97%, at least about 98%, at least about 99% and at least 100% identical to the nucleic acid sequence of SEQ ID NO:1 and/or SEQ ID NO:3. The invention includes a recombinant polypeptide encoded by a nucleic acid comprising at nucleic acid sequence at least about 80%, at least about 85%, at least about 90%, at least about 95%, at least about 96%, at least about 97%, at least about 98%, at least about 99% and at least 100% identical to the nucleic acid sequence of SEQ ID NO:131.

The disclosure relates to an isolated nucleic acid molecule encoding CD123-binding domains, proteins and polypeptides (or portions thereof) described herein, wherein said nucleic acid molecule comprises a nucleotide sequence set forth in SEQ ID NO:1, SEQ ID NO:3, SEQ ID NO:17, SEQ ID NO:19, SEQ ID NO:33, SEQ ID NO:41, SEQ ID NO:49, SEQ ID NO:51, SEQ ID NO:65, SEQ ID NO:67, SEQ ID NO:81, SEQ ID NO:83, SEQ ID NO:97, SEQ ID NO:105, SEQ ID NO:113, SEQ ID NO:121, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, or SEQ ID NO:155.

Polynucleotide molecules comprising a desired polynucleotide sequence are propagated by placing the molecule in a vector. Viral and non-viral vectors can be used, including plasmids. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. The partial or full-length polynucleotide is inserted into a vector typically by means of DNA ligase attachment to a cleaved restriction enzyme site in the vector. Alternatively, the desired nucleotide sequence can be inserted by homologous recombination in vivo. Typically this is accomplished by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence, for example.

For expression, an expression cassette or system may be employed. To express a nucleic acid encoding a polypeptide disclosed herein, a nucleic acid molecule encoding the polypeptide, operably linked to regulatory sequences that control transcriptional expression in an expression vector, is introduced into a host cell. In addition to transcriptional regulatory sequences, such as promoters and enhancers, expression vectors can include translational regulatory sequences and a marker gene which is suitable for selection of cells that carry the expression vector. The gene product encoded by a polynucleotide of the disclosure is expressed in any convenient expression system, including, for example, bacterial, yeast, insect, amphibian and mammalian systems. In the expression vector, the polypeptide-encoding polynucleotide is linked to a regulatory sequence as appropriate to obtain the desired expression properties. These can include promoters, enhancers, terminators, operators, repressors, and inducers. The promoters can be regulated (e.g., the promoter from the steroid inducible pIND vector (Invitrogen)) or constitutive (e.g., promoters from CMV, SV40, Elongation Factor, or LTR sequences). These are linked to the desired nucleotide sequence using the techniques described above for linkage to vectors. Any techniques known in the art can be used. Accordingly, the expression vector will generally provide a transcriptional and translational initiation region, which can be inducible or constitutive, where the coding region is operably linked under the transcriptional control of the transcriptional initiation region, and a transcriptional and translational termination region.

An expression cassette (“expression unit”) can be introduced into a variety of vectors, e.g., plasmid, BAC, YAC, bacteriophage such as lambda, P1, M13, etc., plant or animal viral vectors (e.g., retroviral-based vectors, adenovirus vectors), and the like, where the vectors are normally characterized by the ability to provide selection of cells comprising the expression vectors. The vectors can provide for extrachromosomal maintenance, particularly as plasmids or viruses, or for integration into the host chromosome. Where extrachromosomal maintenance is desired, an origin sequence is provided for the replication of the plasmid, which can be low- or high copy-number. A wide variety of markers are available for selection, particularly those which protect against toxins, more particularly against antibiotics. The particular marker that is chosen is selected in accordance with the nature of the host, where, in some cases, complementation can be employed with auxotrophic hosts. Introduction of the DNA construct can use any convenient method, including, e.g., conjugation, bacterial transformation, calcium-precipitated DNA, electroporation, fusion, transfection, infection with viral vectors, biolistics, and the like. The disclosure relates to an expression vector comprising a nucleic acid segment, wherein said nucleic acid segment may comprise a nucleotide sequence set forth in SEQ ID NO:1, SEQ ID NO:3, SEQ ID NO:17, SEQ ID NO:19, SEQ ID NO:33, SEQ ID NO:41, SEQ ID NO:49, SEQ ID NO:51, SEQ ID NO:65, SEQ ID NO:67, SEQ ID NO:81, SEQ ID NO:83, SEQ ID NO:97, SEQ ID NO:105, SEQ ID NO:113, SEQ ID NO:121, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, or SEQ ID NO:155.

Accordingly, proteins for use within the present disclosure can be produced in genetically engineered host cells according to conventional techniques. Suitable host cells are those cell types that can be transformed or transfected with exogenous DNA and grown in culture, and include bacteria, fungal cells, and cultured higher eukaryotic cells (including cultured cells of multicellular organisms), particularly cultured mammalian cells. Techniques for manipulating cloned DNA molecules and introducing exogenous DNA into a variety of host cells are disclosed by Sambrook and Russell, Molecular Cloning: A Laboratory Manual (3rd ed., Cold Spring Harbor Laboratory Press, Cold Spring Harbor, N Y, 2001), and Ausubel et al., Short Protocols in Molecular Biology (4th ed., John Wiley & Sons, 1999). For instance, the recombinant polypeptides of the invention can be expressed from CHO and HEK293 cells.

For example, for recombinant expression of a homodimeric CD123-binding protein comprising two identical CD123-binding polypeptides as described herein, an expression vector will generally include a nucleic acid segment encoding the CD123-binding polypeptide, operably linked to a promoter. For recombinant expression of a heterodimeric CD123-binding protein, comprising different first and second polypeptide chains, the first and second polypeptide chains can be co-expressed from separate vectors in the host cell for expression of the entire heterodimeric protein. Alternatively, for the expression of heterodimeric CD123-binding proteins, the first and second polypeptide chains are co-expressed from separate expression units in the same vector in the host cell for expression of the entire heterodimeric protein. The expression vector(s) are transferred to a host cell by conventional techniques, and the transfected cells are then cultured by conventional techniques to produce the encoded polypeptide(s) to produce the corresponding CD123-binding protein.

To direct a recombinant protein into the secretory pathway of a host cell, a secretory signal sequence (also known as a leader sequence) is provided in the expression vector. The secretory signal sequence can be that of the native form of the recombinant protein, or can be derived from another secreted protein or synthesized de novo. The secretory signal sequence is operably linked to the polypeptide-encoding DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences can be positioned elsewhere in the DNA sequence of interest (see, e.g., Welch et al., U.S. Pat. No. 5,037,743; Holland et al., U.S. Pat. No. 5,143,830). In certain variations, a secretory signal sequence for use in accordance with the present disclosure has the amino acid sequence MEAPAQLLFLLLLWLPDTTG (SEQ ID NO:198).

Cultured mammalian cells are suitable hosts for production of recombinant proteins for use within the present disclosure. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., supra), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44; CHO DXB11 (Hyclone, Logan, UT); see also, e.g., Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658). Additional suitable cell lines are known in the art and available from public depositories such as the American Type Culture Collection, Manassas, Virginia. Strong transcription promoters can be used, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants.” Cells that have been cultured in the presence of the selective agent and are able to pass the gene of interest to their progeny are referred to as “stable transfectants.” Exemplary selectable markers include a gene encoding resistance to the antibiotic neomycin, which allows selection to be carried out in the presence of a neomycin-type drug, such as G-418 or the like; the gpt gene for xanthine-guanine phosphoribosyl transferase, which permits host cell growth in the presence of mycophenolic acid/xanthine; and markers that provide resistance to zeocin, bleomycin, blastocidin, and hygromycin (see, e.g., Gatignol et al., Mol. Gen. Genet. 207:342, 1987; Drocourt et al., Nucl. Acids Res. 18:4009, 1990). Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. An exemplary amplifiable selectable marker is dihydrofolate reductase, which confers resistance to methotrexate. Other drug resistance genes (e.g., hygromycin resistance, multi-drug resistance, puromycin acetyltransferase) can also be used.

Other higher eukaryotic cells can also be used as hosts, including insect cells, plant cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WO 94/06463.

Insect cells can be infected with recombinant baculovirus, commonly derived from Autographa californica nuclear polyhedrosis virus (AcNPV). See King and Possee, The Baculovirus Expression System: A Laboratory Guide (Chapman & Hall, London); O'Reilly et al., Baculovirus Expression Vectors: A Laboratory Manual (Oxford University Press., New York 1994); and Baculovirus Expression Protocols. Methods in Molecular Biology (Richardson ed., Humana Press, Totowa, N J, 1995). Recombinant baculovirus can also be produced through the use of a transposon-based system described by Luckow et al. (J. Virol. 67:4566-4579, 1993). This system, which utilizes transfer vectors, is commercially available in kit form (BAC-TO-BAC kit; Life Technologies, Gaithersburg, MD). The transfer vector (e.g., PFASTBAC1; Life Technologies) contains a Tn7 transposon to move the DNA encoding the protein of interest into a baculovirus genome maintained in E. coli as a large plasmid called a “bacmid.” See Hill-Perkins and Possee, J. Gen. Virol. 71:971-976, 1990; Bonning et al., J. Gen. Virol. 75:1551-1556, 1994; and Chazenbalk and Rapoport, J. Biol. Chem. 270:1543-1549, 1995. In addition, transfer vectors can include an in-frame fusion with DNA encoding a polypeptide extension or affinity tag as disclosed above. Using techniques known in the art, a transfer vector containing a protein-encoding DNA sequence is transformed into E. coli host cells, and the cells are screened for bacmids which contain an interrupted lacZ gene indicative of recombinant baculovirus. The bacmid DNA containing the recombinant baculovirus genome is isolated, using common techniques, and used to transfect Spodoptera frugiperda cells, such as Sf9 cells. Recombinant virus that expresses the protein or interest is subsequently produced. Recombinant viral stocks are made by methods commonly used in the art.

For protein production, a recombinant virus can be used to infect host cells, typically a cell line derived from the fall armyworm, Spodoptera frugiperda (e.g., Sf9 or Sf21 cells) or Trichoplusia ni (e.g., HIGH FIVE cells; Invitrogen, Carlsbad, CA). See generally Glick and Pasternak, Molecular Biotechnology, Principles & Applications of Recombinant DNA (ASM Press, Washington, D.C., 1994). See also U.S. Pat. No. 5,300,435. Serum-free media are used to grow and maintain the cells. Suitable media formulations are known in the art and can be obtained from commercial suppliers. The cells are grown up from an inoculation density of approximately 2-5×10⁵ cells to a density of 1-2×10⁶ cells, at which time a recombinant viral stock is added at a multiplicity of infection (MOI) of 0.1 to 10, more typically near 3. Procedures used are generally described in available laboratory manuals (see, e.g., King and Possee, supra; O'Reilly et al., supra; Richardson, supra).

Fungal cells, including yeast cells, can also be used within the present disclosure. Yeast species of in this regard include, e.g., Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). An exemplary vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936; and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii, and Candida maltosa are known in the art. See, e.g., Gleeson et al., J. Gen. Microbiol. 132:3459-3465, 1986; Cregg, U.S. Pat. No. 4,882,279; and Raymond et al., Yeast 14:11-23, 1998. Aspergillus cells can be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. Production of recombinant proteins in Pichia methanolica is disclosed in U.S. Pat. Nos. 5,716,808; 5,736,383; 5,854,039; and 5,888,768.

Prokaryotic host cells, including strains of the bacteria Escherichia coli, Bacillus, and other genera are also useful host cells within the present disclosure. Techniques for transforming these hosts and expressing foreign DNA sequences cloned therein are well-known in the art (see, e.g., Sambrook and Russell, supra). When expressing a recombinant protein in bacteria such as E. coli, the protein can be retained in the cytoplasm, typically as insoluble granules, or can be directed to the periplasmic space by a bacterial secretion sequence. In the former case, the cells are lysed, and the granules are recovered and denatured using, for example, guanidine isothiocyanate or urea. The denatured protein can then be refolded and dimerized by diluting the denaturant, such as by dialysis against a solution of urea and a combination of reduced and oxidized glutathione, followed by dialysis against a buffered saline solution. In the alternative, the protein can be recovered from the cytoplasm in soluble form and isolated without the use of denaturants. The protein is recovered from the cell as an aqueous extract in, for example, phosphate buffered saline. To capture the protein of interest, the extract is applied directly to a chromatographic medium, such as an immobilized antibody or heparin-Sepharose column. Secreted proteins can be recovered from the periplasmic space in a soluble and functional form by disrupting the cells (by, for example, sonication or osmotic shock) to release the contents of the periplasmic space and recovering the protein, thereby obviating the need for denaturation and refolding. Antibodies, including single-chain antibodies, can be produced in bacterial host cells according to known methods. See, e.g., Bird et al., Science 242:423-426, 1988; Huston et al., Proc. Natl. Acad. Sci. USA 85:5879-5883, 1988; and Pantoliano et al., Biochem. 30:10117-10125, 1991.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media can also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

CD123-binding proteins may be purified by conventional protein purification methods, typically by a combination of chromatographic techniques. See generally Affinity Chromatography: Principles & Methods (Pharmacia LKB Biotechnology, Uppsala, Sweden, 1988); Scopes, Protein Purification: Principles and Practice (Springer-Verlag, New York 1994). Proteins comprising an immunoglobulin Fc region can be purified by affinity chromatography on immobilized protein A or protein G. Additional purification steps, such as gel filtration, can be used to obtain the desired level of purity or to provide for desalting, buffer exchange, and the like.

The present disclosure provides methods for treating a subject with a disorder characterized by over-expression of CD123. Generally, such methods include administering to a subject in need of such treatment the polypeptide or CD123-binding protein as described herein. In some embodiments, the CD123-binding protein comprises at least one effector function selected from antibody-dependent cell-mediated cytotoxicity (ADCC) and complement-dependent cytotoxicity (CDC), such that the CD123-binding protein induces ADCC and/or CDC against CD123-expressing cells in the subject.

In other embodiments, where the polypeptide comprises a second binding domain that specifically binds a T-cell (e.g., to a TCR complex or component thereof, such as CD3ε), the polypeptide or CD123-binding protein induces redirected T-cell cytotoxicity (RTCC) against CD123-expressing cells in the subject. In some embodiments, RTCC polypeptides (e.g., polypeptides that induce RTCC) comprise a modified constant domain to reduce or remove ADCC and/or CDC activity.

In certain variations of the method, the disorder is a cancer. Exemplary cancers amenable to treatment in accordance with the present disclosure include, for example, acute myeloid leukemia (AML), B-lymphoid leukemia, blastic plasmocytoid dendritic neoplasm (BPDCN), hairy cell leukemia, myelodysplastic syndrome, acute lymphoblastic leukemia, refractory anemia with excess blasts, chronic myeloid leukemia and Hodgkin's lymphoma.

The disclosure also encompasses a use of a CD123-binding polypeptide for the manufacture of a medicament for treatment of a disorder (e.g., cancer) characterized by over-expression of CD123. In one embodiment, the CD123-binding polypeptide has RTCC activity, e.g., it comprises an anti-CD123 and anti-CD3 binding domain. In one embodiment, the disclosure includes a CD123-binding polypeptide for use in treating a disorder (e.g., cancer) characterized by over-expression of CD123.

In one embodiment, the disclosure provides a method of treating a patient diagnosed with acute myeloid leukemia, B-lymphoid leukemia, blastic plasmocytoid dendritic neoplasms, hairy cell leukemia, myelodysplastic syndrome, acute lymphoblastic leukemia, refractory anemia with excess blasts, chronic myeloid leukemia, Hodgkin's lymphoma or other cancer associated with the expression of CD123 by administering a therapeutically effective amount of a pharmaceutical composition comprising a recombinant polypeptide at least about 80%, at least about 85%, at least about 90%, at least about 95%, at least about 96%, at least about 97%, at least about 98%, at least about 99% or at least about 100% identical to the amino acid sequence of SEQ ID NO:130 or 132.

In some embodiments, the disclosure provides a method of treating a patient with a cancer, comprising administering to the patient a CD123-binding polypeptide comprising the amino acid sequence set forth in SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, or SEQ ID NO:156.

In some embodiments, for treatment methods and uses described herein, a polypeptide of the invention is delivered in a manner consistent with conventional methodologies associated with management of the disease or disorder for which treatment is sought. In accordance with the disclosure herein, a therapeutically effective amount of the polypeptide or CD123-binding protein in dimer form is administered to a subject in need of such treatment for a time and under conditions sufficient to prevent or treat the disease or disorder.

Subjects for administration of polypeptides as described herein include patients at high risk for developing a particular disorder characterized by CD123 over-expression as well as patients presenting with an existing such disorder. Typically, the subject has been diagnosed as having the disorder for which treatment is sought. Further, subjects can be monitored during the course of treatment for any change in the disorder (e.g., for an increase or decrease in clinical symptoms of the disorder). Also, in some variations, the subject does not suffer from another disorder requiring treatment that involves targeting CD123-expressing cells.

In prophylactic applications, pharmaceutical compositions or medicants are administered to a patient susceptible to, or otherwise at risk of, a particular disorder in an amount sufficient to eliminate or reduce the risk or delay the onset of the disorder. In therapeutic applications, compositions or medicants are administered to a patient suspected of, or already suffering from such a disorder in an amount sufficient to cure, or at least partially arrest, the symptoms of the disorder and its complications. An amount adequate to accomplish this is referred to as a therapeutically effective dose or amount. In both prophylactic and therapeutic regimes, agents are usually administered in several dosages until a sufficient response (e.g., inhibition of inappropriate angiogenesis activity) has been achieved. Typically, the response is monitored and repeated dosages are given if the desired response starts to fade.

To identify subject patients for treatment according to the methods of the disclosure, accepted screening methods can be employed to determine risk factors associated with specific disorders or to determine the status of an existing disorder identified in a subject. Such methods can include, for example, determining whether an individual has relatives who have been diagnosed with a particular disorder. Screening methods can also include, for example, conventional work-ups to determine familial status for a particular disorder known to have a heritable component. For example, various cancers are also known to have certain inheritable components. Inheritable components of cancers include, for example, mutations in multiple genes that are transforming (e.g., Ras, Raf, EGFR, cMet, and others), the presence or absence of certain HLA and killer inhibitory receptor (KIR) molecules, or mechanisms by which cancer cells are able to modulate immune suppression of cells like NK cells and T-cells, either directly or indirectly (see, e.g., Ljunggren and Malmberg, Nature Rev. Immunol. 7:329-339, 2007; Boyton and Altmann, Clin. Exp. Immunol. 149:1-8, 2007). Toward this end, nucleotide probes can be routinely employed to identify individuals carrying genetic markers associated with a particular disorder of interest. In addition, a wide variety of immunological methods are known in the art that are useful to identify markers for specific disorder. For example, various ELISA immunoassay methods are available and well-known in the art that employ monoclonal antibody probes to detect antigens associated with specific tumors. Screening can be implemented as indicated by known patient symptomology, age factors, related risk factors, etc. These methods allow the clinician to routinely select patients in need of the methods described herein for treatment. In accordance with these methods, targeting pathological, CD123-expressing cells can be implemented as an independent treatment program or as a follow-up, adjunct, or coordinate treatment regimen to other treatments.

For administration, the polypeptide of the invention (e.g., in dimer form) may be formulated as a pharmaceutical composition. A pharmaceutical composition may comprise: (i) a CD123-binding polypeptide; and (ii) a pharmaceutically acceptable carrier, diluent or excipient. A pharmaceutical composition comprising a CD123-binding protein can be formulated according to known methods to prepare pharmaceutically useful compositions, whereby the therapeutic molecule is combined in a mixture with a pharmaceutically acceptable carrier, diluent or excipient. A carrier is said to be a “pharmaceutically acceptable carrier” if its administration can be tolerated by a recipient patient. Sterile phosphate-buffered saline is one example of a pharmaceutically acceptable carrier. Other suitable carriers, diluents or excipients are well-known to those in the art. (See, e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc.

A pharmaceutical composition may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form. The oral unit dosage form may be selected from the group consisting of: tablets, pills, pellets, capsules, powders, lozenges, granules, solutions, suspensions, emulsions, syrups, elixirs, sustained-release formulations, aerosols, and sprays.

A pharmaceutical composition comprising a polypeptide of the invention may be administered to a subject in a therapeutically effective amount. According to the methods of the present disclosure, a CD123-binding protein can be administered to subjects by a variety of administration modes, including, for example, by intramuscular, subcutaneous, intravenous, intra-atrial, intra-articular, parenteral, intranasal, intrapulmonary, transdermal, intrapleural, intrathecal, and oral routes of administration.

Determination of effective dosages in this context is typically based on animal model studies followed up by human clinical trials and is guided by determining effective dosages and administration protocols that significantly reduce the occurrence or severity of the subject disorder in model subjects. Effective doses of the compositions of the present disclosure vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Usually, the patient is a human, but in some diseases, the patient can be a nonhuman mammal. Typically, dosage regimens are adjusted to provide an optimum therapeutic response, i.e., to optimize safety and efficacy. Accordingly, a therapeutically effective amount is also one in which any undesired collateral effects are outweighed by the beneficial effects of administering a CD123-binding protein as described herein. For administration of the CD123-binding protein, a dosage may range from, for instance, about 0.1 μg to 100 mg/kg or 1 μg/kg to about 50 mg/kg, or 10 μg to 5 mg/kg of the subject's body weight.

Dosage of the pharmaceutical composition can be varied by the attending clinician to maintain a desired concentration at a target site.

With particular regard to treatment of solid tumors, protocols for assessing endpoints and anti-tumor activity are well-known in the art. While each protocol may define tumor response assessments differently, the RECIST (Response evaluation Criteria in solid tumors) criteria is currently considered to be the recommended guidelines for assessment of tumor response by the National Cancer Institute (see Therasse et al., J. Natl. Cancer Inst. 92:205-216, 2000). According to the RECIST criteria tumor response means a reduction or elimination of all measurable lesions or metastases. Disease is generally considered measurable if it comprises lesions that can be accurately measured in at least one dimension as >20 mm with conventional techniques or >10 mm with spiral CT scan with clearly defined margins by medical photograph or X-ray, computerized axial tomography (CT), magnetic resonance imaging (MRI), or clinical examination (if lesions are superficial). Non-measurable disease means the disease comprises of lesions <20 mm with conventional techniques or <10 mm with spiral CT scan, and truly non-measurable lesions (too small to accurately measure). Non-measureable disease includes pleural effusions, ascites, and disease documented by indirect evidence.

The criteria for objective status are required for protocols to assess solid tumor response. Representative criteria include the following: (1) Complete Response (CR), defined as complete disappearance of all measurable disease; no new lesions; no disease related symptoms; no evidence of non-measurable disease; (2) Partial Response (PR) defined as 30% decrease in the sum of the longest diameter of target lesions (3) Progressive Disease (PD), defined as 20% increase in the sum of the longest diameter of target lesions or appearance of any new lesion; (4) Stable or No Response, defined as not qualifying for CR, PR, or Progressive Disease. (See Therasse et al., supra.)

Additional endpoints that are accepted within the oncology art include overall survival (OS), disease-free survival (DFS), objective response rate (ORR), time to progression (TTP), and progression-free survival (PFS) (see Guidance for Industry: Clinical Trial Endpoints for the Approval of Cancer Drugs and Biologics, April 2005, Center for Drug Evaluation and Research, FDA, Rockville, MD.)

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition.

The disclosure will be further clarified by the following examples, which are intended to be purely exemplary of the disclosure and in no way limiting.

### EXAMPLES

### Example 1: Generation of Humanized Variants of Anti-CD123 Antibodies and Construction of Monospecific and Bispecific CD123-Binding Molecules

### Isolation of Monospecific CD123-Binding Molecules by Phage Display

anti-CD123-specific scFv binding molecules were isolated from SuperHuman library, Distributed Bio Inc. (South San Francisco, CA), using soluble phage display panning technique against biotinylated recombinant CD123 protein (SEQ ID NO:200) following Distributed Bio protocols and as described previously by Ayriss, J et al, (2007) J Proteome Res., p 1072-82. Binding specificity of clones was characterized by ELISA using CD123 and unrelated recombinant protein with identical purification tag. Flow cytometry using HEK293 cells transfected with human and cynomolgus monkey variants of CD123, SEQ ID NO: 201 and SEQ ID NO:203, respectively, was used to identify clones binding to CD123 in the context of cell surface. Nucleotide and amino acid sequences of binding molecules referred to in these Examples can be found in Table 5.

### Isolation of Monospecific CD123-Binding Molecules by Hybridoma Generation

Anti-CD123-specific antibodies were isolated from a hybridoma library generated after immunizing OmniMice (Ligand Inc, San Diego, CA) with recombinant human CD123 ectodomain (SEQ ID NO:200). Binding specificity of individual clones was confirmed by testing binding using flow cytometry on HEK293 cells transfected with human and cynomolgus monkey variants of CD123, SEQ ID NO:201 and 203, respectively, and further confirmed by lack of binding to parental HEK293 cells. Sequences were obtained by RT-PCR using the OneStep RT-PCR Kit (QIAGEN Inc., Valencia, CA), following a modified version of the manufacturer's protocol. Briefly, cells from each clone were scraped from frozen cell bank vials and resuspended in RNase-free water. This cell suspension was then used as template in a RT-PCR reaction using sets of gene-specific primers that flank the heavy, kappa or lambda variable domains. Sequencing was performed using a reverse primer in the constant domains for each of these fragments. Sequences were then converted to scFv format by amplifying the variable domains using primers that contain overlapping sequences and were assembled into a mammalian expression vector using NEBuilder® HiF DNA Assembly Cloning Kit (New England Biolabs, Beverly, MA).

TABLE 5
CD123 sequences used for immunization and screening

| | | | SEQ ID NOs: |
|---|---|---|---|
| | | Amino Acid | nucleotide |
| Name | Nucleotide Sequence | Sequence | (amino acid) |
| human | atggaagcaccagcgcagcttctcttcctcctgctactctggctcccagat | kedpnppitnlrmkaka | SEQ ID NO: |
| CD123 | accaccggtaaggaggaccccaacccccccatcaccaacctgaggatga | qqltwdlnrnvtdiecvk | 199 |
| ectodomain, | aggccaaggcccagcagctgacctgggacctgaacaggaacgtgacag | dadysmpavnnsycqf | (SEQ ID |
| with Avi- | acatcgaatgcgtgaaggatgccgactacagcatgcccgccgtgaacaa | gaislcevtnytvrvanp | NO: 200) |
| 3xFLAG-His | ctcctactgccagttcggcgccatcagcctgtgcgaggtgacaaactaca | pfstwilfpensgkpwa | |
| affinity tag | ccgtgagagtggccaacccccccttcagcacctggatcctgtttcccgaga | gaenltcwihdvdflscs | |
| TRI032 | acagcggcaaaccctgggctggcgctgagaacctgacctgctggatccac | wavgpgapadvqydly | |
| | gacgtggactttctgtcctgcagctgggctgtgggacccggagctcctgcc | lnvanrrqqyeclhyktd | |
| | gatgtgcagtacgacctgtacctgaatgtggccaacagaagacagcagt | aqgtrigcrfddisrlssgs | |
| | acgagtgcctgcattacaagaccgacgcccagggaaccaggatcggctg | qsshilvrgrsaafgipct | |
| | caggtttgatgacatcagcaggctgtcctccggcagccagtccagccaca | dkfvvfsqieiltppnmt | |
| | tcctggtgagaggcagatccgccgccttcggcattccctgcacagacaag | akcnkthsfmhwkmr | |
| | ttcgtcgtcttcagccagatcgagattctgaccccccccaacatgaccgcc | shfnrkfryelqiqkrmq | |
| | aagtgtaacaagacccacagcttcatgcactggaagatgaggagccact | pviteqvrdrtsfqllnpg | |
| | tcaacaggaagttcaggtacgagctccagatccagaagaggatgcagcc | tytvqirarervyeflsaw | |
| | cgtgatcaccgagcaggtgagggacaggacatccttccagctgctgaatc | stpqrfecdqeegantr | |
| | ccggcacatacaccgtgcagatcagggccagggaaagggtgtacgagtt | awrssslndifeaqkie | |
| | cctgtccgcctggagcaccccccagaggttcgagtgtgaccaggaggag | whedykddddkdykd | |
| | ggagccaataccagggcctggagatcctcgagtctcaacgatatttttga | dddkdykddddkhhh | |
| | agcccaaaaaattgagtggcatgaagattacaaggacgatgacgacaa | hhhhhhh | |
| | agactataaggacgacgacgataaggattacaaggatgacgatgataag | | |
| | caccatcatcatcaccatcaccaccaccactga | | |
| Full-length | atggtcctcctttggctcacgctgctcctgatcgccctgccctgtctcctgca | ggkpwagaenltcwih | SEQ ID NO: |
| human | aacgaaggaaggtgggaagccttgggcaggtgcggagaatctgacctgc | dvdflscswavgpgapa | 201 |
| CD123 | tggattcatgacgtggatttcttgagctgcagctgggcggtaggcccgggg | dvqydlylnvanrrqqy | (SEQ ID |
| sequence, | gcccccgcggacgtccagtacgacctgtacttgaacgttgccaacaggcg | eclhyktdaqgtrigcrfd | NO: 202) |
| isoform 2 | tcaacagtacgagtgtcttcactacaaaacggatgctcagggaacacgta | disrlssgsqsshilvrgrs | |
| TRI074 | tcgggtgtcgtttcgatgacatctctcgactctccagcggttctcaaagttc | aafgipctdkfvvfsqieil | |
| | ccacatcctggtgcggggcaggagcgcagccttcggtatcccctgcacag | tppnmtakcnkthsfm | |
| | ataagtttgtcgtcttttcacagattgagatattaactccacccaacatgac | hwkmrshfnrkfryelq | |
| | tgcaaagtgtaataagacacattcctttatgcactggaaaatgagaagtc | iqkrmqpviteqvrdrts | |
| | atttcaatcgcaaatttcgctatgagcttcagatacaaaagagaatgcag | fqllnpgtytvqirarerv | |
| | cctgtaatcacagaacaggtcagagacagaacctccttccagctactcaa | yeflsawstpqrfecdq | |
| | tcctggaacgtacacagtacaaataagagcccgggaaagagtgtatgaa | eegantrawrtsllialgtl | |
| | ttcttgagcgcctggagcaccccccagcgcttcgagtgcgaccaggagga | lalvcvfvicrrylvmqrlf | |
| | gggcgcaaacacacgtgcctggcggacgtcgctgctgatcgcgctgggg | priphmkdpigdsfqn | |
| | acgctgctggccctggtctgtgtcttcgtgatctgcagaaggtatctggtga | dklwweagkagleecl | |
| | tgcagagactctttccccgcatccctcacatgaaagaccccatcggtgaca | vtevqvvqkttrtrpleq | |
| | gcttccaaaacgacaagctggtggtctgggaggcgggcaaagccggcct | kliseedlaandildykd | |
| | ggaggagtgtctggtgactgaagtacaggtcgtgcagaaaactacgcgt | dddkv | |
| | acgcggccgctcgagcagaaactcatctcagaagaggatctggcagcaa | | |
| | atgatatcctggattacaaggatgacgacgataaggtttaa | | |
| Full-length | atggaagcccccgcccagctgctcttcctgctgctcctgtggctgcctgac | kedpnapirnlrmkeka | SEQ ID NO: |
| cynomolgus | accaccggcaaggaagaccccaatgcccccatcaggaacctgagaatga | qqlmwdlnrnvtdveci | 203 |
| CD123 | aggagaaggcccagcagctcatgtgggatctgaacaggaacgtgaccga | kgtdysmpamnnsyc | (SEQ ID |
| sequence, | cgtggagtgtatcaagggcaccgactactccatgcccgccatgaataaca | qfgaislcevtnytvrvas | NO: 204) |
| isoform 1 | gctattgccagttcggcgccatcagcctgtgcgaggtcaccaactacacc | ppfstwilfpensgtpra | |
| TRI114 | gtgagagtggccagcccccccttctccacctggattctgttccctgagaac | gaenltcwvhdvdflscs | |
| | agcggcacccctagggctggcgctgagaatctgacatgctgggtccatga | wvvgpaapadvqydly | |
| | cgtggacttcctgagctgcagctgggtggtgggacctgctgctcccgctga | Innpnsheqyrclhykt | |
| | cgtgcagtacgatctgtatctgaacaaccccaactcccacgagcagtaca | dargtqigcrfddiaplsr | |
| | ggtgcctgcactacaagacagacgctagaggcacccagatcggctgcag | gsqsshilvrgrsaavsip | |
| | gttcgatgatatcgcccctctgagcaggggatcccagagctcccatatcct | ctdkfvffsqierltppn | |
| | ggtgaggggcaggtccgccgctgtgagcattccttgcaccgacaagttcg | mtgecnethsfmhwk | |
| | tcttcttcagccagatcgagaggctgaccccccctaacatgacaggcgag | mkshfnrkfryelriqkr | |
| | tgcaacgagacccacagcttcatgcactggaagatgaagagccatttcaa | mqpvrteqvrdttsfql | |
| | caggaaattcaggtacgaactgaggattcagaagagaatgcagcccgtg | pnpgtytvqiraretvye | |
| | aggacagagcaggtgagggatacaaccagcttccagctgcccaatcctg | flsawstpqrfecdqee | |
| | gcacctataccgtgcagatcagggctagagagaccgtgtacgagtttctg | gassrawrtsllialgtllal | |
| | tccgcctggagcaccccccagaggtttgaatgtgaccaggaggagggag | Icvflicrrylvmqrlfpri | |
| | cctccagcagggcttggagaaccagcctcctcatcgccctgggcacactg | phmkdpigdtfqqdkl | |
| | ctggctctgctgtgtgtgttcctgatctgcagaaggtacctggtgatgcaga | vvweagkagleeclvse | |
| | ggctcttccctaggattccccacatgaaggaccccatcggcgacaccttcc | vqvvekt | |
| | agcaggacaaactggtggtgtgggaagccggaaaggccggcctggagg | | |
| | aatgcctcgtgtccgaggtgcaggtggtggagaagacctaa | | |

### Preparation of Bispecific CD123-Binding Molecules

Bispecific 00123-binding molecules targeting 00123 and 003 epsilon, TRI129 (SEQ ID NO:129 (nucleic acid), SEQ ID NO:130 (amino acid)); TRI130 (SEQ ID NO:131 (nucleic acid), SEQ ID NO: 132 (amino acid)); TRI11123 (SEQ ID NO: 133 (nucleic acid), SEQ ID NO: 134 (amino acid)); TRI124 (SEQ ID NO:135 (nucleic acid), SEQ ID NO:136 (amino acid)); TRI137 (SEQ ID NO: 137 (nucleic acid), SEQ ID NO: 138 (amino acid)); TRI125 (SEQ ID NO: 139 (nucleic acid), SEQ ID NO: 140 (amino acid)); TRI126 (SEQ ID NO: 141 (nucleic acid), SEQ ID NO:142 (amino acid)); TRI127 (SEQ ID NO:143 (nucleic acid), SEQ ID NO:144 (amino acid)); TRI134 (SEQ ID NO:145 (nucleic acid), SEQ ID NO:146 (amino acid)); TRI128 (SEQ ID NO:147 (nucleic acid), SEQ ID NO: 148 (amino acid)); TRI131 (SEQ ID NO: 149 (nucleic acid), SEQ ID NO:150 (amino acid)); TRI132 (SEQ ID NO:151 (nucleic acid), SEQ ID NO:152 (amino acid)); TRI138 (SEQ ID NO: 153 (nucleic acid), SEQ ID NO: 154 (amino acid)); and TRI139 (SEQ ID NO: 155 (nucleic acid), SEQ ID NO: 156 (amino acid)), were made using standard molecular biology techniques, starting with existing bispecific binding molecules as templates and using the methods generally disclosed in, e.g., POT Application Publication No. WO 2007/146968, U.S. Patent Application Publication No. 2006/0051844, POT Application Publication No. WO 2010/040105, POT Application Publication No. WO 2010/003108, and U.S. Pat. No. 7,166,707 (see also Table 3). Insertion of the N-terminal anti-CD123 scFv binding domain was accomplished through digestion of the parental template and scFv insert with the restriction enzymes HindIII and XhoI, desired fragments were identified and isolated by agarose gel purification, and ligated. Insertion of the C-terminal anti-CD3 epsilon scFv binding domain was accomplished through digestion of the parental template and scFv insert with the restriction enzymes EcoRI and NotI, desired fragments were identified and isolated by agarose gel purification, and ligated.

Assembly of constructs with human scFv domains was accomplished by a three piece ligation using a HindIII/BamHI fragment, a BamHI/XhoI fragment, and a destination vector cut with HindIII/XhoI. This was used to produce the gene sequences corresponding to the humanized bispecific molecules shown in Table 4.

TABLE 6
Composition of Initial Humanized Constructs

| | scFv | Nucleotide | Amino acid |
|---|---|---|---|
| Construct ID | Orientation | SEQ ID NO | SEQ ID NO |
| TRI129 | VHVL | 129 | 130 |
| TRI130 | VLVH | 131 | 132 |
| TRI123 | VHVL | 133 | 134 |
| TRI124 | VLVH | 135 | 136 |
| TRI139 | VHVL | 155 | 156 |
| TRI137 | VHVL | 137 | 138 |
| TRI125 | VHVL | 139 | 140 |
| TRI126 | VHVL | 141 | 142 |
| TRI127 | VLVH | 143 | 144 |
| TRI131 | VHVL | 149 | 150 |
| TRI132 | VHVL | 151 | 152 |
| TRI134 | VHVL | 145 | 146 |
| TRI128 | VHVL | 147 | 148 |
| TRI138 | VHVL | 153 | 154 |

### Expression and Purification of CD123-Binding Molecules and Antibodies

Bispecific CD123-binding molecules disclosed herein were produced by both transient transfection of human HEK293 cells and, in some instances, also stable transfection of CHO cells. Transfected cells were purified from cell culture supernatants by Protein A affinity chromatography. If aggregates were detected after affinity chromatography, secondary size exclusion chromatography was also performed to ensure homogeneity of the protein.

### Example 2: Binding of CD123-Binding Molecules to CD123(+) Cell Lines

To confirm that binding activity to CD123 on the surface of cancer cells was retained anti-CD123×anti-CD3ε molecules and cross-reactivity to cynomolgus CD123, flow cytometry was used to quantitate binding of constructed CD123-binding molecules to cell lines expressing CD123.

### Binding of Monospecific and Bispecific Proteins to CD123(+) Cell Lines

Binding studies on the CD123(+) Molm-13 (Matsuo, Y et al, 1997, Leukemia 11, 1469-1477.) cancer cell line and cynomolgus CD123 expressing CHO cells were performed by standard flow cytometry-based staining procedures. The Molm-13 cell line was obtained from DSMZ (Braunschweig, Germany). The Molm-13 cell line was cultured according to the provided protocols. Chinese hamster ovary (CHO) cells stably expressing the full length cynomolgus CD123 protein were developed in-house. A typical experiment would label 100,000 cells per well, in 96-well plates, with a range of 1,000 nM to 0.012 nM binding molecule in 100 μl of PBS buffer with 0.2% BSA and 2 mM EDTA, for 30 min on ice, followed by washes and incubation with PE-labeled minimum cross species reactive secondary antibody, goat anti-human IgG Fcγ, F(ab′)2 (Jackson Laboratory) for 30 minutes on ice. Signal from bound molecules was detected using a LSR-II™ flow cytometer (BD Biosciences) and analyzed by FlowJo flow cytometry analysis software. Mean fluorescence intensity (MFI) of bound molecules on cells was determined after exclusion of doublets. Nonlinear regression analysis to determine EC50 values was performed in GraphPad Prism 7© graphing and statistics software.

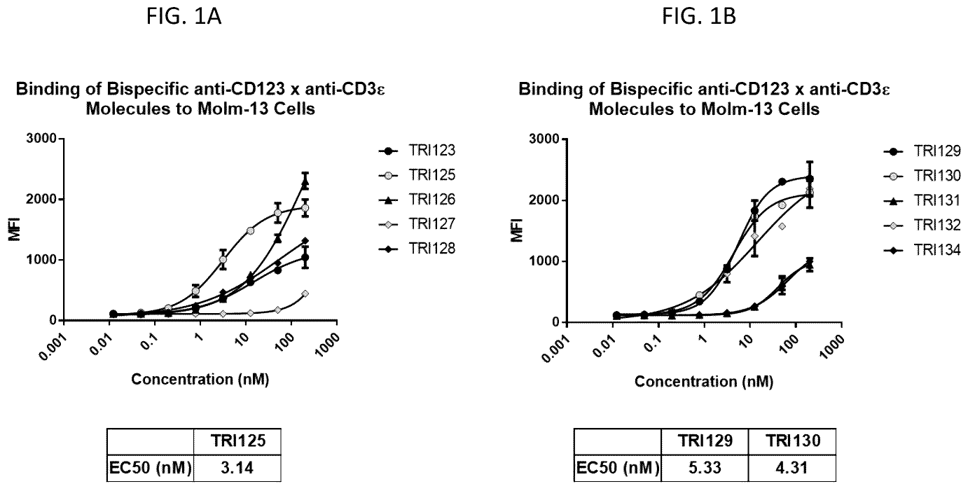

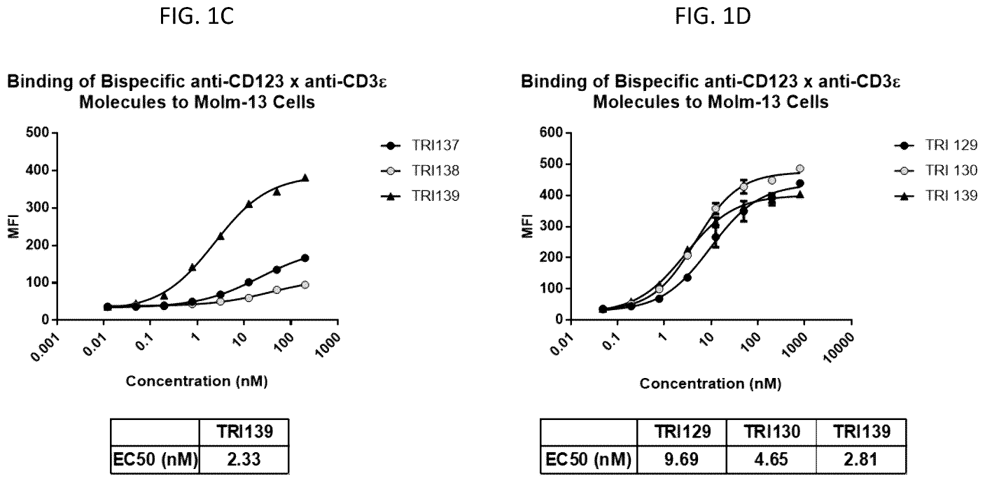

FIGS. 1A, 1B, 1C and 1D show the binding of 13 different bispecific anti-CD123×anti-CD3ε molecules (TRI123, TRI125, TRI126, TRI127, TRI128, TRI129, TRI130, TRI131, TRI132, TRI134, TRI137, TRI138 and TRI139) in three independent experiments to the CD123 (+) Molm-13 human tumor cell line. These experiments utilized bispecific constructs prepared from transiently transfected HEK293 cells. Four of the molecules demonstrated comparable maximum levels of binding at saturating concentrations. Observed EC₅₀ values for TRI125, TRI129, TRI130 and TRI139 were between 2-10 nM. Experiments were repeated for TRI129 and TRI130 from stably transfected CHO cells with similar results (data not shown).

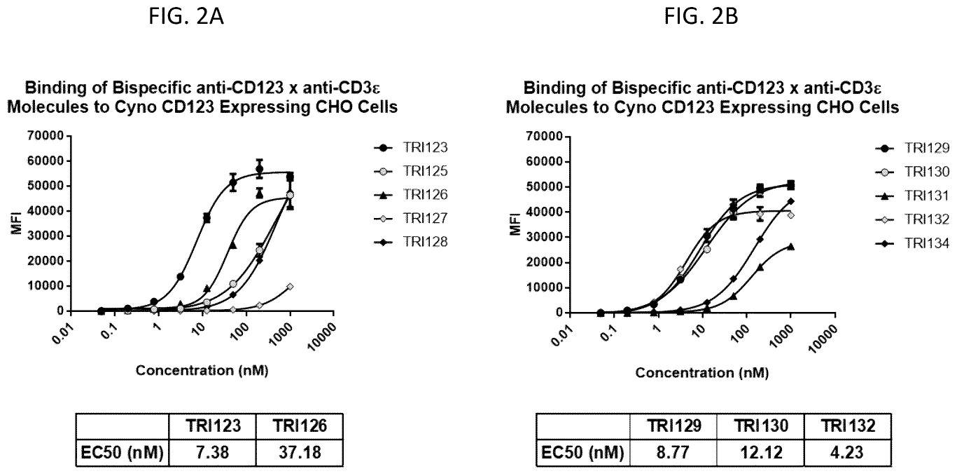

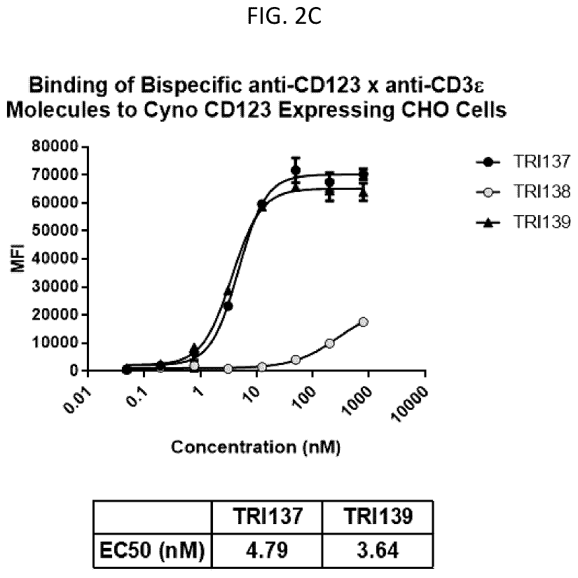

FIGS. 2A, 2B and 2C show the binding of 13 different bispecific anti-CD123×anti-CD3ε molecules (TRI123, TRI125, TRI126, TRI127, TRI128, TRI129, TRI130, TRI131, TRI132, TRI134, TRI137, TRI138 and TRI139) in two independent experiments to CHO cells stably expressing cynomolgus CD123 protein. Seven of the molecules demonstrated comparable maximum levels of binding at saturating concentrations. Observed EC₅₀ values for TRI123, TRI126, TRI129, TRI130, TRI132, TRI137 and TRI139 were between 3-38 nM.

These results confirm the constructed anti-CD123×anti-CD3ε molecules retain binding activity to human CD123 on the surface of cancer cells and are cross-reactive with cynomolgus CD123.

### Example 3: Redirected T Cell Cytotoxicity Assays with CD123(+) Cell Lines

To confirm that bispecific molecules binding to both CD123 and CD3ε could redirect T-cell cytotoxicity against a CD123(+) cell line, chromium-51 release assays were used to quantify target cell lysis induced by T-cells.

Chromium-51 Release Assays with CD123(+) Cell Lines

The Molm-13 human tumor cell line was cultured according to the provided protocols. Peripheral blood mononuclear cells (PBMC) were isolated from human blood using standard ficoll gradients. The isolated cells were washed in saline buffer. PBMC were cultured for 24 hours with Human T-Activator CD3/CD28 Dynabeads® (catalogue #11131D, Gibco Life Technologies, Carlsbad, California, USA) to activate T-cells using the manufacturers protocols. After 24 hours of culture PBMC were harvested and placed in a magnetic field to remove the Dynabeads. The isolated cells were washed in RPMI media+10% human serum. During the assay, concentrations of bispecific molecules with final concentration ranging from 1000 pM to 0.061 pM were added to the activated PBMC (approximately 100,000 per well).

Approximately 2.5×10⁶ Molm-13 target cells were treated with 0.125 mCi of ⁵¹Cr and incubated for 90 minutes in a 37° C., 5% CO₂ humidified incubator. After incubation, cells were washed 3 times with assay media (RPMI with 10% human serum) and re-suspended in 12.5 mL of assay media. From this suspension, 50 μL was dispensed per well into 96 well U-bottom plates (approximately 10,000 cells per well) to bring the total volume to 200 μL per well, and the PBMC to target cell ratio to 10:1. A zero lysis control was generated by target cells only, omitting the PBMC. A total lysis control was generated by including 0.20% NP-40 as the treatment with target cells only. A background lysis control was generated by target cells with PBMC in the absence of bispecific molecules.

Plates were incubated for 4 hours at 37° C., 5% CO₂ in a humidified incubator, after which they were centrifuged at 1000 rpm for 3 minutes, and 25 μL of supernatant was transferred from each well to the corresponding well of a 96-well Luma sample plate. Sample plates were allowed to air dry in a chemical safety hood for 18 hours, and then radioactivity was read on a TopCount microplate scintillation counter (PerkinElmer) using a standard protocol.

Percent specific lysis was calculated using the formula: ((signal in drug treated sample−background signal from samples with Target Cell only)/(signal in total lysis wells−background signal from samples with Target Cell only))×100.

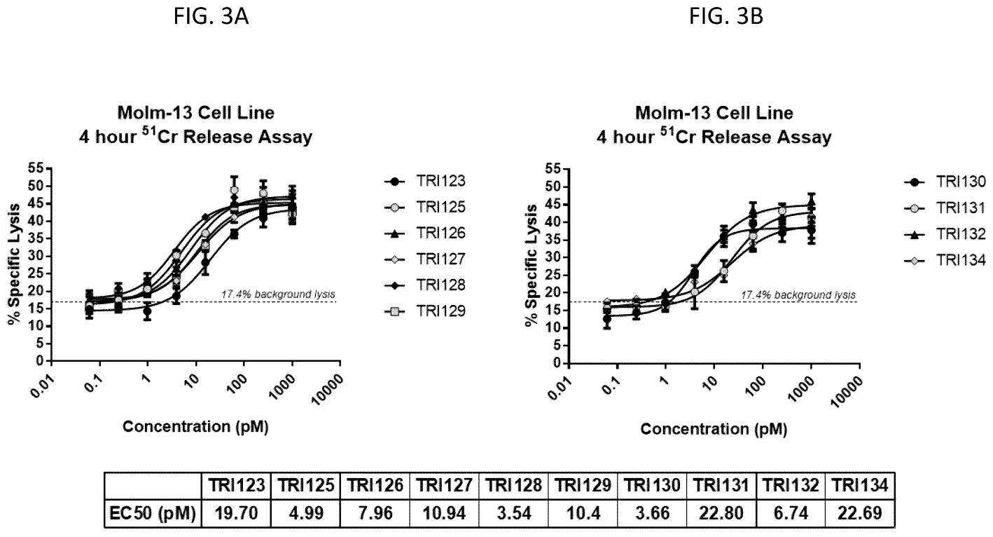

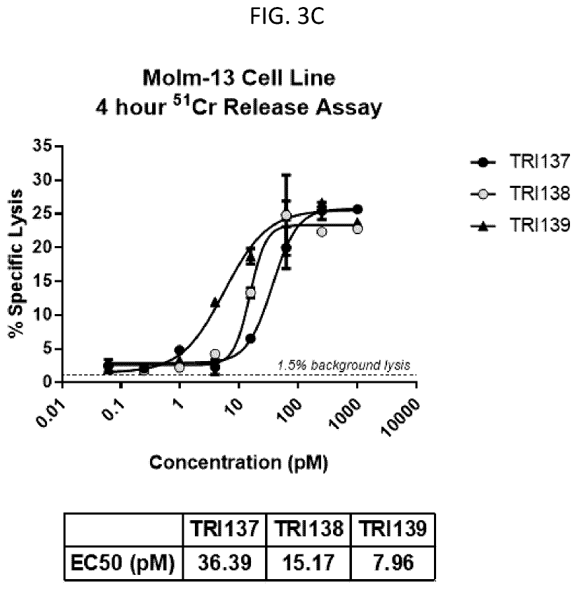

FIGS. 3A, 3B and 3C show chromium-51 release assays with the Molm-13 cell line measured at 4 hours using 13 different bispecific anti-CD123×anti-CD3ε molecules (TRI123, TRI125, TRI126, TRI127, TRI128, TRI129, TRI130, TRI131, TRI132, TRI134, TRI137, TRI138 and TRI139) in two independent experiments. All of the bispecific anti-CD123×anti-CD3ε molecules showed efficient target cell lysis at 4 hours ranging between 24-48% maximum specific lysis. Measured EC₅₀ values ranged between 3-37 pM at 4 hours.

Redirected T cell cytotoxicity assays were also performed with CHO cells stably expressing cynomolgus CD123 protein (TRI129 and TRI130) with (i) Molm-13 (human CD123+ cells) and human T-cells, (ii) Molm-13 (human CD123+ cells) and cynomolgous T-cells and (iii) CHO cells stably expressing cynomolgous CD123 and human T-cells. The data show that TRI129 and TRI130 are cross-reactive to human and cynomolgous CD123+ cells and T-cells.

These results confirm that the bispecific molecules binding to both CD123 and CD3ε could redirect T-cell cytotoxicity against a CD123(+) cell line.

### Example 4: Target-Dependent T-Cell Proliferation Induced Against CD123(+) Cell Line by Anti-CD123 Bispecific Molecules

To compare the effectiveness of different bispecific CD123-binding molecules at inducing target-dependent T-cell proliferation, six different anti-CD123×anti-CD3ε bispecific molecules including TRI123, TRI126, TRI129, TRI130, TRI132 and TRI139 were tested in two independent experiments.

T-cell proliferation was assessed by flow cytometry using a CD123(+) cell line, Molm-13. Peripheral blood mononuclear cells (PBMC) were isolated from human blood using standard density-gradient separation methods. The isolated cells were washed in saline buffer. T-cells were further isolated using a Pan T-cell Isolation Kit II from Miltenyi Biotec (Bergisch Gladbach, Germany) using the manufacturer's protocol. Molm-13 cells were irradiated to prevent cell division using a Faxitron-CellRad X-Ray Irradiation System from Faxitron Bioptics LLC (Tucson, Arizona, USA).

Proliferation was assessed by labeling isolated T-cell populations with CFSE. CFSE-labeled T-cells were plated in U-bottom 96-well plates at 120,000 cells/well, respectively, with 30,000 Molm-13 tumor cells/well, to achieve approximate T-cell to tumor cell ratios of 4:1. Concentrations of test molecules ranging from 2,000 pM to 0.002 pM were added to the cell mixtures to a final volume of 200 I/well in RPMI 1640 media supplemented with 10% human AB serum, sodium pyruvate, antibiotics and non-essential amino acids. Plates were incubated at 37° C., 5% CO₂ in humidified incubators. After 4 days, cells were labeled with antibodies for flow cytometric analysis in original plates to minimize cell losses, using saline buffer with 0.1% bovine serum albumin and 2 mM EDTA. After centrifugation and removal of supernatant, the cell pellets were resuspended with a mixture of the following dye-labeled antibodies in 50 μl volumes: CD5-PE, CD8-Pacific Blue, CD25-PE-Cy7, and 7AAD, and incubated for 30 min on ice. Cells were washed twice and resuspended in 120 μl volumes immediately prior to acquisition of 50% of each well in a BD LSRII flow cytometer. The sample files were analyzed using FlowJo software to calculate the percentages of CD4⁺ (CD8⁻) or CD8⁺ T-cells that had undergone at least one cell division, according to their CFSE profile, by gating sequentially on forward vs side scatter, 7AAD⁻, CD5⁺, CD4⁺ or CD8⁺ T-cells (7AAD⁻, CD5⁺ CD8⁻ or 7AAD⁻ CD5⁺ CD8⁺, respectively). Graphs were plotted using GraphPad Prism 7.0.

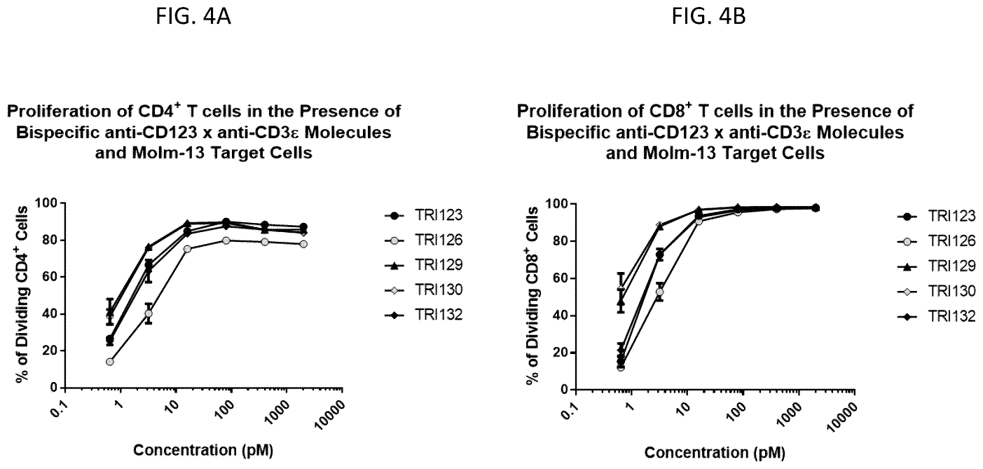

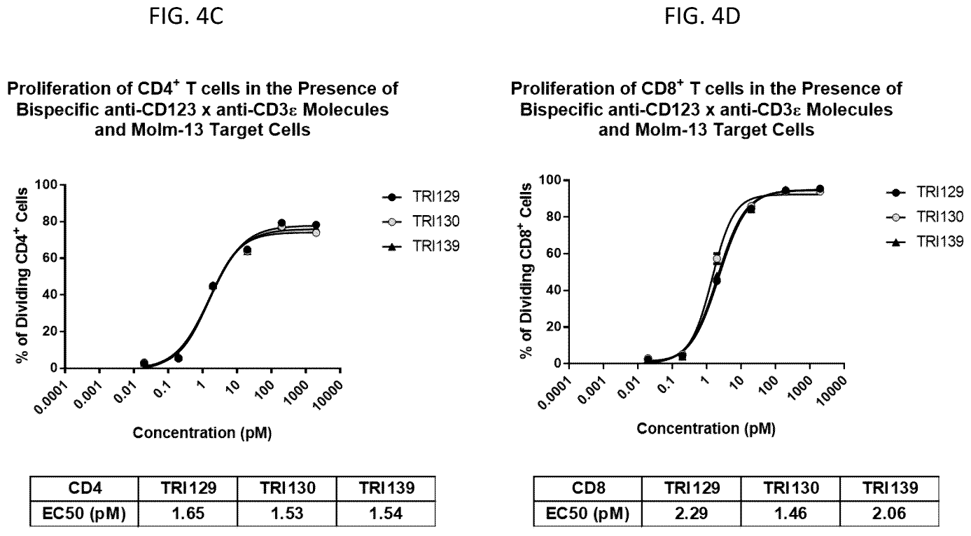

Analysis of dividing T-cell populations (FIGS. 4A and 4B) revealed a significant increase in the percent of proliferating cells in the presence of Molm-13 cells. All molecules showed robust induction of T-cell proliferation at low concentrations (10 pM), and proliferation was slightly higher in the CD8⁺ (FIG. 4B) than the CD4⁺ (CD8⁻) (FIG. 4A) T-cell population. EC₅₀ values were determined with TRI129, TRI130 and TRI139 for both CD4 (FIG. 4C) and CD8 (FIG. 4D) populations demonstrating similar potency for all three anti-CD123×anti-CD3ε bispecific molecules.

These results confirm that bispecific molecules binding to both CD123 and CD3ε could induce target-dependent T-cell proliferation against a CD123(+) cell line.

### Example 5: Determination of Pharmacokinetics in a Representative Non-Clinical Species

To determine the pharmacokinetics in a relevant non-clinical species, a bispecific anti-CD123×anti-CD3 molecule is tested in a Non-Human Primate (NHP) species (e.g., cynomolgus monkeys) for pharmacokinetic (PK) and toxicology assessment of the anti-CD123×anti-CD3 molecule. Cross-reactivity of the anti-CD123 binding domain to NHP CD123 is tested by ELISA or surface plasmon resonance assays (similar to Example 2). Since there is 85.7% sequence identity between NHP and human CD123, equivalent binding is expected. PK and immunogenicity assays can be developed and used to detect and quantify the anti-CD123×anti-CD3 molecule in the presence of cyno serum, as well as to evaluate the immunogenicity response in NHP. Upon successful completion of these tasks to justify cynomolgus monkeys as a relevant tox species for CD123-targeting molecules, a single dose PK/tolerability experiment is performed. The study can be performed at a qualified clinical research organization (CRO). The study results are examined for PK parameters and any adverse events including unexplainable adverse events that cannot be attributed to the mechanism of action of the drug or to anti-drug antibodies (ADA). A toxicologist verifies and interprets the study results and supports continued development of the lead. PK parameters derived from this study are used to design future toxicology studies in non-human primates and/or clinical studies in human patients.

### Example 6: Testing of Safety and Tolerability Upon Human Clinical Administration

To determine the safety and tolerability in humans of a lead bispecific molecule targeting CD123 and CD3, the following phase 1 clinical trial could be conducted. The first-in-human study of an anti-CD123×anti-CD3 bispecific molecule can be a dose-escalation study to identify the maximum tolerated dose (MTD) in human administration.

As bispecific anti-CD123×anti-CD3 molecules are agonistic, the starting dose is set at a dose producing the Minimum Anticipated Biological Effect Level (MABEL) based on in vitro activity of anti-CD123×anti-CD3. Human pharmacokinetics (PK) are estimated using PK values determined in non-clinical models (mice or non-human primates) and allometric scaling to predict a dose yielding the MABEL. Bispecific molecule is dosed using either intravenous infusion or subcutaneous injection. Dose escalation follows a standard 3+3 design, with an anticipated 12 dose cohorts (N=24-72 patients). Dose frequency is dependent on observed PK in non-clinical studies, but could be weekly (QW), every other week (Q2WV), every third week (Q3W), or monthly (Q4W). Dosing continues until disease progression (as defined by either the immune-related response criteria (irRC) or the response criteria in solid tumors (RECIST)).

Primary endpoint of the study is safety, defining an MTD and any dose limiting toxicities. Secondary endpoints of the study is PK, immunogenicity, and objective responses in tumor volume assessed either by irRC or RECIST criteria. For patients with hematologic malignancies, additional criteria may be assessed, such as the presence of minimal residual disease (MRD status). As appropriate or feasible, biomarker samples are taken from whole blood as well as from lymph node or bone marrow biopsies to monitor the effects on the immune system as well as effects on the cancer or malignancy.

Inclusion criteria for a phase 1 study may be broad and allow for inclusion of patients with refractory or relapsed disease from multiple indications where CD123 expression has been previously shown to be high, such as acute myeloid leukemia (AML), B-lymphoid leukemia, blastic plasmocytoid dendritic neoplasm (BPDCN), and hairy cell leukemia. Patients entering the study require evidence of CD123 expression from immunohistochemical (IHC) analysis from either archival biopsy samples of the primary tumor or from a pre-treatment biopsy of a recurrent tumor from a metastatic site.

A bispecific anti-CD123×anti-CD3 molecule is determined to be sufficiently safe if patients dosed at the MTD or dose levels below the MTD show evidence of clinical benefit, either from objective responses from the irRC or RECIST criteria or changes in potentially prognostic serum biomarkers, such as CTC, PSA, or TAG72.

TABLE 7
Human CD123 isoform sequences (National Center for Biotechnology
Information)

| | | | SEQ ID NO |
|---|---|---|---|
| | Nucleotide Sequence | Amino Acid | (Amino Acid SEQ |
| Name | (mRNA) | Sequence (complete) | ID NO) |
| CD123 | GTCAGGTTCATGGTTACGAAGCTG | MVLLWLTLLLIALPCLLQ | SEQ ID NO: 205 |
| isoform 1 | CTGACCCCAGGATCCCAGCCCGTG | TKEDPNPPITNLRMKAKA | (SEQ ID NO: 206) |
| NM_002183.3 | GGAGAGAAGGGGGTCTCTGACA | QQLTWDLNRNVTDIECVK | |
| NP_002174.1 | GCCCCCACCCCTCCCCACTGCCAG | DADYSMPAVNNSYCQF | |
| | ATCCTTATTGGGTCTGAGTTTCAG | GAISLCEVTNYTVRVANP | |
| | GGGTGGGGCCCCAGCTGGAGGT | PFSTWILFPENSGKPWAG | |
| | TATAAAACAGCTCAATCGGGGAGT | AENLTCWIHDVDFLSCSW | |
| | ACAACCTTCGGTTTCTCTTCGGGG | AVGPGAPADVQYDLYL | |
| | AAAGCTGCTTTCAGCGCACACG | NVANRRQQYECLHYKTDA | |
| | GGAAGATATCAGAAACATCCTAGG | QGTRIGCRFDDISRLSSG | |
| | ATCAGGACACCCCAGATCTTCTCA | SQSSHILVRGRSAAFGIP | |
| | ACTGGAACCACGAAGGCTGTTT | CTDKFVVFSQIEILTP | |
| | CTTCCACACAGTACTTTGATCTCC | PNMTAKCNKTHSFMHWKM | |
| | ATTTAAGCAGGCACCTCTGTCCTG | RSHFNRKFRYELQIQKRM | |
| | CGTTCCGGAGCTGCGTTCCCGA | QPVITEQVRDRTSFQLLN | |
| | TGGTCCTCCTTTGGCTCACGCTGC | PGTYTVQIRARERVYE | |
| | TCCTGATCGCCCTGCCCTGTCTCC | FLSAWSTPQRFECDQEEG | |
| | TGCAAACGAAGGAAGATCCAAA | ANTRAWRTSLLIALGTLL | |
| | CCCACCAATCACGAACCTAAGGAT | ALVCVFVICRRYLVMQRL | |
| | GAAAGCAAAGGCTCAGCAGTTGAC | FPRIPHMKDPIGDSFQ | |
| | CTGGGACCTTAACAGAAATGTG | NDKLVVWEAGKAGLEECL | |
| | ACCGATATCGAGTGTGTTAAAGAC | VTEVQVVQKT | |
| | GCCGACTATTCTATGCCGGCAGTG | | |
| | AACAATAGCTATTGCCAGTTTG | | |
| | GAGCAATTTCCTTATGTGAAGTGA | | |
| | CCAACTACACCGTCCGAGTGGCCA | | |
| | ACCCACCATTCTCCACGTGGAT | | |
| | CCTCTTCCCTGAGAACAGTGGGAA | | |
| | GCCTTGGGCAGGTGCGGAGAATCT | | |
| | GACCTGCTGGATTCATGACGTG | | |
| | GATTTCTTGAGCTGCAGCTGGGCG | | |
| | GTAGGCCCGGGGGCCCCCGCGGAC | | |
| | GTCCAGTACGACCTGTACTTGA | | |
| | ACGTTGCCAACAGGCGTCAACAGT | | |
| | ACGAGTGTCTTCACTACAAAACGG | | |
| | ATGCTCAGGGAACACGTATCGG | | |
| | GTGTCGTTTCGATGACATCTCTCG | | |
| | ACTCTCCAGCGGTTCTCAAAGTTC | | |
| | CCACATCCTGGTGCGGGGCAGG | | |
| | AGCGCAGCCTTCGGTATCCCCTGC | | |
| | ACAGATAAGTTTGTCGTCTTTTCA | | |
| | CAGATTGAGATATTAACTCCAC | | |
| | CCAACATGACTGCAAAGTGTAATA | | |
| | AGACACATTCCTTTATGCACTGGA | | |
| | AAATGAGAAGTCATTTCAATCG | | |
| | CAAATTTCGCTATGAGCTTCAGAT | | |
| | ACAAAAGAGAATGCAGCCTGTAAT | | |
| | CACAGAACAGGTCAGAGACAGA | | |
| | ACCTCCTTCCAGCTACTCAATCCT | | |
| | GGAACGTACACAGTACAAATAAGA | | |
| | GCCCGGGAAAGAGTGTATGAAT | | |
| | TCTTGAGCGCCTGGAGCACCCCCC | | |
| | AGCGCTTCGAGTGCGACCAGGAGG | | |
| | AGGGCGCAAACACACGTGCCTG | | |
| | GCGGACGTCGCTGCTGATCGCGCT | | |
| | GGGGACGCTGCTGGCCCTGGTCTG | | |
| | TGTCTTCGTGATCTGCAGAAGG | | |
| | TATCTGGTGATGCAGAGACTCTTT | | |
| | CCCCGCATCCCTCACATGAAAGAC | | |
| | CCCATCGGTGACAGCTTCCAAA | | |
| | ACGACAAGCTGGTGGTCTGGGAGG | | |
| | CGGGCAAAGCCGGCCTGGAGGAGT | | |
| | GTCTGGTGACTGAAGTACAGGT | | |
| | CGTGCAGAAAACTTGAGACTGGGG | | |
| | TTCAGGGCTTGTGGGGGTCTGCCT | | |
| | CAATCTCCCTGGCCGGGCCAGG | | |
| | CGCCTGCACAGACTGGCTGCTGGA | | |
| | CCTGCGCACGCAGCCCAGGAATGG | | |
| | ACATTCCTAACGGGTGGTGGGC | | |
| | ATGGGAGATGCCTGTGTAATTTCG | | |
| | TCCGAAGCTGCCAGGAAGAAGAAC | | |
| | AGAACTTTGTGTGTTTATTTCA | | |
| | TGATAAAGTGATTTTTTTTTTTTT | | |
| | AACCCAAAA | | |
| CD123 | GTCAGGTTCATGGTTACGAAGCTG | MVLLWLTLLLIALPCLLQ | SEQ ID NO: 207 |
| isoform 2 | CTGACCCCAGGATCCCAGCCCGTG | TKEGGKPWAGAENLTCWI | (SEQ ID NO: 208) |
| NM_001267713.1 | GGAGAGAAGGGGGTCTCTGACA | HDVDFLSCSWAVGPGAPA | |
| NP_001251642.1 | GCCCCCACCCCTCCCCACTGCCAG | DVQYDLYLNVANRRQQ | |
| | ATCCTTATTGGGTCTGAGTTTCAG | YECLHYKTDAQGTRIGCR | |
| | GGGTGGGGCCCCAGCTGGAGGT | FDDISRLSSGSQSSHILV | |
| | TATAAAACAGCTCAATCGGGGAGT | RGRSAAFGIPCTDKFWF | |
| | ACAACCTTCGGTTTCTCTTCGGGG | SQIEILTPPNMTAKCN | |
| | AAAGCTGCTTTCAGCGCACACG | KTHSFMHWKMRSHFNRKF | |
| | GGAAGATATCAGAAACATCCTAGG | RYELQIQKRMQPVITEQV | |
| | ATCAGGACACCCCAGATCTTCTCA | RDRTSFQLLNPGTYTVQI | |
| | ACTGGAACCACGAAGGCTGTTT | RARERVYEFLSAWSTP | |
| | CTTCCACACAGTACTTTGATCTCC | QRFECDQEEGANTRAWRT | |
| | ATTTAAGCAGGCACCTCTGTCCTG | SLLIALGTLLALVCVFVI | |
| | CGTTCCGGAGCTGCGTTCCCGA | CRRYLVMQRLFPRIPHMK | |
| | TGGTCCTCCTTTGGCTCACGCTGC | DPIGDSFQNDKLVVWE | |
| | TCCTGATCGCCCTGCCCTGTCTCC | AGKAGLEECLVTEVQVVQ | |
| | TGCAAACGAAGGAAGGTGGGAA | KT | |
| | GCCTTGGGCAGGTGCGGAGAATCT | | |
| | GACCTGCTGGATTCATGACGTGGA | | |
| | TTTCTTGAGCTGCAGCTGGGCG | | |
| | GTAGGCCCGGGGGCCCCCGCGGAC | | |
| | GTCCAGTACGACCTGTACTTGAAC | | |
| | GTTGCCAACAGGCGTCAACAGT | | |
| | ACGAGTGTCTTCACTACAAAACGG | | |
| | ATGCTCAGGGAACACGTATCGGGT | | |
| | GTCGTTTCGATGACATCTCTCG | | |
| | ACTCTCCAGCGGTTCTCAAAGTTC | | |
| | CCACATCCTGGTGCGGGGCAGGAG | | |
| | CGCAGCCTTCGGTATCCCCTGC | | |
| | ACAGATAAGTTTGTCGTCTTTTCA | | |
| | CAGATTGAGATATTAACTCCACCC | | |
| | AACATGACTGCAAAGTGTAATA | | |
| | AGACACATTCCTTTATGCACTGGA | | |
| | AAATGAGAAGTCATTTCAATCGCA | | |
| | AATTTCGCTATGAGCTTCAGAT | | |
| | ACAAAAGAGAATGCAGCCTGTAAT | | |
| | CACAGAACAGGTCAGAGACAGAAC | | |
| | CTCCTTCCAGCTACTCAATCCT | | |
| | GGAACGTACACAGTACAAATAAGA | | |
| | GCCCGGGAAAGAGTGTATGAATTC | | |
| | TTGAGCGCCTGGAGCACCCCCC | | |
| | AGCGCTTCGAGTGCGACCAGGAGG | | |
| | AGGGCGCAAACACACGTGCCTGGC | | |
| | GGACGTCGCTGCTGATCGCGCT | | |
| | GGGGACGCTGCTGGCCCTGGTCTG | | |
| | TGTCTTCGTGATCTGCAGAAGGTA | | |
| | TCTGGTGATGCAGAGACTCTTT | | |
| | CCCCGCATCCCTCACATGAAAGAC | | |
| | CCCATCGGTGACAGCTTCCAAAAC | | |
| | GACAAGCTGGTGGTCTGGGAGG | | |
| | CGGGCAAAGCCGGCCTGGAGGAGT | | |
| | GTGTGGTGACTGAAGTACAGGTCG | | |
| | TGCAGAAAACTTGAGACTGGGG | | |
| | TTCAGGGCTTGTGGGGGTCTGCCT | | |
| | CAATCTCCCTGGCCGGGCCAGGCG | | |
| | CCTGCACAGACTGGCTGCTGGA | | |
| | CCTGCGCACGCAGCCCAGGAATGG | | |
| | ACATTCCTAACGGGTGGTGGGCAT | | |
| | GGGAGATGCCTGTGTAATTTCG | | |
| | TCCGAAGCTGCCAGGAAGAAGAAC | | |
| | AGAACTTTGTGTGTTTATTTCATG | | |
| | ATAAAGTGATTTTTTTTTTTTT | | |
| | AACCCAAAA | | |

### Example 7: Target-Dependent T-Cell Activation Induced Against CD123(+) Cell Line by Anti-CD123 Bispecific Molecules

To compare the effectiveness of different bispecific 00123-binding molecules at inducing target-dependent T-cell activation of CD4⁺ and CD8⁺ T-cells, six different anti-CD123×anti-CD3ε bispecific molecules including TRI123, TRI126, TRI129, TRI130, TRI132 and TRI139 were tested in two independent experiments.

T-cell activation was assessed by flow cytometry using a CD123(+) cell line, Molm-13. Peripheral blood mononuclear cells (PBMC) were isolated from human blood using standard density-gradient separation methods. The isolated cells were washed in saline buffer. T-cells were further isolated using a Pan T-cell Isolation Kit II from Miltenyi Biotec (Bergisch Gladbach, Germany) using the manufacturer's protocol.

T-cells were plated in U-bottom 96-well plates at 120,000 cells/well, respectively, with 30,000 Molm-13 tumor cells/well, to achieve approximate T-cell to tumor cell ratios of 4:1. Concentrations of test molecules ranging from 2,000 pM to 0.002 pM were added to the cell mixtures to a final volume of 200 l/well in RPMI 1640 media supplemented with 10% human AB serum, sodium pyruvate, antibiotics and non-essential amino acids. Plates were incubated at 37° C., 5% CO₂ in humidified incubators. After 20 hours, cells were labeled with antibodies for flow cytometric analysis in original plates to minimize cell losses, using saline buffer with 0.1% bovine serum albumin and 2 mM EDTA. After centrifugation and removal of supernatant, the cell pellets were resuspended with a mixture of the following dye-labeled antibodies in 50 μl volumes: CD69-FITC, CD5-PE, CD8-Pacific Blue, CD4-ΔPC, CD25-PE-Cy7, and 7AAD, and incubated for 30 min on ice. Cells were washed twice and resuspended in 120 μl volumes immediately prior to acquisition of 50% of each well in a BD LSRII flow cytometer. The sample files were analyzed using FlowJo software to calculate the percentages of CD4⁺ (CD8⁻) or CD8⁺ T-cells that had upregulated CD69 and CD25, by gating sequentially on forward vs side scatter, 7AAD⁻, CD5⁺, CD4⁺ or CD8⁺ T-cells (7AAD⁻, CD5⁺ CD4⁺ or 7AAD⁻ CD5⁺ CD8⁺, respectively). Graphs were plotted using GraphPad Prism 7.0.

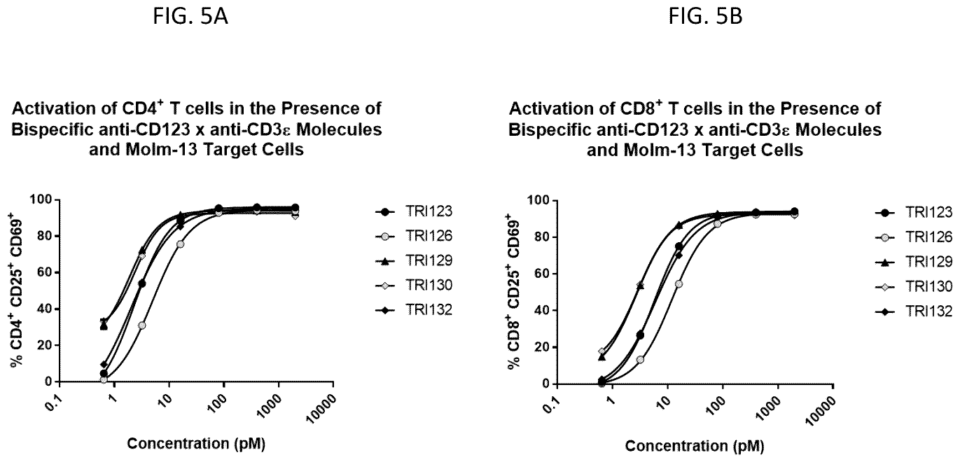

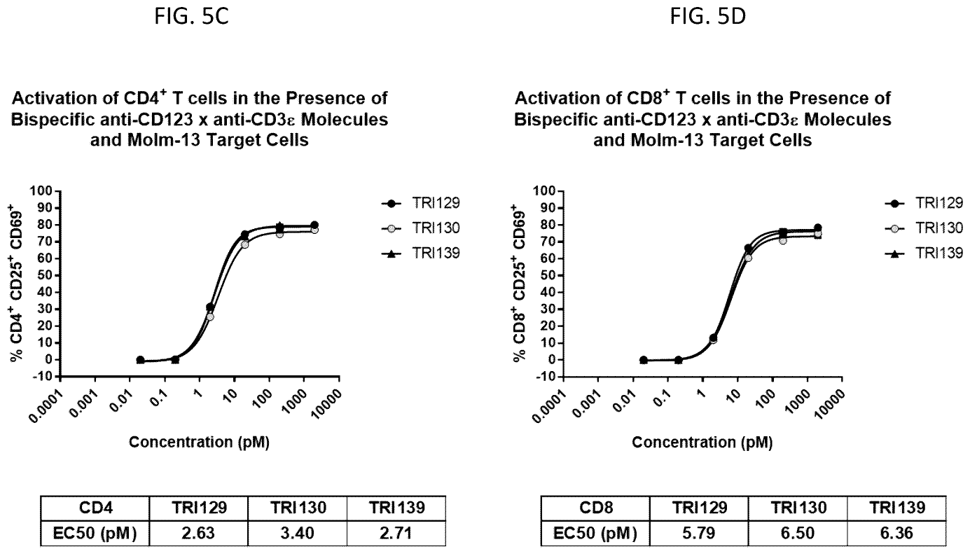

Analysis of activated T-cells after 20 hours (FIG. 5) revealed a significant increase in the percent of activated CD4⁺ and CD8⁺ T-cells in the presence of Molm-13 cells. All molecules tested induced maximal activation of T-cells in the presence of Molm-13 target cells at low concentrations ranging from 1-100 pM (FIG. 5). EC₅₀ values were determined with TRI129, TRI130 and TRI139 for both CD4⁺ (FIG. 5C) and CD8⁺ (FIG. 5D) CD25⁺ CD69⁺ populations demonstrating similar potency for all three anti-CD123×anti-CD3ε bispecific molecules.

These results demonstrate that bispecific molecules binding to both CD123 and CD3ε could induce target-dependent T-cell activation against a CD123(+) cell line.

### Example 8: Binding of Bispecific Proteins to CD123

To determine the kinetics and affinity of the bispecific to CD123, the TRI-129 and TRI-130 bispecific proteins were expressed via transient transfection in HEK293 cells and purified using a combination of affinity purification and size exclusion purification. The extracellular domain of CD123 was transiently expressed with an affinity tag on the C-terminus and purified using a combination of affinity purification and size exclusion purification. The analyses were performed using a BIACORE™ T200 instrument (Biacore Inc., Piscataway, NJ). The BIACORE™ T200 is a surface plasmon resonance (SPR)-based biosensor system that is designed to provide real-time kinetic binding data.

SPR binding studies of bispecific molecules to recombinant monomeric CD123 ectodomain (ECD) were conducted at 25° C. in HBS-EP+ buffer. AffiniPure F(ab′)2 fragment Goat anti-human IgG Fcγ fragment-specific (Jackson Immuno Research) at 20 μg/mL in 10 mM sodium acetate (pH 4.5) was immobilized at a density of 3600 response units (RU) on the surface of a CM5 research-grade sensor chip (R-1005-30, GE Healthcare) by standard amine coupling chemistry. The bispecific molecules at 200 nM in HBS-EP+ buffer were captured by the immobilized anti-Fc F(ab′)2 fragment at a flow rate of 30 μL/min for 120 sec to reach a stable 2000 RU response. Different concentrations of CD123 ECD (3-48 nM by 2-fold dilutions, including buffer as blank) were flowed over the captured bispecific molecules at 30 μL/min for 120 sec followed by a 300 sec dissociation period. Optimal regeneration was achieved by one injection of 10 mM glycine (pH 1.7) at a flow rate of 30 μL/min for 15 sec followed by one injection of 50 mM NaOH at 30 μL/min for 15 sec. A two minute stabilization with HBS-EP+ buffer was completed before the subsequent run.

Sensorgrams obtained from kinetic SPR measurements were analyzed by the double subtraction method. The signal from the reference flow cell was subtracted from the analyte binding response obtained from flow cells with immobilized or captured ligands. Buffer reference responses were then averaged from multiple injections. The averaged buffer reference responses were then subtracted from analyte binding responses, and the final double-referenced data were analyzed with BIACORE™ T200 Evaluation software (2.0, GE Healthcare), globally fitting data to derive kinetic parameters (Table 8). All sensorgrams were fitted using a simple one-to-one binding model.

TABLE 8
Derived kinetic parameters for αCD123 bispecific molecules
to CD123 ECD.

| Molecule | Kₐ (1/Ms) | k_{d} (1/s) | KD (nM) |
|---|---|---|---|
| TRI-129 | 1.79 × 10⁵ | 2.05 × 10⁻³ | 11.4 |
| TRI-130 | 1.72 × 10⁵ | 4.72 × 10⁻⁴ | 2.7 |

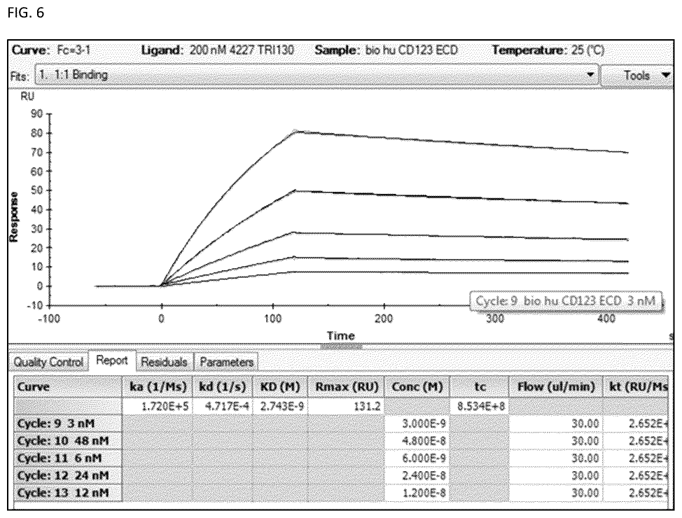

An example of the SPR analysis of TRI-130 at different ECD concentrations ranging from 3 nM to 48 nM is shown in FIG. 6. Thermodynamic and kinetic rate constants of binding were calculated using the BIACORE™ Evaluation software. For example, the affinity (KD) of TRI-130 for CD123 extracellular domain (ECD) was determined to be 2.74×10⁻⁹ M with a ka of 1.72×10⁵ M⁻¹s⁻¹ and a kd of 4.72×10⁻⁴ s⁻¹. The affinity of TRI-129 for the CD123 was determined to be 1.14×10⁻⁸ M with a ka of 1.79×10⁵ M⁻¹s⁻¹ and a kd of 2.05×10⁻³ s⁻¹.

### Example 9: Differential Scanning Calorimetry

Thermal stability of the CD123 binding domain was assessed using Differential Scanning Calorimetry (DSC). DSC measures heat capacity changes associated with the molecule's thermal denaturation when heated at a constant rate. Tm (thermal transition midpoint) value of CD123 binding domain scfv, which is a measure of its stability, can be extracted from the melting curve.

DSC thermal stability study was conducted using a MicroCal VP-Capillary DSC system (Malvern Instrument). An exact match of buffer, PBS pH7.4, was used as the reference. 500 uL of a 0.5 mg/mL solution of each protein sample with reference was loaded on the instrument and heated from 25° C. to 100° C. at a rate of one degree celsius per minute. Melting curve was analyzed using Origin 7 platform software “MicroCal VP-Capillary DSC Automated Analysis Software.” Tm of each melting transition was calculated using “non-2 state curser integration” method.

TABLE 9
Assessment of thermal stability of domains

| | | | Tm2 (αCD123 scFv | Tm3 |
|---|---|---|---|---|
| Sample | TmOnset | Tm1 (αCD3 scFv) | and Fc CH2) | (Fc CH3) |
| TRI129 | 51.19 | 59.59 | 64.48 | 83.87 |
| TRI130 | 53.19 | 59.59 | 65.71 | 84.64 |

### Example 10: Pharmacokinetic Activity

To determine the pharmacokinetic activity of bispecific molecules, female Balb/c mice were injected intravenously (IV) with 200 mg (˜10 mg/kg) of either TRI129 or TRI130 (n=30 for each molecule, TRI129 or TRI130). At each time point, blood was collected by cardiac puncture from 3 mice. Time points were: 15 minutes, 2, 6, 24, 48, 72, 96, 168, 336 and 504 hours. Blood was processed to serum, aliquoted and frozen. TRI129 and TRI130 concentrations in serum samples were determined by enzyme linked immunosorbent assay (ELISA). Serum concentrations over time were used to determine PK parameter estimates by non-compartmental analysis (NCA) and compartmental analysis. Serum concentration over time profiles were analyzed with Phoenix 64 software. Graphs were plotted using GraphPad Prism 7.0. NCA parameter analysis of the resulting data is provided in Table 10.

TABLE 10
NCA parameter analysis of data

| Bispecific | T ½ | Clearance | Volume | AUC |
|---|---|---|---|---|
| TRI-129 | 229 hours (9.5 days) | 0.204 ml/hr/kg | 67.54 ml/kg | 38750 hr * μg/ml |
| TRI-130 | 301 hours (12.5 days) | 0.186 ml/hr/kg | 80.84 ml/kg | 37309 hr * μg/ml |

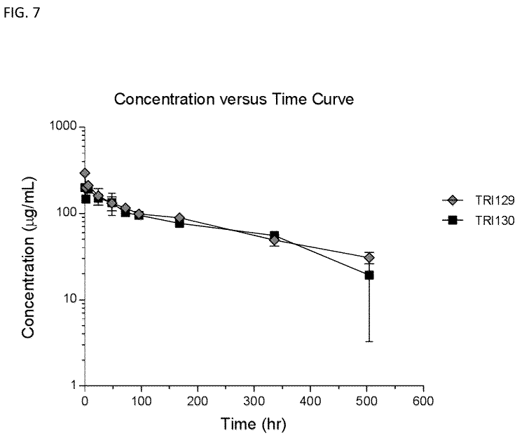

FIG. 7 shows concentration versus time curves for TRI129 and TRI130 bispecific molecules.

### Example 11: In Vivo Efficacy of Anti-CD123×Anti-CD3 Binding Molecules

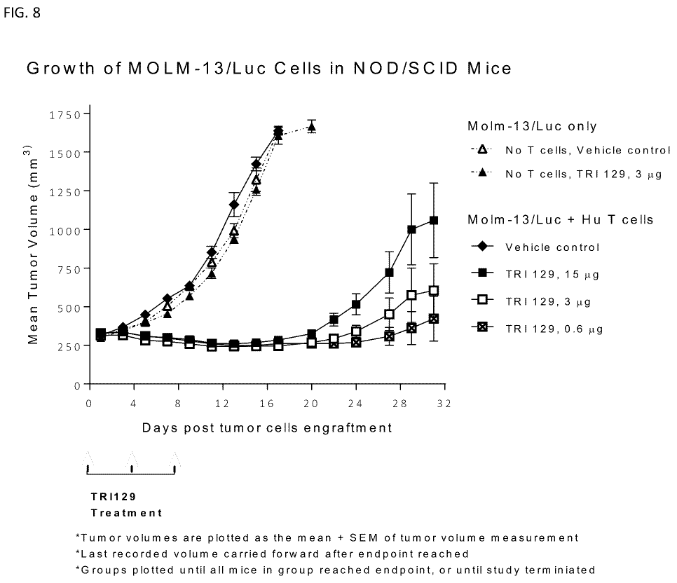

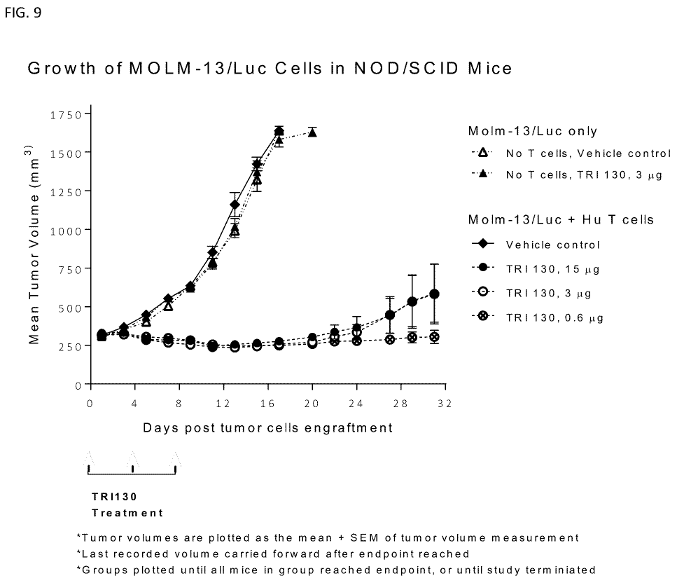

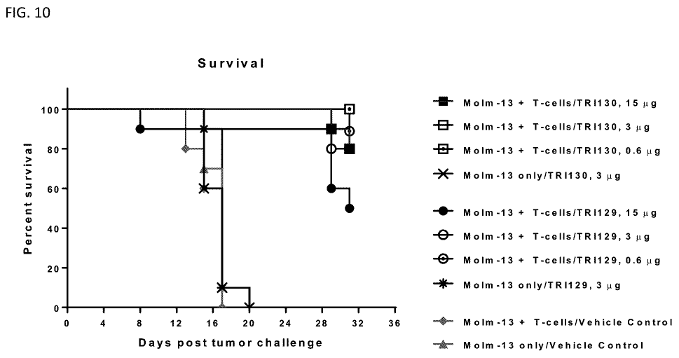

To determine if TRI129 and TRI130 are capable of inhibiting tumor growth and prolonging survival in vivo, a rodent model of human acute myeloid leukemia was utilized. MOLM-13/LUC cells were co-mixed with donor T-cells and matrigel and implanted into the flank of NOD/SCID mice on day 0 of the study. Animals were treated in groups of 10 mice per group with T-cells from one donor with vehicle, TRI129 or TRI130 on study Day 0, 4 and 8 at doses of 15, 3 and 0.6 μg of protein in a total volume of 200 μl. Tumor growth was measured with calipers over time during the study and survival events were recorded each time a mouse reached the endpoint (tumor volume ≥1500 mm³) and was euthanized. As shown FIGS. 8 and 9 there was minimal impact of TRI129 and TRI130 in the absence of donor T-cells and minimal impact of T-cells on tumor growth by the donor T-cells alone. Significant inhibition of tumor growth was seen after treatment with both TRI129 and TRI130 at all doses tested in the presence of donor T-cells. Significant differences in median survival of mice were determined using Kaplan-Meier survival analysis with a log-rank test for comparing survival curves. As shown FIG. 10, the survival of mice treated with all tested doses of TRI129 and TRI130 in the presence of co-implanted human T-cells was significantly prolonged relative to all control groups. Neither the co-implantation of T-cells with vehicle treatment nor the treatment of tumors with TRI129 or TRI130 in the absence of human T-cells extended survival compared to mice implanted with tumor cells alone and treated with vehicle. This result demonstrates both TRI129 and TRI130 are capable of driving inhibition of tumor growth and prolonging survival in a Xenograft model of acute myeloid leukemia.

### Example 12: TRI-130 Cytotoxicity is Specific to CD123 Expressing Cell Lines

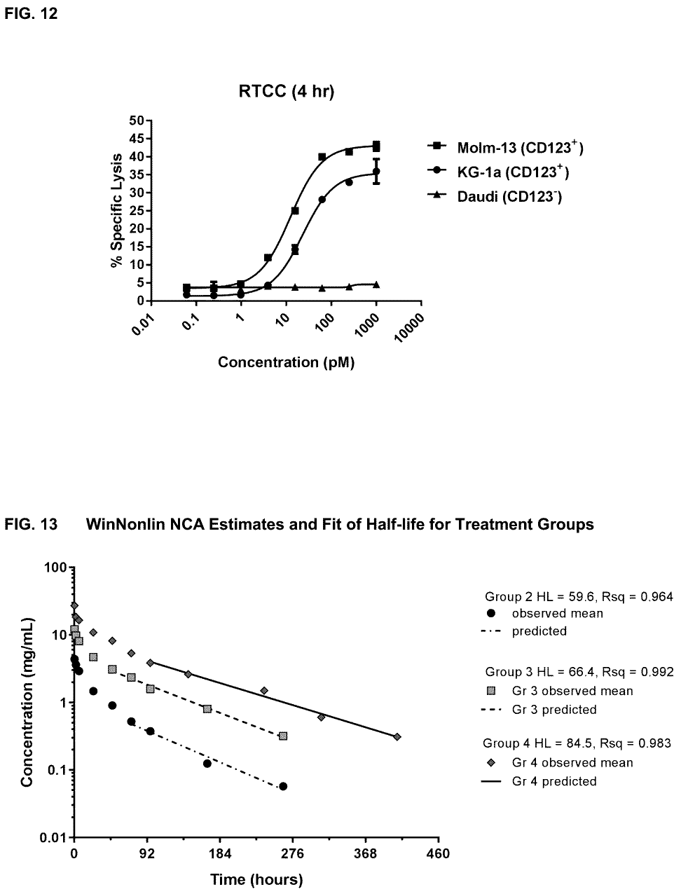

To confirm the specificity of TRI130 and compare its cytotoxic activity on tumor cell lines expressing different levels of CD123, chromium-51 release assays were used to quantify target cell lysis induced by T cells. CD123 expression levels on the CD123 positive MOLM-13 and KG-1a AML tumor cells lines were compared to the CD123 negative Daudi Burkitt's lymphoma tumor cell line by flow cytometry. CD123 expression was detected using a commercially available human CD123 phycoerythrin (PE) conjugated antibody. Positive CD123 expression was detected on MOLM-13 and KG-1a but not on Daudi cells. MOLM-13 and KG-1a cells expressed 10,000 and 2,000 receptors on average per cell respectively (data not shown). Different concentrations of TRI130 were incubated with MOLM-13, KG-1a or Daudi cell lines and purified human T-cells freshly isolated from human peripheral blood then pre-activated with anti-CD3×anti-CD28 coupled beads for 24 hours. TRI130 induced cytotoxicity only in the cell lines which were positive for CD123 expression and demonstrated comparable potency between the MOLM-13 and KG-1a AML tumor cell lines (FIG. 12). Cytotoxicity was measured as percent of specific tumor cell lysis, estimated using a chromium-51 release assay with 4 hr and 24 hr time points (data not shown). These results confirm that TRI130 induced redirected T-cell cytotoxicity is specific to CD123 expressing target cells and demonstrate potent TRI130 cytotoxicity on AML tumor cell lines expressing different levels of the CD123 protein.

### Example 13: TRI130 Induced Cytotoxicity with Naïve T-Cells at Different Effector to Target Cell Ratios

To examine TRI130 induced cytotoxicity activity at different effector to target cell ratios, KG-1a cells which express moderate levels of CD123 were incubated with different concentrations of TRI130 and purified T-cells from five different human donors that were not pre-activated. KG-1a target cells were cultured with T-cells at effector to target cell ratios of 10:1, 5:1 and 1:1. TRI130 induced cytotoxic activity at all effector to target cell ratios tested was comparable across all five donors (data not shown). Cytotoxicity was measured as percent of specific tumor cell lysis, which was estimated using a 24-hour chromium-51 release assay. These results demonstrate potent TRI130 induced cytotoxicity at different effector to target cell ratios with naïve T-cells and minimal variability in activity between normal donors.

### Example 14: Single-Dose Pharmacokinetic and Tolerability Study in Cynomolgus Monkeys

A single-dose pharmacokinetic and tolerability study was conducted to determine the tolerability, immunogenicity and pharmacokinetic characteristics following intravenous administration of TRI130. The study design is shown in Table 11. The intravenous route of administration was selected for the study as this is the intended route for human dosing.

Binding to NHP CD3ε was determined using the anti-CD3 binding domain in a mono-specific scFv-Fc fusion format. The anti-CD3 scFv-Fc was tested on Chinese cynomolgus macaque T-cells by flow cytometry. Binding levels were variable, with individual monkeys showing high, intermediate or low levels of binding compared to human cells (data not shown).

TABLE 11
Experimental Design of Pharmacokinetic Study
in Cynomolgus Monkeys

| | | | | | Dose | |
|---|---|---|---|---|---|---|
| | | | Dosing | Dose | Con- | Dose |
| Group | No. of | Test | Day | Level | centration | Volume |
| No. | Males^{a} | Material | and Time^{b} | (mg/kg) | (mg/mL) | (mL/kg) |
| 1 | 3 | Control | Day 1 | 0 | 0 | 5 |
| 2 | 3 | TRI130 | Day 1 | 0.25 | 0.05 | 5 |
| 3 | 3 | TRI130 | Day 1: 2 hr | 0.5 | 0.1 | 5 |
| 4 | 3 | TRI130 | Day 1: 24 hr | 1.0 | 0.2 | 5 |

^{a}Animals were released from study on Day 36.
^{b}Dosing for Groups 1 and 2 was started consecutively, without a delay between groups. Dosing was delayed by 2 hour from the end of the third animal in Group 2 and the start of the first animal in Group 3; Dosing for Group 4 was delayed by 24 hours from the start of dosing for Group 1.

The following parameters and end points were evaluated in the single dose NHP study: clinical signs, body weights, food consumption, clinical pathology parameters (hematology, coagulation, and clinical chemistry), pharmacokinetics (PK), immunogenicity, cytokine profiles, and flow cytometry.

Dose levels tested ranged from the expected maximum human dose to high multiples of the expected human dose (Table 12). The anticipated pharmacologically active dose (PAD) in humans is in the range of 0.4 mcg/kg based on the in vivo xenograft model (Example 11), which is 625× below the dose of Group 2. However, based on in vitro activity assays (T-cell activation, proliferation and cytokine release), there is a 10 to 100-fold reduction in the response of cynomolgus T-cells to TRI130 compared to that of human T-cells. Therefore, the dose in Group 2 is expected to be −6.25 to 62.5× the biologic equivalent to the human PAD. Groups 3 and 4 evaluate a 2 and 4-fold excess of the latter dose.

TRI130 interacts with CD3 on T-cells, and with the target molecule CD123 on immune cell populations including plasmacytoid dendritic cells (pDC) and basophils, which express high levels of CD123, but make up less than 1% of circulating leukocytes. Published data describing other bispecifics targeting CD123 noted significant cytokine release (Chichili et al. Sci Transl Med. 2015 May 27; 7(289):289ra82).

Due to the expectation for cytokine release, this study was conducted in 2 phases. In Phase 1, two sentinel animals received TRI130 to determine appropriate dose levels for Phase II, which would include treatment Groups 2-4. A dose level of 0.05 mg/kg (initially anticipated as the Phase II mid-dose level) was chosen for the first sentinel, and this animal was closely monitored for signs of cytokine release syndrome, including collection of post-dose observations, body temperatures, and blood samples for cytokine analysis. The dose was well tolerated, and 24 hours later, a second sentinel animal was dosed at 0.25 mg/kg (initially anticipated as the Phase II high dose level) which was also well tolerated.

Dose levels for Phase II were selected in an attempt to produce graded responses to TRI130, and incorporated results of the Phase I sentinels. Because dose levels in Phase I were well tolerated by sentinel animals, with no abnormal clinical observations, elevated cytokine release levels, or increased body temperature, the Phase II dose levels selected were 0.25, 0.5 and 1 mg/kg, which are 2500 times the expected human PAD equivalent (Table 12). Adjusting this margin by up to 100-fold difference in activity, this study achieved up to 25-fold higher doses than the human PAD.

TABLE 12
Dose Equivalents of TRI130 Compared to Expected Pharmacologically Active Dose (PAD)

| | | | Mass Equivalent to | Adjusted Dose |
|---|---|---|---|---|
| | | Dose Level | Expected PAD | Equivalence Based |
| Group No. | Test Material | (mg/kg) | (~0.4 mcg/kg) | on Cyno Response |
| 1 | Control | 0 | 0 | 0 |
| 2 | TRI130 | 0.25 | 625X | 6.25X |
| 3 | TRI130 | 0.5 | 1250X | 12.5X |
| 4 | TRI130 | 1 | 2500X | 25X |

### Pharmacokinetics of Single Dose TRI130 in Cynomolgus Monkeys

Serum concentrations were determined using a standard ELISA method using the extracellular domain of CD123 to capture TRI130, and a biotinylated anti-ID antibody (5H5) recognizing the anti-CD3 binding domain to detect bound TRI130. For most animals a rapid drop in serum concentration at late time points corresponded to the presence of anti-drug antibodies (ADA). A standard bridging ELISA using TRI130+/−biotin was used to measure ADA titers in serum samples collected at late time points. ADA titers were relatively low for 7 of the 9 animals treated with TR130, and titer values tended to increase over time (data not shown). However, titer also appeared to decrease with increasing doses of TRI130, and the onset of ADA was later for higher doses, meaning that higher doses were not correlated to increased levels of immunogenicity, as would normally be expected. Because TRI130 is a human protein, detecting ADA at late time points in NHP serum samples is considered a normal response, and immunogenicity in NHP is not predictive of human responses.

Non-compartmental analysis (NCA) of serum concentrations detected in the ELISA assay calculated a mean terminal half-life for TR8134 of up to 84 hours in animals dosed with 1 mg/kg (Table 13). Individually, one animal in group 4 had apparent subcutaneous accumulation of TRI130, while another in the group had significant early induction of ADA resulting in a much shorter half-life. Sparse sampling and uniform weighting were used for NCA, and serum concentrations clearly influenced by ADA were excluded from the analysis.

TABLE 13
NCA PK Parameter Estimates for Individual Animals Dosed with TRI130

| NCA with Sparse sampling | | Uniform Weighting | | |
|---|---|---|---|---|
| Parameter | Units | Group 2 | Group 3 | Group 4 |
| Rsq adjusted | | 0.964 | 0.992 | 0.983 |
| HL Lambda z | hr | 59.55 | 66.44 | 84.46 |
| Tmax | hr | 0.25 | 0.25 | 0.25 |
| Cmax | ug/ml | 4.386 | 12.124 | 27.075 |
| Cmax/D | kg * ug/ml/ug | 0.018 | 0.024 | 0.027 |
| Tlast | hr | 264 | 264 | 408 |
| Clast | ug/ml | 0.0573 | 0.317 | 0.309 |
| AUCall | hr * ug/ml | 143.7 | 515.8 | 1329.7 |
| AUCINF | hr * ug/ml | 148.6 | 546.2 | 1367.4 |
| AUCINF/D | hr * kg * ug/ml/ug | 0.594 | 1.092 | 1.367 |
| Vz | ml/kg | 144.57 | 87.75 | 89.11 |
| Cl | ml/hr/kg | 1.683 | 0.915 | 0.731 |
| MRTlast | hr | 49.45 | 64.30 | 88.42 |
| MRTINF | hr | 59.41 | 80.75 | 100.58 |
| Vss | ml/kg | 99.96 | 73.92 | 73.56 |

In addition to NCA, compartmental analysis was run for individual animals and the treatment groups, using precompiled WinNonlin® 2 compartment models, with the appropriate dosing and weighting schemes. Results are shown in FIG. 13 and were similar to NCA parameter estimates, with half-life for the one animal in group 4 (4003) without significant ADA or partial subcutaneous dosing, determined to be almost 89 hours. Animal 4002 in group 4 had a gradual increase in serum drug levels over time, and a Tmax at 6 hours (Table 14); therefore, its data was best fit using a 2-compartment model for extravascular dosing, instead of IV bolus dosing.

PK parameter estimates determined using NCA or compartmental analysis, were not similar across treatment groups in that half-life increased and clearance decreased with increasing doses, likely due to target mediated binding of TRI130.

TABLE 14
Compartmental Analysis for NHP Given a Single IV Dose of TRI130

| | | | CL | | | AUC | Alpha | Beta | | |
|---|---|---|---|---|---|---|---|---|---|---|
| Treatment | NHP | V1 | (mL/ | V2 | CLD2 | (hr^{*}μg/ | HL | HL | Cmax | Tmax |
| (Dose/Route) | ID | (mL/hr) | hr/kg) | (mL/hr) | (mL/hr/kg) | mL) | (hr) | (hr) | (μg/mL) | (hr) |
| 0.25 mg/kg | 2001 | 59.32 | 2.463 | 24.36 | 4.427 | 101.5 | 2.568 | 24.79 | 4.22 | .25 |
| IV | | | | | | | | | | |
| 0.25 mg/kg | 2002 | 50.87 | 1.864 | 32.19 | 1.820 | 134.1 | 6.293 | 36.85 | 4.91 | .25 |
| IV | | | | | | | | | | |
| 0.25 mg/kg | 2003 | 61.16 | 1.470 | 43.41 | 3.341 | 170.1 | 4.859 | 53.46 | 4.09 | .25 |
| IV | | | | | | | | | | |
| Group 2 | | 56.54 | 1.917 | 32.7 | 3.028 | 130.4 | 4.32 | 35.45 | 4.42 | .25 |
| mean | | | | | | | | | | |
| 0.5 mg/kg IV | 3001 | 41.664 | 0.874 | 25.193 | 1.293 | 572 | 7.569 | 58.99 | 12.0 | .25 |
| 0.5 mg/kg IV | 3002 | 45.703 | 1.072 | 41.354 | 3.339 | 467 | 4.180 | 60.72 | 10.94 | .25 |
| 0.5 mg/kg IV | 3003 | 38.939 | 1.058 | 28.973 | 2.692 | 473 | 3.966 | 47.98 | 12.84 | .25 |
| Group 3 | | 41.87 | 1.002 | 32.43 | 2.540 | 499 | 4.608 | 55.65 | 11.94 | .25 |
| mean | | | | | | | | | | |
| 1 mg/kg IV | 4101 | 27.93 | 0.926 | 22.08 | 16.533 | 1080 | 0.511 | 37.85 | 35.81 | .25 |
| 1 mg/kg IV | *4002 | 76.83 | 0.854 | 16.024 | 0.133 | 1172 | 46.2 | 112.6 | 11.27 | 6 |
| 1 mg/kg IV | 4003 | 48.22 | 0.706 | 25.34 | 0.492 | 1417 | 18.99 | 88.92 | 20.74 | .25 |
| | | | | | | | 6 | | | |
| Group 4 | | 39.31 | 0.826 | 45.2 | 5.22 | 74.22 | 4.15 | 5.6 | 74.22 | 2.17 |
| mean | | | | | | | | | | |

*Received at least part of the TRI130 dose SC, and parameter estimates were analyzed using a SC model Group means were analyzed using the WinNonlin ^{®} precompiled model #7 with sparse sampling and appropriate weighting
V1: Volume for the 1^{st} (central) compartment
CL: Clearance from the central or 1^{st} compartment
V2: Volume for the 2^{nd} compartment
CLD2: Clearance from the 2^{nd} compartment
AUC: Area under the concentration time curve
Alpha HL: Half-life associated with the macro constant Alpha
Beta HL: Half-life associated with the macro constant Beta
Cmax: Maximum observed concentration

### Tolerability of Single Dose TRI130 in Cynomolgus Monkeys

A single dose administration of TRI130 to cynomolgus monkeys was clinically well-tolerated at dose levels up to and including 1 mg/kg. At these doses, there were no test-article related clinical observations or changes to body weights, food consumption, clinical chemistry or coagulation parameters.

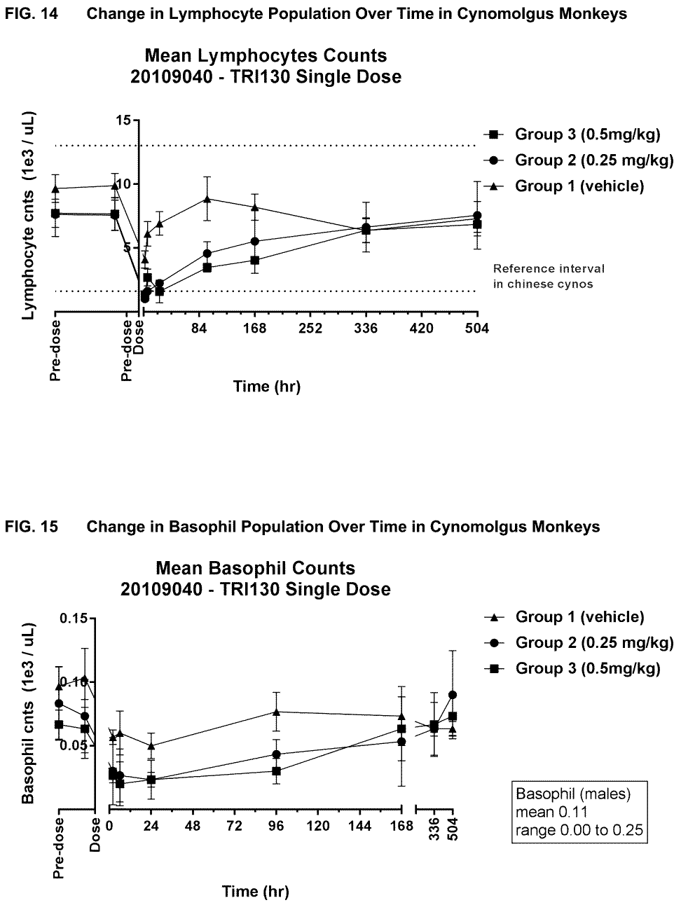

Administration of TRI130 was associated with decreased lymphocytes in peripheral blood (FIG. 14). TRI130-related, transient decreases in lymphocytes were observed at the 2-hour post-dose time point at all doses. In addition, there appeared to be a reduction in basophil counts in Groups 2 and 3 by hematology measurements (FIG. 15), with a slightly longer time to return to baseline levels, compared to the vehicle control (Group 1). The mean reduction in lymphocytes and basophils was less in Group 4, in part due to one animal that had a limited response and one animal that had apparent subcutaneous rather than intravenous injection, therefore Group 4 was excluded from the mean lymphocyte and basophil data shown in FIGS. 14 and 15. All groups dosed with TRI130 had an increase in neutrophils compared to the vehicle control group (data not shown). The changes noted with the hematology panel were mostly back to baseline levels by about 1 week after dosing.

Administration of TRI130 was not associated with changes in clinical chemistry, including CRP, or with changes in coagulation parameters.

The serum cytokine measurements indicated a minimal spike in some cytokines, including the T cell cytokines IL-10 and IL-2 and secondary cytokines such as MCP-1. The peak levels of cytokine detected were at 2 or 6 hours post-dose. In total, a bead based multi-plex cytokine kit was used to determine the levels of 23 individual cytokines, including T-specific cytokines (such as IL-2, IL-4, IL-5, IL-6, IL-10, IL-17, IFNγ, TNFα), and cytokines that are made by other cell types (such as MCP-1). Examples of cytokines detected in the study are presented in Table 15. Notably, the levels detected in the 1 mg/kg dose were lower than the mid and low dose group, however animal 4003, with more normal IV bolus pharmacokinetic parameters had responses more similar to the other groups. The other animal in Group 4 with an apparent subcutaneous dose (4002) had delayed peak response for some cytokines, consistent with a later Tmax. In general, cytokine levels returned to baseline levels by 96 hours post dose. The minimal cytokine secretion did not translate into any observable clinical post-dose event or changes in body weight or food intake (as noted above). The cytokine secretion is consistent with the mechanism of action of TRI130, which, when the target protein (CD123) is present, would result in some activation of T-cells in the system.

Administration of TRI130 by single intravenous (slow bolus) injection was well-tolerated in cynomolgus monkeys at levels of 0.25, 0.5 and 1 mg/kg. Based on these results, the no-observed-adverse-effect level (NOAEL) was considered to be 1 mg/kg. The serum half-life estimate for TRI130 was 84 hours, with normal clearance and volume of distribution in the high dose group. Lower doses had faster clearance and shorter half-life estimates, suggesting target mediated binding of TRI130 to target cells in cynomolgus monkeys. These observations will be confirmed and extended to higher dose levels in the 28-day repeat-dose GLP toxicology study in cynomolgus monkeys.

TABLE 15
Serum Cytokine Measurements of Dose Groups in Pilot Tolerability and PK
IL-2 (pg/mL)
Animal Number

| Time | | | | | | | | | | | | |
|---|---|---|---|---|---|---|---|---|---|---|---|---|
| (hr) | 1001 | 1002 | 1003 | 2001 | 2002 | 2003 | 3001 | 3002 | 3003 | 4002** | 4003 | 4101 |
| −2 | 42.99 | 52.08 | 70.02 | 76.99 | 60.12 | 81.64 | 76.9 | 41.08 | 97.16 | 78.73 | 106.78 | 107.28 |
| 2* | 55.76 | 51.55 | 53.32 | 246.18 | 159.64 | 221.29 | 166.71 | 86.29 | 192.19 | 109.13 | 223.75 | 137.68 |
| 6 | 82.8 | 60.68 | 58.67 | 190.48 | 117.8 | 178.14 | 115.89 | 74.43 | 136.17 | 107.15 | 157.95 | 164.69 |
| 24 | 69.55 | 55.59 | 69.55 | 143.35 | 78.45 | 119.31 | 88.21 | 56.5 | 139.8 | 137.23 | 103.91 | 120.8 |
| 48 | 50.47 | 58.67 | 62.33 | 91.49 | 54.72 | 99.24 | 74.84 | 41.08 | 102.32 | 89.56 | 88.89 | 120.57 |
| 96 | 40.83 | 60.84 | 63.64 | 69.51 | 62.99 | 75.07 | 74.84 | 36.36 | 101.84 | 83.33 | 79.99 | 136.56 |

IL-10 (pg/mL)

| Time | | | | | | | | | | | | |
|---|---|---|---|---|---|---|---|---|---|---|---|---|
| (hr) | 1001 | 1002 | 1003 | 2001 | 2002 | 2003 | 3001 | 3002 | 3003 | 4002 | 4003 | 4101 |
| −2 | 40.27 | 35.8 | 78.98 | 39.22 | 16.6 | 71.08 | 15.15 | | 28.55 | 24.47 | 62.01 | 78.63 |
| 2 | 46.96 | 31.32 | 44.73 | 630.12 | 174.18 | 576.67 | 115.32 | 26.19 | 216.4 | 55.71 | 410.84 | 90.55 |
| 6 | 74.59 | 43.62 | 43.62 | 372.62 | 84.78 | 179.93 | 74.77 | 14.35 | 97.4 | 66.75 | 100.11 | 157.86 |
| 24 | 71.28 | 43.62 | 59.15 | 150.06 | 31.29 | 100.78 | 48.18 | | 123.88 | 69.12 | 63.59 | 60.43 |
| 48 | 38.04 | 34.68 | 56.94 | 49.44 | 29.02 | 71.08 | 21.47 | | 41.12 | 27.57 | 83.39 | 64.38 |
| 96 | 35.8 | 48.07 | 55.83 | 44.9 | 26.76 | 60.82 | 30.13 | | 48.96 | 41.58 | 37.67 | 85.77 |

IL-1ra (pg/mL)

| Time | | | | | | | | | | | | |
|---|---|---|---|---|---|---|---|---|---|---|---|---|
| (hr) | 1001 | 1002 | 1003 | 2001 | 2002 | 2003 | 3001 | 3002 | 3003 | 4002 | 4003 | 4101 |
| −2 | 29.81 | 10.23 | 21.72 | 14.83 | 7.73 | 27.36 | 17.29 | 7.12 | 21.98 | 10.52 | 24.41 | 26.4 |
| 2 | 39.08 | 14.46 | 20.93 | 91.35 | 31.08 | 73.18 | 38.46 | 21.7 | 69.77 | 22.41 | 51.87 | 36.56 |
| 6 | 48.42 | 22.51 | 16.11 | 253.47 | 21.34 | 102.43 | 36.06 | 23.07 | 135.59 | 18.67 | 48.27 | 59.32 |
| 24 | 33.55 | 10.66 | 18.14 | 101.05 | 13.27 | 26.61 | 59.08 | 7.7 | 96.11 | 20.97 | 21.69 | 18.1 |
| 48 | 15.29 | 10.66 | 20.14 | 15.99 | 21.72 | 21.34 | 8.56 | 2.69 | 21.7 | 11.99 | 21.83 | 20.11 |
| 96 | 11.09 | 15.29 | 13.21 | 13.27 | 6.1 | 15.21 | 11.13 | 5.08 | 14.51 | 9.63 | 12.14 | 26.11 |

IL-6 (pg/mL)

| Time | | | | | | | | | | | | |
|---|---|---|---|---|---|---|---|---|---|---|---|---|
| (hr) | 1001 | 1002 | 1003 | 2001 | 2002 | 2003 | 3001 | 3002 | 3003 | 4002 | 4003 | 4101 |
| −2 | | | | | | | | | | | 1.61 | |
| 2 | 4.46 | 8.2 | 28.17 | 40.23 | 11.18 | 46.47 | 7.41 | | 59.97 | 4.71 | 29.12 | 9.24 |

IL-2 (pg/mL) Animal Number

| | | | | | | | | | |
|---|---|---|---|---|---|---|---|---|---|
| 6 | 7.29 | 11.3 | 9.75 | 34.34 | 15.76 | 6.65 | 3.31 | 13.75 | 58.68 |
| 24 | 26.34 | | 0.03 | 5.95 | 135.1 | | 0.42 | | 0.88 |
| 48 | | | | | | | | | |
| 96 | | | | | | | | | 0.3 |

MCP-1 (pg/mL)

| Time | | | | | | | | | | | | |
|---|---|---|---|---|---|---|---|---|---|---|---|---|
| (hr) | 1001 | 1002 | 1003 | 2001 | 2002 | 2003 | 3001 | 3002 | 3003 | 4002 | 4003 | 4101 |
| −2 | 255.62 | 294.77 | 362.89 | 390.54 | 391.05 | 445.7 | 342.49 | 158.31 | 547.6 | 618.1 | 769.48 | 807.72 |
| 2 | 386.37 | 322.52 | 281.46 | 5860.97 | 1690.25 | 3737.21 | 1429.67 | 520.19 | 3744.09 | 918.3 | 5948.11 | 1144.13 |
| 6 | 563.16 | 393.99 | 316.14 | 2747.2 | 936.33 | 1720.26 | 669.27 | 491.71 | 1119.4 | 1063.89 | 1565.33 | 1972.29 |
| 24 | 484.46 | 346.71 | 378.37 | 1188.25 | 571.67 | 874.9 | 508.46 | 288.4 | 1126.98 | 1287.69 | 781.38 | 1043.59 |
| 48 | 285.74 | 398.27 | 379.72 | 518.2 | 435.71 | 634.29 | 358.73 | 211.71 | 672.02 | 789.58 | 706.93 | 1084.58 |
| 96 | 254.56 | 400.97 | 366.57 | 404.11 | 416.68 | 433.94 | 380.1 | 193.43 | 599.64 | 666.4 | 526.84 | 1167.58 |

**animal 4002 had a serum concentration of TRIBO Tmax at 6 hrs that suggested a subcutaneous route of administration, and indicated a slower accumulation of TRI130 in the animal. This animal's peak cytokine levels were somewhat delayed compared to the other animals.

### Example 15: Therapeutic Efficacy of TRI130 in a Disseminated Xenograft Mouse Model of Acute Myeloid Leukemia (AML)

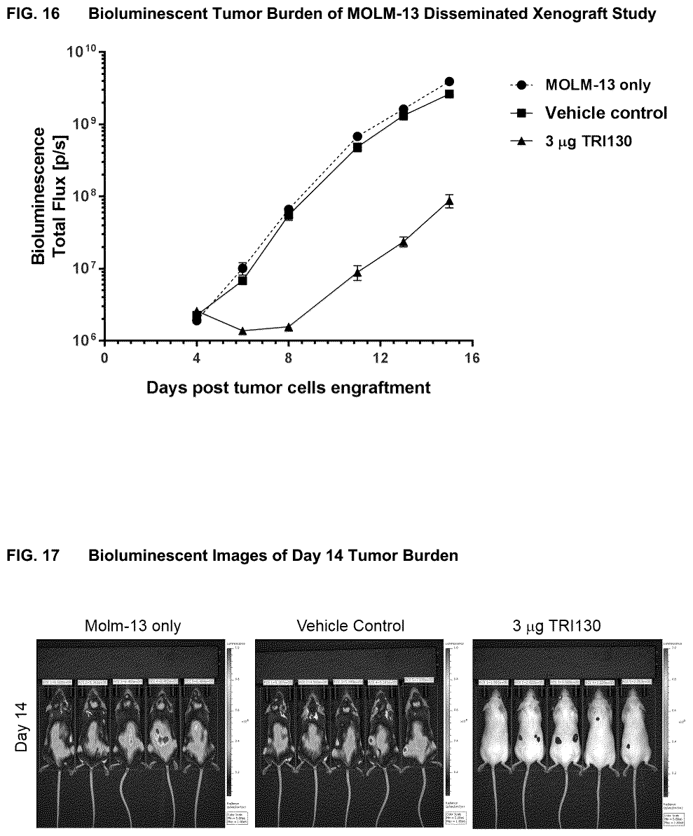

To examine the therapeutic efficacy of TRI130 in mice with established disseminated tumors, a rodent model of human acute myeloid leukemia was utilized. MOLM-13 cells modified to express firefly luciferase were used to allow tumor quantification by bioluminescent imaging. For the study, 100,000 MOLM-13 Luc cells were injected intravenously into the lateral tail vein of 24 NSG male mice. MOLM-13 Luc cells were allowed to establish in the mice for 4 days prior to initiation of treatments. Mice were assigned into 3 groups of 8 mice each. One group received no additional treatments as a control. The remaining two groups received 7 million human T-cells with the first treatments. Treatments consisted of PBS vehicle control or TRI130 at 3 μg on days 4, 8 and 12. Treatments were administered intravenously via the tail vein. Treatments were delivered in 0.2 mL of Dulbecco's Phosphate Buffered Saline (PBS) containing no molecule (vehicle control) or 3 μg of TRI130. The injection included 7 million purified human T-cells for the day 4 treatment only. TRI130 treatment resulted in a rapid significant reduction in skeletal tumor burden (FIG. 16, FIG. 17 and Table 16). Residual non-skeletal tumor burden expanded following TRI130 treatments. Upon necropsy and the end of the study, it was determined this residual tumor burden was associated with the male reproductive tract and was observed in both the vehicle and the TRI130 group with the main difference between the being the absence of skeletal tumor burden. Treatment of established disseminated MOLM-13 Luc tumors in male mice with TRI130 resulted in a significant reduction in tumor burden, p<0.0001, relative to T-cell only controls (Table 16). There was no significant difference between the MOLM-13 Luc only and vehicle control groups. Tumor burden distribution was altered by TRI130 treatment with skeletal sites cleared of bioluminescent signal.

TABLE 16
Statistical Comparison of Mean Tumor Volume through Day 14
JMP Repeated-Measures ANOVA Analysis with Tukey's HSD Method

| Treatment | p-Value |
|---|---|
| Vehicle vs. 3 μg TRI130 | <.0001* |
| Vehicle vs. MOLM-13 only | 0.5385 |
| MOLM-13 only vs. 3 μg TRI130 | <.0001* |

*indicates p-value <0.05 significant difference

### Example 16: Construction of an Anti-CD123×Anti-CD3 MGD006-Like Molecule

In order to compare TRI130 activity to another bispecific format an anti-CD123×anti-CD3 bispecific was generated containing the CD123 and CD3 binding domain sequences of MGD006 obtained from WO 2015/026892 (nucleic acid sequences corresponded to SEQ ID NOs: 2 and 4; amino acid sequences corresponded to SEQ ID Nos: 1 and 3) engineered in the format reported in Chichili et al. Sci Transl Med. 2015 May 27; 7(289):289ra82 with an added Avidin-Flag-HIS sequence to enable purification. The resulting MGD006-like construct is referred to as TRI168.

### Example 17: Human T-Cell Activation, Cytotoxicity and Cytokine Release in Response to TRI130 and TRI168

To compare the activity of TRI130 and the TRI168 proteins in vitro, T-cells were isolated from normal donor peripheral blood mononuclear cells (PBMC) and incubated with various concentrations of TRI130 and TRI168 in the presence of CD123+ tumor cells (MOLM-13).

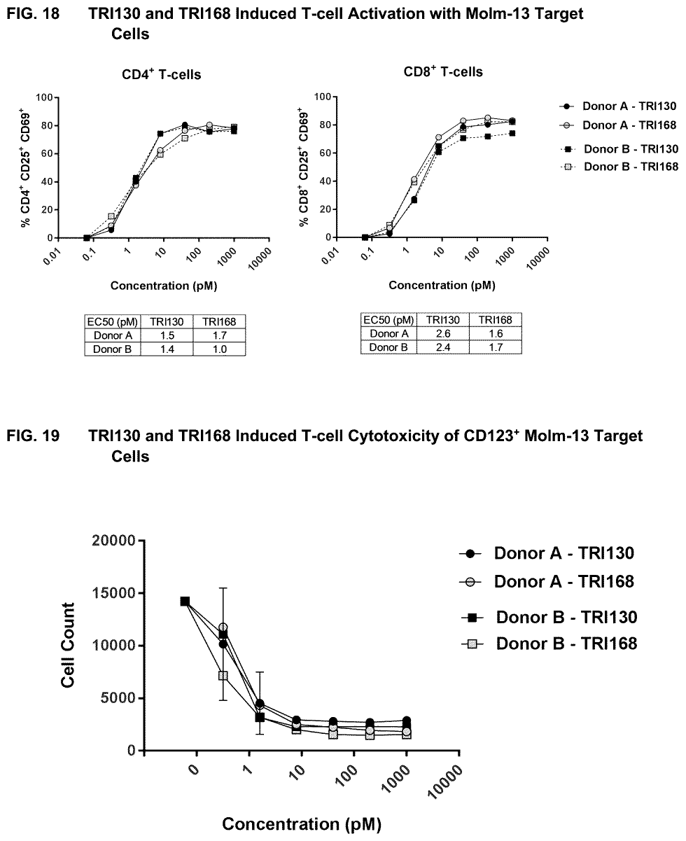

T-cell activation was assessed after 24 hours of culture by flow cytometry. After centrifugation and removal of supernatant, the cell pellets were resuspended with a mixture of the following dye-labeled antibodies in 50 μl volumes: CD69-FITC, CD5-PE, CD8-Pacific Blue, CD4-ΔPC, CD25-PE-Cy7, and 7AAD, and incubated for 30 min on ice. Cells were washed twice and resuspended in 120 μl volumes immediately prior to acquisition of 50% of each well in a BD LSRII flow cytometer. The sample files were analyzed using FlowJo software to calculate the percentages of CD4+(CD8−) or CD8+ T-cells that had upregulated CD69 and CD25, by gating sequentially on forward vs side scatter, 7AAD−, CD5+, CD4+ or CD8+ T-cells (7AAD−, CD5+CD4+ or 7AAD− CD5+CD8+, respectively). Graphs were plotted using GraphPad Prism 7.0. TRI130 and TRI168 induced similar, dose-dependent T-cell activation in the presence of CD123+ target cells with both donors tested (FIG. 18). The figure shows the percentage of CD4+ and CD8+ T-cells that were CD69 and CD25 positive. EC50 values were determined with TRI130 and TRI168 for both CD4+ and CD8+CD25+CD69+ populations demonstrating similar potency for both molecules. No T-cell activation was observed in the absence of CD123+ target cells (Not Shown).

Molm-13 cytotoxicity was assessed after 24 hours of culture by flow cytometry as described above. The sample files were analyzed using FlowJo software to quantitate cytotoxicity by gating sequentially on forward vs side scatter, 7AAD−, CD5− cells. Graphs were plotted using GraphPad Prism 7.0. TRI130 and TRI168 induced similar, dose-dependent Molm-13 cytoxicity with both donors tested (FIG. 19). The figure shows the total number of viable Molm-13 cells after 24 hours of culture with purified T-cells, TRI130 and TRI168.

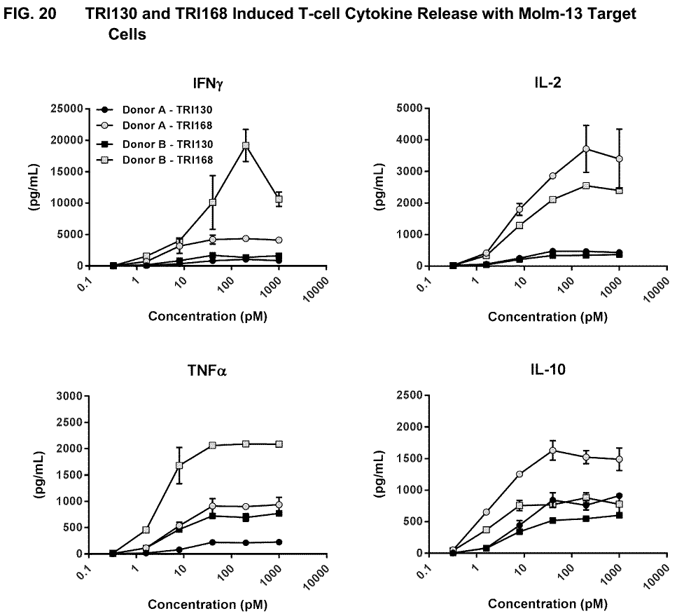

Levels of a selected subset of cytokines commonly produced by activated T-cells including IFNγ, IL-2, TNFα and IL-10 were measured in the 24-hour culture supernatants using multiplexed analyte assays. TRI168 induced higher levels of cytokine secretion compared to TRI130 with both donors tested in the presence of CD123+ Molm-13 target cells (FIG. 20).

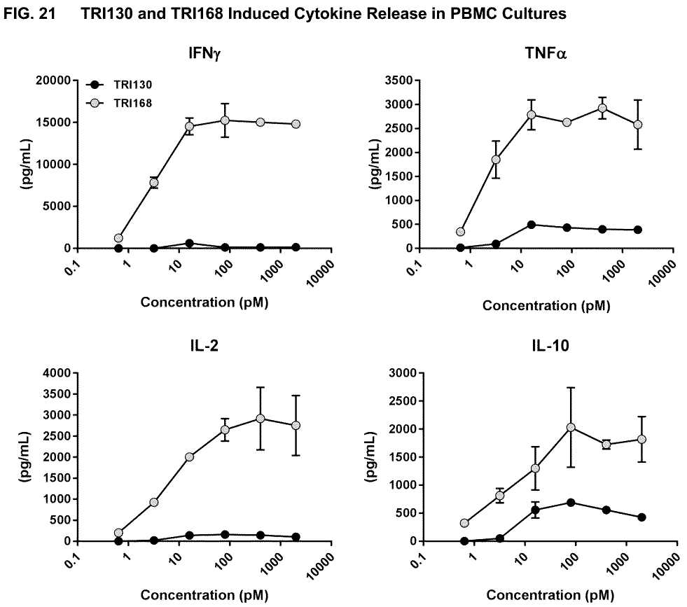

Rare normal immune cell populations including plasmacytoid dendritic cells and basophils expressing high levels of CD123 are present in PBMC samples and represent targets for anti-CD123×anti-CD3 bispecific molecules. To examine the levels of cytokine secretion induced by TRI130 and TRI168 in the absence of exogenously added target cells, normal donor PBMC were cultured for 24 hours with various concentrations of both proteins and cytokines measured in the resulting supernatants using multiplexed analyte assays. Similar to cultures with purified T-cells and MOLM-13 cells, TRI168 induced higher levels of IFNγ, IL-2, TNFα and IL-10 compared to TRI130 (FIG. 21).

Taken together these results demonstrate comparable in vitro T-cell activation and cytotoxicity of exogenously added CD123+ tumor cells with TRI130 and the MGD006-like anti-CD123×anti-CD3 bispecific TRI168. Notably, TRI168 induces much higher levels of secreted T-cell cytokines in cultures of both purified T-cells with Molm-13 tumor cells and normal donor PBMC samples compared to TRI130. In the clinic, drugs that induce strong T-cell activation can cause a series of adverse events, termed “cytokine release syndrome” (CRS), due to excessive cytokine release. Released cytokines cause a systemic inflammatory response that can lead to life-threatening complications which can limit drug administration.

### Example 18: TRI130 Induced Cytotoxicity of AML Cell Samples

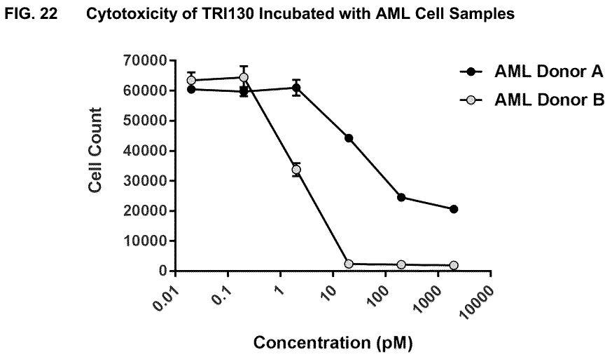

To determine if TRI130 is capable of inducing cytotoxicity in primary AML cells, PBMC samples from AML subjects were cultured for several days with various concentrations of TRI130. Cytotoxicity of AML cells was assessed by flow cytometry after four days of culture. After centrifugation and removal of supernatant, the cell pellets were resuspended with a mixture including the following dye-labeled antibodies in 50 μl volumes: CD69-FITC, CD5-ΔPC, CD19-Pacific Blue, CD25-PE-Cy7, and 7AAD, and incubated for 30 min on ice. Cells were washed twice and resuspended in 120 μl volumes immediately prior to acquisition of 50% of each well in a BD LSRII flow cytometer. The sample files were analyzed using FlowJo software to quantitate cytotoxicity in the non-B, non-T cell compartment by gating sequentially on forward vs side scatter, 7AAD−, CD5−, CD19−, CD25− cells. Graphs were plotted using GraphPad Prism 7.0. TRI130 induced a dose dependent loss of non-B, non-T AML cell samples in both donors tested (FIG. 22). The figure shows the total number of viable non-B, non-T-cells after 96 hours of culture. Similar results were obtained at 24 and 48 hours (data not shown). These results demonstrate that TRI130 is capable of inducing cytotoxicity in cultures of primary human AML PBMC samples.

1.-86. (canceled)

87. A method for treating a disorder in a subject, wherein said disorder is characterized by overexpression of CD123, the method comprising administering to the subject a therapeutically effective amount of a pharmaceutical composition comprising a recombinant polypeptide comprising a CD123 binding domain and a CD3 binding domain;
wherein the CD123-binding domain comprises (i) an immunoglobulin light chain variable region comprising LCDR1, LCDR2, and LCDR3, and (ii) an immunoglobulin heavy chain variable region comprising HCDR1, HCDR2, and HCDR3, wherein the LCDR1 comprises the amino acid sequence set forth in SEQ ID NO:6; the LCDR2 comprises the amino acid sequence set forth in SEQ ID NO:8; the LCDR3 comprises the amino acid sequence set forth in SEQ ID NO:10, the HCDR1 comprises the amino acid sequence set forth in SEQ ID NO:12; the HCDR2 comprises the amino acid sequence set forth in SEQ ID NO:14; and the HCDR3 comprises the amino acid sequence set forth in SEQ ID NO: 16; and
wherein the CD3-binding domain comprises: (i) an immunoglobulin light chain variable region comprising LCDR1, LCDR2, and LCDR3, and (ii) an immunoglobulin heavy chain variable region comprising HCDR1, HCDR2, and HCDR3, wherein
(a) the LCDR1, LCDR2 and LCDR3 of the CD3-binding domain comprise the amino acid sequences set forth in SEQ ID NO:162, SEQ ID NO:163, and SEQ ID NO:164, respectively, and the HCDR1, HCDR2, and HCDR3 of the CD3-binding domain comprise the amino acid sequences set forth in SEQ ID NO: 165, SEQ ID NO:166, and SEQ ID NO:167, respectively;
(b) the LCDR1, LCDR2 and LCDR3 of the CD3-binding domain comprise the amino acid sequences set forth in SEQ ID NO:168, SEQ ID NO:169, and SEQ ID NO:170, respectively, and the HCDR1, HCDR2, and HCDR3 of the CD3-binding domain comprise the amino acid sequences set forth in SEQ ID NO: 171, SEQ ID NO:172, and SEQ ID NO:173, respectively;
(c) the LCDR1, LCDR2 and LCDR3 of the CD3-binding domain comprise the amino acid sequences set forth in SEQ ID NO: 307, SEQ ID NO: 308 and SEQ ID NO:309, respectively, and the HCDR1, HCDR2, and HCDR3 of the CD3-binding domain comprise the amino acid sequences set forth in SEQ ID NO: 174, SEQ ID NO: 175 and SEQ ID NO:176, respectively;
(d) the LCDR1, LCDR2 and LCDR3 of the CD3-binding domain comprise the amino acid sequences set forth in SEQ ID NO: 310, SEQ ID NO: 177 and SEQ ID NO: 178, respectively, and the HCDR1, HCDR2, and HCDR3 of the CD3-binding domain comprise the amino acid sequences set forth in SEQ ID NO: 179, SEQ ID NO: 180 and SEQ ID NO: 181, respectively;
(e) the LCDR1, LCDR2 and LCDR3 of the CD3-binding domain comprise the amino acid sequences set forth in SEQ ID NO: 182, SEQ ID NO: 183 and SEQ ID NO: 184, respectively, and the HCDR1, HCDR2, and HCDR3 of the CD3-binding domain comprise the amino acid sequences set forth in SEQ ID NO: 185, SEQ ID NO: 186 and SEQ ID NO: 187, respectively; or
(f) the LCDR1, LCDR2 and LCDR3 of the CD3-binding domain comprise the amino acid sequences set forth in SEQ ID NO: 188, SEQ ID NO: 189 and SEQ ID NO: 190, respectively, and the HCDR1, HCDR2, and HCDR3 of the CD3-binding domain comprise the amino acid sequences set forth in SEQ ID NO: 191, SEQ ID NO: 192 and SEQ ID NO: 193, respectively.

88. The method of claim 87, wherein the CD3-binding domain comprises an immunoglobulin light chain variable region and an immunoglobulin heavy chain variable region;
wherein the immunoglobulin light chain variable region of the CD3-binding domain comprises the amino acid sequence of SEQ ID NO:160; and
wherein the immunoglobulin heavy chain variable region of the CD3-binding domain comprises the amino acid sequence of SEQ ID NO:161.

89. The method of claim 87, wherein the CD123-binding domain comprises:
(i) an immunoglobulin light chain variable region comprising the amino acid sequence of SEQ ID NO:2; and
(ii) an immunoglobulin heavy chain variable region comprising the amino acid sequence of SEQ ID NO:4.

90. The method of claim 87, wherein the CD123-binding domain is a single chain variable fragment (scFv), and the CD3-binding domain is an scFv.

91. The method of claim 87, wherein the recombinant polypeptide comprises in order from amino terminus to carboxyl terminus, (i) the CD123-binding domain, (ii) a hinge region, (iii) an immunoglobulin constant region, (iv) a carboxyl-terminus linker, and (v) the CD3-binding domain.

92. The method of claim 91, wherein the carboxyl-terminus linker comprises an amino acid sequence comprising (Gly4Ser)ₙ, wherein n=1-7 (SEQ ID NO:314).

93. The method of claim 92, wherein n=3-5.

94. The method of claim 91, wherein the carboxyl-terminus linker comprises SEQ ID NO:288.

95. The method of claim 91, wherein the immunoglobulin constant region comprises immunoglobulin CH2 and CH3 domains of IgG1, IgG2, IgG3, IgG4, IgA1, IgA2 or IgD.

96. The method of claim 91, wherein the immunoglobulin constant region comprises a human IgG1 CH2 domain comprising the substitutions L234A, L235A, G237A, and K322A, according to the EU numbering system.

97. The method of claim 87, wherein the pharmaceutical composition comprises a dimeric protein comprising two identical copies of the recombinant polypeptide.

98. The method of claim 87, wherein the disorder is a cancer.

99. The method of claim 98, wherein the cancer is acute myeloid leukemia (AML), B-lymphoid leukemia, blastic plasmocytoid dendritic neoplasm (BPDCN), hairy cell leukemia, acute lymphoblastic leukemia, refractory anemia with excess blasts, myelodysplastic syndrome, chronic myeloid leukemia or Hodgkin's lymphoma.

100. The method of claim 87, wherein the administration of the pharmaceutical composition does not cause excessive cytokine release.

101. The method of claim 100, wherein the cytokine is IFN-γ, TNF-α, IL-6, IL-2, IL-10, IL-17, IL-4, MCP-1, or any combination thereof.

102. The method of claim 87, wherein the pharmaceutical composition is administered by a single intravenous injection.
